SEQUENCE LISTING (527 sequences)

SEQ ID NO: 1 - DNA, 157 bp, Artificial sequence
  Wt sequence miR405a
tcaaaatgggtaacccaacccaacccaactcataatcaaatgagtttatgattaaatgagttatgggttgacccaactcattttgttaaatgagttgggtctaacccataactcatttcatttgatgggttgagttgttaaatgggttaaccattta

SEQ ID NO: 2 - DNA, 24 bp, Artificial sequence
  Mature sequence miR405a
atgagttgggtctaacccataact

SEQ ID NO: 3 - DNA, 24 bp, Artificial sequence
  Target of miR405a analysed
agttatgggttagacccaactcat

SEQ ID NO: 4 - DNA, 157 bp, Artificial sequence
  Wt sequence Dead miR859
tcaaaatgggtaatccaactcaactcaactcataatcaaatgagtttaggattaaatgagttatgggttgacccaactcattttgttaaatgggttcggtcaacccataactcaattaatttgatggattgagttggtaaatgagttaacccattta

SEQ ID NO: 5 - DNA, 24 bp, Artificial sequence
  Mature sequence Dead miR859
atgggttcggtcaacccataactc

SEQ ID NO: 6 - DNA, 24 bp, Artificial sequence
  Target analysed of Dead miR859
gagttatgggttgaccgaacccat

SEQ ID NO: 7 - DNA, 157 bp, Artificial sequence
  Reactivated (RmiR)  Sequence
ccagattggattgcctcacaccacacacgactcaattcactaagacgaggattaaattgggttatgggtgaccgaactcattttgccaaatgggttcggtcaacccataactcaattttggtgaaggtcgtgggtggaaaaggaggcaacccagtca

SEQ ID NO: 8 - DNA, 24 bp, Artificial sequence
  Reactivated (RmiR) Mature sequence
atgggttcggtcaacccataactc

SEQ ID NO: 9 - DNA, 24 bp, Artificial sequence
  Target analysed   Reactivated (RmiR)
gagttatgggttgaccgaacccat

SEQ ID NO: 10 - DNA, 157 bp, Artificial sequence
  Redirected (Anti PDS-PDSmiR)
tcaaattgggtaaactaccccaacatctctcaaaatccaaggttgttaggaccaaatgtggtttgtggacagagttttcattttgctaaatgaaaattttgatttacgaattgcattatcttgggtgagggaggttgcaaattagtttagccagtta

SEQ ID NO: 11 - DNA, 24 bp, Artificial sequence
  Redirected (Anti PDS-PDSmiR) Mature sequence
atgaaaattttgatttacgaattg

SEQ ID NO: 12 - DNA, 24 bp, Artificial sequence
  Redirected (Anti PDS-PDSmiR) Target analysed (PDS3- At4g14210)
caattcgtaaatcaaaattttaat

SEQ ID NO: 13 - DNA, 23 bp, Artificial sequence
  Redirected (Anti PDS-PDSmiR) sgRNA
attaatttgatggattgagttgg

SEQ ID NO: 14 - DNA, 1200 bp, Artificial sequence
  Redirected (Anti PDS-PDSmiR)  DONOR (1.2 kb)
gtcaaaatatgtcaaaattcatgcgtcaaactcaactcaactcaacccatgaaccctaatgagttaaaaatttggactcaaatgggttgatgagtcaaatgagttattgagtcaattggtttgatgagtaaaatgagttgggttgtaatgattaatggtttcaatggtttacccaattaactcatcaagttttgtaaaattgaactaaaccaactaaaatctttaaaccaatgccaatttaagtttaaccaacatatctaaaccaatttaataaaatcaatatttttccaaatttcttaaatatacaagcgataaaattgagaaaaagtaaactcgtaatttttccaccaaaaaacataaacccgtgattttcccgccaaaaccgtaaacccgtgattttcccgcccaaaacgtaaacccttgatttttccgcccaaaacgtaaatatcctaagtttgatgataatgaattaataattattatttattattttttataataataattaattaaattattacttaactggctaaactaatttgcaacctccctcacccaagataatgcaattcgtaaatcaaaattttcatttagcaaaatgaaaactctgtccacaaaccacatttggtcctaacaaccttggattttgagagatgttggggtagtttacccaatttgacacccctaatgacaatatgagtttaaagttcattagttcatatgtatgacaatataagtttatatgaactaacaaaaataaatactttaagatcatagtaataaatacgtgaatatcataataatatagaaaaatcgtatatatatatacatagacctcaaatgcaacaaaaatactaaagaaaaacttttatcaaattacgtgataaataaataattgttcttttatcaaaattactaaaaacaattcattccttcttcttattttttttaataatactataataactaggatacgacacagcaggttaaatattttatttatttttcttttttataaacgaaatttattgtttattgttatttgtgtttattaataattatctataaaactgtgtatatttttattgagtcgtacttatgatattagtaagtctaataggttattttatcttttaggatttgactcgtgctagaccacaccacgtgataatttttacttttagtgtttttagattaatg

SEQ ID NO: 15 - DNA, 113 bp, Artificial sequence
  Wt sequence miR8174
cggcccatccgttgtctttcctggtacgcatgtgccatggctttctcgtaagggactggattgtccgtatttctcatgtgtatagggaagctaatcgtcttgtagatgggttg

SEQ ID NO: 16 - DNA, 21 bp, Artificial sequence
  Mature sequence miR8174
atgtgtatagggaagctaatc

SEQ ID NO: 17 - DNA, 21 bp, Artificial sequence
  Target of miR8174 analysed
gattagcttccctatacacat

SEQ ID NO: 18 - DNA, 113 bp, Artificial sequence
  Wt sequence Dead miR1334
attcgcattctctgtctttcctagtacgtttatgttatggcttcatttcgaaggactagattgtccgaattactcatgtgtatagggaagctaatcgtctcgcagatgaatta

SEQ ID NO: 19 - DNA, 21 bp, Artificial sequence
  Mature sequence  Dead miR1334
atgtgtatagggaagctaatc

SEQ ID NO: 20 - DNA, 21 bp, Artificial sequence
  Target analysed of Dead miR1334
gattagcttccctatacacat

SEQ ID NO: 21 - DNA, 113 bp, Artificial sequence
  Reactivated (RmiR) Sequence
tcacgcattcgttgacttccctagtacgcatattgaactgctgtaaggtgaaggacgttaatgtaccaaaaacttatgtgtatagggaagctaatcgtcccgcagatgtgtga

SEQ ID NO: 22 - DNA, 21 bp, Artificial sequence
  Reactivated (RmiR) Mature sequence
atgtgtatagggaagctaatc

SEQ ID NO: 23 - DNA, 21 bp, Artificial sequence
  Reactivated (RmiR) Target analysed
gattagcttccctatacacat

SEQ ID NO: 24 - DNA, 113 bp, Artificial sequence
  Redirected (Anti PDS-PDSmiR) Sequence
atgtgcatcgcagtgattggtgtgttatatgactaaaagtctttatcgcgaagggctatatcgacctaggtactttatatgaacattaataactggcccccccagatgcatgt

SEQ ID NO: 25 - DNA, 21 bp, Artificial sequence
  Redirected (Anti PDS-PDSmiR) Mature sequence
tatatgaacattaataactgg

SEQ ID NO: 26 - DNA, 21 bp, Artificial sequence
  Redirected (Anti PDS-PDSmiR) Target analysed (PDS3- At4g14210)
ccagttattaatgttcatata

SEQ ID NO: 27 - DNA, 23 bp, Artificial sequence
  Redirected (Anti PDS-PDSmiR) sgRNA
atgttatggcttcatttcgaagg

SEQ ID NO: 28 - DNA, 1200 bp, Artificial sequence
  Redirected (Anti PDS-PDSmiR) DONOR (1.2 kb)
gttatatgtgttctttacacaatcattgcttgaatgggtatacagtaatttgggagaacaagaacttgtcggaggttatccgtgggctactttattcgctttggcagcatggtggggttggaaacggcgctgcagaaatgtgtttggggagaataggaaatgtcgagatagagttcgtttcctaaaggattcagcgaaagaggtggtggaggctcactcgctgcttgggagtaatcgaggtaatgtaactagggtggagagacaaatagcatgagttccgccaggagatggttggctgaagttaaacacggatggcgcatcacgtggaaatccgggtttagcaatagctggtggtgttttacgggataatgagggtatttggtgtggtggttttgcgggaatctcggagtttgttcggctcctttagttaagttatgaggtgtgtattacgggcttttcatagcttgggagaaaaaggctacgcgggtgtagctggaagtggattcagatatggtggtgggttttcttaaaacatggattagcgatgtgcatcgcagtgattggtgtgttatatgactaaaagtctttatcgcgaagggctatatcgacctaggtactttatatgaacattaataactggcccccccagatgcatgtgcaaaccatgcttttttgttacctttggggtttcatagttttccccttaggcctgattttgctacttcgattatttttgaggatgctagtagtgctacgcgcccacggaatgttcgtgtgtaatttttttattttgttttttaataatatgggagactagtctccctcattctaaaaaaaataaaaaattataattatataaaatagatataaaattattaattacataataatacacacaaaaaatgaatatcaagaaaaatctctctctctctaaatcaaaatcaaatgagagaagagaggcgatacgacgaacgattgcatctcttcgattcctacggctgtctctcgctcgccgagagttttcttcgccagtttccggcggttacttcagggatgaataacggtagaacggttgtggaccccataactgcttctcaaccaaacctatttataccctgcgcatgtctctgttctcgttgggttgatcagagtgaaagtacacaaattcctttgttcatattgacaatggcagataatctc

SEQ ID NO: 29 - DNA, 15 bp, Artificial sequence
  Single strand DNA oligonucleotide
aaacgagctcgctag

SEQ ID NO: 30 - DNA, 15 bp, Artificial sequence
  Single strand DNA oligonucleotide
gcaggtcgactctag

SEQ ID NO: 31 - DNA, 48 bp, Artificial sequence
  Single strand DNA oligonucleotide
aaacgagctcgctagtcaaaatgggtaatccaactcaactcaactcat

SEQ ID NO: 32 - DNA, 52 bp, Artificial sequence
  Single strand DNA oligonucleotide
gcaggtcgactctagtaaatgggttaactcatttaccaactcaatccatcaa

SEQ ID NO: 33 - DNA, 43 bp, Artificial sequence
  Single strand DNA oligonucleotide
aaacgagctcgctagattcgcattctctgtctttcctagtacg

SEQ ID NO: 34 - DNA, 44 bp, Artificial sequence
  Single strand DNA oligonucleotide
gcaggtcgactctagtaattcatctgcgagacgattagcttccc

SEQ ID NO: 35 - DNA, 45 bp, Artificial sequence
  Single strand DNA oligonucleotide
aaacgagctcgctagtcaaaatgggtaacccaacccaacccaact

SEQ ID NO: 36 - DNA, 50 bp, Artificial sequence
  Single strand DNA oligonucleotide
gcaggtcgactctagtaaatggttaacccatttaacaactcaacccatca

SEQ ID NO: 37 - DNA, 32 bp, Artificial sequence
  Single strand DNA oligonucleotide
aaacgagctcgctagcggcccatccgttgtct

SEQ ID NO: 38 - DNA, 40 bp, Artificial sequence
  Single strand DNA oligonucleotide
gcaggtcgactctagcaacccatctacaagacgattagct

SEQ ID NO: 39 - DNA, 45 bp, Artificial sequence
  Single strand DNA oligonucleotide
aaacgagctcgctagtcaaattgggtaaactaccccaacatctct

SEQ ID NO: 40 - DNA, 45 bp, Artificial sequence
  Single strand DNA oligonucleotide
gcaggtcgactctagtaactggctaaactaatttgcaacctccct

SEQ ID NO: 41 - DNA, 36 bp, Artificial sequence
  Single strand DNA oligonucleotide
aaacgagctcgctagatgtgcatcgcagtgattggt

SEQ ID NO: 42 - DNA, 32 bp, Artificial sequence
  Single strand DNA oligonucleotide
gcaggtcgactctagacatgcatctggggggg

SEQ ID NO: 43 - DNA, 37 bp, Artificial sequence
  Single strand DNA oligonucleotide
aaacgagctcgctagccagattggattgcctcacacc

SEQ ID NO: 44 - DNA, 37 bp, Artificial sequence
  Single strand DNA oligonucleotide
gcaggtcgactctagtgactgggttgcctccttttcc

SEQ ID NO: 45 - DNA, 40 bp, Artificial sequence
  Single strand DNA oligonucleotide
aaacgagctcgctagtcacgcattcgttgacttccctagt

SEQ ID NO: 46 - DNA, 38 bp, Artificial sequence
  Single strand DNA oligonucleotide
gcaggtcgactctagtcacacatctgcgggacgattag

SEQ ID NO: 47 - DNA, 9835 bp, Turnip mosaic virus
aaaaaatataaaaactcaacataacatacacaaaacgattaaagcaaacacaaatctttcaaagcattcaagcaatcaaagattctcaaatctttcatcgttatcaaagcaatcaccaacagcaaaccaaatggcagcagttacattcgcatcagctatcaccaacgccatcaccagcaaaccagcactcaccggaatggtgcagtttgggagtttcccaccaatgccattgcgatccaccaccgtcaccacagtcgccacttcagtggcgcaacctaaactgtacacagtgcagtttggaagccttgacccagtagtcgtcaagagtggagcagggtcccttgctaaggcaacacgccagcagcctaacgttgaaatagacgttagcctcagtgaagccgcagctctggaggttgcgaaacctagatcgaatgccgtgttgaggatgcacgaggaagcaaacaaggagagagcactctttttggactgggaggctagtttgaagagaagctcgtatggaattgctgaggacgagaaggttgtaatgacaactcatggcgtcagcaagatagtgcccagaagttcaagggcaatgaagctaaagcgcgcaagggagaggcgtagagcgcagcaaccaattatattaaagtgggagcccaaattgagcgggatctcaatcggaggagggctctctgcgagcgtaatcgaagcagaagaggttcgcacaaagtggccgcttcataagacaccgtcaatgaagaagaggacggtgcacagaatatgcaagatgaacgaccaaggagttgacatgttgacacgatccctggttaagattttcaagactaagagtgccaacattgaatacatcggaaagaagtcgattaaggtcgatttcatcagaaaagaacgaacgaaattcgcaagaatccaagtagcacacttactcgggaagagagcacagcgcgacttgttaactggaatggaagaaaaccattttattgacattctcagtaagtactcaggtaacaaaacaaccataaatcctggagtagtttgcgcaggttggagtggcatagtcgttggaaatggaattctaacccagaaacgaagcagaagtccatcagaggcctttgtaattagaggtgagcacgaaggcaagttgtacgatgccaggatcaaagtcacgaggacaatgagtcacaagattgtgcactttagtgcagcaggagccaacttctggaaaggcttcgacagatgctttctcgcataccgtagtgacaatcgcgagcatacatgctattcagggctagatgtcactgagtgcggcgaggtggcagcactgatgtgtttggctatgttcccatgcggaaagataacctgccctgactgtgtaacagatagtgagctatcccaaggacaagcaagcggaccatctatgaagcacaggttgacacagctacgcgatgtcatcaagtcaagctacccacgcttcaagcatgcagtgcagatactagataggtatgagcaatcactgagcagtgcaaacgagaactaccaagatttcgcagaaatccagagcataagcgatggagttgaaaaagctgcattcccacacgtcaacaagctaaacgcaatattgatcaaaggggccacagtaacaggagaggaattctcgcaggctacgaagcacttgctcgagatagcacgatacctgaagaacagaaccgagaacattgagaagggttcactgaagtcctttcgcaacaagatttcccagaaagcgcacatcaacccaacactaatgtgtgacaaccagctcgatagaaatggaaatttcatatggggtgagagaggataccatgcaaaacgattcttcagcaactactttgaaataatcgatccaaagaaaggctacacccaatacgagacaagagcggtaccaaatgggtcacggaaacttgcaatcggcaaactaatagtcccaacgaacttcgaagttttaagggaacagatgaaaggcgaaccggtagaaccatacccagtaacagtcgagtgtgtgagcaagttacagggtgacttcgtccatgcatgttgttgtgtcacaacagaatcaggcgacccagtcttgtctgagatcaaaatgccaaccaaacaccatctagtgattggtaacagcggtgatccaaagtacatagatctccctgagatcgaggagaataaaatgtacatagcgaaagaaggttattgttacatcaatatcttcctagccatgttggtaaatgtcaaggagtcgcaggcaaaggagttcacgaaagttgttagggacaaactagttggcgaacttggcaagtggcccactctgttagatgtagcaaccgcttgttatttcctgaaagtattttacccagacgttgctaacgccgaattgccacgcatgctagtggaccataagacaaagataattcatgtcgttgattcatatgggtcactgtcaactggatatcatgtccttaagacaaacactgtggaacaactcatcaaattcacgagatgtaatttggagtcaagcttgaaacactaccgcgttggaggaacagaatgggaggacactcatggatccagcaacatagataatccacagtggtgcatcaagaggctcataaaaggagtctacaaaccaaagcaactgaaagaagacatgttggcaaaccctttcttaccactatatgctctactgtcaccaggtgtcatcctggcattttacaatagtggctctctagagtacttgatgaaccattacatcagggtggacagcaacgtcgccgttttgttggtcgttttgaaatctctagcgaagaaggtgtcaactagtcagagtgtgttagcccagcttcaaatcattgaacgaagtctaccagaactcatcgaagcaaaggctaatgttaatgggccagatgacgcagccactcgcgcgtgtaacagattcatgggcatgcttctgcatatggcagaaccaaactgggagcttgcggatggtggatacacaattctgagggatcatagcatctccattttggaaaaaagttatctacaaatcttggacgaagcatggaacgagttaagttggtcggagcgctgtgctataagatactactcgtcaaagcaagcaatctttacacagaaagatttgccaatgaaaagcgaagccgatttaggcggcagatacagcgtgtcagtcatgtcatcttacgaacggagtaagcaatgtatgaaaagcgtgcactctagtataggtaatagattacgtagtagtatgtcttggactagtagcaaggtgtcgaatagtgtgtgtaggactattaactatttagtaccagatgtgttcaagtttatgaatgtactcgtttgtatcagcttactaatcaagatgactgccgaggcgaatcacatcgtcaccacgcaaagaaggctcaaactagatgtcgaggagacagagcgcaggaaaatagaatgggagcttgcattccaccatgccattctgacgcagagtgcaggtcaacacccaacgatagacgagttcagagcgtacatcgccgacaaggcaccacatctaagtgagcatatcgagcctgaagaaaaggcggtggttcatcaagcgaagagacaatccgagcaagaactcgagcgtataatagcatttgttgcattggtgctcatgatgttcgatgcagaacgaagcgactgtgtcacaaagattctcaacaagcttaagggactagtcgccactgtggaacctacagtctaccatcagactctcaatgatatagaggatgacttgagtgagaggaacctcttcgtcgattttgagcttagcagcgatggagatatgctccaacagcttccagccgaaaagacatttgcctcatggtggagtcatcaactaagcagaggattcacaatcccacactacaggacagaagggaagttcatgactttcaccagagcaactgccacggaagtcgcgggtaaaatagcacacgagagtgacaaagacatattactaatgggagcagtaggatcaggtaagtcaactggcttgccatatcatctctccagaaaagggaacgtattactccttgagccgactcggccacttgcagaaaacgtacacaagcagttgtcgcaggcaccgttccatcagaacacaactcttaggatgcgcggactaacagcattcgggtcggcaccaatctcagtgatgaccagtggttttgcactcaattactttgcaaacaacagaatgcgaattgaagaatttgactttgtcatatttgatgaatgtcacgttcatgacgccaatgcaatggcgatgagatgtttgctacatgagtgtgactattctggcaaaattatcaaagtttcagccacaccaccaggtcgagaagttgagttctccactcaataccccgtgtcgataagcacagaagacacactatcgtttcaggattttgtgaacgcacagggtagtggaagcaattgtgatgtgatttcaaaaggagacaatatcctcgtgtatgtagcaagctacaatgaggtagacgcgctttcaaaacttctaattgaaagagacttcaaagtcacgaaggttgatggaagaacgatgaaagttggaaacatcgagatcaccacaagtggaacacctagtaagaagcacttcatagttgcaaccaacatcatagagaacggtgttactctagacatcgatgtggttgctgattttggaacgaaggtactcccatatcttgatacagacagcagaatgctgagcacaactaagacaagcatcaattatggggaacgtatccaaaggctaggaagagtcggaaggcacaagccaggtcacgctctgcgaataggtcacacagagaaggggttgagcgaagttccaagttgtattgcaacagaagcagctttaaagtgcttcacttatgggcttccagtgatcaccaacaacgtctcgacaagtattcttggtaatgtaacggtaaagcaggcacgaacaatgtctgtatttgagataacaccgttctacacaagccaagtggtgagatatgatggctccatgcatccacaggtgcacgcactcttaaagagattcaaactcagagactctgagattgttttgaataaattagccatacctcaccgaggagtgaacgcttggctcacagctagtgagtatgcacgacttggcgcgaatgttgaagataggcgtgacgttcgaattccttttatgtgtcgcgacatcccagaaaaacttcatctagacatgtgggatgtgattgttaaattcaaaggtgatgcaggttttggtcggctttcaagcgccagtgcgagcaaggtagcttatactctacagacggacgtcaactccatacagcgaacagtcactatcatagatacactaatcgctgaggagagaaggaagcaggaatacttcaagacggtaacctccaactgtgtctcttcttcgaacttctcactgcagagcataacaaatgcgataaaatctcgtatgatgaaagatcacacgtgcgagaacatatcagtgcttgaaggagcgaagtcacagttactcgagtttagaaacctgaatgctgatcactcatttgctacaaaaaccgatggaatatctcggcatttcatgagtgagtatggagctcttgaggcagttcaccatcaaaacaccagcgacatgagcaaattcctcaagcttaagggcaaatggaataaaacgctaatcacgcgagatgtgctggtactttgtggagttcttggaggtggattgtggatggttattcagcacctgcggtcaaagatgtccgaacccgtaacccatgaagcgaaaggtaagaggcaaaggcagaaactaaaatttcgcaatgcccgagacaacaaaatgggtagagaagtgtacggagatgatgataccatagagcatttcttcggtgatgcctacacaaagaaagggaagagcaagggtaggacacgtggtatcggacacaaaaacaggaagttcatcaacatgtatgggtttgatcctgaagatttctctgcagttcgtttcgtggatccactcacaggagcgacgttggacgacaacccgctcacagacatcacccttgtgcaagagcacttcggcaacataagaatggacttactcggggaggatgagctggactcaaatgaaatacgtgtgaataagactattcaagcctactacatgaacaataaaacaggcaaggctttgaaggtggatctgacaccacacatacctctcaaggtgtgtgatcttcacgcaaccattgctggattcccagagcgagaaaacgagctgaggcagactggaaaggctcagcccatcaacatagacgaagtgccaagagctaacaacgaactcgtcccagtggaccacgagagtaactccatgttcagagggttgcgtgactacaacccaatatcaaacaacatttgtcatctcacaaatgtttcagatggagcatcaaactcgttatatggagtcggtttcggaccactcatattaacgaaccgacacctctttgagcggaataacggtgaactcgtaataaaatcacgacatggtgagttcgtgattaaaaacacaactcagctacacttgctaccgattccagacagagatcttctgctaatccggttaccaaaggacgtcccaccctttccacagaaattgggtttcaggcaacctgagaaaggtgaacgaatttgcatggtggggtccaatttccaaaccaagagcataacgagtatagtctctgagactagtacaataatgccagtggagaacagtcagttttggaaacactggattagcactaaagacggccaatgcggaagtccaatggtgagcacgaaagacgggaaaatactcggattacacagcctagcgaacttccagaactccatcaattactttgctgctttcccagatgattttgccgagaagtatcttcataccattgaagcacacgagtgggtcaagcactggaagtataacactagcgccatcagttggggctctttgaatatacaagcatcgcaaccgtccggcttgttcaaagtaagcaagctaatctcagacctcgacagcacggcagtctacgcacaaacccagcagaatcggtggatgttcgagcagctcaacgggaacctaaaagcgatagcacactgccctagccagcttgtgacaaagcacacagttaaaggaaaatgtcagatgtttgacttgtatctcaagttgcatgatgaagcacgagagtatttccaaccgatgctgggccagtatcaaaagagcaaactcaatcgagaagcatatgcaaaggatcttctgaaatatgcaacgccaatcgaagcaggaaacatcgactgtgatctgtttgaaaagacagttgaaatagtcgtatcagatctgcgaggttatggtttcgaaacatgcaattatgtcactgatgagaatgacatattcgaagctcttaacatgaaatccgcagttggagcgttgtataaaggaaagaagaaggattacttcgctgagttcacacccgagatgaaagaagaaatactgaaacaaagttgtgaacggctcttcctaggaaagatgggagtgtggaacggctcgctgaaggcagagttgcgaccactagaaaaagtggaagcaaacaaaacacggacgtttactgccgcaccactagacacactgttgggtggaaaagtttgcgtggatgatttcaacaaccagttctatgatcacaaccttagagctccttggagcgttggcatgacaaagttttattgtggttgggatcgcttgttggagtcgttgccagatggttgggtgtattgcgatgctgatggctcacagttcgacagctcgctatcgccatacttgatcaacgcagtactcaacatccgcttaggattcatggaagagtgggacataggggaggtaatgctgagaaatttgtacaccgaaatcgtgtatacccctatttctacaccagatggtacactcgtcaagaagttcaaaggaaacaatagcggacagccatcgactgttgtggacaacacgctcatggtcatattggcagtcaactattcactcaagaaaagcggaattccaagtgagttgcgcgacagcatcatcagattcttcgtcaacggagatgatttactgctaagcgtacacccagagtatgagtatattcttgacactatggcagacaactttcgtgaactgggcctgaagtatactttcgactcaagaaccagggaaaaaggagacctctggtttatgtcgcaccaggggcacaaaagagagggaatctggattcccaagctcgagccagagcgaatagtatcgattctagaatgggatcggtcgaaagagccatgccatcgactagaggcaatctgcgcagcgatgattgagtcgtggggatacgacaagttaactcacgagatacgcaagttctacgcgtggatgattgaacaagctccatttagctccctagcacaagaagggaaagctccttacatagcggaaacagcgctgaggaagctctaccttgataaggaaccagctcaagaggatctcacccattatttgcaagcaatctttgaggattatgaagatggtgctgaggcttgtgtttatcaccaggcaggtgaaacgcttgatgcaggtttgacagacgagcaaaagcaggcagagaaggagaagaaggagagagagaaggcagaaaaggaacgagagaggcaaaagcagttggcactcaagaaaggcaaggatgttgcacaagaagagggaaaacgcgacaaggaagtaaacgctggaacctctggaactttcagtgtacccagactcaagagtctgacaagcaagatgcgcgtgccaagatacgagaaaagagtggctctaaacctcgatcatctaatcctatacacgccggagcagacggatctatccaacacacgttcaacgcgaaagcagtttgacacatggtttgaaggtgtaatggctgattacgaactgacggaggacaaaatgcaaatcattctcaatggtttaatggtctggtgcattgagaacggaacctccccgaacataaacggaatgtgggtgatgatggacggcgacgatcaggtggaattcccgatcaaaccgctcattgaccacgccaaacccacatttaggcagataatggcccatttcagtgacgtagctgaagcgtacattgaaaagcgtaaccaagaccgaccatacatgccacgatatggtcttcagcgcaatttaaccgacatgagcttagctcgatacgcatttgatttctatgaaatgacttctaggactccaatacgtgcgagagaggcacacatccagatgaaagcagcagcactgcgtggcgcaaataataatttgttcggcttggatggaaacgttggtacaacggtagagaacacggagaggcatacgaccgaggacgttaatcggaacatgcataacttactgggcgttcaggggttgtgaagttgtatgctggtagactataagtatttaagtttactcgttagtattctcgcttatgggaaatatgtaagtttgttaaagcagccagtgtgactttgtcatgtgtgttgttgttactttctgtattttcgccgaacattttattggtgttagcgcatgtagtgaggatcgtcctcgattgccttaacatttgataggatgcaagggaca

SEQ ID NO: 48 - DNA, 12651 bp, Artificial sequence
  Arabidopsis Luciferase (AP018660.1 Gateway vector R4L1pMpGWB435 DNA, complete sequence)
tttcacgcccttttaaatatccgattattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgtggatcccccatcacaactttgtatagaaaagttgaacgagaaacgtaaaatgatataaatatcaatatattaaattagattttgcataaaaaacagactacataatactgtaaaacacaacatatccagtcactatggcggccacatttaaatgtcgaggggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaagatctggatccggcttactaaaagccagataacagtatgcgtatttgcgcgctgatttttgcggtataagaatatatactgatatgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccaacgctagcatggatctcgggccccaaataatgattttattttgactgatagtgacctgttcgttgcaacaaattgatgagcaatgcttttttataatgccaactttgtacaaaaaagcaggctcaagcatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaagcttagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggttccggaaccaattcgtaatcatggtcatagctgtttcctgtgtgaaattgttatccgctcacaattccacacaacatacgagccggaagcataaagtgtaaagcctggggtgcctaatgagtgagctaactcacattaggctgaattaggcgcgcctatttctgaattcaacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgactctagctagaggatcgatccgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgtgtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccccatggtcgatcgacagatctgcgaaagctcgagagagatagatttgtagagagagactggtgatttcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgaattcagaaataaattcagcctaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaacct

SEQ ID NO: 49 - DNA, 9181 bp, Artificial Sequence
  AF033819.3 HIV-1, complete genome
ggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaggaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttggctgaagcaatgagccaagtaacaaattcagctaccataatgatgcagagaggcaattttaggaaccaaagaaagattgttaagtgtttcaattgtggcaaagaagggcacacagccagaaattgcagggcccctaggaaaaagggctgttggaaatgtggaaaggaaggacaccaaatgaaagattgtactgagagacaggctaattttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcaggtcactctttggcaacgacccctcgtcacaataaagataggggggcaactaaaggaagctctattagatacaggagcagatgatacagtattagaagaaatgagtttgccaggaagatggaaaccaaaaatgatagggggaattggaggttttatcaaagtaagacagtatgatcagatactcatagaaatctgtggacataaagctataggtacagtattagtaggacctacacctgtcaacataattggaagaaatctgttgactcagattggttgcactttaaattttcccattagccctattgagactgtaccagtaaaattaaagccaggaatggatggcccaaaagttaaacaatggccattgacagaagaaaaaataaaagcattagtagaaatttgtacagagatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaattgggcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactgcccatacaaaaggaaacatgggaaacatggtggacagagtattggcaagccacctggattcctgagtgggagtttgttaatacccctcccttagtgaaattatggtaccagttagagaaagaacccatagtaggagcagaaaccttctatgtagatggggcagctaacagggagactaaattaggaaaagcaggatatgttactaatagaggaagacaaaaagttgtcaccctaactgacacaacaaatcagaagactgagttacaagcaatttatctagctttgcaggattcgggattagaagtaaacatagtaacagactcacaatatgcattaggaatcattcaagcacaaccagatcaaagtgaatcagagttagtcaatcaaataatagagcagttaataaaaaaggaaaaggtctatctggcatgggtaccagcacacaaaggaattggaggaaatgaacaagtagataaattagtcagtgctggaatcaggaaagtactatttttagatggaatagataaggcccaagatgaacatgagaaatatcacagtaattggagagcaatggctagtgattttaacctgccacctgtagtagcaaaagaaatagtagccagctgtgataaatgtcagctaaaaggagaagccatgcatggacaagtagactgtagtccaggaatatggcaactagattgtacacatttagaaggaaaagttatcctggtagcagttcatgtagccagtggatatatagaagcagaagttattccagcagaaacagggcaggaaacagcatattttcttttaaaattagcaggaagatggccagtaaaaacaatacatactgacaatggcagcaatttcaccggtgctacggttagggccgcctgttggtgggcgggaatcaagcaggaatttggaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccatatgtatgtttcagggaaagctaggggatggttttatagacatcactatgaaagccctcatccaagaataagttcagaagtacacatcccactaggggatgctagattggtaataacaacatattggggtctgcatacaggagaaagagactggcatttgggtcagggagtctccatagaatggaggaaaaagagatatagcacacaagtagaccctgaactagcagaccaactaattcatctgtattactttgactgtttttcagactctgctataagaaaggccttattaggacacatagttagccctaggtgtgaatatcaagcaggacataacaaggtaggatctctacaatacttggcactagcagcattaataacaccaaaaaagataaagccacctttgcctagtgttacgaaactgacagaggatagatggaacaagccccagaagaccaagggccacagagggagccacacaatgaatggacactagagcttttagaggagcttaagaatgaagctgttagacattttcctaggatttggctccatggcttagggcaacatatctatgaaacttatggggatacttgggcaggagtggaagccataataagaattctgcaacaactgctgtttatccattttcagaattgggtgtcgacatagcagaataggcgttactcgacagaggagagcaagaaatggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagagctcatcagaacagtcagactcatcaagcttctctatcaaagcagtaagtagtacatgtaatgcaacctataccaatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagaaatatcagcacttgtggagatgggggtggagatggggcaccatgctccttgggatgttgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagaggtaaggtgcagaaagaatatgcatttttttataaacttgatataataccaatagataatgatactaccagctataagttgacaagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctagcagaagaagaggtagtaattagatctgtcaatttcacggacaatgctaaaaccataatagtacagctgaacacatctgtagaaattaattgtacaagacccaacaacaatacaagaaaaagaatccgtatccagagaggaccagggagagcatttgttacaataggaaaaataggaaatatgagacaagcacattgtaacattagtagagcaaaatggaataacactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcaccctcccatgcagaataaaacaaattataaacatgtggcagaaagtaggaaaagcaatgtatgcccctcccatcagtggacaaattagatgttcatcaaatattacagggctgctattaacaagagatggtggtaatagcaacaatgagtccgagatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttagcttgctcaatgccacagccatagcagtagctgaggggacagatagggttatagaagtagtacaaggagcttgtagagctattcgccacatacctagaagaataagacagggcttggaaaggattttgctataagatgggtggcaagtggtcaaaaagtagtgtgattggatggcctactgtaagggaaagaatgagacgagctgagccagcagcagatagggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataagatagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttc

SEQ ID NO: 50 - DNA, 7350 bp, Artificial sequence
  Human Luciferase (FJ376737.1 Cloning vector pmirGLO, complete sequence)
catgcaagctgatccggctgctaacaaagcccgaaaggaagctgagttggctgctgccaccgctgagcaataactagcataaccccttggggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggtttttttaagcaagtaaaacctctacaaatgtggtaaaatcgaattttaacaaaatattaacgcttacaatttcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaaccaaaggctggagccaccatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagaccggtggtgggagcggaggtggcggatcaggtggcggaggctccggagggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatcctctttgcgcttgcgttttcccttgtccagatagcccagtagctgacattcatccggggtcagcaccgtttctgcggactggctttctacgtaatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcactttcaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaactatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaattcgaaatgaccgaccaagcgacgcccaaccggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatttcaagaagatcctttgatcttttctacggggtctgacgctcagtggaacgaaaactcacgttaagggattttggtcatgagattatcaaaaaggatcttcacctagatccttttatagtccggaaatacaggaacgcacgctggatggcccttcgctgggatggtgaaaccatgaaaaatggcagcttcagtggattaagtgggggtaatgtggcctgtaccctctggttgcataggtattcatacggttaaaatttatcaggcgcgattgcggcagtttttcgggtggtttgttgccatttttacctgtctgctgccgtgatcgcgctgaacgcgttttagcggtgcgtacaattaagggattatggtaaatccacttactgtctgccctcgtagccatcgagataaaccgcagtactccggccacgatgcgtccggcgtagaggatcgagatctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttggcaatccggtactgttggtaaagccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaattctagttgtttaaacgagctcgctagcctcgagtctagagtcgacctgcagg

SEQ ID NO: 51 - DNA, 22558 bp, Caenorhabditis elegans
aaaacgatctagataatcgcccgcagagttgctcgatctgcggagtgaatgtgccgtgcgaacatcgtcatagaagagacagtttgatcgaattaaaagtcgtgcgccggcccctccaacttcgaccatggttggcgcaccgcgcttcacccagaaaccgtccattcagcagacacctactggtgatctgctgatggaatgtcatcttgaggcggatccacaaccaacgatcgcctggcaacattctggaaatcttctggaaccatctggaagagttgtgcagacacttaccccgttaggaggaagtctgtacaaggcaacattagtgatcaaggaaccaaatgccggcgacggtggtgcctacaaatgcacagcaagaaatcaattgggtgaatcgaacgccaatatcaatttgaacttcgctggtgccggaggagatgaagccaagtcccgtggcccatcttttgtcggaaaaccacgaattattccaaaagatggtggagctctgattgtgatggaatgcaaagttaaaagtgcatccacaccagtcgcaaaatggatgaaagatggagtcccattgagtatgggcggtctctatcacgccattttcagtgatctcggagatcaaacctacttgtgtcaactggaaattagaggtccgtcatcatctgacgcaggacaatatcgatgtaatattagaaacgatcaaggagaaacgaatgccaatttggcattgaatttcgaagaacccgatccatccgaacgtcaagagagaaaaagatccacagcttcaccaagaccatcgtcccgtggtccaggtagtcgtccgtcttctccgaaaaaatcaatgaaatcgagggaaggaactccaaaacgtaccctgaaaccaagagagggttccccatcgaaaaagttgagatcccgaacttcaaccccagtcaacgaagaagtctctcaatcggagtcccgccgatcgagtagaactgataaaatggaagtggatcaagtatcaggtgcatcgaaacgaaagcctgatggacttccacctccgggaggtgatgagaagaagcttcgagcaggtagtccctctactcgaaagtctccatcaagaaagagcgcatcaccaacaccatctagaaaaggaagcagtgctggaggagctgcttccggtacgacaggagcatctgcatctgcaacatcagcaacatctggtggatctgcatcctctgatgcctcgcgtgacaaatacacaaggccgccaatcgttctcgaagccagtaggtcccagactggtcgaatcggtggatctgtcgttttggaggtacaatggcagtgtcattcatcaacaattattgagtggtatagagatggtacattggtcagaaattcttccgaatattcacagtcgttcaatggatcaatagctaaactgcaagtgaacaagctgaccgaagagaaatcgggtctctataaatgtcatgcaaagtgtgactatggagaaggtcaaagcagtgcaatggtcaaaatcgaacagtctgatgtggaagaagaactcatgaagcatagaaaagacgcggaggatgaatatcaaaaagaagaacagaaatcgcagacgcttcaagctgaaaccaaaaagcgagtggcgagacgaagcaagtcaaagagtaagagtccggcaccccaagccaaaaagagtacaacatctgaaagtggccgtcaagaagcttcggaagtcgaacacaaaagaagctcaagtgttcggcctgatccagatgaggaatctcagttggacgagatcccaagttcgggtctgacgatcccagaggagcgccgtcgagaattattgggtcaggtcggagaaagtgacgacgaggtatccgagtcgatatccgaactaccatcattcgccggaggcaagcctcgccgcaagactgatagtcctccaaagcaggacgatatgttttctcgcgacactcttcttcgcaaaaccactacatccacgaaaaatgagtcgagtactgtagaggaaaagacaaaacttcgcaaaactgtcaaaaaagtcgatggtgaactcgatttcaaagccatggtaaaattgaaaaaagtaaaaaaagaggagggtggaaccaccgaaaagtcgggcttcccactcgatcatgccgattccacgtcatctgtattgtctcaagaatcgaggtcgagacgcggttcaaatgctccatttgcaaaagatggccttcctgagcaaccggcaaacccgtttgcacagctcaaaaaggtgaaatctggcgctggtggactggaaaagtccgattcaatggccagtctcaagaagcttgatttaaagaaaggaaagatcgatgataactcggatggtgcattcaaagtacaactgaaaaaggttgtaaagaaggaagttaaagagtcgacaatcagtgtgaaagagaagaatggcaccgaatcaggcatcaagaccgagtttaaaatggagaaacgtgaacggaccacattgcaaaaatatgagaaaacggatagtgatgggtccaagaaggaggataaaccaaagaaagtcagcattgctccggtttcaaccaacaaatcgtccgacgatgaaccgtctacgccacgtcaccacaaagaagtcgaagagaagtcgacatcagaagaactcaaagcaaaagtcgctggccgtcaagtcggacaaaaacgaaatggcgctcagaagcccgaagagcccaaaaatcttttgtcacagattcaattgaagaaagtgacaaagaaggctcacgacgataccaatgagcttgaaggaatcaaattgaaaaaagtgacgacagtgccaaaacacgtcgccgatgatgacagtcaatcggaatccgagtcacggcgtggatccgtgttcggagaactccgacgtggatcgcgagctccgagagactctgcagacaattctcgcagagactccattagacgctctagtatcgacatgagacgtgaatctgttcaagaaattcttgaaaagacgtcgacaccgctcgtcccaagcggtgcttcaggcagtgctccaaaaatcgtcgaagtcccagaaaacgtcacagtcgttgagaacgaaactgcaatcctaacgtgcaaagtgtcgggtagtcccgcccccactttccgttggttcaaaggtagtcgtgaagttatcagtggaggtcgcttcaagcatattaccgatggaaaagaacatactgtggcgttggcattacttaagtgccgatcccaggatgaaggtccctacaccctgacaatcgaaaacgtgcatggaaccgactcggctgacgtcaaactgttggtcacttcggataatggtctcgatttccgagccatgctaaaacacagagaatcacaagctggcttccaaaaagacggagaaggaggcggtgctggtggtggtggcggtgagaagaagccaatgaccgaagccgagagaagacagtccctcttccccggaaagaaggttgaaaaatgggatattccacttccagagaagaccgttcaacaacaggttgacaaaatctgtgagtggaagtgtacctactcacgtccaaatgccaaaattcgttggtacaaggacagaaaagagatcttctccggaggtctcaaatacaagatcgttatcgaaaagaacgtgtgcactctgattatcaacaatccagaagtcgatgacaccggaaagtacacctgtgaggctaacggagtaccaactcatgctcagcttactgtacttgaaccgccgatgaagtacagtttcctgaacccgttgccgaatacacaggagatctaccgtactaagcaagcagtgctcacatgtaaagtgaacacaccacgtgctccattggtatggtaccgtggaagcaaggctattcaagaaggagatccacgattcattattgaaaaggatgccgtcggtcgttgtacacttacaatcaaggaagttgaggaagacgatcaagctgaatggactgctagaatcacacaagacgtgttctcaaaggttcaagtgtacgttgaggagccacggcatacattcgttgttccaatgaagtcgcaaaaagtcaacgaaagtgatttggcaacattggagactgatgttaacgacaaggatgctgaagttgtttggtggcatgatggaaagagaatcgatattgatggagtgaaattcaaggttgaatcttcaaacagaaagagaagacttattatcaatggagctagaattgaagatcatggagagtataagtgtacaactaaggatgatagaactatggctcagctcatcgttgatgctaagaataagttcatcgttgctctcaaagacactgaagttattgagaaggatgatgttacattgatgtgtcagacaaaggacacaaaaactcctggaatttggttccgtaatggaaaacaaatttccagtatgcccggaggaaagttcgaaactcaatcgagaaacggaactcatactcttaaaatcggaaagatcgagatgaacgaggctgatgtttatgaaatcgatcaggcaggactacgtggatcttgcaatgtgactgttctcgaggcagaaaagcgtccaattctcaactggaagccaaagaaaatcgaagcaaaggctggagaaccatgtgttgtgaaggttccattccaaatcaagggaacacgacgtggagatccaaaggctcaaattctgaagaatggaaagccaatcgatgaagaaatgagaaagctagttgaagttattatcaaggatgatgtggctgagattgttttcaaaaatccacaacttgctgatacaggaaagtgggctctcgaactcggaaactcggctggaacagcacttgctccattcgagttgttcgttaaggacaagccgaaaccaccaaagggtccacttgaaaccaagaatgttactgctgaaggtcttgatctcgtctggggaactccagatccagatgagggagctccagttaaagcatacatcattgaaatgcaagagggaagaagtggaaactgggctaaagttggagagactaagggaacagacttcaaggttaaggatcttaaagaacatggagaatacaagttcagagtcaaggctcttaatgaatgcggactctctgatccactcacaggagaatctgttcttgccaaaaatccatacggcgttcctggaaaaccaaagaacatggacgcaattgatgttgacaaggatcactgtacccttgcatgggaaccgccagaggaggatggaggtgctccaatcactggttacatcattgaaagaagagagaagtccgagaaagattggcatcaagttggacagaccaaaccagattgttgtgaactgactgataagaaggttgtcgaagataaggaatacttgtacagagtaaaagcagtcaacaaggctggaccaggagacccatgtgatcatggaaagccaatcaagatgaaagccaagaaagcttctccagaattcactggtggaggcatcaaggatcttcgtcttaaggtcggagaaactatcaagtacgacgttccaatttctggagaaccactcccagaatgtctttgggtggttaatggaaaaccactgaaggctgttggaagagtcaagatgtcttctgaaagaggaaagcatatcatgaagatcgaaaatgcagttcgtgctgattccggaaagttcactatcactttgaagaactcttctggctcatgcgactcgaccgccacggtcactgtcgttggaagaccaactccaccaaagggtccactcgatattgctgatgtttgtgccgatggtgcaaccctttcctggaatcctccagatgatgatggaggtgatccactcacaggatacatcgttgaagctcaagatatggacaacaagggaaaatacattgaagttggaaaggttgatccaaacaccactaccctcaaagttaatggactccgtaacaagggaaattacaagttccgcgtgaaggcagtcaacaacgaaggagaatctgagccactttctgctgatcagtacactcagatcaaggatccttgggatgaaccaggaaagcctggaagaccagaaattaccgatttcgatgcggatagaattgacattgcctgggagccaccacacaaagatggaggagctccaatcgaggagtatattgtcgaagttcgtgatccagataccaaagaatggaaggaagtcaagagagttccagacaccaatgcatcaatttctggattgaaggaaggaaaggaatatcagttcagagttcgggctgttaacaaggctgggcctggacaaccttccgaaccatcagagaagcaattggctaagccaaaattcatcccggcatggttgaaacacgacaatttgaaatctatcaccgtaaaggctggagccactgttcgttgggaagtcaaaattggaggagaaccaattccagaagtcaaatggtttaaaggcaatcaacaactcgaaaacggaattcaacttacaattgatactcgcaaaaatgagcacactattctgtgcattccatctgcaatgcgatctgatgttggagagtatcgattgactgtcaagaactcgcatggagctgatgaagagaaggctaaccttaccgttttggacagaccaagcaaaccaaatgggccacttgaagtttcagatgtctttgaagataatctgaacctttcttggaagccaccagatgatgacggaggtgagccaatcgaatattatgaagtcgagaagcttgatactgccactggaagatgggttccatgcgccaaagttaaggatacgaaggctcatattgatggtctcaagaagggacaaacatatcagttccgtgtcaaggctgtcaataaggaaggagcttctgatgcattgtctactgataaggacaccaaagccaagaatccatatgatgagccaggaaaaaccggaactccggatgttgtcgactgggatgctgatcgtgtctcacttgaatgggaaccaccaaagtctgacggaggagctccaatcactcaatacgtcattgagaagaagggcaaacatggaagagactggcaagaatgcggaaaggtttctggagatcaaaccaatgctgagattcttggactcaaggagggagaagagtaccagttccgtgtgaaggctgttaacaaggccggaccgggagaggcttcagacccaagccgaaaggttgttgcaaagccaagaaacttgaagccatggattgatcgtgaagcaatgaagacgatcactatcaaggtaggaaacgatgtggaattcgatgttccagtacgcggagaaccaccaccgaagaaggaatggatcttcaatgagaaaccagtcgatgatcaaaagatcaggattgaaagcgaagactacaagacccgatttgtgctccgtggagcaactcgcaagcatgctggtttgtacactcttactgctaccaacgcttctggaagcgacaaacattccgttgaggtcattgtgctcggaaaaccatctagcccattgggacctttggaagtgtcgaatgtctacgaagatcgcgcagatttggagtggaaagtaccagaagatgacggaggtgctccaattgatcattatgaaatcgaaaagatggatttggcaactggaagatgggtcccatgtggaagaagtgaaacaacaaagaccacagttccaaatcttcaacccggacacgaatacaaattccgtgtcagagctgtgaacaaggagggagaatccgatccactcacaaccaacaccgcaatcctcgccaagaacccatacgaggttccaggaaaagttgacaagccggaattggtggactgggataaggatcatgttgatcttgcatggaatgctccagacgatggtggtgcaccaattgaagctttcgtcattgaaaagaaggataagaatggacgatgggaagaagctctcgttgttccaggagatcagaaaacagcaactgttccaaatcttaaggagggagaagaatatcaattcagaatttctgctcgtaacaaggctggaactggagatccttctgatccttctgatcgtgttgttgcgaagccaagaaaccttgctccaagaattcatcgtgaagatctttctgatacaactgtcaaggtcggagccactctcaagttcattgttcatattgatggtgagccagcaccagatgtaacatggtcattcaatggaaaaggaatcggagagagcaaggctcaaattgaaaatgagccatacatctcgagatttgctttgccaaaggcacttcgtaagcaaagtggaaaatataccatcactgcaaccaacattaatggaactgacagtgtcactatcaatatcaaggtaaaaagcaagccaacgaaaccaaagggaccaatcgaggtaactgatgtcttcgaagatcgtgcaactcttgactggaaaccaccagaggatgacggaggagagccaattgagttctatgaaattgaaaagatgaacaccaaggacggaatctgggttccatgtggacgtagtggagatacccacttcacagtcgattcactcaacaagggagatcattacaagttccgtgtcaaggctgtcaacagcgaaggaccttctgatccattggaaactgaaaccgatattttggctaaaaatccatttgatcgtccagatagaccaggtcgtccagagccaactgattgggattctgatcatgttgatctcaagtgggatccaccactttctgatggcggcgctccaattgaggagtaccaaattgagaagagaaccaaatacggaagatgggaaccagccatcactgttcctggcggtcagacaactgcaaccgttccagacctcacaccaaatgaggaatacgaattccgtgttgttgctgttaacaagggaggcccatctgatccatctgatgctagcaaggctgttattgctaaaccaagaaacttgaagccacacatcgacagagatgctctcaagaatctgactatcaaggctggtcaatcaatttccttcgatgttccagtatcaggagaacctgcaccaacagtcacatggcattggccagacaacagggaaatcagaaatggaggacgcgtcaagcttgataacccagaataccaatcaaagctggttgtgaagcaaatggaacgtggagacagtggaactttcactatcaaagctgtcaatgcaaatggagaagatgaagcaactgttaagatcaatgttattgacaagccaacttctccaaatggtccattagatgtttccgatgttcatggtgatcatgtcactttgaattggcgtgcaccagatgatgatggaggtattccaattgaaaactatgtgatcgaaaagtacgatactgcaagtggaagatgggttccagctgcaaaggtcgctggagataagactacagctgttgttgacggtcttattcctggacatgaatataaattccgtgtcgctgccgtcaatgctgaaggagagtccgatccattggagaccttcggaaccacacttgccaaagatccatttgacaagccaggaaagacaaatgctcctgaaattactgattgggataaggatcatgttgaccttgaatggaagccaccagcaaacgacggtggtgctccaatcgaggaatacgttgttgagatgaaggacgagttctcgccattctggaatgacgttgctcatgttccagctggacaaacgaatgctactgttggaaatctcaaggaaggatcaaagtacgaattcagaatccgtgccaagaacaaggcaggattgggagatccatctgattcagcatcagctgttgcaaaggctagaaatgttccaccagtcatcgatcgtaactcgattcaagaaatcaaggtcaaggctggacaagacttctcattgaacattccagtcagtggtgaaccaactccaacaattacttggactttcgaaggaacaccagtcgaatctgatgatcgtatgaagttgaacaatgaagacggcaagactaaattccatgtgaagagagctttgcgttcggatacaggaacctatatcatcaaggcagagaacgagaatggaactgacactgctgaagtgaaggttactgttcttgatcatccatcaagcccacgtggacctctcgatgtcactaatattgtcaaggatggatgtgatcttgcatggaaggaaccagaagatgatggaggagctgaaatcagtcactatgtcattgaaaagcaagatgctgccactggcagatggactgcttgcggagagagcaaggataccaacttccacgttgacgatttgactcaagggcatgaatataaattccgtgtcaaggctgttaacagacatggagattcggatccattggaggctcgtgaagctattatcgccaaggatccattcgatcgtgctgataagccaggaactccagaaattgtggactgggacaaggatcatgcagatctcaagtggactccaccagctgatgatggaggtgctccaatcgaaggatatctcgttgaaatgagaactccatcaggagactgggtaccagctgtcacggttggagccggtgagctcactgctacagttgatggcttgaaaccaggtcagacttaccaattccgtgtcaaggctttgaacaaggctggagaatcgactccatctgatcctagcagaaccatggttgctaagccacgtcatcttgctccaaagatcaacagagatatgtttgttgctcaaagagttaaggctggtcaaactcttaactttgatgtaaatgttgaaggagagccagctccaaagatcgaatggttcttgaacggatctccattgtcatctggtggaaatacccatatcgacaataacactgacaacaacaccaagttgacaacaaagagcactgctcgtgccgatagtggaaaatacaaaattgtggctaccaatgaaagtggaaaagatgaacatgaggttgatgtcaacattcttgacatccctggtgcaccagaagggccacttcgtcacaaggatattaccaaggagagtgttgtgctgaaatgggatgaaccattggatgatggaggttctccaattaccaactacgtagttgagaaacaagaagacggaggtcgatgggtaccatgtggagaaacatctgatacttctctgaaggttaacaaactatccgaaggacatgaatacaagttccgtgtgaaagcagtaaaccgtcaaggaacatctgctccattgacttctgatcatgcaattgttgctaagaatccattcgatgaaccagatgcaccaactgatgttaccccagttgattgggataaggatcatgttgatcttgaatggaagccaccagcaaacgacggtggtgcaccaattgatgcttacatcgttgagaagaaggacaagtttggagactgggttgagtgtgcacgtgttgatggaaagacaacaaaggcaactgctgataatttgactccaggagagacttatcagttccgtgtgaaggctgtcaataaggctgggccaggaaaaccatctgatccaacaggaaatgttgttgccaaaccaagaagaatggctccaaaacttaacctcgccggacttttggatctccgtatcaaggctggaacacccatcaagctcgatatcgcattcgaaggagagccagccccagttgctaaatggaaggccaacgatgcaacaatcgatacaggagcaagagctgatgttacgaacacaccaacatcctcagcaattcacatcttctctgctgttcgtggagatactggagtttacaaaatcattgttgaaaatgagcatggaaaagatactgctcagtgcaatgttactgttcttgatgtaccaggaactccagaaggaccactcaagattgacgagatccataaggaaggatgtacattgaactggaagcctccaactgataacggaggaactgatgttcttcactacattgttgagaagatggatacttctcgtggaacatggcaggaagtcggaactttcccagattgtacagccaaggttaataagcttgttcctggaaaggaatacgcattccgtgtcaaggcagtcaatcttcaaggagaatcaaaaccattggaagctgaagaaccaattattgcaaagaatcaatttgatgttcctgatccagttgacaaaccagaggttactgactgggataaggatagaattgatattaagtggaacccaactgcaaacaatggaggagctccagtcactggatatattgttgagaagaaggagaagggaagcgcaatctggacagaagccggaaagactcctggaacaacattcagtgctgataacctgaaaccgggagttgaatacgagttccgtgttattgctgtcaatgctgcaggaccatctgatccatccgatccaactgacccgcaaatcaccaaggctcgctacttgaaaccaaagatcttaactgctagcagaaaaatcaagataaaggctggattcactcataacttggaagtcgatttcattggagctcccgatccaactgccacttggactgttggagattctggagcagctcttgctccagaacttcttgttgatgccaagagctcgactacgtcaatattcttcccatctgctaaacgtgcagacagtggaaactacaagttgaaggttaagaatgagttgggagaggatgaagctatcttcgaggtcattgttcaagatagaccgtctgctccagaaggaccacttgaagtttctgatgtcactaaggatagttgtgttctcaattggaaaccaccaaaggatgatggaggtgctgaaatcagcaactatgtcgttgagaagagagatacaaagaccaacacctgggttccagtttctgcatttgtcactggaacctcaatcactgttccaaaactcacggaaggccacgaatacgagttccgtgttatggctgagaacacctttggtagatctgattcattgaacaccgatgagccagttcttgcaaaggatccatttggaacaccaggaaagccaggaagaccagaaattgttgatactgataatgatcatatcgatatcaaatgggatcctccacgtgacaacggtggatcaccagttgatcattacgacattgagaggaaggatgcaaagactggacgctggatcaaggttaacacatctccagttcaaggtactgcattctcggatactcgtgttcaaaagggtcatacctatgaataccgtgtcgttgccgtcaacaaagctgggccaggacaaccatcagattcgtctgcggctgctactgctaagccaatgcatgaggctccgaagttcgatttggatttggatggaaaggaattccgtgtgaaggccggagaaccattggttattactattccattcactgcatccccacaaccagacatttcatggaccaaggagggaggaaaaccattggctggagttgaaacaactgattctcagaccaagttggttattccatctaccagaagatctgattctggaccagtcaaaatcaaggccgtcaatccatacggagaagctgaagcaaacatcaagatcacagttatcgacaaaccaggagctccagaaaacattacttacccagctgtcagcagacacacttgcactctcaattgggatgctccaaaggacgatggaggcgccgaaatcgctggatacaaaattgaatatcaagaagtcgggtctcagatttgggataaggttcctggactcatttccggaactgcttatactgttcgtggacttgagcacggtcaacaataccgtttcagaatccgcgcagagaatgcagtcggactttctgattactgccaaggtgttccagttgttatcaaggatccattcgacccaccaggtgcaccaagtactccagaaatcactggatacgataccaatcaagtatccctggcatggaacccaccaagagacgacggtggttctccaattttgggatatgtcgttgaacgttttgagaagagaggtggcggtgattgggctccagtcaagatgccaatggtcaagggaactgaatgcattgtgccaggacttcatgagaatgaaacctaccaattccgagtacgtgccgtgaatgctgctggacatggagaaccaagtaacggatctgagccagtcacctgcagaccttatgtcgagaaaccaggtgcaccagatgctccaagagttggaaagattaccaaaaactctgctgagcttacctggaatagaccattgagagatggaggtgcaccaattgatggatacattgttgaaaagaagaagcttggagataatgattggaccagatgcaatgataaaccagttcgtgatacagcctttgaagtcaagaatcttggtgaaaaggaagaatacgagttccgtgttatcgctgtcaatagcgccggagaaggtgaaccatccaagccatccgatttggtgctcatcgaggaacaaccaggacgaccaatcttcgatatcaacaatctcaaggatatcactgtccgtgctggagaaacaatccaaattcgtattccatatgctggaggaaatccaaaaccaattattgatctgttcaacggaaactctccgatcttcgagaacgagagaactgttgttgatgtcaatccaggagaaatcgttatcaccactaccggatcaaagagatcggatgctggtccatacaagatttctgccactaacaaatacggaaaagatacttgcaagcttaatgtcttcgttcttgatgctccaggaaaaccaactggaccaatccgtgctactgacattcaagccgatgcaatgacgctttcatggagaccaccaaaggataatggaggagatgctatcaccaattatgtcgttgaaaagagaactccaggcggtgactgggtcactgtcggacatcctgttggaacaactcttcgtgtccgcaatttggatgccaatactccttacgaattccgtgttcgcgctgaaaatcaatatggagttggagagccacttgaaactgatgacgcaattgttgccaagaatccgttcgatactccaggagctccaggacaaccagaagccgttgaaacttcggaggaagcaattacacttcaatggacaagaccaacatctgatggaggagctccaattcaaggatatgtgattgagaagagagaagtcggatcaactgaatggacgaaagctgcatttggaaacatccttgacacaaaacatcgtgtcactggactcactccaaagaagacctacgaattcagagttgcagcttacaatgcagctggacaaggagaatacagtgttaactctgttccaattacagctgataatgctccaacaagaccgaagatcaacatgggaatgcttactcgcgatatcctcgcttacgctggagaacgtgcaaagattcttgttccatttgctgcttcacctgctccgaaagtcactttctcgaagggtgagaataagatcagcccaactgatccaagagtgaaggttgagtacagtgacttcttggctacattgacgatcgagaagtctgaacttactgatggtggactctactttgttgaacttgaaaatagtcaaggaagtgattctgctagtatcagattgaaggttgttgataaaccagcttcaccacaacacatccgagtagaggatattgcaccagactgctgcactctttactggatgccaccatcatctgatggaggatcgccaatcacaaactacatcgttgagaagcttgatcttcgtcattctgatggaaagtgggagaaggtttcttcattcgttcgcaatttgaactacactgttggaggacttatcaaggacaaccgttatcgtttccgtgttcgtgccgagacccaatacggagtttcagaaccatgtgagcttgctgatgtcgtcgttgccaagtatcagttcgaggttccaaatcaaccagaagctccaactgttcgcgacaaggactccacttgggccgagttggaatgggatccaccaagagatggaggatcaaagatcattggataccaagttcaatacagagatacttcttctggaagatggatcaatgccaagatggatctttccgaacaatgccatgctcgtgtcactggacttcgtcaaaatggagaatttgaattcagaatcattgcaaagaatgcagctggattctcgaaaccatctccaccatctgaaagatgtcagctcaagtctcgatttggaccaccaggtccaccaattcatgttggagccaagtcaattggccgcaaccattgtacaattacctggatggcgccattggaagatggaggatcaaagatcacaggatacaacgttgaaattcgtgaatacggaagtacattgtggactgttgctagcgactataacgttcgtgaacctgaattcacagttgacaaactcagggagttcaatgattatgaattccgtgttgttgcaatcaatgctgctggaaagggaattccatctcttccatctggaccaatcaagattcaggaatccggtggaagtcgcccacaaatcgttgtcaagccagaagacactgctcaaccatacaacagaagagccgtgttcacttgcgaagctgtcggaagaccagaaccaactgcaagatggcttagaaatggaagagaacttccagagagtagcagatatagattcgaagcaagtgatggagtctacaagttcactatcaaggaagtttgggacattgatgctggagagtacacagtcgaggtgtccaatccatatggaagcgacactgccactgctaaccttgttgttcaagctcccccagtcatcgagaaagatgttccaaacacaattcttccaagtggagatcttgttcgtctgaagatttacttctcgggaaccgcaccattccgtcatagtttggtattgaacagagaagaaattgacatggatcatccaacaattagaatagttgaattcgatgatcatatcttgatcacaattccagccttatcagtgagagaagccggacgttacgagtacaccgtttccaatgactctggagaagctaccactggattctggctcaacgttacaggacttccagaggctccacaaggaccattgcacattagtaacatcggaccaagcactgccacattgtcatggagaccacctgtcacagatggaggttcgaagattaccagctatgtcgttgaaaagagagatctttcaaaggatgaatgggtaactgttacatcaaacgtcaaggacatgaactacatcgttactggattgttcgaaaatcacgagtacgaattcagagtatctgctcaaaatgagaacggaattggagccccacttgttagtgagcatccaattattgctcgtcttccattcgatccaccaacttcaccattgaacttggaaatcgttcaagttggaggagactacgtcacactttcatggcaacgaccattatctgatggtggaggtcgccttcgtggatacattgttgaaaaacaagaggaagagcatgatgaatggttcagatgcaatcagaacccatctccaccaaataactacaatgttccaaatctcatcgacggaagaaagtatagataccgagtatttgctgtcaatgatgcaggactttccgatctagccgagcttgatcaaactttgttccaagcatccggttctggagaaggaccaaagattgtcagtccattgagcgatttgaacgaagaagttggaagatgtgtcacatttgagtgtgaaatcagtggatctccaagacctgaatacagatggttcaagggatgcaaggaacttgttgacaccagcaagtacactcttattaataagggagacaagcaagtccttattatcaatgacttaacgtctgatgatgctgatgaatacacatgccgtgccaccaactcttcaggaactcgtagtaccagagccaatttgcgtatcaagaccaagccacgtgtgttcattccaccaaaatatcatggaggatatgaggcgcagaagggagaaactattgagttgaagattccatacaaagcatatccacaaggagaagccagatggacaaaggatggagagaagattgagaacaacagcaagttcagcatcactactgatgataagtttgctaccctccgaatctcgaatgccagtcgtgaggattatggagaataccgagttgtcgttgagaactcagttggatctgactctggaactgttaatgtcactgtcgccgacgtacctgaaccaccaagattcccgattatcgagaacattcttgatgaagctgtcatcttatcctggaagccaccagcacttgatggaggatcgttggttaccaactataccattgagaagagagaggcgatgggaggatcatggagtccatgtgccaagagtcgttacacctacacaacaattgaaggacttcgtgctggaaagcagtatgaattcagaatcattgctgagaacaaacacggacaatcgaaaccatgtgagccaacagctccagttttgattccaggagatgagcgtaaacgcaggcgaggatatgatgttgatgaacaaggaaagattgtacgtggaaaaggaaccgtttcttctaattatgacaactacgtttttgatatctggaagcaatattacccacaaccagtagaaatcaagcatgatcatgttctggaccactacgatattcatgaagagctcggaacaggagcatttggagttgtgcacagagtaactgaaagagcaactggaaacaactttgctgcgaaattcgtaatgactccacatgaatctgacaaggaaactgtcagaaaagagattcaaaccatgtcagttttgagacacccgacgcttgtgaaccttcatgatgctttcgaggatgacaatgaaatggtcatgatttacgagttcatgagtggtggagaactcttcgagaaggttgctgatgagcacaataagatgagtgaagatgaagctgttgagtacatgagacaagtctgtaagggactctgtcatatgcatgagaacaactacgtacatttggatttgaagccagagaacatcatgttcacaacgaagagaagcaacgagctgaaattgattgattttggactcaccgcccatcttgatccaaaacaatccgtcaaggttacaacaggaactgccgaatttgccgctccagaagttgccgaaggcaagccagtcggttattacaccgatatgtggagcgttggagttctctcttacattcttctttccggactttcaccatttggaggagagaacgatgatgaaacattgaggaatgttaagagctgtgactggaatatggacgattccgctttctctggaatctccgaagacggaaaagatttcatccgaaagcttcttcttgctgatccaaataccagaatgactattcatcaagctttggagcacccatggttgacaccaggaaatgctccaggacgtgactctcaaattccaagcagcagatacactaagatccgtgattcaatcaagaccaaatacgatgcttggccagaaccactgccaccacttggaagaatctcaaattattcatcattgagaaagcatagaccacaagagtactcaattcgtgacgccttctgggatcgatctgaagctcaaccacgattcattgtgaagccttacggaactgaagttggagagggacaatctgctaatttctattgcagagttattgcatcttcaccaccagttgttacttggcataaggatgatagagaattgaagcagagtgtgaaatatatgaagaggtacaatggaaatgattatggacttaccattaaccgagtaaagggagatgataagggagaatacacagtccgtgcaaagaactcatacggaaccaaggaagaaattgtattcttgaatgttacccgtcactctgaaccactcaaattcgagccattggagccgatgaagaaggcaccaagtccaccaagagttgaagaattcaaggagagaagatctgcacccttcttcacattccatctcagaaatcgtttgattcaaaagaaccatcagtgcaaattgacatgttctttgcaaggaaaccctaatccaacaattgaatggatgaaggacggacatccagttgacgaagatcgtgttcaagtttctttccgaagtggtgtatgctcacttgagatctttaatgctcgtgttgatgacgctggaacatacacagtcactgccaccaatgacttgggagttgatgtctccgagtgtgtactcacagttcaaactaaaggaggtgaaccaattccacgtgtttcttcgttcagaccccgaagagcttatgacacattatcaactggaactgatgtcgaaagatcacattcgtatgctgatatgagaagaagatcccttattcgtgatgtatcaccagatgtacgatcagccgctgatgatctcaagacaaagatcacaaatgaacttccatcgttcacagctcaactttcggattctgaaactgaagttggaggatctgctgagttttcggcggcagtctctggacaacctgaaccacttatcgaatggcttcataatggagaaagaatctcggaatccgactcaaggttccgtgcctcgtatgttgctggaaaagcaacgttgcgaatcagtgacgccaagaagtcggatgaaggacaatatctgtgccgtgcttcaaactctgcaggacaagaacaaaccagagcaacattgacagtgaaaggagatcaaccacttctcaatggacacgctggacaggctgttgaaagtgaacttcgtgtaacaaagcacttgggaggagaaattgtgaataatggagagtcagttacatttgaagctagagtgcaaggaacaccagaagaagtgttatggatgagaaatggacaagaattgacaaatggagataagacaagcatcagtcaagatggtgaaacactatctttcactatcaactcagcagacgcatcagatgccggacactaccaactggaagttcgttcaaaaggaacaaatctcgtatctgttgcatccctggtagtagttggagagaaagctgatcctccagtcaccaggctaccatcctctgtctctgctcctctcggcggatcaacagcatttacgattgaatttgaaaatgttgaaggtctgacagtacaatggttccgcggatcggaaaaaattgaaaagaatgaacgggtcaaatcagtcaagaccggaaacacctttaagcttgacattaagaacgtcgaacaggacgacgacggcatctatgtcgccaaagtagtaaaagagaagaaggcaattgcaaaatatgcggcagctcttctccttgtctaatcatcctgccaccaccactttaaacttcattatatttgtatctattttctgtagtcttcacatctagcctcctatgtctctttcttttattttaatttcaatttccattctctcctcctaatttctgaaattttctccgttttcgccaaatttctttcgccatccgatattcgtgtttgttttatcgtttttttttcaatcaagtaataagcccggcccctgtcaaaacgatgctttctttattgtaatcatccaaaattctaattgatttttcttaacatttttccctttaaaaacacacaaacctgcttcattgtaattttcgagaagtttcagtttttttcaatgttttccagcaaaactttacaaaattttaagtcaaatttcaaaacatttttctatgatttatctgtttttatttcttcattttccatttccttcaatgattcaagaattgggacgcttgtttcagaacgtggaattttaatttcaattgtttctcatttctccatgatttctcttttgtttccatctttttcagtcagattttcagtttgggaccctgaatctctctgatccactttccatgattaggtatcaattcttaatatttgctgattatgcttgaattcgggacaaattcttcataaggttgcaatttttatgtctatttcttgagtatttttccaatagctttagtttggtagactccacatatcctggtgttttcttttctcacgattttttccattgtatgcgttctgaaatttgacttgaaaattttctgcttcttgtagatgctcaaaaaacttgaaattctactgaaactgctcatcttctctaaaaactgtaaatctttcatctagtcagtctgaggtgtttttatttctctctattttctctggaattcaaatgttgttctaatacataataatagtaaacgaataatattgt

SEQ ID NO: 52 - DNA, 7350 bp, Artificial sequence; identical to SEQ ID NO: 50
  C. elegans Luciferase ( FJ376737.1 Cloning vector pmirGLO, complete sequence)

SEQ ID NO: 53 - DNA, 24 bp, Artificial sequence
  oligonucleotide
augaguugggucuaacccauaacu

SEQ ID NO: 54 - DNA, 24 bp, Artificial sequence
  oligonucleotide
auggguucggucaacccauaacuc

SEQ ID NO: 55 - DNA, 24 bp, Artificial sequence
  oligonucleotide
augaaaauuuugauuuacgaauug

SEQ ID NO: 56 - DNA, 24 bp, Artificial sequence
  oligonucleotide
aguuauggguuagacccaacucau

SEQ ID NO: 57 - DNA, 24 bp, Artificial sequence
  oligonucleotide
gaguuauggguugaccgaacccau

SEQ ID NO: 58 - DNA, 24 bp, Artificial sequence
  oligonucleotide
caauucguaaaucaaaauuuuaau

SEQ ID NO: 59 - DNA, 21 bp, Artificial sequence
  oligonucleotide
auguguauagggaagcuaauc

SEQ ID NO: 60 - DNA, 21 bp, Artificial sequence
  oligonucleotide
auguguauagggaagcuaauc

SEQ ID NO: 61 - DNA, 21 bp, Artificial sequence
  oligonucleotide
uauaugaacauuaauaacugg

SEQ ID NO: 62 - DNA, 21 bp, Artificial sequence
  oligonucleotide
gauuagcuucccuauacacau

SEQ ID NO: 63 - DNA, 21 bp, Artificial sequence
  oligonucleotide
gauuagcuucccuauacacau

SEQ ID NO: 64 - DNA, 21 bp, Artificial sequence
  oligonucleotide
ccaguuauuaauguucauaua

SEQ ID NO: 65 - DNA, 78 bp, Artificial sequence
  oligonucleotide
tagccgaattccgtatgagtctttgtctatgtatcttctaacaaggatacaatatttaggcttaaaagatccgttgcg

SEQ ID NO: 66 - DNA, 81 bp, Artificial sequence
  oligonucleotide
gtgtagccgaagtccgtatgagtcttcgtctttgtatcttctaacaagaatacaatacttaggcttttaagatctggttgc

SEQ ID NO: 67 - DNA, 83 bp, Artificial sequence
  oligonucleotide
gtgtagccgaagtccgtacgagtctttgtctttgtatcttctaacaaggacacaatacttaggcttttaagatctggttgcga

SEQ ID NO: 68 - DNA, 83 bp, Artificial sequence; identical to SEQ ID NO: 67
  oligonucleotide

SEQ ID NO: 69 - DNA, 151 bp, Artificial sequence
  oligonucleotide
tcaaaatgggtaacccaactcaactcaactcataatcaaatgagtttagggttaaatgagttatgggttgacccaactcattttgttaaatgggttggatcaacccataactcatttaatttaatgggtgaaattgttaaatgggttaacc

SEQ ID NO: 70 - DNA, 76 bp, Artificial sequence
  oligonucleotide
aactcatttgacccatcaactcatttgagttaaaaatcaacccattagggttcatgggttgagttgagttgagttg

SEQ ID NO: 71 - DNA, 76 bp, Artificial sequence; identical to SEQ ID NO: 70
  oligonucleotide

SEQ ID NO: 72 - DNA, 324 bp, Artificial sequence
  oligonucleotide
gaaaaatgttatttaatacctgaactttcaaaaagtggccaaattaaccgtgaactcttaaaataaccgttttatacctcaacaaaaagttgacttctaatttaatctataagttatcgttgacctaaccaaatcgactcaccattaacagtcatgaacaactttcctaactgcgtaactaacagctgttttcgtccttaaaccaacgataacgactgttagcccatgttttgtaatggctgttaggagcgttgttaagaagtgttaacggtgaatcgatttgccctgatcaacgataacttataggttaaattagaagtcaac

SEQ ID NO: 73 - DNA, 182 bp, Artificial sequence
  oligonucleotide
gacaaataaattgtaaagtttttgttgactttcgaaataaatgacaaagtttttgttgacttgtcatttgagtcatgtcgttaaataggttaacaaaaaaaatttacggcgttaatgtctcgtttatctactctgttagaacaaaacaacgtcgtttattgcagatcattttgtctgttgaa

SEQ ID NO: 74 - DNA, 83 bp, Artificial sequence
  oligonucleotide
tctgcaagtgaaaacttcttacccttttcatatcctagcagctcagaagtctttatgttagttttgaattgaagtgcttgaat

SEQ ID NO: 75 - DNA, 78 bp, Artificial sequence
  oligonucleotide
aactcatttgaccaatcaactcatttgagtcaaaaattttaactcattagggttcatgggttgagttgagttgagttg

SEQ ID NO: 76 - DNA, 157 bp, Artificial sequence
  oligonucleotide
tcaaaatggataactcaactcaactcaactcataattaaatgagtttagggttaaatgagttatgggatgacccaactcattttgttaaatgggttgggtcaacccataactcatttaatttgatgggttgagttgttaaatgggttaacccattta

SEQ ID NO: 77 - DNA, 195 bp, Artificial sequence
  oligonucleotide
taaatgacaaagtttttgttgatttgccatttgagtcatgtcgttaaatagattaacaaaactttttacgatgttaatgttccgtttatctgctctattagaacaaaacgatgtcgtttattgctagacaaaagacggcgttttgtctgttgaaacaaatttaaaccctaaatcctcaaattgatttcattatct

SEQ ID NO: 78 - DNA, 230 bp, Artificial sequence
  oligonucleotide
gacaaataaatcgtaatatttttgttgactttctaaataaatgacaaagtttttgttgatttgccatttgagtcatgtcgttaaatagattaacaaaactttttacgatgttaatgttccgtttatctgctctattagaacaaaacgatgtcgtttattgctagacaaaagacggcgttttgtctgttgaaacaaatttaaaccctaaatcctcaaattgatttcattat

SEQ ID NO: 79 - DNA, 261 bp, Artificial sequence
  oligonucleotide
tttcgaaataaatgacaaagtttttgttgatttcccatttgaatcatgtcgttaaataggttaacagaattattttacggcgttaatgtctcgtgtatatgctctgttagaacaaaacgacgtcgtttattgagagacaaaagaacgtcgttttgtctgttaaaacaaatttaaaccctgaatctccaattcgattttattatctgcgattttgaggtcaaggtgggtctgtgatttcgagataatgaaatcgatttgggg

SEQ ID NO: 80 - DNA, 242 bp, Artificial sequence
  oligonucleotide
taaatgacaaagtttttgttgacttgtcatttaagtcatgtcattagatcggttaacataattttttacggcgttaatgtctcatttatctcctctgttagaacaaaacaatgttgtttattgcggctcgttttgtttgttgaaataaatttaaaccctaaatccccaaatcgacttcaatatctgcgagtttgaggtcaatgtgggtctgtgattttgagataacgaaatcgatttgggga

SEQ ID NO: 81 - DNA, 84 bp, Artificial sequence
  oligonucleotide
tttcgaaataaatgacaaagtttttgtttacttgccatttgactcatatcgttaaataggttaacagattttttttacggcgtt

SEQ ID NO: 82 - DNA, 81 bp, Artificial sequence
  oligonucleotide
aataactcatttgacccatcaactcatttgagtcaaaaattttaactcattaaagttcatgggttgagttgagttgagttg

SEQ ID NO: 83 - DNA, 78 bp, Artificial sequence
  oligonucleotide
aactcatttgacccatcaactcatttaagtcaaaattttcaattcattagggttcatgggttgagttgagttgagttg

SEQ ID NO: 84 - DNA, 84 bp, Artificial sequence
  oligonucleotide
cccactagctcatttgatccatcaactcatttgagtcaaaaaatttaactcattagggttcatgggttgagttgagttgagttg

SEQ ID NO: 85 - DNA, 85 bp, Artificial sequence
  oligonucleotide
acccatcaaattaaatgagttatgggttgacccaactcattatgttaaataggttgggtcaacccataacttatttaattctaaa

SEQ ID NO: 86 - DNA, 84 bp, Artificial sequence
  oligonucleotide
cccaataactcatttgacccatcaactcatttaagtcaaaaatttcaactcattagggtttttgggttgagttgagttgagttg

SEQ ID NO: 87 - DNA, 204 bp, Artificial sequence
  oligonucleotide
tttctaaataaatgacaaagtttttgttgacttgtcatttgagtcatgtcgttaaataggttgacaaaaaaatttagtcgttaatgtcccgtttatctgctctgttagaacaaaacaacgtcctttattgcagagacaaaacaaaatcgttttgtctctagaaacaaatttaaaccctaaatccccaaatcgatttcatgatct

SEQ ID NO: 88 - DNA, 212 bp, Artificial sequence
  oligonucleotide
ttgttgactttctaaataaatgacaaagtttttgttgacttgtcatttgagtcatgtcgttaaataggttgacaaaaaaatttagtcgttaatgtcccgtttatctgctctgttagaacaaaacaacgtcctttattgcagagacaaaacaaaatcgttttgtctctagaaacaaatttaaaccctaaatccccaaatcgatttcatgatct

SEQ ID NO: 89 - DNA, 188 bp, Artificial sequence
  oligonucleotide
aataaatgtcaaagtttttgttaacttgtcatttcagtcatgtcgttaagtaggttaacataatttttttacggcgttaatgtctcgtgtatcacctctgttagaacaaaacaacgttgtttattgcagctcgttttgtctgttgaaacaaatataaaccctaaatacccaaatcgatttcattatct

SEQ ID NO: 90 - DNA, 204 bp, Artificial sequence
  oligonucleotide
tttctaaataaatgacaaagtttttgttgacttgccatttgagtcatgtcgttaaatagattaacaagattttttacgatgttaatgttccgtttatttgctttgttagaacaaaacgatgtcgtttattgcatacacaaacgacgtcgttttgtttgttgaaacaaatttaaactctaaatccccaagttgattttattatct

SEQ ID NO: 91 - DNA, 85 bp, Artificial sequence
  oligonucleotide
cccaataactcatttgacccatcaactcatttgagtcaaaaaaaaataactcattagagttcatgggttgagttgagttgagttg

SEQ ID NO: 92 - DNA, 228 bp, Artificial sequence
  oligonucleotide
aaataaatcgtaaagtgtttgttcactttctaaataaatgacaaagtttttgttgacttgccatttgagtcatgtcgttaaatagattaacaagattttttacgatgttaatgttccgtttatttgctttgttagaacaaaacgatgtcgtttattgcatacacaaacgacgtcgttttgtttgttgaaacaaatttaaactctaaatccccaagttgattttattat

SEQ ID NO: 93 - DNA, 82 bp, Artificial sequence
  oligonucleotide
gtgtagccgtagtccgtatgagtctttgtctttgtatcttcgaacaaggatataatacttaggcttttaagatccagttgcg

SEQ ID NO: 94 - DNA, 82 bp, Artificial sequence; identical to SEQ ID NO: 93
  oligonucleotide

SEQ ID NO: 95 - DNA, 81 bp, Artificial sequence
  oligonucleotide
gtgtagctgaagtccgtatgagtctttgtctttgtatcttctaacaaggaaacactacttatgcttttatgatccggttgc

SEQ ID NO: 96 - DNA, 81 bp, Artificial sequence; identical to SEQ ID NO: 95
  oligonucleotide

SEQ ID NO: 97 - DNA, 157 bp, Artificial sequence
  oligonucleotide
tcaaaatgggtaacccaactcaactcaactcataatcaaatgagtttagggttaaatgagttatgggttgacccaactcattttgttaaatgggttgggtcaacccataactcatttaatttgatgggttgagttgttaaatgagttaacctattta

SEQ ID NO: 98 - DNA, 80 bp, Artificial sequence
  oligonucleotide
gtgtagtcgaattccgtatgagtctttgtctttgtatcttctaacaagggaacaatacataggcttttaagatcaggttg

SEQ ID NO: 99 - DNA, 84 bp, Artificial sequence
  oligonucleotide
cccaataactcatttgacccatcaactcatttgagtcaaaaaatttaactcattagggttcatgggttgagttaaattgagttg

SEQ ID NO: 100 - DNA, 80 bp, Artificial sequence
  oligonucleotide
gtgtagtcgaatttcatatgagtctttgtctttgtatcttctaacaagggaacaatacttaggcttttaagatcaggttg

SEQ ID NO: 101 - DNA, 81 bp, Artificial sequence
  oligonucleotide
gtgtagctgaaggccatatgagtctttgtattgtataatctaacaaggatataatacttaggcttttaagatgcggttgcg

SEQ ID NO: 102 - DNA, 85 bp, Artificial sequence
  oligonucleotide
acccatcaaattaaatgagttatgggttgacccaactcattttgttaaatgagttgggtcaacccataactcatttaattctaaa

SEQ ID NO: 103 - DNA, 155 bp, Artificial sequence
  oligonucleotide
aaaatgggtaacccaactcaactcaactcataatcaaatgagtttagggttaaatgagttatgggttgacccaactcattttgttaaatgggttaggtcaacccataactcatttaatttgatgggttgagttgttaaatggattaacccattta

SEQ ID NO: 104 - DNA, 446 bp, Artificial sequence
  oligonucleotide
catgaattagagttgtgaatgtaaaggtatcaggtttatatcccatttcgaccatttgatcaaccaaagctacggcatcagaaatcctcttactgtgacagtagccattgagcagcgaagaaagcgtgacaatatcgggctcatagccgagtttcatcatcttggcaagaacagctaaagcaagagagagctgagagcgtcggcaaaagcagttgatgaaaatactgtatgtgtaaagatcatgtgaaattcccaaagtttgcatctgctcgccgagagagatgacaagttcaaacttgttcatcttagcaacggcactcaacagtttattgaattcaacaatggaaggaaatggacgagacttgaccatgtcaccgaacagatcaaccgcatcatctaccttaataatatcacttagcctatttctcaatatctctctgtaatca

SEQ ID NO: 105 - DNA, 446 bp, Artificial sequence; identical to SEQ ID NO: 104
  oligonucleotide

SEQ ID NO: 106 - DNA, 409 bp, Artificial sequence
  oligonucleotide
catgaatcaaagttgtaaatgtgattgtgtcaggtctataccccatttccaccatttgatcaaccaaagctacagcatctgaaatcctcttaccgtgacagtatccattgagaagagaagaaagcgtgacaatgctgggctcatagccgagtttcatcatcttgccaagaagagctaaagcaagagagatttgagagcgtcggcagaaacagttgatcaaaatattgtaagtatatagattatgtgaaattcctaacctctgcatcttctcacctagagagatgacaagatcaaactttttcatcttagcaatggcactcaacagtttattgaactcaaaaatggaaggtaaaggacgtgacttgaccatgccaccgaacaaaccaattgcatcatctaacttcaaatc

SEQ ID NO: 107 - DNA, 443 bp, Artificial sequence
  oligonucleotide
catgaatcaaagttgtaaatgtgattgtgtcaggtctataccccatttccaccatttgatcaaccaaagctacagcatctgaaatcctcttaccgtgacagtatccattgagaagagaagaaagcgtgacaatgctgggctcatagccgagtttcatcatcttgccaagaagagctaaagcaagagagatttgagagcgtcggcagaaacagttgatcaaaatattgtaagtatatagattatgtgaaattcctaacctctgcatcttctcacctagagagatgacaagatcaaactttttcatcttagcaatggcactcaacagtttattgaactcaaaaatggaaggtaaaggacgtgacttgaccatgccaccgaacaaaccaattgcatcatctaacttcatactatgtaacccatttctcaatatctctctgtaatca

SEQ ID NO: 108 - DNA, 78 bp, Artificial sequence
  oligonucleotide
aactcatttgacccatcaactcatttgagtcaaaattttcaactcattagggttcatgagttgagttgagttgagttg

SEQ ID NO: 109 - DNA, 75 bp, Artificial sequence
  oligonucleotide
catttttgagattttacacagtagtcatggagtttttggaagagagaaagtggagatgtggagatcgtggggatg

SEQ ID NO: 110 - DNA, 75 bp, Artificial sequence; identical to SEQ ID NO: 109
  oligonucleotide

SEQ ID NO: 111 - DNA, 75 bp, Artificial sequence; identical to SEQ ID NO: 109
  oligonucleotide

SEQ ID NO: 112 - DNA, 75 bp, Artificial sequence; identical to SEQ ID NO: 109
  oligonucleotide

SEQ ID NO: 113 - DNA, 75 bp, Artificial sequence; identical to SEQ ID NO: 109
  oligonucleotide

SEQ ID NO: 114 - DNA, 75 bp, Artificial sequence; identical to SEQ ID NO: 109
  oligonucleotide

SEQ ID NO: 115 - DNA, 78 bp, Artificial sequence
  oligonucleotide
aactcatttgacccatcaactcatttgagtcaaaattttgaactcattagagttcatgggttgagttgaattgagttg

SEQ ID NO: 116 - DNA, 84 bp, Artificial sequence
  oligonucleotide
cccaataactcatttgacccatcaactcatttgagtcaaaaatttcaactcattagggttcaaaagttgcgttgagttgagttg

SEQ ID NO: 117 - DNA, 85 bp, Artificial sequence
  oligonucleotide
cccaataactcatttgacccatcaactcatttgagtcaaaattttctaactcattagagttcatgagttgagttgagttgagttg

SEQ ID NO: 118 - DNA, 75 bp, Artificial sequence
  oligonucleotide
gttaaatgagttatgggttgatccataactcatttaatttgatgggttgagttgttaaatgggttaacccattta

SEQ ID NO: 119 - DNA, 157 bp, Artificial sequence; identical to SEQ ID NO: 4
  oligonucleotide

SEQ ID NO: 120 - DNA, 157 bp, Artificial sequence
  oligonucleotide
tggctccgaaaggaaatctgcggttaaattacggataaaggacagtgcggttcaaataataacaaaaatatatacatacatatatatatgcagaaaatttcgttactatttgaaatgcaccgcactgtgcagttcagtcaccatttgaagcctatat

SEQ ID NO: 121 - DNA, 82 bp, Artificial sequence
  oligonucleotide
gtgtagccgaagtccgtatgagtctttgtctttgtatcttctagcaaggatacaatacttaggcttttaagatctggttacg

SEQ ID NO: 122 - DNA, 82 bp, Artificial sequence; identical to SEQ ID NO: 121
  oligonucleotide

SEQ ID NO: 123 - DNA, 20 bp, Artificial sequence
  oligonucleotide
aggcttaaaagatccgttgc

SEQ ID NO: 124 - DNA, 21 bp, Artificial sequence
  oligonucleotide
aggcttttaagatctggttgc

SEQ ID NO: 125 - DNA, 21 bp, Artificial sequence
  oligonucleotide
aggcttttaagatctggttgc

SEQ ID NO: 126 - DNA, 21 bp, Artificial sequence
  oligonucleotide
aggcttttaagatctggttgc

SEQ ID NO: 127 - DNA, 23 bp, Artificial sequence
  oligonucleotide
atgggttggatcaacccataact

SEQ ID NO: 128 - DNA, 22 bp, Artificial sequence
  oligonucleotide
tgggttgagttgagttgagttg

SEQ ID NO: 129 - DNA, 22 bp, Artificial sequence
  oligonucleotide
tgggttgagttgagttgagttg

SEQ ID NO: 130 - DNA, 21 bp, Artificial sequence
  oligonucleotide
tgttaagaagtgttaacggtg

SEQ ID NO: 131 - DNA, 20 bp, Artificial sequence
  oligonucleotide
ttgaattgaagtgcttgaat

SEQ ID NO: 132 - DNA, 22 bp, Artificial sequence
  oligonucleotide
tgggttgagttgagttgagttg

SEQ ID NO: 133 - DNA, 23 bp, Artificial sequence
  oligonucleotide
atgggttgggtcaacccataact

SEQ ID NO: 134 - DNA, 22 bp, Artificial sequence
  oligonucleotide
tgggttgagttgagttgagttg

SEQ ID NO: 135 - DNA, 22 bp, Artificial sequence
  oligonucleotide
tgggttgagttgagttgagttg

SEQ ID NO: 136 - DNA, 22 bp, Artificial sequence
  oligonucleotide
tgggttgagttgagttgagttg

SEQ ID NO: 137 - DNA, 23 bp, Artificial sequence
  oligonucleotide
ataggttgggtcaacccataact

SEQ ID NO: 138 - DNA, 22 bp, Artificial sequence
  oligonucleotide
tgggttgagttgagttgagttg

SEQ ID NO: 139 - DNA, 22 bp, Artificial sequence
  oligonucleotide
tgggttgagttgagttgagttg

SEQ ID NO: 140 - DNA, 21 bp, Artificial sequence
  oligonucleotide
aggcttttaagatccagttgc

SEQ ID NO: 141 - DNA, 21 bp, Artificial sequence
  oligonucleotide
aggcttttaagatccagttgc

SEQ ID NO: 142 - DNA, 21 bp, Artificial sequence
  oligonucleotide
atgcttttatgatccggttgc

SEQ ID NO: 143 - DNA, 21 bp, Artificial sequence
  oligonucleotide
atgcttttatgatccggttgc

SEQ ID NO: 144 - DNA, 23 bp, Artificial sequence
  oligonucleotide
atgggttgggtcaacccataact

SEQ ID NO: 145 - DNA, 20 bp, Artificial sequence
  oligonucleotide
aggcttttaagatcaggttg

SEQ ID NO: 146 - DNA, 22 bp, Artificial sequence
  oligonucleotide
tgggttgagttaaattgagttg

SEQ ID NO: 147 - DNA, 20 bp, Artificial sequence
  oligonucleotide
aggcttttaagatcaggttg

SEQ ID NO: 148 - DNA, 21 bp, Artificial sequence
  oligonucleotide
aggcttttaagatgcggttgc

SEQ ID NO: 149 - DNA, 23 bp, Artificial sequence
  oligonucleotide
atgagttgggtcaacccataact

SEQ ID NO: 150 - DNA, 23 bp, Artificial sequence
  oligonucleotide
atgggttaggtcaacccataact

SEQ ID NO: 151 - DNA, 22 bp, Artificial sequence
  oligonucleotide
tgagttgagttgagttgagttg

SEQ ID NO: 152 - DNA, 22 bp, Artificial sequence
  oligonucleotide
agatgtggagatcgtggggatg

SEQ ID NO: 153 - DNA, 22 bp, Artificial sequence
  oligonucleotide
agatgtggagatcgtggggatg

SEQ ID NO: 154 - DNA, 22 bp, Artificial sequence
  oligonucleotide
agatgtggagatcgtggggatg

SEQ ID NO: 155 - DNA, 22 bp, Artificial sequence
  oligonucleotide
agatgtggagatcgtggggatg

SEQ ID NO: 156 - DNA, 22 bp, Artificial sequence
  oligonucleotide
agatgtggagatcgtggggatg

SEQ ID NO: 157 - DNA, 22 bp, Artificial sequence
  oligonucleotide
agatgtggagatcgtggggatg

SEQ ID NO: 158 - DNA, 22 bp, Artificial sequence
  oligonucleotide
tgggttgagttgaattgagttg

SEQ ID NO: 159 - DNA, 22 bp, Artificial sequence
  oligonucleotide
aaagttgcgttgagttgagttg

SEQ ID NO: 160 - DNA, 22 bp, Artificial sequence
  oligonucleotide
tgagttgagttgagttgagttg

SEQ ID NO: 161 - DNA, 25 bp, Artificial sequence
  oligonucleotide
atgagttatgggttgatccataact

SEQ ID NO: 162 - DNA, 23 bp, Artificial sequence
  oligonucleotide
atgggttcggtcaacccataact

SEQ ID NO: 163 - DNA, 21 bp, Artificial sequence
  oligonucleotide
aggcttttaagatctggttac

SEQ ID NO: 164 - DNA, 21 bp, Artificial sequence
  oligonucleotide
aggcttttaagatctggttac

SEQ ID NO: 165 - DNA, 20 bp, Artificial sequence
  oligonucleotide
atttgagtcatgtcgttaaa

SEQ ID NO: 166 - DNA, 20 bp, Artificial sequence
  oligonucleotide
atttgagtcatgtcgttaaa

SEQ ID NO: 167 - DNA, 20 bp, Artificial sequence
  oligonucleotide
atttgagtcatgtcgttaaa

SEQ ID NO: 168 - DNA, 20 bp, Artificial sequence
  oligonucleotide
atttgaatcatgtcgttaaa

SEQ ID NO: 169 - DNA, 20 bp, Artificial sequence
  oligonucleotide
atttaagtcatgtcattaga

SEQ ID NO: 170 - DNA, 20 bp, Artificial sequence
  oligonucleotide
atttgactcatatcgttaaa

SEQ ID NO: 171 - DNA, 20 bp, Artificial sequence
  oligonucleotide
atttgagtcatgtcgttaaa

SEQ ID NO: 172 - DNA, 20 bp, Artificial sequence
  oligonucleotide
atttgagtcatgtcgttaaa

SEQ ID NO: 173 - DNA, 20 bp, Artificial sequence
  oligonucleotide
atttcagtcatgtcgttaag

SEQ ID NO: 174 - DNA, 20 bp, Artificial sequence
  oligonucleotide
atttgagtcatgtcgttaaa

SEQ ID NO: 175 - DNA, 20 bp, Artificial sequence
  oligonucleotide
atttgagtcatgtcgttaaa

SEQ ID NO: 176 - DNA, 21 bp, Artificial sequence
  oligonucleotide
gtgaatgtaaaggtatcaggt

SEQ ID NO: 177 - DNA, 21 bp, Artificial sequence
  oligonucleotide
gtgaatgtaaaggtatcaggt

SEQ ID NO: 178 - DNA, 22 bp, Artificial sequence
  oligonucleotide
gtaaatgtgattgtgtcaggtc

SEQ ID NO: 179 - DNA, 22 bp, Artificial sequence
  oligonucleotide
gtaaatgtgattgtgtcaggtc

SEQ ID NO: 180 - DNA, 21 bp, Artificial sequence
  oligonucleotide
cagtgcggttcaaataataac

SEQ ID NO: 181 - DNA, 79 bp, Artificial sequence
  oligonucleotide
tagccgtaatccgtatgagtctttgtctttgtatcttctaacaaggatacaatatttaggcttttaagatctggttgcg

SEQ ID NO: 182 - DNA, 81 bp, Artificial sequence
  oligonucleotide
gtgtagccgaagtccgtatgagtctttgtctttgtatcctctaacaaggatataatacttaggcttttaagatctggttgc

SEQ ID NO: 183 - DNA, 83 bp, Artificial sequence
  oligonucleotide
gtgtagccgtaatccgtatgagtctttgtctttgtatcttctaacaaggatacaatatttaggcttttaagatctggttgcga

SEQ ID NO: 184 - DNA, 83 bp, Artificial sequence
  oligonucleotide
gtgtagccgaagtccgtatgagtctttgtctttgtatcctctaacaaggatataatacttaggcttttaagatctggttgcga

SEQ ID NO: 185 - DNA, 152 bp, Artificial sequence
  oligonucleotide
tcaaaatgggtaacccaacccaacccaactcataatcaaatgagtttatgattaaatgagttatgggttgacccaactcattttgttaaatgagttgggtctaacccataactcatttcatttgatgggttgagttgttaaatgggttaacc

SEQ ID NO: 186 - DNA, 78 bp, Artificial sequence
  oligonucleotide
aactcatttgacccatcaactcatttgagtcaaaaaatttaactcattagggttcatgggttgagttgagttgagttg

SEQ ID NO: 187 - DNA, 78 bp, Artificial sequence; identical to SEQ ID NO: 186
  oligonucleotide

SEQ ID NO: 188 - DNA, 324 bp, Artificial sequence
  oligonucleotide
gaaaaatgttattttatacctgaactttcaaaaagtggccaaattaaccgtgaatttttgaaatgatcgttttatacctcaacaaaaagttgacttctaatttaacctataagttatcgttgacccggccaaatcgactcaccattaacacttcttaacagctctcctaacagcgtaactaacaactgtttttgtccttaaaccaacaataacggctgttaggccatgttttgtaacggctgttaggaacgctgttaaggagtgttaacggtgagtatatttggccgggtcaacgacaaattataggtcaaattagaagtcaac

SEQ ID NO: 189 - DNA, 182 bp, Artificial sequence
  oligonucleotide
gacaaataaatcgtaaacgttttgttgacttttgaaataaatgacaaagtttttgttgacttgtcatttgagtcatgtcgttaagtaggttaacataaaaaatttaagacgttaatgtctcgtttatctcctccgtaaaaacaaaacaacgttgtttattgaagctcgttttgtctgttgaa

SEQ ID NO: 190 - DNA, 83 bp, Artificial sequence
  oligonucleotide
tctgcaagtggaaacttcttacttttatcatatcccatcagctcgaaagtcttgatgttagttttgaattgaagtgcttgaat

SEQ ID NO: 191 - DNA, 78 bp, Artificial sequence; identical to SEQ ID NO: 186
  oligonucleotide

SEQ ID NO: 192 - DNA, 157 bp, Artificial sequence; identical to SEQ ID NO: 1
  oligonucleotide

SEQ ID NO: 193 - DNA, 186 bp, Artificial sequence
  oligonucleotide
taaatgacaaagtttatgttgacttttcatttgagtcatgtcgttaagtaggttaatataattatttcccggcgttaagttctcgtttatctcctctattagaacaaaacaacattgtttattgcatctcgttttgtctgttgaaacaaatttaaaccctaaatccccaaatcgatttcattatct

SEQ ID NO: 194 - DNA, 221 bp, Artificial sequence
  oligonucleotide
gacaaataaatcgtaaacgttttgttgacttttgaaataaatgacaaagtttttgttgacttgtcatttgagtcatgtcgttaagtaggttaacataaaaaatttaagacgttaatgtctcgtttatctcctccgtaaaaacaaaacaacgttgtttattgaagctcgttttgtctgttgaaacaaatttaaaccctaaatcctcaaatcgatttcattat

SEQ ID NO: 195 - DNA, 251 bp, Artificial sequence
  oligonucleotide
tttcgaaataaatgacaaagtttttgttgacttgtcatttgagtcatgtcgttaagtaggttgacataatttttttacggcgttaatgtctcgtttatctcctctgttagaacaaaacaacgttgtttattgcagctcgttttctctattgaaacaaatttaaaccttaaattcccaaatcgattttattatctgcgattttgaggtcaatgtgggtatgtgattttgagataatgaaatcgatttagggg

SEQ ID NO: 196 - DNA, 242 bp, Artificial sequence
  oligonucleotide
taaatgacaaagtttatgttgacttttcatttgagtcatgtcgttaagtaggttaatataattatttcccggcgttaagttctcgtttatctcctctattagaacaaaacaacattgtttattgcatctcgttttgtctgttgaaacaaatttaaaccctaaatccccaaatcgatttcattatctgcgattttgaggtcaggtgggtttgtgattttgagataatgaaatcgatataggga

SEQ ID NO: 197 - DNA, 84 bp, Artificial sequence
  oligonucleotide
tttcgaaataaatgacaaagtttttgttgacttgtcatttgagtcatgtcgttaagtaggttgacataatttttttacggcgtt

SEQ ID NO: 198 - DNA, 81 bp, Artificial sequence
  oligonucleotide
aataactcatttgacccatcaactcatttgagtcaaaaaatttaactcattagggttcatgggttgagttgagttgagttg

SEQ ID NO: 199 - DNA, 78 bp, Artificial sequence; identical to SEQ ID NO: 186
  oligonucleotide

SEQ ID NO: 200 - DNA, 84 bp, Artificial sequence
  oligonucleotide
cccaataactcatttgacccatcaactcatttgagtcaaaaaatttaactcattagggttcatgggttgagttgagttgagttg

SEQ ID NO: 201 - DNA, 86 bp, Artificial sequence
  oligonucleotide
acccatcaaatgaaatgagttatgggttgacccaactcattttgttaaatgagttgggtctaacccataactcatttaatcataaa

SEQ ID NO: 202 - DNA, 84 bp, Artificial sequence; identical to SEQ ID NO: 200
  oligonucleotide

SEQ ID NO: 203 - DNA, 194 bp, Artificial sequence
  oligonucleotide
tttcgaaataaatgacaaagtttttgttgacttgtcatttgagtcatgtcgttaagtaggttgacataatttttttacggcgttaatgtctcgtttatctcctctgttagaacaaaacaacgttgtttattgcagctcgttttctctattgaaacaaatttaaaccttaaattcccaaatcgattttattatct

SEQ ID NO: 204 - DNA, 202 bp, Artificial sequence
  oligonucleotide
ttgttgactttcgaaataaataacaaagtttttgttgacttgtcatttgagtcatgtcgttaagtaggttaacaaaaaaaatttacggcgttaatgtctcctttatctcctctgttagaacaaaaaaacgttgtttattgcagttcgttttgtctgttgacacaaatttaaaccctaaatccccaaatcgattttattatct

SEQ ID NO: 205 - DNA, 188 bp, Artificial sequence
  oligonucleotide
aataaatgacaaagtttttgttgacttgtcatttgagtcatgtcgttaagtaggttgacataatttttttacggcgttaatgtctcgtttatctcctctgttagaacaaaacaacgttgtttattgcagctcgttttctctattgaaacaaatttaaaccttaaattcccaaatcgattttattatct

SEQ ID NO: 206 - DNA, 194 bp, Artificial sequence; identical to SEQ ID NO: 203
  oligonucleotide

SEQ ID NO: 207 - DNA, 84 bp, Artificial sequence; identical to SEQ ID NO: 200
  oligonucleotide

SEQ ID NO: 208 - DNA, 218 bp, Artificial sequence
  oligonucleotide
aaataaatcgtaaacgttttgttgacttttgaaataaatgacaaagtttttgttgacttgtcatttgagtcatgtcgttaagtaggttaacataaaaaatttaagacgttaatgtctcgtttatctcctccgtaaaaacaaaacaacgttgtttattgaagctcgttttgtctgttgaaacaaatttaaaccctaaatcctcaaatcgatttcattat

SEQ ID NO: 209 - DNA, 82 bp, Artificial sequence
  oligonucleotide
gtgtagccgtaatccgtatgagtctttgtctttgtatcttctaacaaggatacaatatttaggcttttaagatctggttgcg

SEQ ID NO: 210 - DNA, 82 bp, Artificial sequence
  oligonucleotide
gtgtagccgaagtccgtatgagtctttgtctttgtatcctctaacaaggatataatacttaggcttttaagatctggttgcg

SEQ ID NO: 211 - DNA, 81 bp, Artificial sequence
  oligonucleotide
gtgtagccgtaatccgtatgagtctttgtctttgtatcttctaacaaggatacaatatttaggcttttaagatctggttgc

SEQ ID NO: 212 - DNA, 81 bp, Artificial sequence; identical to SEQ ID NO: 182
  oligonucleotide

SEQ ID NO: 213 - DNA, 157 bp, Artificial sequence; identical to SEQ ID NO: 1
  oligonucleotide

SEQ ID NO: 214 - DNA, 80 bp, Artificial sequence
  oligonucleotide
gtgtagccgtaatccgtatgagtctttgtctttgtatcttctaacaaggatacaatatttaggcttttaagatctggttg

SEQ ID NO: 215 - DNA, 84 bp, Artificial sequence; identical to SEQ ID NO: 200
  oligonucleotide

SEQ ID NO: 216 - DNA, 80 bp, Artificial sequence; identical to SEQ ID NO: 214
  oligonucleotide

SEQ ID NO: 217 - DNA, 82 bp, Artificial sequence; identical to SEQ ID NO: 210
  oligonucleotide

SEQ ID NO: 218 - DNA, 86 bp, Artificial sequence; identical to SEQ ID NO: 201
  oligonucleotide

SEQ ID NO: 219 - DNA, 155 bp, Artificial sequence
  oligonucleotide
aaaatgggtaacccaacccaacccaactcataatcaaatgagtttatgattaaatgagttatgggttgacccaactcattttgttaaatgagttgggtctaacccataactcatttcatttgatgggttgagttgttaaatgggttaaccattta

SEQ ID NO: 220 - DNA, 443 bp, Artificial sequence
  oligonucleotide
catgaattagagtgttgaatgtgaatgaatcgggctgatatcccatttccaccatctgaccaacaagagatacggcatctgaaatcctattcccgtgacagaaaccattgagcagagagttaagcgtgacaatatcgggctcatagccgagtttcatcatcttggcaagaacagctaaagcaagagagagctgagagcgtcggcagaaacagttgatgagaatactgtatgtgtaaagattatgtgaaattcccaaattttgcatctgctcgccgagagagatgacaagatcgaacttgttcatcttagcaatggcactcaacagtttactgaactcaacaatcgaagggaaaggacgagacttgaccatgtcaccgaacagattaaccgcatcatctagcttcaaatcatttagcctatttatcgatatttttctgtaatca

SEQ ID NO: 221 - DNA, 443 bp, Artificial sequence; identical to SEQ ID NO: 220
  oligonucleotide

SEQ ID NO: 222 - DNA, 409 bp, Artificial sequence
  oligonucleotide
catgaattagagtgttgaatgtgaatgaatcgggctgatatcccatttccaccatctgaccaacaagagatacggcatctgaaatcctattcccgtgacagaaaccattgagcagagagttaagcgtgacaatatcgggctcatagccgagtttcatcatcttggcaagaacagctaaagcaagagagagctgagagcgtcggcagaaacagttgatgagaatactgtatgtgtaaagattatgtgaaattcccaaattttgcatctgctcgccgagagagatgacaagatcgaacttgttcatcttagcaatggcactcaacagtttactgaactcaacaatcgaagggaaaggacgagacttgaccatgtcaccgaacagattaaccgcatcatctagcttcaaatc

SEQ ID NO: 223 - DNA, 443 bp, Artificial sequence; identical to SEQ ID NO: 220
  oligonucleotide

SEQ ID NO: 224 - DNA, 78 bp, Artificial sequence; identical to SEQ ID NO: 186
  oligonucleotide

SEQ ID NO: 225 - DNA, 75 bp, Artificial sequence
  oligonucleotide
catctttgagattttacacagtagtcatggagtttttggaagagagaaagtggagatgtggagatcgtggggatg

SEQ ID NO: 226 - DNA, 75 bp, Artificial sequence; identical to SEQ ID NO: 225
  oligonucleotide

SEQ ID NO: 227 - DNA, 75 bp, Artificial sequence; identical to SEQ ID NO: 225
  oligonucleotide

SEQ ID NO: 228 - DNA, 75 bp, Artificial sequence; identical to SEQ ID NO: 225
  oligonucleotide

SEQ ID NO: 229 - DNA, 75 bp, Artificial sequence; identical to SEQ ID NO: 225
  oligonucleotide

SEQ ID NO: 230 - DNA, 75 bp, Artificial sequence; identical to SEQ ID NO: 225
  oligonucleotide

SEQ ID NO: 231 - DNA, 78 bp, Artificial sequence; identical to SEQ ID NO: 186
  oligonucleotide

SEQ ID NO: 232 - DNA, 84 bp, Artificial sequence; identical to SEQ ID NO: 200
  oligonucleotide

SEQ ID NO: 233 - DNA, 84 bp, Artificial sequence; identical to SEQ ID NO: 200
  oligonucleotide

SEQ ID NO: 234 - DNA, 73 bp, Artificial sequence
  oligonucleotide
gttaaatgagttgggtctaacccataactcatttcatttgatgggttgagttgttaaatgggttaaccattta

SEQ ID NO: 235 - DNA, 157 bp, Artificial sequence; identical to SEQ ID NO: 1
  oligonucleotide

SEQ ID NO: 236 - DNA, 155 bp, Artificial sequence
  oligonucleotide
tggctccgaatggagacgtgcagttcaactgcggataaagtgttgtgcggttcaaatagtaacaaaaatatatacatacatatatatgtaaagaatttcgttactatttgaatcgtactgcactgtacggttcggtcaccattcgaagcctatat

SEQ ID NO: 237 - DNA, 82 bp, Artificial sequence; identical to SEQ ID NO: 209
  oligonucleotide

SEQ ID NO: 238 - DNA, 82 bp, Artificial sequence; identical to SEQ ID NO: 210
  oligonucleotide

SEQ ID NO: 239 - DNA, 21 bp, Artificial sequence
  oligonucleotide
aggcttttaagatctggttgc

SEQ ID NO: 240 - DNA, 21 bp, Artificial sequence
  oligonucleotide
aggcttttaagatctggttgc

SEQ ID NO: 241 - DNA, 21 bp, Artificial sequence
  oligonucleotide
aggcttttaagatctggttgc

SEQ ID NO: 242 - DNA, 21 bp, Artificial sequence
  oligonucleotide
aggcttttaagatctggttgc

SEQ ID NO: 243 - DNA, 24 bp, Artificial sequence
  oligonucleotide
atgagttgggtctaacccataact

SEQ ID NO: 244 - DNA, 24 bp, Artificial sequence
  oligonucleotide
tgggttgagttgagttgagttggc

SEQ ID NO: 245 - DNA, 24 bp, Artificial sequence
  oligonucleotide
tgggttgagttgagttgagttggc

SEQ ID NO: 246 - DNA, 21 bp, Artificial sequence
  oligonucleotide
tgttaaggagtgttaacggtg

SEQ ID NO: 247 - DNA, 21 bp, Artificial sequence
  oligonucleotide
ttgaattgaagtgcttgaatt

SEQ ID NO: 248 - DNA, 24 bp, Artificial sequence
  oligonucleotide
tgggttgagttgagttgagttggc

SEQ ID NO: 249 - DNA, 24 bp, Artificial sequence
  oligonucleotide
atgagttgggtctaacccataact

SEQ ID NO: 250 - DNA, 24 bp, Artificial sequence
  oligonucleotide
tgggttgagttgagttgagttggc

SEQ ID NO: 251 - DNA, 24 bp, Artificial sequence
  oligonucleotide
tgggttgagttgagttgagttggc

SEQ ID NO: 252 - DNA, 24 bp, Artificial sequence
  oligonucleotide
tgggttgagttgagttgagttggc

SEQ ID NO: 253 - DNA, 24 bp, Artificial sequence
  oligonucleotide
atgagttgggtctaacccataact

SEQ ID NO: 254 - DNA, 24 bp, Artificial sequence
  oligonucleotide
tgggttgagttgagttgagttggc

SEQ ID NO: 255 - DNA, 24 bp, Artificial sequence
  oligonucleotide
tgggttgagttgagttgagttggc

SEQ ID NO: 256 - DNA, 21 bp, Artificial sequence
  oligonucleotide
aggcttttaagatctggttgc

SEQ ID NO: 257 - DNA, 21 bp, Artificial sequence
  oligonucleotide
aggcttttaagatctggttgc

SEQ ID NO: 258 - DNA, 21 bp, Artificial sequence
  oligonucleotide
aggcttttaagatctggttgc

SEQ ID NO: 259 - DNA, 21 bp, Artificial sequence
  oligonucleotide
aggcttttaagatctggttgc

SEQ ID NO: 260 - DNA, 24 bp, Artificial sequence
  oligonucleotide
atgagttgggtctaacccataact

SEQ ID NO: 261 - DNA, 21 bp, Artificial sequence
  oligonucleotide
aggcttttaagatctggttgc

SEQ ID NO: 262 - DNA, 24 bp, Artificial sequence
  oligonucleotide
tgggttgagttgagttgagttggc

SEQ ID NO: 263 - DNA, 21 bp, Artificial sequence
  oligonucleotide
aggcttttaagatctggttgc

SEQ ID NO: 264 - DNA, 21 bp, Artificial sequence
  oligonucleotide
aggcttttaagatctggttgc

SEQ ID NO: 265 - DNA, 24 bp, Artificial sequence
  oligonucleotide
atgagttgggtctaacccataact

SEQ ID NO: 266 - DNA, 24 bp, Artificial sequence
  oligonucleotide
atgagttgggtctaacccataact

SEQ ID NO: 267 - DNA, 24 bp, Artificial sequence
  oligonucleotide
tgggttgagttgagttgagttggc

SEQ ID NO: 268 - DNA, 22 bp, Artificial sequence
  oligonucleotide
agatgtggagatcgtggggatg

SEQ ID NO: 269 - DNA, 22 bp, Artificial sequence
  oligonucleotide
agatgtggagatcgtggggatg

SEQ ID NO: 270 - DNA, 22 bp, Artificial sequence
  oligonucleotide
agatgtggagatcgtggggatg

SEQ ID NO: 271 - DNA, 22 bp, Artificial sequence
  oligonucleotide
agatgtggagatcgtggggatg

SEQ ID NO: 272 - DNA, 22 bp, Artificial sequence
  oligonucleotide
agatgtggagatcgtggggatg

SEQ ID NO: 273 - DNA, 22 bp, Artificial sequence
  oligonucleotide
agatgtggagatcgtggggatg

SEQ ID NO: 274 - DNA, 24 bp, Artificial sequence
  oligonucleotide
tgggttgagttgagttgagttggc

SEQ ID NO: 275 - DNA, 24 bp, Artificial sequence
  oligonucleotide
tgggttgagttgagttgagttggc

SEQ ID NO: 276 - DNA, 24 bp, Artificial sequence
  oligonucleotide
tgggttgagttgagttgagttggc

SEQ ID NO: 277 - DNA, 24 bp, Artificial sequence
  oligonucleotide
atgagttgggtctaacccataact

SEQ ID NO: 278 - DNA, 24 bp, Artificial sequence
  oligonucleotide
atgagttgggtctaacccataact

SEQ ID NO: 279 - DNA, 21 bp, Artificial sequence
  oligonucleotide
aggcttttaagatctggttgc

SEQ ID NO: 280 - DNA, 21 bp, Artificial sequence
  oligonucleotide
aggcttttaagatctggttgc

SEQ ID NO: 281 - DNA, 20 bp, Artificial sequence
  oligonucleotide
atttgagtcatgtcgttaag

SEQ ID NO: 282 - DNA, 21 bp, Artificial sequence
  oligonucleotide
cattcaaggacttctattcag

SEQ ID NO: 283 - DNA, 20 bp, Artificial sequence
  oligonucleotide
atttgagtcatgtcgttaag

SEQ ID NO: 284 - DNA, 20 bp, Artificial sequence
  oligonucleotide
atttgagtcatgtcgttaag

SEQ ID NO: 285 - DNA, 20 bp, Artificial sequence
  oligonucleotide
atttgagtcatgtcgttaag

SEQ ID NO: 286 - DNA, 20 bp, Artificial sequence
  oligonucleotide
atttgagtcatgtcgttaag

SEQ ID NO: 287 - DNA, 20 bp, Artificial sequence
  oligonucleotide
atttgagtcatgtcgttaag

SEQ ID NO: 288 - DNA, 20 bp, Artificial sequence
  oligonucleotide
atttgagtcatgtcgttaag

SEQ ID NO: 289 - DNA, 20 bp, Artificial sequence
  oligonucleotide
atttgagtcatgtcgttaag

SEQ ID NO: 290 - DNA, 20 bp, Artificial sequence
  oligonucleotide
atttgagtcatgtcgttaag

SEQ ID NO: 291 - DNA, 20 bp, Artificial sequence
  oligonucleotide
atttgagtcatgtcgttaag

SEQ ID NO: 292 - DNA, 20 bp, Artificial sequence
  oligonucleotide
atttgagtcatgtcgttaag

SEQ ID NO: 293 - DNA, 21 bp, Artificial sequence
  oligonucleotide
ttgaatgtgaatgaatcgggc

SEQ ID NO: 294 - DNA, 21 bp, Artificial sequence
  oligonucleotide
ttgaatgtgaatgaatcgggc

SEQ ID NO: 295 - DNA, 21 bp, Artificial sequence
  oligonucleotide
ttgaatgtgaatgaatcgggc

SEQ ID NO: 296 - DNA, 21 bp, Artificial sequence
  oligonucleotide
ttgaatgtgaatgaatcgggc

SEQ ID NO: 297 - DNA, 21 bp, Artificial sequence
  oligonucleotide
ttgtgcggttcaaatagtaac

SEQ ID NO: 298 - DNA, 116 bp, Artificial sequence
  oligonucleotide
tctactttgatctacaaaaaatgcgcggggactgatttcgcatggttaagaaagcgctgacgtcacaacatttttgggcgaaaaactcccgcattttttgtagatcaaaccgtcga

SEQ ID NO: 299 - DNA, 66 bp, Artificial sequence
  oligonucleotide
cccatagaaatattttctatgtcaacctctacaacggttgccgtagttaaattttttctttgggaa

SEQ ID NO: 300 - DNA, 155 bp, Artificial sequence
  oligonucleotide
tgcggcaaatttgccgaatttgccgaatttgccgtttgccgagctcggcaaatttcaaaaaagtagatttgccgaatttgccgtgctcggcaaatattggaaaaatagatttgccgaatttgccgagctcggcaaattttgagatttgccgcaca

SEQ ID NO: 301 - DNA, 122 bp, Artificial sequence
  oligonucleotide
tctacgttgatctacaaaaaatgcggcacagctctcaactgatttcatatggttaggaacgtgctgacgtcacattttttcgggcaaaaatttcccgcattttttgtagatcaaaccgcctg

SEQ ID NO: 302 - DNA, 122 bp, Artificial sequence
  oligonucleotide
tacggtttgatctacaaaaaatgcggcagagatctcaactgatttcgtatggttaggaacgtgctgacgtcacatttttttggggaaaaaaatcccgcattttttgtagaccaaaccgtaat

SEQ ID NO: 303 - DNA, 163 bp, Artificial sequence
  oligonucleotide
tgtagtttgtagtccagcagaccaataccaaaagctttgggtctgccagagactacaaactacacaaatcactagcagaccatacggtttttttttttgcgtagtttgtagtctagcagaccgaaaataggatgcgttggtctgccacactacaaactacaaa

SEQ ID NO: 304 - DNA, 126 bp, Artificial sequence
  oligonucleotide
cgtacgttgatctacaaaaaatgcgggaattgttgatcaactgatttcgcatggttaagaacgtgctgacgtcacattttttggggcgaaaaaaattcccgcattttttgtagatcaaaccgtcaa

SEQ ID NO: 305 - DNA, 112 bp, Artificial sequence
  oligonucleotide
tctactttgatctacaaaaaatgcggcggagttctcaactgattttgcacgtgccgacgtcacattttcttggacaaaaaatccccgcaatttttgtagatcaaaccgtacc

SEQ ID NO: 306 - DNA, 123 bp, Artificial sequence
  oligonucleotide
tacgtcttgatctacaaaaaatgcggcagagttctcaactgatttcgtatggttaggaacgtgctgacgtcacatatttttgggcaaaaaaactcccgcgttttttgtagatcacaccgtgaa

SEQ ID NO: 307 - DNA, 23 bp, Artificial sequence
  oligonucleotide
ttttttgtagatcaaaccgtcga

SEQ ID NO: 308 - DNA, 24 bp, Artificial sequence
  oligonucleotide
gtagttaaattttttctttgggaa

SEQ ID NO: 309 - DNA, 22 bp, Artificial sequence
  oligonucleotide
aaattttgagatttgccgcaca

SEQ ID NO: 310 - DNA, 23 bp, Artificial sequence
  oligonucleotide
ttttttgtagatcaaaccgcctg

SEQ ID NO: 311 - DNA, 23 bp, Artificial sequence
  oligonucleotide
ttttttgtagaccaaaccgtaat

SEQ ID NO: 312 - DNA, 23 bp, Artificial sequence
  oligonucleotide
tctgccacactacaaactacaaa

SEQ ID NO: 313 - DNA, 23 bp, Artificial sequence
  oligonucleotide
ttttttgtagatcaaaccgtcaa

SEQ ID NO: 314 - DNA, 23 bp, Artificial sequence
  oligonucleotide
atttttgtagatcaaaccgtacc

SEQ ID NO: 315 - DNA, 23 bp, Artificial sequence
  oligonucleotide
ttttttgtagatcacaccgtgaa

SEQ ID NO: 316 - DNA, 24 bp, Artificial sequence
  oligonucleotide
tctactttgatctacaaaaaatgc

SEQ ID NO: 317 - DNA, 23 bp, Artificial sequence
  oligonucleotide
cccatagaaatattttctatgtc

SEQ ID NO: 318 - DNA, 23 bp, Artificial sequence
  oligonucleotide
tgcggcaaatttgccgaatttgc

SEQ ID NO: 319 - DNA, 24 bp, Artificial sequence
  oligonucleotide
tctacgttgatctacaaaaaatgc

SEQ ID NO: 320 - DNA, 24 bp, Artificial sequence
  oligonucleotide
tacggtttgatctacaaaaaatgc

SEQ ID NO: 321 - DNA, 23 bp, Artificial sequence
  oligonucleotide
tgtagtttgtagtccagcagacc

SEQ ID NO: 322 - DNA, 24 bp, Artificial sequence
  oligonucleotide
cgtacgttgatctacaaaaaatgc

SEQ ID NO: 323 - DNA, 24 bp, Artificial sequence
  oligonucleotide
tctactttgatctacaaaaaatgc

SEQ ID NO: 324 - DNA, 24 bp, Artificial sequence
  oligonucleotide
tacgtcttgatctacaaaaaatgc

SEQ ID NO: 325 - DNA, 110 bp, Artificial sequence
  oligonucleotide
ggtgaggggtgtgtcccatgccggtttgatctacaaaaaatgcgggagcatagaaaaatcccgcacttttgtagatcaaaccgacatgggacaacctgacactgtgttca

SEQ ID NO: 326 - DNA, 65 bp, Artificial sequence
  oligonucleotide
cccatagaaatattttctatgtcaacctcttgtacggttgctgtagttaattttttctttgggaa

SEQ ID NO: 327 - DNA, 94 bp, Artificial sequence
  oligonucleotide
agtcggaggcaggggtgtgcggcaaatttgccgaatttgccgtttgtcgagctcggcaaattttgagattttccgcacacccctgatcggagcc

SEQ ID NO: 328 - DNA, 110 bp, Artificial sequence; identical to SEQ ID NO: 325
  oligonucleotide

SEQ ID NO: 329 - DNA, 110 bp, Artificial sequence; identical to SEQ ID NO: 325
  oligonucleotide

SEQ ID NO: 330 - DNA, 110 bp, Artificial sequence
  oligonucleotide
ggtctgctaattatttttgtagtttgtagtctagcagaccaataccaaaagctttgggtctgccagactacaaaccacacaaatcactagcagacccttttttttttttt

SEQ ID NO: 331 - DNA, 110 bp, Artificial sequence; identical to SEQ ID NO: 325
  oligonucleotide

SEQ ID NO: 332 - DNA, 110 bp, Artificial sequence; identical to SEQ ID NO: 325
  oligonucleotide

SEQ ID NO: 333 - DNA, 110 bp, Artificial sequence; identical to SEQ ID NO: 325
  oligonucleotide

SEQ ID NO: 334 - DNA, 23 bp, Artificial sequence
  oligonucleotide
acttttgtagatcaaaccgacat

SEQ ID NO: 335 - DNA, 24 bp, Artificial sequence
  oligonucleotide
tgtagttaattttttctttgggaa

SEQ ID NO: 336 - DNA, 22 bp, Artificial sequence
  oligonucleotide
aaattttgagattttccgcaca

SEQ ID NO: 337 - DNA, 23 bp, Artificial sequence
  oligonucleotide
acttttgtagatcaaaccgacat

SEQ ID NO: 338 - DNA, 23 bp, Artificial sequence
  oligonucleotide
acttttgtagatcaaaccgacat

SEQ ID NO: 339 - DNA, 23 bp, Artificial sequence
  oligonucleotide
tctgccagactacaaaccacaca

SEQ ID NO: 340 - DNA, 23 bp, Artificial sequence
  oligonucleotide
acttttgtagatcaaaccgacat

SEQ ID NO: 341 - DNA, 23 bp, Artificial sequence
  oligonucleotide
acttttgtagatcaaaccgacat

SEQ ID NO: 342 - DNA, 23 bp, Artificial sequence
  oligonucleotide
acttttgtagatcaaaccgacat

SEQ ID NO: 343 - DNA, 24 bp, Artificial sequence
  oligonucleotide
gccggtttgatctacaaaaaatgc

SEQ ID NO: 344 - DNA, 23 bp, Artificial sequence
  oligonucleotide
cccatagaaatattttctatgtc

SEQ ID NO: 345 - DNA, 23 bp, Artificial sequence
  oligonucleotide
tgcggcaaatttgccgaatttgc

SEQ ID NO: 346 - DNA, 24 bp, Artificial sequence
  oligonucleotide
gccggtttgatctacaaaaaatgc

SEQ ID NO: 347 - DNA, 24 bp, Artificial sequence
  oligonucleotide
gccggtttgatctacaaaaaatgc

SEQ ID NO: 348 - DNA, 23 bp, Artificial sequence
  oligonucleotide
tgtagtttgtagtctagcagacc

SEQ ID NO: 349 - DNA, 24 bp, Artificial sequence
  oligonucleotide
gccggtttgatctacaaaaaatgc

SEQ ID NO: 350 - DNA, 24 bp, Artificial sequence
  oligonucleotide
gccggtttgatctacaaaaaatgc

SEQ ID NO: 351 - DNA, 24 bp, Artificial sequence
  oligonucleotide
gccggtttgatctacaaaaaatgc

SEQ ID NO: 352 - DNA, 85 bp, Artificial sequence
  oligonucleotide
gtcatgctgtggccctccagagggaagcgctttctgttgtctgaaagaaaacaaagcgctcccctttagaggtttacggtttgag

SEQ ID NO: 353 - DNA, 97 bp, Artificial sequence
  oligonucleotide
tttttctagttattaggttggtgcaaaagtaattgtggtttttgccattactttaaatggcaagaacaacaattacttttgcaccaacctaatagat

SEQ ID NO: 354 - DNA, 101 bp, Artificial sequence
  oligonucleotide
gcaagaagatctcaggctgtcgtcctctagagggaagcactttctgttgtctgaaagaaaagaaagtgcttccttttagagggttaccgtttgagaaaagc

SEQ ID NO: 355 - DNA, 81 bp, Artificial sequence
  oligonucleotide
catgctgtggccctccagagggaagcgctttctgttgtctgaaagaaaacaaagcgctcccctttagaggtttacggtttg

SEQ ID NO: 356 - DNA, 70 bp, Artificial sequence
  oligonucleotide
actggtgcaaaagtaattgcggtttttgccattacttttcttggcaaaaaccgcaattacttgtgcacca

SEQ ID NO: 357 - DNA, 87 bp, Artificial sequence
  oligonucleotide
tgtcatgctgtggccctccagagggaagcgctttctgttgtctgaaagaaaacaaagcgctcccctttagaggtttacggtttgagt

SEQ ID NO: 358 - DNA, 83 bp, Artificial sequence
  oligonucleotide
tctgcaggtcctggtgaacgccatcatcaacagtggtccccgggaggactccatacgtattgggcgagccgagcctgggccgt

SEQ ID NO: 359 - DNA, 70 bp, Artificial sequence
  oligonucleotide
tggtgcaaaagtaattgcggtttttgccattaaaggtaaaatggcaattacttttgtaccaacctatatt

SEQ ID NO: 360 - DNA, 61 bp, Artificial sequence
  oligonucleotide
ccctccagagggaagcgctttctgttgtctgaaagaaaacaaagcgctcccctttagaggt

SEQ ID NO: 361 - DNA, 115 bp, Artificial sequence
  oligonucleotide
ggggccgagggccgtccggcgtcccaggcggggcgccgcgggaccgccctcgtgtctgtggcggtgggatcccgcggccgtgttttcctggtggcccggccgtgcctgaggtttc

SEQ ID NO: 362 - DNA, 98 bp, Artificial sequence
  oligonucleotide
ctctcaccaagcaagtgcagtggggcttgctggcttgcactgtgactccctctcaccaagcaagtgcagtggggcttgctggcttgcaccgtgactca

SEQ ID NO: 363 - DNA, 83 bp, Artificial sequence
  oligonucleotide
tctgcaggtcctggtgaacgccatcatcaacagtggtccccgggaggactccacactcactgggcgagccaggatcgtgagac

SEQ ID NO: 364 - DNA, 83 bp, Artificial sequence
  oligonucleotide
tctgcaggtcctggtgaacgccatcatcaacagtggtccccgggaggactccacacgcattgggtgcgccgggactgtgagac

SEQ ID NO: 365 - DNA, 115 bp, Artificial sequence; identical to SEQ ID NO: 361
  oligonucleotide

SEQ ID NO: 366 - DNA, 116 bp, Artificial sequence
  oligonucleotide
tcaggaggctgaggtggaaggatcgcttgagcctgggaggtcaaggctgcagtgacccgagatcatgccactgtactcaagcctggactacagagtgagaccctgtctcaaaaacg

SEQ ID NO: 367 - DNA, 87 bp, Artificial sequence; identical to SEQ ID NO: 357
  oligonucleotide

SEQ ID NO: 368 - DNA, 93 bp, Artificial sequence
  oligonucleotide
ccgtccggcatcctaggcgggtcgctgcgggacctccctcgtgtctgtggcggtgggatcccgtggccgtgttttcctggtggcccggccgtg

SEQ ID NO: 369 - DNA, 87 bp, Artificial sequence; identical to SEQ ID NO: 357
  oligonucleotide

SEQ ID NO: 370 - DNA, 87 bp, Artificial sequence; identical to SEQ ID NO: 357
  oligonucleotide

SEQ ID NO: 371 - DNA, 180 bp, Artificial sequence
  oligonucleotide
accgccgcgactgcggcggtggtggggggagccgcggggatcgccggagggccggtcggcgccccgggtgccgcgcggtgccgccggggcgtgaggccccgcgcgtgtgtcccggctgcggtcggccgcgctcgaggggtccccgtggcgtccccttccccgccggccgcctttctcgcg

SEQ ID NO: 372 - DNA, 180 bp, Artificial sequence; identical to SEQ ID NO: 371
  oligonucleotide

SEQ ID NO: 373 - DNA, 70 bp, Artificial sequence
  oligonucleotide
tgatgcaaaagtaattgcggtttttgccattacttttgcaccaaccttgtagtaacaaaaggagccagta

SEQ ID NO: 374 - DNA, 61 bp, Artificial sequence
  oligonucleotide
tctcgaggaaagaagcactttctgttgtctgaaagaaaagaaagtgcttcctttcagaggg

SEQ ID NO: 375 - DNA, 87 bp, Artificial sequence
  oligonucleotide
tctcagcctgtgaccctctagagggaagcgctttctgttgtctgaaagaaaagaaagtgcatctttttagaggattacagtttgaga

SEQ ID NO: 376 - DNA, 87 bp, Artificial sequence
  oligonucleotide
tctcatgctgtgaccctctagagggaagcgctttctgttgtctgaaagaaaagaacgcgcttccctatagagggttaccctttgaga

SEQ ID NO: 377 - DNA, 87 bp, Artificial sequence
  oligonucleotide
tctcaggctgtgtccctctacagggaagcgctttctgttgtctgaaagaaaggaaagtgcatccttttagagtgttactgtttgaga

SEQ ID NO: 378 - DNA, 88 bp, Artificial sequence
  oligonucleotide
ttattaggtgggtgcaaaggtaattgcagtttttcccattattttaattgcgaaaacagcaattacctttgcaccaacctgatggagt

SEQ ID NO: 379 - DNA, 85 bp, Artificial sequence
  oligonucleotide
tctcaagctgtgactgcaaagggaagccctttctgttgtctgaaagaagagaaagcgcttccctttgctggattacggtttgaga

SEQ ID NO: 380 - DNA, 115 bp, Artificial sequence
  oligonucleotide
  misc_feature (1)..(67): n is a, c, g, t or u
nnnnnnnnnnnnnnnnnnnnnnnnnnnnnnnnnnnnnnnnnnnnnnnnnnnnnnnnnnnnnnnnnnngatcccgcggccgtgttttcctggtggcccggccgtgcctgaggtttc

SEQ ID NO: 381 - DNA, 61 bp, Artificial sequence; identical to SEQ ID NO: 374
  oligonucleotide

SEQ ID NO: 382 - DNA, 118 bp, Artificial sequence
  oligonucleotide
gtagatcatgattgtattaggttggtgcaaaagtaatcgcagtttttgtcattactttcaatggcaaaaacagcgattgcttttgcaccaacctatacttccttgacatatggacctg

SEQ ID NO: 383 - DNA, 85 bp, Artificial sequence
  oligonucleotide
tcaggctgtgaccctctagagggaagcgctttctgttggctaaaagaaaagaaagcgcttcccttcagagtgttaacgctttgag

SEQ ID NO: 384 - DNA, 87 bp, Artificial sequence
  oligonucleotide
cctcaggctgtgacactctagagggaagcgctttctgttgtctgaaagaaaggaaagtgcatccttttagagtgttactgtttgaga

SEQ ID NO: 385 - DNA, 87 bp, Artificial sequence
  oligonucleotide
tctcaggctgtgaccctccaaagggaagaactttctgttgtctaaaagaaaagaacgcacttccctttagagtgttaccgtgtgaga

SEQ ID NO: 386 - DNA, 87 bp, Artificial sequence
  oligonucleotide
tcccatgctgtgaccctctagagggaagcgctttctgttgtctgaaagaaaagaaagtgcatccttttagaggtttactgtttgagg

SEQ ID NO: 387 - DNA, 129 bp, Artificial sequence
  oligonucleotide
gggatgccacattcagccattcagcgtacagtgcctttctcaaggaggtgtcgtttatgtgaactaaaatataaatttcacctttctgagaagagtaatgtacagcatgcactgcatatgtggtgtccc

SEQ ID NO: 388 - DNA, 127 bp, Artificial sequence
  oligonucleotide
ggatgccacattcagccattcagcgtacagtgcctttctcaaggaggtgtcgtttatgtgaactaaaatataaatttcacctttctgagaagagtaatgtacagcatgcactgcatatgtggtgtcc

SEQ ID NO: 389 - DNA, 87 bp, Artificial sequence
  oligonucleotide
tctcaggctgtgtccctctagagggaagcgctttctgttgtctgaaagaaaagaaaatggttccctttagagtgttacgctttgaga

SEQ ID NO: 390 - DNA, 87 bp, Artificial sequence
  oligonucleotide
tctcaagctgtgagtctacaaaggaaagcgctttctgttgtctgaaagaaaagaaatcgcttccctttggagtgttacggtttgaga

SEQ ID NO: 391 - DNA, 87 bp, Artificial sequence
  oligonucleotide
ctcaggctgtgaccctctagagggaagcgctttctgttggctaaaagaaaagaaagcgcttcccttcagagtgttaacgctttgaga

SEQ ID NO: 392 - DNA, 101 bp, Artificial sequence
  oligonucleotide
gcaagatctcaggctgtgaccttctcgaggaaagaagcactttctgttgtctgaaagaaaagaaagtgcttcctttcagagggttacggtttgagaaaagc

SEQ ID NO: 393 - DNA, 22 bp, Artificial sequence
  oligonucleotide
ggtggcccggccgtgcctgagg

SEQ ID NO: 394 - DNA, 23 bp, Artificial sequence
  oligonucleotide
tgggtgcgccgggactgtgagac

SEQ ID NO: 395 - DNA, 22 bp, Artificial sequence
  oligonucleotide
ggtggcccggccgtgcctgagg

SEQ ID NO: 396 - DNA, 21 bp, Artificial sequence
  oligonucleotide
aaagtgcttcctttcagaggg

SEQ ID NO: 397 - DNA, 22 bp, Artificial sequence
  oligonucleotide
ggtggcccggccgtgcctgagg

SEQ ID NO: 398 - DNA, 21 bp, Artificial sequence
  oligonucleotide
aaagtgcttcctttcagaggg

SEQ ID NO: 399 - DNA, 22 bp, Artificial sequence
  oligonucleotide
gaaagcgcttcccttcagagtg

SEQ ID NO: 400 - DNA, 22 bp, Artificial sequence
  oligonucleotide
aaagtgcatccttttagagtgt

SEQ ID NO: 401 - DNA, 22 bp, Artificial sequence
  oligonucleotide
aacgcacttccctttagagtgt

SEQ ID NO: 402 - DNA, 23 bp, Artificial sequence
  oligonucleotide
taaatttcacctttctgagaaga

SEQ ID NO: 403 - DNA, 23 bp, Artificial sequence
  oligonucleotide
taaatttcacctttctgagaaga

SEQ ID NO: 404 - DNA, 21 bp, Artificial sequence
  oligonucleotide
gaaatcgcttccctttggagt

SEQ ID NO: 405 - DNA, 22 bp, Artificial sequence
  oligonucleotide
ctccagagggaagcgctttctg

SEQ ID NO: 406 - DNA, 22 bp, Artificial sequence
  oligonucleotide
aaaagtaattgtggtttttgcc

SEQ ID NO: 407 - DNA, 23 bp, Artificial sequence
  oligonucleotide
cctctagagggaagcactttctg

SEQ ID NO: 408 - DNA, 22 bp, Artificial sequence
  oligonucleotide
ctccagagggaagcgctttctg

SEQ ID NO: 409 - DNA, 22 bp, Artificial sequence
  oligonucleotide
aaaagtaattgcggtttttgcc

SEQ ID NO: 410 - DNA, 21 bp, Artificial sequence
  oligonucleotide
cctccagagggaagcgctttc

SEQ ID NO: 411 - DNA, 24 bp, Artificial sequence
  oligonucleotide
tctgcaggtcctggtgaacgccat

SEQ ID NO: 412 - DNA, 22 bp, Artificial sequence
  oligonucleotide
aaaagtaattgcggtttttgcc

SEQ ID NO: 413 - DNA, 21 bp, Artificial sequence
  oligonucleotide
cctccagagggaagcgctttc

SEQ ID NO: 414 - DNA, 22 bp, Artificial sequence
  oligonucleotide
tgcagtggggcttgctggcttg

SEQ ID NO: 415 - DNA, 24 bp, Artificial sequence
  oligonucleotide
tctgcaggtcctggtgaacgccat

SEQ ID NO: 416 - DNA, 24 bp, Artificial sequence
  oligonucleotide
tctgcaggtcctggtgaacgccat

SEQ ID NO: 417 - DNA, 21 bp, Artificial sequence
  oligonucleotide
ctgggaggtcaaggctgcagt

SEQ ID NO: 418 - DNA, 22 bp, Artificial sequence
  oligonucleotide
ctccagagggaagcgctttctg

SEQ ID NO: 419 - DNA, 22 bp, Artificial sequence
  oligonucleotide
aggcgggtcgctgcgggacctc

SEQ ID NO: 420 - DNA, 22 bp, Artificial sequence
  oligonucleotide
ctccagagggaagcgctttctg

SEQ ID NO: 421 - DNA, 22 bp, Artificial sequence
  oligonucleotide
ctccagagggaagcgctttctg

SEQ ID NO: 422 - DNA, 21 bp, Artificial sequence
  oligonucleotide
agccgcggggatcgccggagg

SEQ ID NO: 423 - DNA, 21 bp, Artificial sequence
  oligonucleotide
agccgcggggatcgccggagg

SEQ ID NO: 424 - DNA, 22 bp, Artificial sequence
  oligonucleotide
aaaagtaattgcggtttttgcc

SEQ ID NO: 425 - DNA, 22 bp, Artificial sequence
  oligonucleotide
ctctagagggaagcgctttctg

SEQ ID NO: 426 - DNA, 22 bp, Artificial sequence
  oligonucleotide
ctctagagggaagcgctttctg

SEQ ID NO: 427 - DNA, 22 bp, Artificial sequence
  oligonucleotide
ctctacagggaagcgctttctg

SEQ ID NO: 428 - DNA, 22 bp, Artificial sequence
  oligonucleotide
aaaggtaattgcagtttttccc

SEQ ID NO: 429 - DNA, 20 bp, Artificial sequence
  oligonucleotide
ctgcaaagggaagccctttc

SEQ ID NO: 430 - DNA, 22 bp, Artificial sequence
  oligonucleotide
aaaagtaatcgcagtttttgtc

SEQ ID NO: 431 - DNA, 22 bp, Artificial sequence
  oligonucleotide
ctctagagggaagcgctttctg

SEQ ID NO: 432 - DNA, 22 bp, Artificial sequence
  oligonucleotide
ctctagagggaagcgctttctg

SEQ ID NO: 433 - DNA, 22 bp, Artificial sequence
  oligonucleotide
ctctagagggaagcgctttctg

SEQ ID NO: 434 - DNA, 21 bp, Artificial sequence
  oligonucleotide
cctctagagggaagcgctttc

SEQ ID NO: 435 - DNA, 22 bp, Artificial sequence
  oligonucleotide
ctacaaaggaaagcgctttctg

SEQ ID NO: 436 - DNA, 22 bp, Artificial sequence
  oligonucleotide
ctctagagggaagcgctttctg

SEQ ID NO: 437 - DNA, 23 bp, Artificial sequence
  oligonucleotide
ctcgaggaaagaagcactttctg

SEQ ID NO: 438 - DNA, 85 bp, Artificial sequence
  oligonucleotide
ctcaggctgtgacactctagagggaagcgctttctgttgtctgaaagaaaggaaagtgcatccttttagagtgttactgtttgag

SEQ ID NO: 439 - DNA, 97 bp, Artificial sequence
  oligonucleotide
aaacaagttatattaggttggtgcaaaagtaattgtggtttttgcctgtaaaagtaatggcaaaaaccacagtttcttttgcaccagactaataaag

SEQ ID NO: 440 - DNA, 101 bp, Artificial sequence
  oligonucleotide
gcgagaagatctcatgctgtgactctctggagggaagcactttctgttgtctgaaagaaaacaaagcgcttctctttagagtgttacggtttgagaaaagc

SEQ ID NO: 441 - DNA, 81 bp, Artificial sequence
  oligonucleotide
catgctgtgaccctctagagggaagcgctttctgttgtctgaaagaaaagaaagtgcatccttttagaggtttactgtttg

SEQ ID NO: 442 - DNA, 70 bp, Artificial sequence
  oligonucleotide
tggtgcaaaagtaattgcggtttttgccattaaaagtaatgcggccaaaactgcagttacttttgcaccc

SEQ ID NO: 443 - DNA, 87 bp, Artificial sequence; identical to SEQ ID NO: 377
  oligonucleotide

SEQ ID NO: 444 - DNA, 83 bp, Artificial sequence
  oligonucleotide
tctgcaggtcctggtgaacgccatcatcaacagtggtccccgggaggactccacacgcattgggcgcgccgggactgtgagac

SEQ ID NO: 445 - DNA, 70 bp, Artificial sequence; identical to SEQ ID NO: 442
  oligonucleotide

SEQ ID NO: 446 - DNA, 61 bp, Artificial sequence
  oligonucleotide
ccctctacagggaagcgctttctgttgtctgaaagaaaagaaagtgcttccttttagaggg

SEQ ID NO: 447 - DNA, 115 bp, Artificial sequence
  oligonucleotide
ggtgccgagggccgtccggcatcctaggcgggtcgctgcggtacctccctcctgtctgtggcggtgggatcccgtggccgtgttttcctggtggcccggccgtgcctgaggtttc

SEQ ID NO: 448 - DNA, 98 bp, Artificial sequence
  oligonucleotide
ctctcaccaagcaagtgcagtggggcttgctggcttgcaccgtgactccctctcaccaagcaagtgtcgtggggcttgctggcttgcactgtgaagat

SEQ ID NO: 449 - DNA, 83 bp, Artificial sequence; identical to SEQ ID NO: 444
  oligonucleotide

SEQ ID NO: 450 - DNA, 83 bp, Artificial sequence; identical to SEQ ID NO: 444
  oligonucleotide

SEQ ID NO: 451 - DNA, 115 bp, Artificial sequence; identical to SEQ ID NO: 447
  oligonucleotide

SEQ ID NO: 452 - DNA, 116 bp, Artificial sequence
  oligonucleotide
tacttgggtgactaaggcaggattgcttgagcctgggaggtcaaggctgcagtgtcgtggtcacagcttgctgcagactcgacctcccaggcttaagcaatcctcctgctcgagtg

SEQ ID NO: 453 - DNA, 87 bp, Artificial sequence; identical to SEQ ID NO: 389
  oligonucleotide

SEQ ID NO: 454 - DNA, 93 bp, Artificial sequence
  oligonucleotide
ccttccggcgtcccaggcggggcgccgcgggaccgccctcgtgtctgtggcggtgggatcccgcggccgtgttttcctggtggcccggccatg

SEQ ID NO: 455 - DNA, 87 bp, Artificial sequence; identical to SEQ ID NO: 376
  oligonucleotide

SEQ ID NO: 456 - DNA, 87 bp, Artificial sequence; identical to SEQ ID NO: 375
  oligonucleotide

SEQ ID NO: 457 - DNA, 180 bp, Artificial sequence
  oligonucleotide
cgcgactgcggcggcggtggtggggggagccgcggggatcgccgagggccggtcggccgccccgggtgccgcgcggtgccgccggcggcggtgaggccccgcgcgtgtgtcccggctgcggtcggccgcgctcgaggggtccccgtggcgtccccttccccgccggccgcctttctcgcg

SEQ ID NO: 458 - DNA, 180 bp, Artificial sequence; identical to SEQ ID NO: 457
  oligonucleotide

SEQ ID NO: 459 - DNA, 70 bp, Artificial sequence; identical to SEQ ID NO: 442
  oligonucleotide

SEQ ID NO: 460 - DNA, 61 bp, Artificial sequence; identical to SEQ ID NO: 446
  oligonucleotide

SEQ ID NO: 461 - DNA, 87 bp, Artificial sequence; identical to SEQ ID NO: 376
  oligonucleotide

SEQ ID NO: 462 - DNA, 87 bp, Artificial sequence; identical to SEQ ID NO: 375
  oligonucleotide

SEQ ID NO: 463 - DNA, 87 bp, Artificial sequence; identical to SEQ ID NO: 376
  oligonucleotide

SEQ ID NO: 464 - DNA, 88 bp, Artificial sequence
  oligonucleotide
gtattaggttggtgcaaaggtaattgcagtttttcccatttaaaatatggaaaaaaaaatcacaattacttttgcatcaacctaataa

SEQ ID NO: 465 - DNA, 85 bp, Artificial sequence
  oligonucleotide
tctcaagctgtgactgcaaagggaagccctttctgttgtctaaaagaaaagaaagtgcttccctttggtgaattacggtttgaga

SEQ ID NO: 466 - DNA, 115 bp, Artificial sequence; identical to SEQ ID NO: 447
  oligonucleotide

SEQ ID NO: 467 - DNA, 61 bp, Artificial sequence; identical to SEQ ID NO: 446
  oligonucleotide

SEQ ID NO: 468 - DNA, 118 bp, Artificial sequence
  oligonucleotide
tctgattctgcatgtattaggttggtgcaaaagtaatcgcggtttttgtcattgaaagtaatagcaaaaactgcaattacttttgcaccaacctaaaagtagtcactgtcttcagata

SEQ ID NO: 469 - DNA, 85 bp, Artificial sequence
  oligonucleotide
ctcaggctgtgaccctctagagggaagcactttctgttgcttgaaagaagagaaagcgcttccttttagaggattactctttgag

SEQ ID NO: 470 - DNA, 87 bp, Artificial sequence; identical to SEQ ID NO: 375
  oligonucleotide

SEQ ID NO: 471 - DNA, 87 bp, Artificial sequence
  oligonucleotide
tctcgggctgtgactctccaaagggaagaattttctcttgtctaaaagaaaagaacgcacttccctttagagtgttaccgtgtgaga

SEQ ID NO: 472 - DNA, 87 bp, Artificial sequence
  oligonucleotide
tcccatgctgtgaccctctagagggaagcactttctgttgtctgaaagaaaccaaagcgcttccctttggagcgttacggtttgaga

SEQ ID NO: 473 - DNA, 129 bp, Artificial sequence
  oligonucleotide
gggatgccacattcagccattcagcgtacagtgcctttcacagggaggtgtcatttatgtgaactaaaatataaatttcacctttctgagaagggtaatgtacagcatgcactgcatatgtggtgtccc

SEQ ID NO: 474 - DNA, 127 bp, Artificial sequence
  oligonucleotide
ggatgccacattcagccattcagtgtgcagtgcctttcacagggaggtgtcatttatgtgaactaaaatataaatttcacctttctgagaagggtaatgtacagcatgcactgcatatgtggtgtcc

SEQ ID NO: 475 - DNA, 87 bp, Artificial sequence; identical to SEQ ID NO: 377
  oligonucleotide

SEQ ID NO: 476 - DNA, 87 bp, Artificial sequence
  oligonucleotide
tctcatgctgtgaccctacaaagggaagcactttctcttgtccaaaggaaaagaaggcgcttccctttggagtgttacggtttgaga

SEQ ID NO: 477 - DNA, 87 bp, Artificial sequence; identical to SEQ ID NO: 376
  oligonucleotide

SEQ ID NO: 478 - DNA, 101 bp, Artificial sequence; identical to SEQ ID NO: 440
  oligonucleotide

SEQ ID NO: 479 - DNA, 23 bp, Artificial sequence
  oligonucleotide
caaagcgcttctctttagagtgt

SEQ ID NO: 480 - DNA, 22 bp, Artificial sequence
  oligonucleotide
aaagtgcatccttttagaggtt

SEQ ID NO: 481 - DNA, 23 bp, Artificial sequence
  oligonucleotide
tgggcgcgccgggactgtgagac

SEQ ID NO: 482 - DNA, 21 bp, Artificial sequence
  oligonucleotide
aaagtgcttccttttagaggg

SEQ ID NO: 483 - DNA, 23 bp, Artificial sequence
  oligonucleotide
tgggcgcgccgggactgtgagac

SEQ ID NO: 484 - DNA, 23 bp, Artificial sequence
  oligonucleotide
tgggcgcgccgggactgtgagac

SEQ ID NO: 485 - DNA, 22 bp, Artificial sequence
  oligonucleotide
ctgcagactcgacctcccaggc

SEQ ID NO: 486 - DNA, 22 bp, Artificial sequence
  oligonucleotide
aaaatggttccctttagagtgt

SEQ ID NO: 487 - DNA, 23 bp, Artificial sequence
  oligonucleotide
gaacgcgcttccctatagagggt

SEQ ID NO: 488 - DNA, 22 bp, Artificial sequence
  oligonucleotide
aaagtgcatctttttagaggat

SEQ ID NO: 489 - DNA, 21 bp, Artificial sequence
  oligonucleotide
aaagtgcttccttttagaggg

SEQ ID NO: 490 - DNA, 23 bp, Artificial sequence
  oligonucleotide
gaacgcgcttccctatagagggt

SEQ ID NO: 491 - DNA, 22 bp, Artificial sequence
  oligonucleotide
aaagtgcatctttttagaggat

SEQ ID NO: 492 - DNA, 23 bp, Artificial sequence
  oligonucleotide
gaacgcgcttccctatagagggt

SEQ ID NO: 493 - DNA, 21 bp, Artificial sequence
  oligonucleotide
aaagtgcttccttttagaggg

SEQ ID NO: 494 - DNA, 22 bp, Artificial sequence
  oligonucleotide
aaagtgcatctttttagaggat

SEQ ID NO: 495 - DNA, 21 bp, Artificial sequence
  oligonucleotide
caaagcgcttccctttggagc

SEQ ID NO: 496 - DNA, 21 bp, Artificial sequence
  oligonucleotide
gaaggcgcttccctttggagt

SEQ ID NO: 497 - DNA, 23 bp, Artificial sequence
  oligonucleotide
gaacgcgcttccctatagagggt

SEQ ID NO: 498 - DNA, 23 bp, Artificial sequence
  oligonucleotide
caaagcgcttctctttagagtgt

SEQ ID NO: 499 - DNA, 23 bp, Artificial sequence
  oligonucleotide
tctctggagggaagcactttctg

SEQ ID NO: 500 - DNA, 22 bp, Artificial sequence
  oligonucleotide
ctctagagggaagcgctttctg

SEQ ID NO: 501 - DNA, 21 bp, Artificial sequence
  oligonucleotide
cctctacagggaagcgctttc

SEQ ID NO: 502 - DNA, 24 bp, Artificial sequence
  oligonucleotide
tctgcaggtcctggtgaacgccat

SEQ ID NO: 503 - DNA, 21 bp, Artificial sequence
  oligonucleotide
cctctacagggaagcgctttc

SEQ ID NO: 504 - DNA, 22 bp, Artificial sequence
  oligonucleotide
ggtggcccggccgtgcctgagg

SEQ ID NO: 505 - DNA, 22 bp, Artificial sequence
  oligonucleotide
tgtcgtggggcttgctggcttg

SEQ ID NO: 506 - DNA, 24 bp, Artificial sequence
  oligonucleotide
tctgcaggtcctggtgaacgccat

SEQ ID NO: 507 - DNA, 24 bp, Artificial sequence
  oligonucleotide
tctgcaggtcctggtgaacgccat

SEQ ID NO: 508 - DNA, 22 bp, Artificial sequence
  oligonucleotide
ggtggcccggccgtgcctgagg

SEQ ID NO: 509 - DNA, 21 bp, Artificial sequence
  oligonucleotide
ctgggaggtcaaggctgcagt

SEQ ID NO: 510 - DNA, 22 bp, Artificial sequence
  oligonucleotide
ctctagagggaagcgctttctg

SEQ ID NO: 511 - DNA, 22 bp, Artificial sequence
  oligonucleotide
aggcggggcgccgcgggaccgc

SEQ ID NO: 512 - DNA, 22 bp, Artificial sequence
  oligonucleotide
ctctagagggaagcgctttctg

SEQ ID NO: 513 - DNA, 22 bp, Artificial sequence
  oligonucleotide
ctctagagggaagcgctttctg

SEQ ID NO: 514 - DNA, 21 bp, Artificial sequence
  oligonucleotide
cctctacagggaagcgctttc

SEQ ID NO: 515 - DNA, 22 bp, Artificial sequence
  oligonucleotide
ctctagagggaagcgctttctg

SEQ ID NO: 516 - DNA, 22 bp, Artificial sequence
  oligonucleotide
ctctagagggaagcgctttctg

SEQ ID NO: 517 - DNA, 22 bp, Artificial sequence
  oligonucleotide
ctctagagggaagcgctttctg

SEQ ID NO: 518 - DNA, 22 bp, Artificial sequence
  oligonucleotide
aaaggtaattgcagtttttccc

SEQ ID NO: 519 - DNA, 20 bp, Artificial sequence
  oligonucleotide
ctgcaaagggaagccctttc

SEQ ID NO: 520 - DNA, 22 bp, Artificial sequence
  oligonucleotide
ggtggcccggccgtgcctgagg

SEQ ID NO: 521 - DNA, 21 bp, Artificial sequence
  oligonucleotide
cctctacagggaagcgctttc

SEQ ID NO: 522 - DNA, 22 bp, Artificial sequence
  oligonucleotide
ctctagagggaagcgctttctg

SEQ ID NO: 523 - DNA, 22 bp, Artificial sequence
  oligonucleotide
ctctagagggaagcactttctg

SEQ ID NO: 524 - DNA, 21 bp, Artificial sequence
  oligonucleotide
cctctacagggaagcgctttc

SEQ ID NO: 525 - DNA, 22 bp, Artificial sequence
  oligonucleotide
ctacaaagggaagcactttctc

SEQ ID NO: 526 - DNA, 22 bp, Artificial sequence
  oligonucleotide
ctctagagggaagcgctttctg

SEQ ID NO: 527 - DNA, 23 bp, Artificial sequence
  oligonucleotide
tctctggagggaagcactttctg

(19) United States
(12) Patent Application Publication
(10) Pub. No.: US 2022/0154187 A1
(43) Pub. Date: May 19, 2022

(54) INTRODUCING SILENCING ACTIVITY TO DYSFUNCTIONAL RNA MOLECULES AND MODIFYING THEIR SPECIFICITY AGAINST A GENE OF INTEREST

(71) Applicant: Tropic Biosciences UK Limited, Colney, Norwich (GB)

(72) Inventors: Eyal MAORI, Cambridge, Cambridge (GB); Yaron GALANTY, Coton, Cambridge (GB); Cristina PIGNOCCHI, Hethersett, Norwich (GB); Angela CHAPARRO GARCIA, Norwich (GB); Ofir MEIR, Norwich, Norfolk (GB)

(73) Assignee: Tropic Biosciences UK Limited, Colney, Norwich (GB)

(21) Appl. No.: 17/439,158

(22) PCT Filed: Mar. 12, 2020
(86) PCT No.: PCT/IB2020/052248

§ 371 Date: Sep. 14, 2021

(30) Foreign Application Priority Data
Mar. 14, 2019 (GB) 1903519.5

(51) Int. Cl. C12N 15/113; C12N 15/11; C12N 9/22; C12N 15/90; C12N 15/82; A61K 31/7088; A61K 38/46

(52) U.S. Cl. CPC C12N 15/1132; C12N 15/11; C12N 9/22; C12N 15/907; C12N 15/8218; C12N 2800/80; C12N 15/8285; A61K 31/7088; A61K 38/465; C12N 2310/141; C12N 2310/20; C12N 15/8283

(57) ABSTRACT

A method of generating an RNA molecule having a silencing activity in a cell is provided, comprising: (a) identifying nucleic acid sequences encoding RNA molecules exhibiting predetermined sequence homology range, not including complete identity, with respect to nucleic acid sequences encoding RNA molecules engaged with RISC, (b) determining transcription of nucleic acid sequences encoding RNA molecules so as to select transcribable nucleic acid sequences encoding RNA molecules; (c) determining processability into small RNAs of transcripts of transcribable nucleic acid sequences encoding RNA molecules exhibiting predetermined sequence homology range so as to select transcribable nucleic acid sequences encoding aberrantly processed RNA molecules exhibiting predetermined sequence homology range; (d) modifying a nucleic acid sequence of aberrantly processed, transcribable nucleic acid sequences so as to impart processability into small RNAs that are engaged with RISC and are complementary to a first target RNA or to a target RNA of interest.

### RELATED APPLICATION/S

This application claims the benefit of priority of UK Patent Application No. 1903519.5 filed on 14 Mar. 2019, the contents of which are incorporated herein by reference in their entirety.

### SEQUENCE LISTING STATEMENT

The ASCII file, entitled 81320 Sequence Listing.txt, created on 12 Mar. 2020, comprising 221,283 bytes, submitted concurrently with the filing of this application is incorporated herein by reference.

### FIELD AND BACKGROUND OF THE INVENTION

The present invention, in some embodiments thereof, relates to imparting a silencing activity to silencing-dysfunctional RNA molecules (e.g. miRNA-like molecules) in eukaryotic cells and possibly modifying the silencing specificity of the RNA molecules towards silencing of endogenous or exogenous target RNAs of interest.

Recent advances in genome editing techniques have made it possible to alter DNA sequences in living cells by editing only a few of the billions of nucleotides in their genome. In the past decade, the tools and expertise for using genome editing, such as in human somatic cells and pluripotent cells, have increased to such an extent that the approach is now being developed widely as a strategy to treat human disease. The fundamental process depends on creating a site-specific DNA double-strand break (DSB) in the genome and then allowing the cell's endogenous DSB repair machinery to fix the break (such as by non-homologous end-joining (NHEJ) or homologous recombination (HR) in which the latter can allow precise nucleotide changes to be made to the DNA sequence using an exogenously provided donor template [Porteus, Annu Rev Pharmacol Toxicol. (2016) 56:163-90].

Three primary approaches use mutagenic genome editing (NHEJ) of cells, such as for 30 potential therapeutics: (a) knocking out functional genetic elements by creating spatially precise insertions or deletions, (b) creating insertions or deletions that compensate for underlying frameshift mutations; hence reactivating partly functional or non-functional genes, and (c) creating defined genetic deletions. Although several different applications use editing by NHEJ, genome editing by homologous recombination (HR) will most likely offer the broadest application scope. This is because HR, although a rare event, is highly accurate as it relies on an exogenously provided template to copy a specific, predetermined sequence during the repair process.

Currently the four major types of applications to HR-mediated genome editing are: (a) gene correction (i.e. correction of diseases that are caused by point mutations in single genes), (b) functional gene correction (i.e. correction of diseases that are caused by mutations scattered throughout the gene), (c) safe harbor gene addition (i.e. when precise regulation is not required or when non-physiological levels of a transgene are desired), and (d) targeted transgene addition (i.e. when precise regulation is required) [Porteus (2016), supra].

Previous work on genome editing of RNA molecules in various eukaryotic organisms (e.g. murine, human, shrimp, plants), focused on knocking-out miRNA gene activity or changing their binding site in target RNAs, for example:

With regard to genome editing in human cells, Jiang et al. [Jiang et al., RNA Biology (2014) 11 (10): 1243-9] used CRISPR/Cas9 to delete human miR-93 from a cluster by targeting its 5′ region in HeLa cells. Various small indels were induced in the targeted region containing the Drosha processing site (i.e. the position at which Drosha, a double-stranded RNA-specific RNase III enzyme, binds, cleaves and thereby processes primary miRNAs (pri-miRNAs) into pre-miRNA in the nucleus of a host cell) and seed sequences (i.e. the conserved heptametrical sequences which are essential for the binding of the miRNA to mRNA, typically situated at positions 2-7 from the miRNA 5′-end). According to Jiang et al. even a single nucleotide deletion led to complete knockout of the target miRNA with high specificity.

With regard to genome editing in murine species, Zhao et al. [Zhao et al., Scientific Reports (2014) 4:3943] provided a miRNA inhibition strategy employing the CRISPR-Cas9 system in murine cells. Zhao used specifically designed sgRNAs to cut the miRNA gene at a single site by the Cas9 nuclease, resulting in knockout of the miRNA in these cells.

With regard to plant genome editing, Bortesi and Fischer [Bortesi and Fischer, Biotechnology Advances (2015) 33: 41-52] discussed the use of CRISPR-Cas9 technology in plants as compared to ZFNs and TALENs, and Basak and Nithin [Basak and Nithin, Front Plant Sci. (2015) 6: 1001] teach that CRISPR-Cas9 technology has been applied for knockdown of protein-coding genes in model plants such as Arabidopsis and tobacco and crops including wheat, maize, and rice.

In addition to disruption of miRNA activity or target binding sites, gene silencing using artificial miRNAs (amiRNAs) mediated gene silencing of endogenous and exogenous target genes has been achieved [Tiwari et al. Plant Mol Biol (2014) 86: 1]. Similar to miRNAs, amiRNAs are single-stranded, approximately 21 nucleotides (nt) long, and designed by replacing the mature miRNA sequences of the duplex within pre-miRNAs [Tiwari et al. (2014) supra]. These amiRNAs are introduced as a transgene within an artificial expression cassette (including a promoter, terminator etc.) [Carbonell et al., Plant Physiology (2014) pp. 113.234989], are processed via small RNA biogenesis and silencing machinery and downregulate target expression. According to Schwab et al. [Schwab et al. The Plant Cell (2006) Vol. 18, 1121-1133], amiRNAs are active when expressed under tissue-specific or inducible promoters and can be used for specific gene silencing in plants, especially when several related, but not identical, target genes need to be downregulated.

Senis et al. [Senis et al., Nucleic Acids Research (2017) Vol. 45(1): e3] disclose engineering of a promoterless anti-viral RNAi hairpin into an endogenous miRNA locus. Specifically, Senis et al. insert an amiRNA precursor transgene (hairpin pri-amiRNA) adjacent to a naturally occurring miRNA gene (e.g. miR122) by homology-directed DNA recombination that is induced by sequence-specific nuclease such as Cas9 or TALEN nucleases. This approach uses promoter- and terminator-free amiRNAs by utilizing transcriptionally active DNA that expresses a natural miRNA (miR122), that is, the endogenous promoter and terminator drove and regulated the transcription of the inserted amiRNA transgene.

Various DNA-free methods of introducing RNA and/or proteins into cells have been previously described. For example, RNA transfection using electroporation and lipofection has been described in U.S. Patent Application No. 20160289675. Direct delivery of Cas9/sgRNA ribonucleoprotein (RNPs) complexes to cells by microinjection of the Cas9 protein and sgRNA complexes was described by Cho [Cho et al., “Heritable gene knockout in Caenorhabditis elegans by direct injection of Cas9-sgRNA ribonucleoproteins,” Genetics (2013) 195:1177-1180]. Delivery of Cas9 protein/sgRNA complexes via electroporation was described by Kim [Kim et al., “Highly efficient RNA-guided genome editing in human cells via delivery of purified Cas9 ribonucleoproteins” Genome Res. (2014) 24:1012-1019]. Delivery of Cas9 protein-associated sgRNA complexes via liposomes was reported by Zuris [Zuris et al., “Cationic lipid-mediated delivery of proteins enables efficient protein-based genome editing in vitro and in vivo” Nat Biotechnol. (2014) doi: 10.1038/nbt.3081].

### SUMMARY OF THE INVENTION

According to an aspect of some embodiments of the present invention there is provided a method of generating an RNA molecule having a silencing activity in a cell, the method comprising: (a) identifying nucleic acid sequences encoding RNA molecules exhibiting a predetermined sequence homology range, not including complete identity, with respect to nucleic acid sequences encoding RNA molecules engaged with RNA-induced silencing complex (RISC); (b) determining transcription of the nucleic acid sequences encoding the RNA molecules so as to select transcribable nucleic acid sequences encoding the RNA molecules exhibiting the predetermined sequence homology range; (c) determining processability into small RNAs of transcripts of the transcribable nucleic acid sequences encoding the RNA molecules exhibiting the predetermined sequence homology range so as to select transcribable nucleic acid sequences encoding the RNA molecules exhibiting the predetermined sequence homology range, wherein the RNA molecules are aberrantly processed: (d) modifying a nucleic acid sequence of the transcribable nucleic acid sequences encoding the aberrantly processed RNA molecules exhibiting the predetermined sequence homology range so as to impart processability into small RNAs that are engaged with RISC and are complementary to a first target RNA, thereby generating the RNA molecule having the silencing activity in the cell.

According to an aspect of some embodiments of the present invention there is provided a genetically modified cell comprising a genome comprising a polynucleotide sequence encoding an RNA molecule having a nucleic acid sequence alteration which results in processing of the RNA molecules into small RNAs that are engaged with RISC, the processing of the RNA molecules being absent from a wild type cell of the same origin devoid of the nucleic acid sequence alteration.

According to an aspect of some embodiments of the present invention there is provided a plant cell generated according to the method of some embodiments of the invention.

According to an aspect of some embodiments of the present invention there is provided a plant comprising the plant cell of some embodiments of the invention.

According to an aspect of some embodiments of the present invention there is provided a method of producing a plant with reduced expression of a target gene, the method comprising: (a) breeding the plant of some embodiments of the invention; and (b) selecting for progeny plants that have reduced expression of the target RNA of interest, or progeny that comprise a silencing specificity in the RNA molecule towards the target RNA of interest, and which do not comprise the DNA editing agent, thereby producing the plant with reduced expression of a target gene.

According to an aspect of some embodiments of the present invention there is provided a method of producing a plant comprising an RNA molecule having a silencing activity towards a target RNA of interest, the method comprising: (a) breeding the plant of some embodiments of the invention; and (b) selecting for progeny plants that comprise the RNA molecule having the silencing activity towards the target RNA of interest, or progeny that comprise a silencing specificity in the RNA molecule towards the target RNA of interest, and which do not comprise the DNA editing agent, thereby producing the plant comprising the RNA molecule having the silencing activity towards the target RNA of interest.

According to an aspect of some embodiments of the present invention there is provided a method producing a plant or plant cell of some embodiments of the invention comprising growing the plant or plant cell under conditions which allow propagation.

According to an aspect of some embodiments of the present invention there is provided a seed of the plant of some embodiments of the invention, or of the plant produced by some embodiments of the invention.

According to an aspect of some embodiments of the present invention there is provided a method of treating a disease in a subject in need thereof, the method comprising generating an RNA molecule having a silencing activity and/or specificity according to the method of some embodiments of the invention, wherein the RNA molecule comprises a silencing activity towards a transcript of a gene associated with an onset or progression of the disease, thereby treating the subject.

According to an aspect of some embodiments of the present invention there is provided a method of introducing silencing activity to a first RNA molecule in a cell, the method comprising:
(a) selecting a first nucleic acid sequence within the cell, wherein:
   i. the first nucleic acid sequence is transcribed into the first RNA molecule within the cell;
   ii. the sequence of the first RNA molecule has a partial homology to the sequence of a second RNA molecule, excluding sequence identity; wherein the second RNA molecule is processable to a third RNA molecule having a silencing activity; and wherein the second RNA molecule is encoded by a second nucleic acid sequence in the cell; and
   iii. the first RNA molecule is not processable, or is processable differently than the second RNA molecule, such that the first RNA molecule is not processed to an RNA molecule having a silencing activity of the same nature as the third RNA molecule;
(b) modifying the first nucleic acid sequence such that it encodes a modified first RNA molecule, the modified first RNA molecule being processable to a fourth RNA in the same way that the second RNA molecule is processable to the third RNA molecule, such that the fourth RNA molecule has a silencing activity of the same nature as the third RNA molecule,

thereby introducing a silencing activity to the first RNA molecule.

According to some embodiments of the invention, the RNA molecules of step (a) encoded by the identified nucleic acid sequences exhibit a predetermined sequence homology range, not including complete identity, with respect to RNA molecules that are engaged with—and/or that are processed into molecules engaged with RISC.

According to some embodiments of the invention, imparting processability in step (d) comprises imparting canonical processing relative to an RNA molecule encoded by a nucleic acid sequence of the nucleic acid sequences encoding RNA molecules engaged with RNA-induced silencing complex (RISC);

According to some embodiments of the invention, the method further comprises determining the genomic location of the nucleic acid sequences encoding the RNA molecules exhibiting the predetermined sequence homology range of step (a).

According to some embodiments of the invention, the genomic location is in a non-coding gene.

According to some embodiments of the invention, the genomic location is within an intron of a non-coding gene.

According to some embodiments of the invention, the genomic location is in a coding gene.

According to some embodiments of the invention, the genomic location is within an exon of coding gene.

According to some embodiments of the invention, the genomic location is within an exon encoding an untranslated region (UTR) of a coding gene.

According to some embodiments of the invention, the genomic location is within an intron of a coding gene.

According to some embodiments of the invention, the RNA molecule is encoded by a nucleic acid sequence positioned in a non-coding gene.

According to some embodiments of the invention, the RNA molecule is encoded by a nucleic acid sequence positioned in a coding gene.

According to some embodiments of the invention, the RNA molecule is encoded by a nucleic acid sequence positioned within an exon of coding gene.

According to some embodiments of the invention, the RNA molecule is encoded by a nucleic acid sequence positioned within an exon encoding an untranslated region (UTR) of coding gene.

According to some embodiments of the invention, the RNA molecule is encoded by a nucleic acid sequence positioned within an intron of coding gene.

According to some embodiments of the invention, the genomic location is within an intron of non-coding gene.

According to some embodiments of the invention, the sequence homology range comprises 75%-99.6% identity with respect to the nucleic acid sequence encoding the RNA molecule engaged with the RISC.

According to some embodiments of the invention, step (b) and/or (c) are affected by alignment of small RNA expression data to a genome of the cell and determining the amount of reads that map to each genomic location.

According to some embodiments of the invention, the alignment of the small RNAs is alignment to a predetermined location in the genome of the cell with no mismatches.

According to some embodiments of the invention, modifying the nucleic acid sequence of the transcribable nucleic acid sequences imparts a structure of the aberrantly processed RNA molecules, which results in processing of the RNA molecules into small RNAs that are engaged with RISC.

According to some embodiments of the invention, modifying the nucleic acid sequence of the transcribable nucleic acid sequences encoding the aberrantly processed RNA molecules exhibiting the predetermined sequence homology range is affected at nucleic acids other than those corresponding to the binding site to the first target RNA.

According to some embodiments of the invention, the processability is affected by cellular nucleases selected from the group consisting of Dicer, Argonaute, tRNA cleavage enzymes, and Piwi-interacting RNA (piRNA) related proteins.

According to some embodiments of the invention, modifying in step (d) comprises introducing into the cell a DNA editing agent which reactivates silencing activity in the aberrantly processed RNA molecule towards the first target RNA, thereby generating an RNA molecule having a silencing activity in the cell.

According to some embodiments of the invention, the method further comprises modifying the specificity of the RNA molecule having the silencing activity in the cell, the method comprising introducing into the cell a DNA editing agent which redirects a silencing specificity of the RNA molecule towards a target RNA of interest, the target RNA of interest being distinct from the first target RNA, thereby modifying the specificity of the RNA molecule having the silencing activity in the cell.

According to some embodiments of the invention, the method further comprises modifying the specificity of the RNA molecule having the silencing activity in the cell, wherein the DNA editing agent redirects a silencing specificity of the RNA molecule towards a target RNA of interest, the target RNA of interest being distinct from the first target RNA, thereby modifying the specificity of the RNA molecule having the silencing activity in the cell.

According to some embodiments of the invention, the method further comprising modifying the specificity of the RNA molecule having the silencing activity in a cell, the method comprising introducing into the cell a DNA editing agent which redirects a silencing specificity of the RNA molecule towards a target RNA of interest, the target RNA of interest being distinct from the first target RNA, thereby modifying the specificity of the RNA molecule having the silencing activity in the cell.

According to some embodiments of the invention, the identified nucleic acid sequences encoding RNA molecules of step (a) are homologous to genes encoding silencing RNA molecules whose silencing activity and/or processing into small silencing RNA is dependent on their secondary structure.

According to some embodiments of the invention, the nucleic acid sequences encoding RNA molecules of step (a) are homologous to genes encoding miRNA precursors.

According to some embodiments of the invention, the silencing RNA molecule whose silencing activity and/or processing into small silencing RNA is dependent on secondary structure is selected from the group consisting of: microRNA (miRNA), short-hairpin RNA (shRNA), small nuclear RNA (snRNA or U-RNA), small nucleolar RNA (snoRNA), Small Cajal body RNA (scaRNA), transfer RNA (tRNA), ribosomal RNA (rRNA), repeat-derived RNA, autonomous and non-autonomous transposable and retro-transposable element-derived RNA, autonomous and non-autonomous transposable and retro-transposable element RNA and long non-coding RNA (lncRNA).

According to some embodiments of the invention, the processing is canonical processing.

According to some embodiments of the invention, the RNA molecule has a silencing activity.

According to some embodiments of the invention, the RNA molecule is selected from the group consisting of a microRNA (miRNA), a small interfering RNA (siRNA), a short hairpin RNA (shRNA), a Piwi-interacting RNA (piRNA), phased small interfering RNA (phasiRNA), trans-acting siRNA (tasiRNA), a transfer RNA fragment (tRF), a small nuclear RNA (snRNA), transposable and/or retro-transpossable derived RNA, autonomous and non-autonomous transposable and/or retro-transpossable RNA.

According to some embodiments of the invention, the method further comprises introducing into the cell donor oligonucleotides.

According to some embodiments of the invention, the DNA editing agent comprises at least one sgRNA.

According to some embodiments of the invention, the DNA editing agent does not comprise an endonuclease.

According to some embodiments of the invention, the DNA editing agent comprises an endonuclease.

According to some embodiments of the invention, the DNA editing agent is of a DNA editing system selected from the group consisting of a meganuclease, a zinc finger nucleases (ZFN), a transcription-activator like effector nuclease (TALEN), CRISPR-endonuclease, dCRISPR-endonuclease and a homing endonuclease.

According to some embodiments of the invention, the endonuclease comprises Cas9.

According to some embodiments of the invention, the DNA editing agent is applied to the cell as DNA, RNA or RNP.

According to some embodiments of the invention, the DNA editing agent is linked to a reporter for monitoring expression in a cell.

According to some embodiments of the invention, the reporter is a fluorescent protein.

According to some embodiments of the invention, the target RNA of interest is endogenous to the cell.

According to some embodiments of the invention, the target RNA of interest is exogenous to the cell.

According to some embodiments of the invention, the silencing specificity of the RNA molecule is determined by measuring a RNA or protein level of the target RNA of interest.

According to some embodiments of the invention, the silencing specificity of the RNA molecule is determined phenotypically.

According to some embodiments of the invention, the specificity of the RNA molecule is determined phenotypically by determination of at least one phenotype selected from the group consisting of a cell size, a growth rate/inhibition, a cell shape, a cell membrane integrity, a tumor size, a tumor shape, a pigmentation of an organism, a size of an organism, a crop yield, metabolic profile, a fruit trait, a biotic stress resistance, an abiotic stress resistance, an infection parameter, and an inflammation parameter.

According to some embodiments of the invention, the silencing specificity of the RNA molecule is determined genotypically.

According to some embodiments of the invention, the cell is a eukaryotic cell.

According to some embodiments of the invention, the eukaryotic cell is obtained from a eukaryotic organism selected from the group consisting of a plant, a mammal, an invertebrate, an insect, a nematode, a bird, a reptile, a fish, a crustacean, a fungi and an algae.

According to some embodiments of the invention, the eukaryotic cell is a plant cell.

According to some embodiments of the invention, the plant cell is a protoplast.

According to some embodiments of the invention, the plant is non-transgenic.

According to some embodiments of the invention, the plant is a transgenic plant.

According to some embodiments of the invention, the plant is non-genetically modified (non-GMO).

According to some embodiments of the invention, the plant is genetically modified (GMO).

According to some embodiments of the invention, the breeding comprises crossing or selfing.

According to some embodiments of the invention, the eukaryotic cell is a non-human animal cell.

According to some embodiments of the invention, the eukaryotic cell is a non-human mammalian cell.

According to some embodiments of the invention, the eukaryotic cell is a human cell.

According to some embodiments of the invention, the nucleic acid sequences encoding RNA molecules are selected from the group consisting of the nucleic acid sequences as set forth in any of SEQ ID NOs. 352 to 392.

According to some embodiments of the invention, the eukaryotic cell is a totipotent stem cell.

According to some embodiments of the invention, the gene associated with the onset or progression of the disease comprises a gene of a pathogen.

According to some embodiments of the invention, the gene associated with the onset or progression of the disease comprises a gene of the subject.

According to some embodiments of the invention, the disease is selected from the group consisting of an infectious disease, a monogenic recessive disorder, an autoimmune disease and a cancerous disease.

According to some embodiments of the invention, the second RNA molecule is an RNA molecule which has a secondary structure that enables it to be processed into an RNA having a silencing activity, optionally wherein the silencing activity is mediated through engaging RISC.

According to some embodiments of the invention, the RNA molecule which has a secondary structure that enables it to be processed into an RNA having a silencing activity is selected from the group consisting of: microRNA (miRNA), short-hairpin RNA (shRNA), small nuclear RNA (snRNA or URNA), small nucleolar RNA (snoRNA), Small Cajal body RNA (scaRNA), transfer RNA (tRNA), ribosomal RNA (rRNA), repeat-derived RNA, autonomous and non-autonomous transposable and retro-transposable element-derived RNA, autonomous and non-autonomous transposable and retro-transposable element RNA and long non-coding RNA (lncRNA).

According to some embodiments of the invention, the first nucleic acid sequence results in a secondary structure which enables the modified first RNA molecule to be processed into the fourth RNA molecule.

According to some embodiments of the invention, modifying the first nucleic acid sequence comprises modifying the sequence such that the modified first RNA molecule has essentially the same secondary structure as that of the second RNA molecule.

According to some embodiments, the secondary structure is at least 95%, 96%, 97%, 98%, 99%, 99.5%, 99.9% or 100% identical to the secondary structure of the second RNA molecule (e.g. when the secondary structure of the first RNA molecule is translated to a linear string form and is compared to a string form of a secondary structure of the second RNA molecule).

According to some embodiments of the invention, the first nucleic acid molecule is a gene from H. sapiens, wherein the gene is selected from the group consisting of the genes having the sequences set forth in any of SEQ ID NOs. 352 to 392.

According to some embodiments of the invention, the subject is a human subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flow chart of an embodiment computational pipeline for imparting a silencing activity of dysfunctional non-coding RNA molecules and redirecting their silencing specificity. Of note, a computational Genome Editing Induced Gene Silencing (GEiGS) pipeline applies biological metadata and enables an automatic generation of GEiGS DNA templates that are used to minimally edit miRNA genes, leading to a new gain of function, i.e. redirection of their silencing capacity to a target sequence of interest.

FIG. 2 is a photograph illustrating the miRbase presentation of small RNAseq profiling of a functional miRNA. Note the different detection of the two mature miRNA strands. The miRNA with high number of reads is typically the functional one (guide strand) and the other with little or no reads is typically degraded in the cell (passenger strand). However, there are some cases in which both strands of the mature miRNA are functional (each target different transcript).

FIG. 3 is graph illustrating the number of RNA-seq reads covering miRNA-like sequences. The x-axis denotes expressed miRNA-like sequences in different species. The y-axis depicts the number of distinct RNAseq reads that cover the miRNA-like sequences, where ‘has’ stands for H. sapiens, ‘ath’ for A. thaliana and ‘cel’ for C. elegans.

FIG. 4 is an embodiment flow chart of computational pipeline to generate GEiGS templates. The computational GEiGS pipeline applies biological metadata and enables an automatic generation of GEiGS DNA donor templates that are used to minimally edit endogenous non-coding RNA genes (e.g. miRNA genes), leading to a new gain of function, i.e. redirection of their silencing capacity to target gene expression of interest.

FIG. 5 is an embodiment flow chart of Genome Editing Induced Gene Silencing (GEiGS) replacement of endogenous miRNA with siRNA targeting the PDS gene, hence inducing gene silencing of the endogenous PDS gene. To introduce the modification, a 2-component system is being used. First, a CRISPR/CAS9 system, in a GFP containing vector, generates a cleavage in the chosen loci, through designed specific guide RNAs to promote homologous DNA repair (HDR) in the site. Second, A DONOR sequence, with the desired modification of the miRNA sequence, to target the newly assigned genes, is introduced as a template for the HDR. This system is being used in protoplast transformation, enriched by FACS due to the GFP signal in the CRISPR/CAS9 vector, recovered, and regenerated to plants.

FIGS. 6A-C are photographs illustrating that silencing of the PDS gene causes photobleaching. Silencing of the PDS gene in Nicotiana (FIGS. 6A-B) and Arabidopsis (FIG. 6C) plants causes photobleaching in N. benthamiana (FIG. 6B) and Arabidopsis (FIG. 6C, right side). Photographs were taken 3% weeks after PDS silencing.

FIG. 7 provides a schematic representation of an embodiment of the process for reactivating or redirecting silencing activity in an RNA transcript according to the invention.

FIGS. 8A-B provide a schematic representation of the vectors used to transfect A. thaliana protoplasts as described in Example 2 herein below, in order to test processability and silencing activity of: (FIG. 8A) a precursor of a wild type miRNA, a precursor of a “dead” miRNA-like molecule and a precursor of a “dead” miRNA-like molecule in which the silencing activity has been reactivated, and (FIG. 8B) a precursor of a “dead” miRNA-like molecule in which the silencing activity has been reactivated, and a precursor of a “dead” miRNA-like molecule in which the silencing activity has been redirected to target the PDS3 gene.

FIGS. 9A-H provide: (FIG. 9A) Schematic representation of predicted secondary structure for the following A. thaliana precursors encoded by the following miRNA or miRNA-like genes: wild-type miR405a, miRNA-like miR859_Dead, miRNA-like miR859_Dead in which silencing activity has been reactivated (miR859_Reactivated) and miRNA-like miR859_Dead in which silencing activity has been activated and redirected towards the PDS3 gene (miR859_Redirected). The grey box on each structure marks the guide strand of the mature miRNA or the corresponding location in the miRNA-like precursor—each guide strand and its alignment to its target sequence is further presented in FIG. 9B. (FIG. 9C) and (FIG. 9D) Bar graphs comparing silencing activity (as measured by reduction in the ratio between the Luciferase, LUC, and normalizing Fluorescent Protein, FP) observed when A. thaliana protoplasts were transfected with vectors expressing the vectors depicted in (FIG. 9A). Dark coloured bars represent experimental treatments and light-coloured bars represent their respective controls; p-value written within brackets in the graph according to student's t-test; Error bars represent standard error. (FIG. 9E) Schematic representation of predicted secondary structure for the following A. thaliana precursors encoded by the following miRNA or miRNA-like genes: wild-type miR8174, miRNA-like miR1334_Dead, miRNA-like miR1334_Dead in which silencing activity has been reactivated (miR1334_Reactivated) and miRNA-like miR1334_Dead in which silencing activity has been activated and redirected towards the PDS3 gene (miR1334_Redirected). The grey box on each structure marks the guide strand of the mature miRNA or the corresponding location in the miRNA-like precursor—each guide strand and its alignment to its target sequence is further presented in FIG. 9F. (FIG. 9G) and (FIG. 9H) Bar graphs comparing silencing activity (as measured by reduction in the ratio between the Luciferase, LUC, and normalizing Fluorescent Protein, FP) observed when A. thaliana protoplasts were transfected with vectors expressing the vectors depicted in (FIG. 9E). Dark coloured bars represent experimental treatments and light-coloured bars represent their respective controls; p-value written within brackets in the graph according to student's t-test; Error bars represent standard error.

FIGS. 10A-N provide small RNA distribution and secondary structure plots of miRNA-like gene ath_dead_mir1334 from Arabidopsis thaliana and its corresponding WT miRNA ath-mir-8174 (MI0026804). For each mir-like gene and its corresponding WT miRNA, seven different read size groups, 19-24 bp long, and a group denoted small, which depicts small RNA seq reads of all sizes, were used to plot the distribution of the reads that perfectly match the corresponding precursor sequence. Read counts were normalized to RPKM and a plot was generated for a certain size group if there were at least 10 reads that perfectly matched the corresponding precursor sequence. The secondary structures of each precursor sequence were generated using the RNAplot module from the ViennaRNA package. Specifically, FIG. 10A shows the distribution plot for all root 20 bp long small RNA seq reads that perfectly matched the WT precursor sequence (miRNA gene ath-mir-8174, located in chr3 positions 16589414-16589527). The lower bar plot in each plot marks the location of the mature sequences of the plotted precursor and the legend indicates the size of the mature sequences. FIG. 10G shows the secondary structure of the aforementioned WT miRNA precursor. FIG. 10H depicts the distribution plot of all root 20 bp small RNA seq reads that perfectly matched the mir-like gene precursor sequence, located in chr5 positions 13644905-1364500. FIG. 10N shows the secondary structure of the mir-like precursor ath_dead_mir1334.

FIGS. 11A-J provide small RNA distribution and secondary structure plots of miRNA-like gene ath_dead_mir247 from Arabidopsis thaliana and its corresponding WT miRNA ath-mir-8180 (MI0026810). For each mir-like gene and its corresponding WT miRNA, seven different read size groups, 19-24 bp long, and a group denoted small, which depicts small RNA seq reads of all sizes, were used to plot the distribution of the reads that perfectly match the corresponding precursor sequence. Read counts were normalized to RPKM and a plot was generated for a certain size group if there were at least 10 reads that perfectly matched the corresponding precursor sequence. The secondary structures of each precursor sequence were generated using the RNAplot module from the ViennaRNA package. Specifically, FIG. 11E shows the secondary structure of the aforementioned WT miRNA precursor. FIG. 11F depicts the distribution plot of all root 21 bp long small RNA seq reads that perfectly matched the mir-like gene precursor sequence. FIG. 11J shows the secondary structure of the mir-like precursor ath_dead_mir247.

FIGS. 12A-I provide small RNA distribution and secondary structure plots of miRNA-like gene ath_dead_mir859 from Arabidopsis thaliana and its corresponding WT miRNA ath-mir-405a (MI0001074). For each mir-like gene and its corresponding WT miRNA, seven different read size groups, 19-24 bp long, and a group denoted small, which depicts small RNA seq reads of all sizes, were used to plot the distribution of the reads that perfectly match the corresponding precursor sequence. Read counts were normalized to RPKM and a plot was generated for a certain size group if there were at least 10 reads that perfectly matched the corresponding precursor sequence. The secondary structures of each precursor sequence were generated using the RNAplot module from the ViennaRNA package. Specifically. FIG. 12A shows the distribution plot for all 24 bp long root small RNA seq reads that perfectly matched the WT precursor sequence (miRNA gene ath-mir-405a). The lower bar plot in each plot marks the location of the mature sequences of the plotted precursor and the legend indicates the size of the mature sequences. FIG. 12D shows the secondary structure of the aforementioned WT miRNA precursor. FIG. 12E depicts the distribution plot of all 23 bp long root small RNA seq reads that perfectly matched the mir-like gene precursor sequence. FIG. 12I shows the secondary structure of the mir-like precursor ath_dead_mir859.

FIGS. 13A-H provide small RNA distribution and secondary structure plots of miRNA-like gene cel_dead_mir219 from C. elegans and its corresponding WT miRNA cel-mir-5545 (MI0019066). For each mir-like gene and its corresponding WT miRNA, seven different read size groups, 19-24 bp long, and a group denoted small, which depicts small RNA seq reads of all sizes, were used to plot the distribution of the reads that perfectly match the corresponding precursor sequence. Read counts were normalized to RPKM and a plot was generated for a certain size group if there were at least 10 reads that perfectly matched the corresponding precursor sequence. The secondary structures of each precursor sequence were generated using the RNAplot module from the ViennaRNA package. Specifically, FIG. 13A depicts the distribution plot of all embryo 21 bp long small RNA seq reads that perfectly matched the precursor sequence of the WT miRNA gene cel-mir-5545. The lower bar plot in each plot marks the location of the mature sequences of the plotted precursor and the legend indicates the size of the mature sequences. Similarly, FIG. 13B shows the distribution plot for all 22 bp long embryo small RNA seq reads that perfectly matched the WT precursor sequence. FIG. 13E shows the secondary structure of the aforementioned WT miRNA precursor. FIG. 13F depicts the distribution plot of all young adult 22 bp long small RNA seq reads that perfectly matched the mir-like gene precursor sequence. FIG. 13H shows the secondary structure of the mir-like precursor cel_dead_mir219.

FIGS. 14A-H provide small RNA distribution and secondary structure plots of miRNA-like gene cel_dead_mir363 from C. elegans and its corresponding WT miRNA cel-mir-5545 (MI0019066). For each mir-like gene and its corresponding WT miRNA, seven different read size groups, 19-24 bp long, and a group denoted small, which depicts small RNA seq reads of all sizes, were used to plot the distribution of the reads that perfectly match the corresponding precursor sequence. Read counts were normalized to RPKM and a plot was generated for a certain size group if there were at least 10 reads that perfectly matched the corresponding precursor sequence. The secondary structures of each precursor sequence were generated using the RNAplot module from the ViennaRNA package. Specifically, FIG. 14A depicts the distribution plot of all embryo 21 bp long small RNA seq reads that perfectly matched the precursor sequence of the WT miRNA gene cel-mir-5545. The lower bar plot in each plot marks the location of the mature sequences of the plotted precursor and the legend indicates the size of the mature sequences. Similarly, FIG. 14B shows the distribution plot for all 22 bp long embryo small RNA seq reads that perfectly matched the WT precursor sequence. FIG. 14E shows the secondary structure of the aforementioned WT miRNA precursor. FIG. 14F depicts the distribution plot of all L4 22 bp long small RNA seq reads that perfectly matched the mir-like gene precursor sequence. FIG. 14H shows the secondary structure of the mir-like precursor cel_dead_mir363.

FIGS. 15A-H provide small RNA distribution and secondary structure plots of miRNA-like gene cel_dead_mir537 from C. elegans and its corresponding WT miRNA cel-mir-8196b (MI0026837). For each mir-like gene and its corresponding WT miRNA, seven different read size groups, 19-24 bp long, and a group denoted small, which depicts small RNA seq reads of all sizes, were used to plot the distribution of the reads that perfectly match the corresponding precursor sequence. Read counts were normalized to RPKM and a plot was generated for a certain size group if there were at least 10 reads that perfectly matched the corresponding precursor sequence. The secondary structures of each precursor sequence were generated using the RNAplot module from the ViennaRNA package. Specifically, FIG. 15A shows the distribution plot for all 23 bp long embryo small RNA seq reads that perfectly matched the WT precursor sequence (miRNA gene cel-mir-8196b). The lower bar plot in each plot marks the location of the mature sequences of the plotted precursor and the legend indicates the size of the mature sequences. FIG. 15F shows the secondary structure of the aforementioned WT miRNA precursor. FIG. 15G depicts the distribution plot of all embryo small RNA seq reads that perfectly matched the mir-like gene precursor sequence. FIG. 15H shows the secondary structure of the mir-like precursor cel_dead_mir537. Of note, the WT sequence and mir-like sequence differ only in a very small number of bases. Thus, it is expected that their secondary structure will be very similar or even identical.

FIGS. 16A-J provide small RNA distribution and secondary structure plots of miRNA-like gene hsa_dead_mir54024 from H. sapiens and its corresponding WT miRNA hsa-mir-523 (MI0003153). For each mir-like gene and its corresponding WT miRNA, seven different read size groups, 19-24 bp long, and a group denoted small, which depicts small RNA seq reads of all sizes, were used to plot the distribution of the reads that perfectly match the corresponding precursor sequence. Read counts were normalized to RPKM and a plot was generated for a certain size group if there were at least 10 reads that perfectly matched the corresponding precursor sequence. The secondary structures of each precursor sequence were generated using the RNAplot module from the ViennaRNA package. Specifically. FIG. 16A depicts the distribution plot of all 21 bp long brain small RNA seq reads that perfectly matched the precursor sequence of the WT miRNA gene hsa-mir-523. The lower bar plot in each plot marks the location of the mature sequences of the plotted precursor and the legend indicates the size of the mature sequences. Similarly, FIG. 16B shows the distribution plot for all 22 bp long brain small RNA seq reads that perfectly matched the WT precursor sequence. FIG. 16E shows the secondary structure of the aforementioned WT miRNA precursor. FIG. 16I depicts the distribution plot of all lung small RNA seq reads that perfectly matched the mir-like gene precursor sequence. FIG. 16F shows the secondary structure of the mir-like precursor hsa_dead_mir54024.

FIGS. 17A-J provide small RNA distribution and secondary structure plots of miRNA-like gene hsa_dead_mir54573 from H. sapiens and its corresponding WT miRNA hsa-mir-663b (MI0006336). For each mir-like gene and its corresponding WT miRNA, seven different read size groups, 19-24 bp long, and a group denoted small, which depicts small RNA seq reads of all sizes, were used to plot the distribution of the reads that perfectly match the corresponding precursor sequence. Read counts were normalized to RPKM and a plot was generated for a certain size group if there were at least 10 reads that perfectly matched the corresponding precursor sequence. The secondary structures of each precursor sequence were generated using the RNAplot module from the ViennaRNA package. Specifically, FIG. 17A depicts the distribution plot of all 21 bp long brain small RNA seq reads that perfectly matched the precursor sequence of the WT miRNA gene hsa-mir-663b. The lower bar plot in each plot marks the location of the mature sequences of the plotted precursor and the legend indicates the size of the mature sequences. Similarly, FIG. 17B shows the distribution plot for all brain small RNA seq reads that perfectly matched the WT precursor sequence. FIG. 17C shows the secondary structure of the WT miRNA precursor hsa-mir-663b. FIG. 17D depicts the distribution plot of all 22 bp long brain small RNA seq reads that perfectly matched the mir-like gene precursor sequence. FIG. 17J shows the secondary structure of the mir-like precursor hsa_dead_mir54573.

FIGS. 18A-E provide small RNA distribution and secondary structure plots of miRNA-like gene hsa_dead_mir50078 from H. sapiens and its corresponding WT miRNA hsa-mir-1273h (MI0025512). For each mir-like gene and its corresponding WT miRNA, seven different read size groups, 19-24 bp long, and a group denoted small, which depicts small RNA seq reads of all sizes, were used to plot the distribution of the reads that perfectly match the corresponding precursor sequence. Read counts were normalized to RPKM and a plot was generated for a certain size group if there were at least 10 reads that perfectly matched the corresponding precursor sequence. The secondary structures of each precursor sequence were generated using the RNAplot module from the ViennaRNA package. Specifically, FIG. 18A depicts the distribution plot of all 23 bp long brain small RNA seq reads that perfectly matched the precursor sequence of the WT miRNA gene hsa-mir-1273h. The lower bar plot in each plot marks the location of the mature sequences of the plotted precursor and the legend indicates the size of the mature sequences. Similarly, FIG. 18B shows the distribution plot for all brain small RNA seq reads that perfectly matched the WT precursor sequence. FIG. 18C shows the secondary structure of the aforementioned WT miRNA precursor. FIG. 18D depicts the distribution plot of all brain small RNA seq reads that perfectly matched the mir-like gene precursor sequence. FIG. 18E shows the secondary structure of the mir-like precursor hsa_dead_mir50078.

FIGS. 19A-H provide small RNA distribution and secondary structure plots of miRNA cel-mir-71 (MI0000042) from C. elegans. Seven different read size groups, 19-24 bp long, and a group denoted small, which depicts small RNA seq reads of all sizes, were used to plot the distribution of the reads that perfectly match the miRNA precursor sequence. Read counts were normalized to RPKM and a plot was generated for a certain size group if there were at least 10 reads that perfectly matched the corresponding precursor sequence. The secondary structures of each precursor sequence were generated using the RNAplot module from the ViennaRNA package. Specifically, FIG. 19A depicts the distribution plot of all 21 bp long embryo small RNA seq reads that perfectly matched the precursor sequence of the WT miRNA gene cel-mir-71. The lower bar plot in each plot marks the location of the mature sequences of the plotted precursor and the legend indicates the size of the mature sequences. Similarly, FIG. 19B shows the distribution plot for all 23 bp long embryo small RNA seq reads that perfectly matched the precursor sequence. FIG. 19H shows the secondary structure of the miRNA cel-mir-71.

### DESCRIPTION OF SPECIFIC EMBODIMENTS OF THE INVENTION

The present invention, in some embodiments thereof, relates to imparting a silencing activity to silencing-dysfunctional RNA molecules (e.g. miRNA-like molecules) in eukaryotic cells and possibly modifying the silencing specificity of the RNA molecules towards silencing of endogenous or exogenous target RNAs of interest.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways and in different organisms. Also, it is to be understood that the phraseology and terminology employed herein is for the purpose of description and should not be regarded as limiting.

Previous work on genome editing of RNA molecules in various organisms (e.g. murine, human, plants), focused on disruption of miRNA activity or target binding sites using transgenesis. Genome editing in plants has concentrated on the use of nucleases such as CRISPR-Cas9 technology, ZFNs and TALENs, for knockdown of genes or insertions in model plants. Furthermore, gene silencing in plants using artificial miRNA transgenes to silence endogenous and exogenous target genes has been described [Molnar A et al. Plant J. (2009) 58(1):165-74. Doi: 10.1111/j.1365-313X.2008.03767.x. Epub 2009 Jan. 19; Borges and Martienssen, Nature Reviews Molecular Cell Biology|AOP, published online 4 Nov. 2015; doi:10.1038/nrm4085]. The artificial miRNA transgenes are introduced into plant cells within an artificial expression cassette (including a promoter, terminator, selection marker, etc.) and downregulate target expression.

Genetic therapeutic technologies developed in mammalian organisms (e.g. for human treatment) include gene therapy, which enables restoration of missing gene function by viral transgene expression, and RNAi, which mediates repression of defective genes by knockdown of the target mRNA. Recent advances in genome editing techniques have also made it possible to alter DNA sequences in living cells by editing a one or more nucleotides in cells of human patients such as by genome editing (NHEJ and HR) following induction of site-specific double-strand breaks (DSBs) at desired locations in the genome. While NHEJ is mainly, if not exclusively, used for knockout purposes, HR is used for introducing precision editing of specific sites such as point mutations or correcting deleterious mutations that are naturally occurring or hereditarily transmitted.

The present invention is based in part on the identification of genes encoding RNA molecules, wherein: (1) the RNA molecules encoded by the identified genes demonstrate a homology to corresponding canonical silencing RNA molecules (e.g. miRNAs and/or miRNA precursors) from the same organism; (2) the identified genes are transcribed into RNA molecules; and (3) the RNA expressed by the identified genes is not processed into RNA like the corresponding homologous canonical silencing molecules (i.e. the RNA expressed by the identified genes, is aberrantly processed or non-processed). As exemplified herein below, such genes have been identified in various organisms. Without wishing to be bound by theory or mechanism, such an aberrantly processed RNA is not processed into an RNA molecule having a silencing activity, and thus the identified genes encode silencing-dysfunctional RNA molecules.

While reducing the present invention to practice, the present inventors have devised a gene editing technology directed at imparting canonical processability to dysfunctional RNA molecules (e.g processing by RNAi factors, such as Dicer), wherein the dysfunctional RNA molecules comprise at least one nucleic acid sequence alteration with respect to a homologous nucleic acid sequence encoding a canonically processed RNA molecule in the same organism, and further wherein the dysfunctional RNA molecules are transcribed in the cell.

The present inventors have further utilized a gene editing technology which redirects the silencing specificity of the processable RNA molecules to target and interfere with expression of target genes of interest (endogenous or exogenous to the cell) that were not originally targeted by the silencing RNAs. Specifically, the present inventors have designed a Genome Editing Induced Gene Silencing (GEiGS) platform capable of utilizing an eukaryotic cell's endogenous RNA molecules including e.g. non-coding RNA molecules (e.g. RNA silencing molecules, e.g. siRNA, miRNA, piRNA, tasiRNA, tRNA, rRNA, antisense RNA, etc.) and modifying them to target any RNA target of interest. Using GEiGS, the present method enables editing a few nucleotides in these endogenous RNA molecules, and thereby redirecting their activity and/or specificity to effectively and specifically target any RNA of interest. The gene editing technology described herein does not necessitate the classical molecular genetic and transgenic tools comprising expression cassettes that have a promoter, terminator, selection marker. Moreover, the gene editing technology of some embodiments of the invention comprises genome editing of an RNA molecule (e.g. endogenous) yet it is stable and heritable.

Thus, according to one aspect of the present invention there is provided a method of generating an RNA molecule having a silencing activity in a cell, the method comprising: (a) identifying nucleic acid sequences encoding RNA molecules exhibiting a predetermined sequence homology range, not including complete identity, with respect to a nucleic acid sequence encoding an RNA molecule engaged with RNA-induced silencing complex (RISC); (b) determining transcription of the nucleic acid sequences encoding the RNA molecules so as to select transcribable nucleic acid sequences encoding the RNA molecules exhibiting the predetermined sequence homology range; (c) determining processability into small RNAs of transcripts of the transcribable nucleic acid sequences encoding the RNA molecules exhibiting the predetermined sequence homology range so as to select, transcribable nucleic acid sequences encoding the RNA molecules exhibiting the predetermined sequence homology range, wherein the RNA molecules are aberrantly processed; (d) modifying a nucleic acid sequence of the transcribable nucleic acid sequences encoding the aberrantly processed RNA molecules exhibiting the predetermined sequence homology range so as to impart processability into small RNAs that are engaged with RISC and are complementary to a first target RNA, thereby generating the RNA molecule having the silencing activity in the cell.

According to some embodiment, provided herein is a method of generating an RNA molecule having a silencing activity in a cell, the method comprising: (a) selecting nucleic acid sequences encoding RNA molecules, exhibiting a predetermined sequence homology range, not including complete identity, with respect to nucleic acid sequences encoding RNA molecules engaged with RNA-induced silencing complex (RISC); wherein selecting comprises: (1) determining transcription of the nucleic acid sequences encoding the RNA molecules so as to select transcribable nucleic acid sequences encoding the RNA molecules, exhibiting the predetermined sequence homology range; and (2) determining processability into small RNAs of transcripts of the transcribable nucleic acid sequences encoding the RNA molecules exhibiting the predetermined sequence homology range so as to select transcribable nucleic acid sequences encoding the RNA molecules exhibiting the predetermined sequence homology range, wherein the RNA molecules are aberrantly processed; and (b) modifying a nucleic acid sequence of the transcribable nucleic acid sequences encoding the aberrantly processed RNA molecules exhibiting the predetermined sequence homology range so as to impart processability into small RNAs that are engaged with RISC and are complementary to a first target RNA, thereby generating the RNA molecule having the silencing activity in the cell.

According to one embodiment, the cell is a eukaryotic cell.

The term “eukaryotic cell” as used herein refers to any cell of a eukaryotic organism. Eukaryotic organisms include single- and multi-cellular organisms. Single cell eukaryotic organisms include, but are not limited to, yeast, protozoans, slime molds and algae. Multi-cellular eukaryotic organisms include, but are not limited to, animals (e.g. mammals, insects, invertebrates, nematodes, birds, fish, reptiles and crustaceans), plants, fungi and algae (e.g. brown algae, red algae, green algae).

According to one embodiment, the cell is a plant cell.

According to a specific embodiment, the plant cell is a protoplast.

The protoplasts are derived from any plant tissue e.g., fruit, flowers, roots, leaves, embryos, embryonic cell suspension, calli or seedling tissue (as discussed below).

According to a specific embodiment, the plant cell is an embryogenic cell.

According to a specific embodiment, the plant cell is a somatic embryogenic cell.

According to one embodiment, the eukaryotic cell is not a cell of a plant.

According to a one embodiment, the eukaryotic cell is an animal cell (e.g. non-human animal cell).

According to a one embodiment, the eukaryotic cell is a cell of a vertebrate.

According to a one embodiment, the eukaryotic cell is a cell of an invertebrate.

According to a specific embodiment, the invertebrate cell is a cell of an insect, a snail, a clam, an octopus, a starfish, a sea-urchin, a jellyfish, and a worm.

According to a specific embodiment, the invertebrate cell is a cell of a crustacean. Exemplary crustaceans include, but are not limited to, shrimp, prawns, crabs, lobsters and crayfishes.

According to a specific embodiment, the invertebrate cell is a cell of a fish. Exemplary fish include, but are not limited to, Salmon, Tuna, Pollock, Catfish, Cod, Haddock, Prawns, Sea bass, Tilapia, Arctic char and Carp.

According to a one embodiment, the eukaryotic cell is a mammalian cell (e.g. non-human mammalian cell).

According to a specific embodiment, the mammalian cell is a cell of a non-human organism, such as but not limited to, a rodent, a rabbit, a pig, a goat, a ruminant (e.g. cattle, sheep, antelope, deer, and giraffe), a dog, a cat, a horse, and non-human primate.

According to a specific embodiment, the eukaryotic cell is a cell of human being.

According to one embodiment, the eukaryotic cell is a primary cell, a cell line, a somatic cell, a germ cell, a stem cell, an embryonic stem cell, an adult stem cell, a hematopoietic stem cell, a mesenchymal stem cell, an induced pluripotent stem cell (iPS), a gamete cell, a zygote cell, a blastocyst cell, an embryo, a fetus and/or a donor cell.

As used herein, the phrase “stem cells” refers to cells which are capable of remaining in an undifferentiated state (e.g., totipotent, pluripotent or multipotent stem cells) for extended periods of time in culture until induced to differentiate into other cell types having a particular, specialized function (e.g., fully differentiated cells). Totipotent cells, such as embryonic cells within the first couple of cell divisions after fertilization are the only cells that can differentiate into embryonic and extra-embryonic cells and are able to develop into a viable human being. Preferably, the phrase “pluripotent stem cells” refers to cells which can differentiate into all three embryonic germ layers, i.e., ectoderm, endoderm and mesoderm or remaining in an undifferentiated state. The pluripotent stem cells include embryonic stem cells (ESCs) and induced pluripotent stem cells (iPS). The multipotent stem cells include adult stem cells and hematopoietic stem cells.

The phrase “embryonic stem cells” refers to embryonic cells which are capable of differentiating into cells of all three embryonic germ layers (i.e., endoderm, ectoderm and mesoderm), or remaining in an undifferentiated state. The phrase “embryonic stem cells” may comprise cells which are obtained from the embryonic tissue formed after gestation (e.g., blastocyst) before implantation of the embryo (i.e., a pre-implantation blastocyst), extended blastocyst cells (EBCs) which are obtained from a post-implantation/pre-gastrulation stage blastocyst (see WO2006/040763), embryonic germ (EG) cells which are obtained from the genital tissue of a fetus any time during gestation, preferably before 10 weeks of gestation, and cells originating from an unfertilized ova which are stimulated by parthenogenesis (parthenotes).

The embryonic stem cells of some embodiments of the invention can be obtained using well-known cell-culture methods. For example, human embryonic stem cells can be isolated from human blastocysts. Human blastocysts are typically obtained from human in vivo preimplantation embryos or from in vitro fertilized (IVF) embryos. Alternatively, a single cell human embryo can be expanded to the blastocyst stage.

It will be appreciated that commercially available stem cells can also be used according to some embodiments of the invention. Human ES cells can be purchased from the NIH human embryonic stem cells registry [www(dot)grants(dot)nih(dot) gov/stem_cells/registry/current(dot)html].

In addition, embryonic stem cells can be obtained from various species, including mouse (Mills and Bradley, 2001), golden hamster [Doetschman et al., 1988, Dev Biol. 127: 224-7], rat [Iannaccone et al., 1994, Dev Biol. 163: 288-92] rabbit [Giles et al. 1993, Mol Reprod Dev. 36: 130-8; Graves & Moreadith, 1993, Mol Reprod Dev. 1993, 36: 424-33], several domestic animal species [Notarianni et al., 1991, J Reprod Fertil Suppl. 43: 255-60; Wheeler 1994, Reprod Fertil Dev. 6: 563-8; Mitalipova et al., 2001, Cloning. 3: 59-67] and non-human primate species (Rhesus monkey and marmoset) [Thomson et al., 1995, Proc Natl Acad Sci USA. 92: 7844-8; Thomson et al., 1996, Biol Reprod. 55: 254-9].

“Induced pluripotent stem cells” (iPS; embryonic-like stem cells) refers to cells obtained by de-differentiation of adult somatic cells which are endowed with pluripotency (i.e., being capable of differentiating into the three embryonic germ cell layers, i.e., endoderm, ectoderm and mesoderm). According to some embodiments of the invention, such cells are obtained from a differentiated tissue (e.g., a somatic tissue such as skin) and undergo de-differentiation by genetic manipulation which reprogram the cell to acquire embryonic stem cells characteristics. According to some embodiments of the invention, the induced pluripotent stem cells are formed by inducing the expression of Oct-4, Sox2, Kfl4 and c-Myc in a somatic stem cell.

Induced pluripotent stem cells (iPS) (embryonic-like stem cells) can be generated from somatic cells by genetic manipulation of somatic cells, e.g., by retroviral transduction of somatic cells such as fibroblasts, hepatocytes, gastric epithelial cells with transcription factors such as Oct-3/4, Sox2, c-Myc, and KLF4 [such as described in Park et al. Reprogramming of human somatic cells to pluripotency with defined factors. Nature (2008) 451:141-146].

The phrase “adult stem cells” (also called “tissue stem cells” or a stem cell from a somatic tissue) refers to any stem cell derived from a somatic tissue [of either a postnatal or prenatal animal (especially the human)]. The adult stem cell is generally thought to be a multipotent stem cell, capable of differentiation into multiple cell types. Adult stem cells can be derived from any adult, neonatal or fetal tissue such as adipose tissue, skin, kidney, liver, prostate, pancreas, intestine, bone marrow and placenta.

According to one embodiment, the stem cells utilized by some embodiments of the invention are bone marrow (BM)-derived stem cells including hematopoietic, stromal or mesenchymal stem cells [Dominici, M et al., (2001) J. Biol. Regul. Homeost. Agents. 15: 28-37]. BM-derived stem cells may be obtained from iliac crest, femora, tibiae, spine, rib or other medullar spaces.

Hematopoietic stem cells (HSCs), which may also referred to as adult tissue stem cells, include stem cells obtained from blood or bone marrow tissue of an individual at any age or from cord blood of a newborn individual. Preferred stem cells according to this aspect of some embodiments of the invention are embryonic stem cells, preferably of a human or primate (e.g., monkey) origin.

Placental and umbilical cord blood stem cells may also be referred to as “young stem cells”.

Mesenchymal stem cells (MSCs), the formative pluripotent blast cells, give rise to one or more mesenchymal tissues (e.g., adipose, osseous, cartilaginous, elastic and fibrous connective tissues, myoblasts) as well as to tissues other than those originating in the embryonic mesoderm (e.g., neural cells) depending upon various influences from bioactive factors such as cytokines. Although such cells can be isolated from embryonic yolk sac, placenta, umbilical cord, fetal and adolescent skin, blood and other tissues, their abundance in the BM far exceeds their abundance in other tissues and as such isolation from BM is presently preferred.

Adult tissue stem cells can be isolated using various methods known in the art such as those disclosed by Alison, M. R. [J Pathol. (2003) 200(5): 547-50]. Fetal stem cells can be isolated using various methods known in the art such as those disclosed by Eventov-Friedman S, et al. [PloS Med. (2006) 3: e215].

Hematopoietic stem cells can be isolated using various methods known in the arts such as those disclosed by “Handbook of Stem Cells” edit by Robert Lanze, Elsevier Academic Press, 2004, Chapter 54, pp 609-614, isolation and characterization of hematopoietic stem cells, by Gerald J Spangrude and William B Stayton.

Methods of isolating, purifying and expanding mesenchymal stem cells (MSCs) are known in the arts and include, for example, those disclosed by Caplan and Haynesworth in U.S. Pat. No. 5,486,359 and Jones E. A. et al., 2002, Isolation and characterization of bone marrow multipotential mesenchymal progenitor cells, Arthritis Rheum. 46(12): 3349-60.

According to one embodiment, the eukaryotic cell is isolated from its natural environment (e.g. human body).

According to one embodiment, the eukaryotic cell is a healthy cell.

According to one embodiment, the eukaryotic cell is a diseased cell or a cell prone to a disease.

According to one embodiment, the eukaryotic cell is a cancer cell.

According to one embodiment, the eukaryotic cell is an immune cell (e.g. T cell, B cell, macrophage, NK cell, etc.).

According to one embodiment, the eukaryotic cell is a cell infected by a pathogen (e.g. by a bacterial, viral or fungal pathogen).

The term “RNA molecule having a silencing activity” or “RNA silencing molecule” refers to a non-coding RNA (ncRNA) molecule, i.e. an RNA sequence that is not translated into an amino acid sequence and does not encode a protein, capable of mediating RNA silencing or RNA interference (RNAi).

The term “RNA silencing” or “RNAi” refers to a cellular regulatory mechanism in which non-coding RNA molecules (the “RNA molecule having a silencing activity” or “RNA silencing molecule”) mediate, in a sequence specific manner, co- or post-transcriptional inhibition of gene expression or translation.

According to one embodiment, the RNA silencing molecule is capable of mediating RNA repression during transcription (co-transcriptional gene silencing).

According to a specific embodiment, co-transcriptional gene silencing includes epigenetic silencing (e.g. chromatic state that prevents functional gene expression).

According to one embodiment, the RNA silencing molecule is capable of mediating RNA repression after transcription (post-transcriptional gene silencing).

Post-transcriptional gene silencing (PTGS) typically refers to the process (typically occurring in the cell cytoplasm) of degradation or cleavage of messenger RNA (mRNA) molecules which decrease their activity by preventing translation. For example, and as discussed in detail below, a guide strand of an RNA silencing molecule pairs with a complementary sequence in a mRNA molecule and induces cleavage by e.g. Argonaute 2 (Ago2). Specifically, a member of the Argonaute (Ago) protein family serves as the direct interaction partner of the RNA silencing molecule within the RNA-induced silencing complex (RISC). The RNA silencing molecule acts to guide the RISC to its target mRNA while the Ago protein complex represses mRNA translation or induces deadenylation-dependent mRNA decay, leading to silencing of gene expression.

Co-transcriptional gene silencing typically refers to inactivation of gene activity (i.e. transcription repression) and typically occurs in the cell nucleus. Such gene activity repression is mediated by epigenetic-related factors, such as e.g. methyl-transferases, that methylate target DNA and histones. Thus, in co-transcriptional gene silencing, the association of a small RNA with a target RNA (small RNA-transcript interaction) destabilizes the target nascent transcript and recruits DNA- and histone-modifying enzymes (i.e. epigenetic factors) that induce chromatin remodeling into a structure that repress gene activity and transcription. Also, in co-transcriptional gene silencing, chromatin-associated long non-coding RNA scaffolds may recruit chromatin-modifying complexes independently of small RNAs. These co-transcriptional silencing mechanisms form RNA surveillance systems that detect and silence inappropriate transcription events, and provide a memory of these events via self-reinforcing epigenetic loops [as described in D. Hoch and D. Moazed, RNA-mediated epigenetic regulation of gene expression, Nat Rev Genet. (2015) 16(2): 71-84].

Following is a detailed description of RNA silencing molecules which are engaged with RNA-induced silencing complex (RISC) and comprise an intrinsic RNAi activity (e.g. are RNA silencing molecules) that can be used according to specific embodiments of the present invention.

Perfect and imperfect based paired RNA (i.e. double stranded RNA; dRNA), siRNA and shRNA—The presence of long dsRNAs in cells stimulates the activity of a ribonuclease III enzyme referred to as dicer. Dicer (also known as endoribonuclease Dicer or helicase with Rnase motif) is an enzyme that in plants is typically referred to as Dicer-like (DCL) protein. Different plants have different numbers of DCL genes, thus for example, Arabidopsis genome typically has four DCL genes, rice has eight DCL genes, and maize genome has five DCL genes. Dicer is involved in the processing of the dsRNA into short pieces of dsRNA known as short interfering RNAs (siRNAs). siRNAs derived from dicer activity are typically about 21 to about 23 nucleotides in length and comprise about 19 base pair duplexes with two 3′ nucleotides overhangs.

According to one embodiment dsRNA precursors longer than 21 bp are used. Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl Acad. Sci. USA. 2002:99:1443-1448; Tran N., et al., FEBS Lett. 2004; 573:127-134].

The term “siRNA” refers to small inhibitory RNA duplexes (generally between 18-30 base pairs) that induce the RNA interference (RNAi) pathway. Typically, siRNAs are chemically synthesized as 21 mers with a central 19 bp duplex region and symmetric 2-base 3′-overhangs on the termini, although it has been recently described that chemically synthesized RNA duplexes of 25-30 base length can have as much as a 100-fold increase in potency compared with 21 mers at the same location. The observed increased potency obtained using longer RNAs in triggering RNAi is suggested to result from providing Dicer with a substrate (27 mer) instead of a product (21 mer) and that this improves the rate or efficiency of entry of the siRNA duplex into RISC.

It has been found that position, but not the composition, of the 3′-overhang influences potency of a siRNA and asymmetric duplexes having a 3′-overhang on the antisense strand are generally more potent than those with the 3′-overhang on the sense strand (Rose et al., 2005).

The strands of a double-stranded interfering RNA (e.g., a siRNA) may be connected to form a hairpin or stem-loop structure (e.g., a shRNA). Thus, as mentioned, the RNA silencing molecule of some embodiments of the invention may also be a short hairpin RNA (shRNA).

The term short hairpin RNA, “shRNA”, as used herein, refers to an RNA molecule having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-CAAGAGA-3′ and 5′-UUACAA-3′ (International Patent Application Nos. WO2013126963 and WO2014107763). It will be recognized by one of skill in the art that the resulting single chain oligonucleotide forms a stem-loop or hairpin structure comprising a double-stranded region capable of interacting with the RNAi machinery.

The RNA silencing molecule of some embodiments of the invention need not be limited to those molecules containing only RNA, but further encompasses chemically-modified nucleotides and non-nucleotides.

Various types of siRNAs are contemplated by the present invention, including trans-acting siRNAs (Ta-siRNAs or TasiRNA), repeat-associated siRNAs (Ra-siRNAs) and natural-antisense transcript-derived siRNAs (Nat-siRNAs).

According to one embodiment, silencing RNA includes “piRNA” which is a class of Piwi-interacting RNAs of about 26 and 31 nucleotides in length. piRNAs typically form RNA-protein complexes through interactions with Piwi proteins, i.e. antisense piRNAs are typically loaded into Piwi proteins (e.g. Piwi, Ago3 and Aubergine (Aub)).

miRNA—According to another embodiment the RNA silencing molecule may be a miRNA.

The term “microRNA”, “miRNA”, and “miR” are synonymous and refer to a collection of non-coding single-stranded RNA molecules of about 19-24 nucleotides in length, which regulate gene expression. miRNAs are found in a wide range of organisms (e.g. insects, mammals, plants, nematodes) and have been shown to play a role in development, homeostasis, and disease etiology.

Initially the pre-miRNA is present as a long non-perfect double-stranded stem loop RNA that is further processed by Dicer into a siRNA-like duplex, comprising the mature guide strand (miRNA) and a similar-sized fragment known as the passenger strand (miRNA*). The miRNA and miRNA* may be derived from opposing arms of the pri-miRNA and pre-miRNA. miRNA* sequences may be found in libraries of cloned miRNAs but typically at lower frequency than the miRNAs.

Although initially present as a double-stranded species with miRNA*, the miRNA eventually becomes incorporated as a single-stranded RNA into a ribonucleoprotein complex known as the RNA-induced silencing complex (RISC). Various proteins can form the RISC, which can lead to variability in specificity for miRNA/miRNA* duplexes, binding site of the target gene, activity of miRNA (repress or activate), and which strand of the miRNA/miRNA* duplex is loaded in to the RISC.

When the miRNA strand of the miRNA:miRNA* duplex is loaded into the RISC, the miRNA* is removed and degraded. The strand of the miRNA:miRNA* duplex that is loaded into the RISC is the strand whose 5′ end is less tightly paired. In cases where both ends of the miRNA:miRNA* have roughly equivalent 5′ pairing, both miRNA and miRNA* may have gene silencing activity.

The RISC identifies target nucleic acids based on high levels of complementarity between the miRNA and the mRNA, especially by nucleotides 2-8 of the miRNA (referred as “seed sequence”).

A number of studies have looked at the base-pairing requirement between miRNA and its mRNA target for achieving efficient inhibition of translation (reviewed by Bartel 2004, Cell 116-281). Computational studies, analyzing miRNA binding on whole genomes have suggested a specific role for bases 2-8 at the 5′ of the miRNA (also referred to as “seed sequence”) in target binding but the role of the first nucleotide, found usually to be “A” was also recognized (Lewis et al. 2005 Cell 120-15). Similarly, nucleotides 1-7 or 2-8 were used to identify and validate targets by Krek et al. (2005, Nat Genet 37-495). The target sites in the mRNA may be in the 5′ UTR, the 3′ UTR or in the coding region. Interestingly, multiple miRNAs may regulate the same mRNA target by recognizing the same or multiple sites. The presence of multiple miRNA binding sites in most genetically identified targets may indicate that the cooperative action of multiple RISCs provides the most efficient translational inhibition.

miRNAs may direct the RISC to downregulate gene expression by either of two mechanisms: mRNA cleavage or translational repression. The miRNA may specify cleavage of the mRNA if the mRNA has a certain degree of complementarity to the miRNA. When a miRNA guides cleavage, the cut is typically between the nucleotides pairing to residues 10 and 11 of the miRNA. Alternatively, the miRNA may repress translation if the miRNA does not have the requisite degree of complementarity to the miRNA. Translational repression may be more prevalent in animals since animals may have a lower degree of complementarity between the miRNA and binding site.

It should be noted that there may be variability in the 5′ and 3′ ends of any pair of miRNA and miRNA*. This variability may be due to variability in the enzymatic processing of Drosha and Dicer with respect to the site of cleavage. Variability at the 5′ and 3′ ends of miRNA and miRNA* may also be due to mismatches in the stem structures of the pri-miRNA and pre-miRNA. The mismatches of the stem strands may lead to a population of different hairpin structures. Variability in the stem structures may also lead to variability in the products of cleavage by Drosha and Dicer.

According to one embodiment, miRNAs can be processed independently of Dicer, e.g. by Argonaute 2.

It will be appreciated that the pre-miRNA sequence may comprise from 45-90, 60-80 or 60-70 nucleotides while the pri-miRNA sequence may comprise from 45-30,000, 50-25,000, 100-20,000, 1,000-1,500 or 80-100 nucleotides.

Antisense—Antisense is a single stranded RNA designed to prevent or inhibit expression of a gene by specifically hybridizing to its mRNA. Downregulation of a target RNA can be effected using an antisense polynucleotide capable of specifically hybridizing with an mRNA transcript encoding the target RNA.

Transposable Element RNA

Transposable genetic elements (Tes) comprise a vast array of DNA sequences, all having the ability to move to new sites in genomes either directly by a cut-and-paste mechanism (transposons) or indirectly through an RNA intermediate (retrotransposons). Tes are divided into autonomous and non-autonomous classes depending on whether they have ORFs that encode proteins required for transposition. RNA-mediated gene silencing is one of the mechanisms in which the genome control Tes activity and deleterious effects derived from genome genetic and epigenetic instability.

According to one embodiment, the RNA silencing molecule may be engaged with RISC yet may not comprise a canonical (intrinsic) RNAi activity (e.g. is not a canonical RNA silencing molecule, or its target has not been identified). Such RNA silencing molecule includes the following:

According to one embodiment, the RNA silencing molecule is a transfer RNA (tRNA) or a transfer RNA fragment (tRF). The term “tRNA” refers to an RNA molecule that serves as the physical link between nucleotide sequence of nucleic acids and the amino acid sequence of proteins, formerly referred to as soluble RNA or sRNA. tRNA is typically about 76 to 90 nucleotides in length. According to one embodiment, the RNA silencing molecule is a ribosomal RNA (rRNA). The term “rRNA” refers to the RNA component of the ribosome i.e. of either the small ribosomal subunit or the large ribosomal subunit.

According to one embodiment, the RNA silencing molecule is a small nuclear RNA (snRNA or U-RNA). The terms “sRNA” or “U-RNA” refer to the small RNA molecules found within the splicing speckles and Cajal bodies of the cell nucleus in eukaryotic cells. snRNA is typically about 150 nucleotides in length.

According to one embodiment, the RNA silencing molecule is a small nucleolar RNA (snoRNA). The term “snoRNA” refers to the class of small RNA molecules that primarily guide chemical modifications of other RNAs, e.g. rRNAs, tRNAs and snRNAs. snoRNA is typically classified into one of two classes: the C/D box snoRNAs are typically about 70-120 nucleotides in length and are associated with methylation, and the H/ACA box snoRNAs are typically about 100-200 nucleotides in length and are associated with pseudouridylation.

Similar to snoRNAs are the scaRNAs (i.e. Small Cajal body RNA genes) which perform a similar role in RNA maturation to snoRNAs, but their targets are spliceosomal snRNAs and they perform site-specific modifications of spliceosomal snRNA precursors (in the Cajal bodies of the nucleus).

According to one embodiment, the RNA silencing molecule is an extracellular RNA (exRNA). The term “exRNA” refers to RNA species present outside of the cells from which they were transcribed (e.g. exosomal RNA).

According to one embodiment, the RNA silencing molecule is a long non-coding RNA (lncRNA). The term “lncRNA” or “long ncRNA” refers to non-protein coding transcripts typically longer than 200 nucleotides.

According to a specific embodiment, non-limiting examples of RNA molecules engaged with RISC include, but are not limited to, microRNA (miRNA), piwi-interacting RNA (piRNA), short interfering RNA (siRNA), short-hairpin RNA (shRNA), phased small interfering RNA (phasiRNA), trans-acting siRNA (tasiRNA), small nuclear RNA (snRNA or URNA), transposable element RNA (e.g. autonomous and non-autonomous transposable RNA), transfer RNA (tRNA), small nucleolar RNA (snoRNA), Small Cajal body RNA (scaRNA), ribosomal RNA (rRNA), extracellular RNA (exRNA), repeat-derived RNA, and long non-coding RNA (lncRNA).

According to a specific embodiment, non-limiting examples of RNAi molecules engaged with RISC include, but are not limited to, small interfering RNA (siRNA), short hairpin RNA (shRNA), microRNA (miRNA), Piwi-interacting RNA (piRNA), phased small interfering RNA (phasiRNA), and trans-acting siRNA (tasiRNA).

According to one embodiment, the method comprises identifying nucleic acid sequences encoding RNA molecules exhibiting a predetermined sequence homology range, not including complete identity, with respect to a nucleic acid sequence encoding an RNA molecule engaged with RISC (e.g. RNAi-like or miRNA-like sequences).

According to one embodiment, the RNA molecules of step (a) exhibit a predetermined sequence homology range, not including complete identity, with respect to an RNA molecule that is engaged with—and/or that is processed into a molecule engaged with RISC.

The term “RNAi-like” refers to sequences in the genome that comprise a sequence homology to RNA silencing molecules but are not identical to the sequences of the RNA silencing molecules.

The term “miRNA-like” refers to sequences in the genome that comprise a sequence homology to miRNA but are not identical to miRNA sequences.

Such non-coding RNA-related molecules (i.e. miRNA-like molecules) can be functional (e.g. being processable and/or having a silencing activity, as discussed below), or alternatively, can be dysfunctional (e.g. are non-processable, or processed aberrantly and/or do not have a silencing activity, as discussed below). According to one embodiment, the sequence homology range comprises 50%-99.9%, 60%-99.9%, 70%-99.9%, 75%-99.9%, 80%-99.9%, 85%-99.9%, 90%-99.9%, 95%-99.9% identity with respect to the nucleic acid sequence encoding the RNA molecule engaged with RISC.

According to a specific embodiment, the sequence homology range comprises 50%-75% identity with respect to the nucleic acid sequence encoding the RNA molecule engaged with RISC.

According to a specific embodiment, the sequence homology range comprises 50%-99.9% identity with respect to the nucleic acid sequence encoding the RNA molecule engaged with RISC.

According to a specific embodiment, the sequence homology range comprises 70%-99.9% identity with respect to the nucleic acid sequence encoding the RNA molecule engaged with RISC.

According to a specific embodiment, the sequence homology range comprises 75%-99.6% identity with respect to the nucleic acid sequence encoding the RNA molecule engaged with RISC.

According to a specific embodiment, the sequence homology range comprises 85%-99.6% identity with respect to the nucleic acid sequence encoding the RNA molecule engaged with RISC.

According to one embodiment, the sequence homology comprises 50%, 60%, 70%, 75%, 80%, 85%, 90%, 91%, 92%, 93%, 94%, 95%, 96%, 97%, 98%, 99%, 99.6% or 99.9% identity with respect to the nucleic acid sequence encoding the RNA molecule engaged with RISC.

According to one embodiment, the sequence homology range comprises 50%-99.9%, 60%-99.9%, 70%-99.9%, 75%-99.9%, 80%-99.9%, 85%-99.9%, 90%-99.9%, 95%-99.9% identity with respect to a nucleic acid sequence encoding and processed into a RISC-engaged RNA molecule.

According to a specific embodiment, the sequence homology range comprises 50%-75% identity with respect to a nucleic acid sequence encoding and processed into a RISC-engaged RNA molecule.

According to a specific embodiment, the sequence homology range comprises 50%-99.6% identity with respect to a nucleic acid sequence encoding and processed into a RISC-engaged RNA molecule.

According to a specific embodiment, the sequence homology range comprises 70%-99.9% identity with respect to a nucleic acid sequence encoding and processed into a RISC-engaged RNA molecule.

According to a specific embodiment, the sequence homology range comprises 75%-99.6% identity with respect to a nucleic acid sequence encoding and processed into a RISC-engaged RNA molecule.

According to a specific embodiment, the sequence homology range comprises 85%-99.6% identity with respect to a nucleic acid sequence encoding and processed into a RISC-engaged RNA molecule.

According to one embodiment, the sequence homology comprises 50%, 60%, 70%, 75%, 80%, 85%, 90%, 91%, 92%, 93%, 94%, 95%, 96%, 97%, 98%, 99%, 99.6% or 99.9% identity with respect to a nucleic acid sequence encoding and processed into a RISC-engaged RNA molecule.

According to one embodiment, the sequence homology range comprises 50%-99.9%, 60%-99.9%, 70%-99.9%, 75%-99.9%, 80%-99.9%, 85%-99.9%, 90%-99.9%, 95%-99.9% identity with respect to a nucleic acid sequence of a mature RNA silencing molecule engaged with RISC.

According to a specific embodiment, the sequence homology range comprises 50%-75% identity with respect to a nucleic acid sequence of a mature RNA silencing molecule engaged with RISC.

According to a specific embodiment, the sequence homology range comprises 50%-99.6% identity with respect to a nucleic acid sequence of a mature RNA silencing molecule engaged with RISC.

According to a specific embodiment, the sequence homology range comprises 70%-99.9% identity with respect to a nucleic acid sequence of a mature RNA silencing molecule engaged with RISC.

According to a specific embodiment, the sequence homology range comprises 75%-99.6% identity with respect to a nucleic acid sequence of a mature RNA silencing molecule engaged with RISC.

According to a specific embodiment, the sequence homology range comprises 85%-99.6% identity with respect to a nucleic acid sequence of a mature RNA silencing molecule engaged with RISC.

According to one embodiment, the sequence homology comprises 50%, 60%, 70%, 75%, 80%, 85%, 90%, 91%, 92%, 93%, 94%, 95%, 96%, 97%, 98%, 99%, 99.6% or 99.9% identity with respect to a nucleic acid sequence of a mature RNA silencing molecule engaged with RISC.

According to some embodiments, the phrase “predetermined sequence homology range” as used herein refers to a combination of sequence coverage and sequence homology. As known to the skilled person, the term “sequence coverage” refers to the length of a query sequence which contains at least some nucleotides that perfectly match a second sequence, such as a genomic region (e.g. if only the last 90 bases of a 100 bases query sequence contain nucleotides that match the second sequence, there is 90% coverage). As known to the skilled person, there might be different degrees of homology within the covered sequence (e.g. a sequence with 90% coverage might have a different number of identical nucleotides, different gaps etc, and thus a different degree of homology). Any method known in the art can be used to assess sequence coverage and sequence homology, e.g. sequence alignment programs such as Blast provide the length of the sequences and the length of the alignment region, from which the sequence coverage can be extracted.

According to some embodiments, the predetermined sequence homology range comprises a sequence coverage of between about 50%-100% of the aligned sequences, possibly between about 70%-100% of the aligned sequences. According to other embodiments, the predetermined sequence homology range comprises a sequence coverage of between about 5%-100%, 25%-100%, 40%-100%, 50%-100%, 7004-100% or 75%-100. Each possibility represents a separate embodiment of the present invention.

According to some embodiments, the predetermined sequence homology range comprises: (1) a sequence coverage of between about 50%-100% of the aligned sequences, possibly between about 70%-100% of the aligned sequences; and (2) a sequence homology of between about 75%-100%, possibly between about 85%-100%. Each possibility represents a separate embodiment of the present invention. According to some embodiments, the predetermined sequence homology range comprises at least a coverage of about 50% with a homology of at least about 75%.

According to some embodiments, a nucleic acid sequence encoding an RNA molecule has a predetermined sequence homology range to a nucleic acid sequence encoding a corresponding silencing RNA (e.g. miRNA) if. (a) it is found in a blast search with the corresponding silencing RNA (or part thereof) using default parameters (e.g. www(dot)arabidopsis(dot)org/Blast/BLASToptions(dot)jsp) with respect to a corresponding ncRNA (e.g. miRNA); and (b) its sequence covers at least 50% of a mature sequence of that corresponding silencing RNA (e.g. a mature miRNA sequence), wherein the mature sequence is possibly 19-24 nt long, possibly 19-21 nt long. Each possibility represents a separate embodiment of the present invention.

According to one embodiment, the sequence homology does not include 100% identity.

Homology (e.g., percent homology, sequence identity+sequence similarity) can be determined using any homology comparison software computing a pairwise sequence alignment.

As used herein, “sequence identity” or “identity” in the context of two nucleic acid or polypeptide sequences includes reference to the residues in the two sequences which are the same when aligned. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are considered to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Henikoff S and Henikoff J G. [Amino acid substitution matrices from protein blocks. Proc. Natl. Acad. Sci. U.S.A. 1992, 89(22): 10915-9].

Identity (e.g., percent homology) can be determined using any homology comparison software, including for example, the BlastN software of the National Center of Biotechnology Information (NCBI) such as by using default parameters.

According to some embodiments of the invention, the identity is a global identity, i.e., an identity over the entire amino acid or nucleic acid sequences of the invention and not over portions thereof.

According to some embodiments of the invention, the term “homology” or “homologous” refers to identity of two or more nucleic acid sequences; or identity of two or more amino acid sequences; or the identity of an amino acid sequence to one or more nucleic acid sequence.

According to some embodiments of the invention, the homology is a global homology, i.e., a homology over the entire amino acid or nucleic acid sequences of the invention and not over portions thereof.

The degree of homology or identity between two or more sequences can be determined using various known sequence comparison tools. Following is a non-limiting description of such tools which can be used along with some embodiments of the invention.

When starting with a polynucleotide sequence and comparing to other polynucleotide sequences the EMBOSS-6.0.1 Needleman-Wunsch algorithm (available from emboss(dot)sourceforge(dot)net/apps/cvs/emboss/apps/needle(dot)html) can be used with the following default parameters: (EMBOSS-6.0.1) gapopen=10; gapextend=0.5; datafile=EDNAFULL; brief=YES.

According to some embodiments of the invention, the parameters used with the EMBOSS-6.0.1 Needleman-Wunsch algorithm are gapopen=10; gapextend=0.2; datafile=EDNAFULL; brief=YES.

According to some embodiments of the invention, the threshold used to determine homology using the EMBOSS-6.0.1 Needleman-Wunsch algorithm for comparison of polynucleotides with polynucleotides is 80%, 81%, 82%, 83%, 84%, 85%, 86%, 87%, 88%, 89%, 90%, 91%, 92%, 93%, 94%, 95%, 96%, 97%, 98%, 99%, or 100%.

According to some embodiment, determination of the degree of homology further requires employing the Smith-Waterman algorithm (for protein-protein comparison or nucleotide-nucleotide comparison).

Default parameters for GenCore 6.0 Smith-Waterman algorithm include: model=sw.model.

According to some embodiments of the invention, the threshold used to determine homology using the Smith-Waterman algorithm is 80%, 81%, 82%, 83%, 84%, 85%, 86%, 87%, 88%, 89%, 90%, 91%, 92%, 93%, 94%, 95%, 96%, 97%, 98%, 99%, or 100%.

According to some embodiments of the invention, the global homology is performed on sequences which are pre-selected by local homology to the polypeptide or polynucleotide of interest (e.g., 60% identity over 60% of the sequence length), prior to performing the global homology to the polypeptide or polynucleotide of interest (e.g., 80% global homology on the entire sequence). For example, homologous sequences are selected using the BLAST software with the Blastp and tBlastn algorithms as filters for the first stage, and the needle (EMBOSS package) or Frame+ algorithm alignment for the second stage. Local identity (Blast alignments) is defined with a very permissive cutoff—60% Identity on a span of 60% of the sequences lengths because it is used only as a filter for the global alignment stage. In this specific embodiment (when the local identity is used), the default filtering of the Blast package is not utilized (by setting the parameter “-F F”).

In the second stage, homologs are defined based on a global identity of at least 80% to the core gene polypeptide sequence. According to some embodiments the homology is a local homology or a local identity.

Local alignments tools include, but are not limited to the BlastP, BlastN, BlastX or TBLASTN software of the National Center of Biotechnology Information (NCBI), FASTA, and the Smith-Waterman algorithm.

According to a specific embodiment, homology is determined using BlastN version 2.7.1+ with the following default parameters: task=blastn, evalue=10, strand=both, gap opening penalty=5, gap extension penalty=2, match=1, mismatch=−1, word size=11, max scores—25, max alignments=15, query filter=dust, query genetic code—n/a, matrix=no default.

According to one embodiment, the method further comprises determining the genomic location of the nucleic acid sequences encoding the RNA molecules exhibiting the predetermined sequence homology range of step (a).

According to one embodiment, the nucleic acid sequence encoding the RNAi-like molecule (e.g. miRNA-like molecule) is positioned in a non-coding gene (e.g. non-protein coding gene). Exemplary non-coding parts of the genome include, but are not limited to, genes of non-coding RNAs, enhancers and locus control regions, insulators, S/MAR sequences, non-coding pseudogenes, non-autonomous transposons and retrotransposons, and non-coding simple repeats of centromeric and telomeric regions of chromosomes.

According to one embodiment, the nucleic acid sequence encoding the RNAi-like molecule (e.g. miRNA-like molecule) is positioned within an intron of a non-coding gene.

According to one embodiment, the nucleic acid sequence encoding the RNAi-like molecule (e.g. miRNA-like molecule) is positioned in a non-coding gene that is ubiquitously expressed.

According to one embodiment, the nucleic acid sequence encoding the RNAi-like molecule (e.g. miRNA-like molecule) is positioned in a non-coding gene that is expressed in a tissue-specific manner.

According to one embodiment, the nucleic acid sequence encoding the RNAi-like molecule (e.g. miRNA-like molecule) is positioned in a non-coding gene that is expressed in an inducible manner.

According to one embodiment, the nucleic acid sequence encoding the RNAi-like molecule (e.g. miRNA-like molecule) is positioned in a non-coding gene that is developmentally regulated.

According to one embodiment, the nucleic acid sequence encoding the RNAi-like molecule (e.g. miRNA-like molecule) is positioned between genes, i.e. intergenic region.

According to one embodiment, the nucleic acid sequence encoding the RNAi-like molecule (e.g. miRNA-like molecule) is positioned in a coding gene (e.g. protein-coding gene).

According to one embodiment, the nucleic acid sequence encoding the RNAi-like molecule (e.g. miRNA-like molecule) is positioned within an exon of a coding gene (e.g. protein-coding gene).

According to one embodiment, the nucleic acid sequence encoding the RNAi-like molecule (e.g. miRNA-like molecule) is positioned within an exon encoding an untranslated region (UTR) of a coding gene (e.g. protein-coding gene).

According to one embodiment, the nucleic acid sequence encoding the RNAi-like molecule (e.g. miRNA-like molecule) is positioned within a translated exon of a coding gene (e.g. protein-coding gene).

According to one embodiment, the nucleic acid sequence encoding the RNAi-like molecule (e.g. miRNA-like molecule) is positioned within an intron of a coding gene (e.g. protein-coding gene).

According to one embodiment, the nucleic acid sequence encoding the RNAi-like molecule (e.g. miRNA-like molecule) is positioned within a coding gene that is ubiquitously expressed.

According to one embodiment, the nucleic acid sequence encoding the RNAi-like molecule (e.g. miRNA-like molecule) is positioned within a coding gene that is expressed in a tissue-specific manner.

According to one embodiment, the nucleic acid sequence encoding the RNAi-like molecule (e.g. miRNA-like molecule) is positioned within coding gene that is expressed in an inducible manner.

According to one embodiment, the nucleic acid sequence encoding the RNAi-like molecule (e.g. miRNA-like molecule) is positioned within coding gene that is developmentally regulated.

According to one embodiment, the method comprises determining transcription of the nucleic acid sequences encoding the RNA molecules so as to select transcribable nucleic acid sequences encoding the RNA molecules exhibiting the predetermined sequence homology range.

The phrase “transcribable nucleic acid sequence” refers to a DNA segment capable of being transcribed into RNA.

Assessment of transcription of a nucleic acid sequence can be carried out using any method known in the art, such as by, RT-PCR, Northern-blot, RNA-seq, small RNA seq.

As mentioned, the method of some embodiments of the invention enables identification of RNA silencing molecules capable of being transcribed yet not processed into small RNAs engaged with RISC.

According to one embodiment, the method comprises determining processability into small RNAs of transcripts of the transcribable nucleic acid sequences encoding the RNA molecules exhibiting the predetermined sequence homology range so as to select aberrantly processed (e.g. non-processable), transcribable nucleic acid sequences encoding the RNA molecules exhibiting the predetermined sequence homology range.

The terms “processing” or “processability” refer to the biogenesis by which RNA molecules are cleaved into small RNA form capable of engaging with RNA-induced silencing complex (RISC). Exemplary processing mechanisms include e.g., Dicer and Argonaute, as further discussed below. For example, pre-miRNA is processed into a mature miRNA by Dicer.

The term “canonical processing” is used herein with respect to an RNA precursor for a silencing RNA of a certain class (e.g. miRNA) and refers to processing of an RNA molecule into small RNA molecules, wherein the processing pattern (e.g. number, size and/or location of resulting small RNA molecules) is typical of a precursor in that class of silencing RNA molecules. Typically, a small RNA molecule which is a result of canonical processing is capable of engaging with RISC and binding to its natural target RNA (i.e. first target RNA). According to some embodiments, reference to wild-type processing as used herein refers to canonical processing. According to some embodiments, reference to a wild-type silencing molecule refers to a canonical silencing molecule (i.e. which acts, has a structure and/or is processed according to known behavior of a silencing molecule of that class in the art).

The term “aberrantly processed” as used herein, is a comparative term and refers to processing of an RNA molecule into small RNA molecules, such that the processing is not canonical processing with respect to an RNA precursor of a silencing RNA in a certain class (e.g. miRNA). In a non-limiting example, an RNA molecule homologous to a precursor for a silencing RNA molecule of a certain class (e.g. a miRNA precursor), which is processed differently than that precursor (which is canonically processed), is aberrantly processed.

According to some embodiments, aberrantly processed is selected from the group consisting of: non-processed (i.e. not generating any small RNA molecules) and differently processed compared to canonical processing (i.e. processed to small RNA molecules in a number, size and/or location which is different than that achieved in canonical processing). Small RNA molecules resulting from aberrant processing are typically of an aberrant size (as compared to small RNA molecules resulting from canonical processing), are not engaged with RISC and/or are not complementary to their natural target RNA (i.e. first target RNA). Each possibility represents a separate embodiment of the present invention.

As used herein, the term “small RNA form” or “small RNAs” or “small RNA molecule” refers to the mature small RNA being capable of hybridizing with a target RNA (or fragment thereof).

As used herein, the phrase “dysfunctional RNA molecule” refers to an RNA molecule (e.g. non-coding RNA molecule, e.g. RNAi molecule) which is not processed into small RNAs capable of engaging with RISC and does not silence a natural target RNA (i.e. first target RNA). According to one embodiment, the dysfunctional RNA molecule comprises a sequence alternation (e.g. sequence alteration in a precursor sequence) which alters its secondary RNA structure and renders it aberrantly processed (e.g. non-processable).

According to one embodiment, the small RNA form has a silencing activity.

According to one embodiment, the small RNAs comprise no more than 250 nucleotides in length, e.g. comprise 15-250, 15-200, 15-150, 15-100, 15-50, 15-40, 15-30, 15-25, 15-20, 20-30, 20-25, 30-100, 30-80, 30-60, 30-50, 30-40, 30-35, 50-150, 50-100, 50-80, 50-70, 50-60, 100-250, 100-200, 100-150, 150-250, 150-200 nucleotides.

According to a specific embodiment, the small RNA molecules comprise 20-50 nucleotides.

According to a specific embodiment, the small RNA molecules comprise 20-30 nucleotides.

According to a specific embodiment, the small RNA molecules comprise 21-29 nucleotides.

According to a specific embodiment, the small RNA molecules comprise 21-23 nucleotides.

According to a specific embodiment, the small RNA molecules comprise 21 nucleotides.

According to a specific embodiment, the small RNA molecules comprise 22 nucleotides.

According to a specific embodiment, the small RNA molecules comprise 23 nucleotides.

According to a specific embodiment, the small RNA molecules comprise 24 nucleotides.

According to a specific embodiment, the small RNA molecules comprise 25 nucleotides.

According to a specific embodiment, the small RNA molecules consist of 20-50 nucleotides.

According to a specific embodiment, the small RNA molecules consist of 20-30 nucleotides.

According to a specific embodiment, the small RNA molecules consist of 21-29 nucleotides.

According to a specific embodiment, the small RNA molecules consist of 21-23 nucleotides.

According to a specific embodiment, the small RNA molecules consist of 21 nucleotides.

According to a specific embodiment, the small RNA molecules consist of 22 nucleotides.

According to a specific embodiment, the small RNA molecules consist of 23 nucleotides.

According to a specific embodiment, the small RNA molecules consist of 24 nucleotides.

According to a specific embodiment, the small RNA molecules consist of 25 nucleotides.

Typically, processability depends on a structure of an RNA molecule, also referred to herein as originality of structure, i.e. the secondary RNA structure (i.e. base pairing profile). The secondary RNA structure is important for correct and efficient processing of the RNA molecule into small RNAs (such as siRNA or miRNA) that is structure- and not purely sequence-dependent.

Thus, according to one embodiment, the selected or identified nucleic acid sequences encoding RNA molecules of step (a) are homologous to genes encoding silencing RNA molecules whose silencing activity and/or processing into small silencing RNA is dependent on their secondary structure.

According to some embodiments, a silencing RNA molecule whose silencing activity and/or processing into small silencing RNA is dependent on secondary structure is selected from the group consisting of: microRNA (miRNA), short-hairpin RNA (shRNA), small nuclear RNA (snRNA or U-RNA), small nucleolar RNA (snoRNA), Small Cajal body RNA (scaRNA), transfer RNA (tRNA), ribosomal RNA (rRNA), repeat-derived RNA, autonomous and non-autonomous transposable and retro-transposable element-derived RNA, autonomous and non-autonomous transposable and retro-transposable element RNA and long non-coding RNA (lncRNA).

According to one embodiment, the cellular RNAi processing machinery, i.e. cellular RNAi processing and executing factors, process the RNA molecules into small RNAs.

According to one embodiment, the cellular RNAi processing machinery comprises ribonucleases, including but not limited to, the DICER protein family (e.g. DCR1 and DCR2), DICER-LIKE protein family (e.g. DCL1, DCL2, DCL3, DCL4), ARGONAUTE protein family (e.g. AGO1, AGO2, AGO3, AGO4), tRNA cleavage enzymes (e.g. RNY1, ANGIOGENIN, Rnase P, Rnase P-like, SLFN3, ELAC1 and ELAC2), and Piwi-interacting RNA (piRNA) related proteins (e.g. AGO3, AUBERGINE, HIWI, HIWI2, HIWI3, PIWI, ALG1 and ALG2).

According to one embodiment, the cellular RNAi processing machinery generates the RNA silencing molecule, but no specific target has been identified.

According to one embodiment, the small RNA molecule is processed from a precursor.

According to one embodiment, the small RNA molecule is processed from a single stranded RNA (ssRNA) precursor.

According to one embodiment, the small RNA molecule is processed from a duplex-structured single-stranded RNA precursor.

According to one embodiment, the small RNA molecule is processed from a non-structured RNA precursor.

According to one embodiment, the small RNA molecule is processed from a protein-coding RNA precursor.

According to one embodiment, the small RNA molecule is processed from a non-coding RNA precursor.

According to one embodiment, the small RNA molecule is processed from a dsRNA precursor (e.g. comprising perfect and imperfect base pairing).

According to one embodiment, the dsRNA can be derived from two different complementary RNAs, or from a single RNA that folds on itself to form dsRNA.

Assessment of processing can be carried out using any method known in the art, such as by, small RNA seq, Northern-blot, small RNA qRT-PCR and Rapid Amplification of cDNA Ends (RACE).

For example, for selection for aberrantly processed (e.g. non-processable) nucleic acid sequences a small RNA seq, Northern-blot, small RNA qRT-PCR and Rapid Amplification of cDNA Ends (RACE) method can be applied.

Functional processability can also be determined by comparative structure analysis. For example, the structure of the dysfunctional pre-miRNA-like is compared to the corresponding pre-miRNA capable of processability into small RNA molecules engaged with RISC (e.g. compare precursor structures). An altered dysfunctional structure suggests that it will not be processed, or processed differently than the corresponding pre-miRNA capable of processability into small RNA molecules engaged with RISC. Processing can be validated by small RNA analysis.

According to one embodiment, step (b) and/or (c) are affected by alignment of small RNA expression data to a genome of the cell and determining the amount of reads that map to each genomic location.

According to some embodiment, small RNA analysis for determining processing comprises aligning the sequences of small RNAs expressed in a certain cell or tissue with their corresponding genomic location (e.g. within a gene encoding a potential dysfunctional pre-miRNA-like molecule), to determine the location from which each sRNA is expressed and the number of sRNA reads at each location. According to a specific embodiment, the alignment of the sequences of expressed small RNAs with their corresponding genomic location (i.e. a predetermined location) to determine processing is an alignment with no mismatches.

As mentioned, the aberrantly processed, transcribable nucleic acid sequences encoding the RNA molecules exhibiting the predetermined sequence homology range are selected.

According to one embodiment, the method comprises modifying a nucleic acid sequence of the aberrantly processed (e.g. non-processable), transcribable nucleic acid sequences so as to impart processability into small RNAs that are engaged with RISC and are complementary to a first target RNA (e.g., a natural target RNA as discussed below), also referred to herein as “reactivation” of silencing activity.

According to one embodiment, modifying in step (d) comprises introducing into the cell a DNA editing agent which reactivates silencing activity in the aberrantly processed RNA molecule towards the first target RNA, thereby generating an RNA molecule having a silencing activity in the cell.

According to one embodiment, the method further comprises modifying the specificity of the RNA molecule having the silencing activity in the cell, wherein the DNA editing agent redirects a silencing specificity of the RNA molecule towards a target RNA of interest, the target RNA of interest being distinct from the first target RNA, thereby modifying the specificity of the RNA molecule having the silencing activity in the cell.

According to one embodiment, the difference between modifying to activate silencing towards the first target RNA and modifying specificity might be the use of a different GEiGS oligo when performing GEiGS (i.e. the GEiGS oligo for modifying specificity will further include modifications in the mature miRNA sequence to change specificity).

Following is a description of various non-limiting examples of methods and DNA editing agents used to introduce nucleic acid alterations to a gene encoding an RNA silencing molecule and agents for implementing same that can be used according to specific embodiments of the present disclosure.

Genome Editing using engineered endonucleases—this approach refers to a reverse genetics method using artificially engineered nucleases to typically cut and create specific double-stranded breaks (DSBs) at a desired location(s) in the genome, which are then repaired by cellular endogenous processes such as, homologous recombination (HR) or non-homologous end-joining (NHEJ). NHEJ directly joins the DNA ends in a double-stranded break (DSB) with or without minimal ends trimming, while HR utilizes a homologous donor sequence as a template (i.e. the sister chromatid formed during S-phase) for regenerating/copying the missing DNA sequence at the break site. In order to introduce specific nucleotide modifications to the genomic DNA, a donor DNA repair template containing the desired sequence must be present during HR (exogenously provided single stranded or double stranded DNA).

Genome editing cannot be performed using traditional restriction endonucleases since most restriction enzymes recognize a few base pairs on the DNA as their target and these sequences often will be found in many locations across the genome resulting in multiple cuts which are not limited to a desired location. To overcome this challenge and create site-specific single- or double-stranded breaks (DSBs), several distinct classes of nucleases have been discovered and bioengineered to date. These include the meganucleases, Zinc finger nucleases (ZFNs), transcription-activator like effector nucleases (TALENs) and CRISPR/Cas9 system.

Meganucleases—Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location.

This can be exploited to make site-specific double-stranded breaks (DSBs) in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence.

Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs—Two distinct classes of engineered nucleases, zinc-finger nucleases (ZFNs) and transcription activator-like effector nucleases (TALENs), have both proven to be effective at producing targeted double-stranded breaks (DSBs) (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fokl. Additionally Fokl has the advantage of requiring dimerization to have nuclease activity and this means the specificity increases dramatically as each nuclease partner recognizes a unique DNA sequence. To enhance this effect, Fokl nucleases have been engineered that can only function as heterodimers and have increased catalytic activity. The heterodimer functioning nucleases avoid the possibility of unwanted homodimer activity and thus increase specificity of the double-stranded break (DSB).

Thus, for example to target a specific site, ZFNs and TALENs are constructed as nuclease pairs, with each member of the pair designed to bind adjacent sequences at the targeted site. Upon transient expression in cells, the nucleases bind to their target sites and the FokI domains heterodimerize to create a double-stranded break (DSB). Repair of these double-stranded breaks (DSBs) through the non-homologous end-joining (NHEJ) pathway often results in small deletions or small sequence insertions (Indels). Since each repair made by NHEJ is unique, the use of a single nuclease pair can produce an allelic series with a range of different insertions or deletions at the target site.

In general NHEJ is relatively accurate (about 75-85% of DSBs in human cells are repaired by NHEJ within about 30 min from detection) in gene editing erroneous NHEJ is relied upon as when the repair is accurate the nuclease will keep cutting until the repair product is mutagenic and the recognition/cut site/PAM motif is gone/mutated or that the transiently introduced nuclease is no longer present.

The deletions typically range anywhere from a few base pairs to a few hundred base pairs in length, but larger deletions have been successfully generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break (DSB) can be repaired via homologous recombination (HR) (e.g. in the presence of a donor template) to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers are typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, Calif.).

Method for designing and obtaining TALENs are described in e.g. Reyon et al. Nature Biotechnology 2012 May; 30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www(dot)talendesign(dot)org). TALEN can also be designed and obtained commercially from e.g., Sangamo Biosciences™ (Richmond, Calif.).

T-GEE system (TargetGene's Genome Editing Engine)—A programmable nucleoprotein molecular complex containing a polypeptide moiety and a specificity conferring nucleic acid (SCNA) which assembles in-vivo, in a target cell, and is capable of interacting with the predetermined target nucleic acid sequence is provided. The programmable nucleoprotein molecular complex is capable of specifically modifying and/or editing a target site within the target nucleic acid sequence and/or modifying the function of the target nucleic acid sequence. Nucleoprotein composition comprises (a) polynucleotide molecule encoding a chimeric polypeptide and comprising (i) a functional domain capable of modifying the target site, and (ii) a linking domain that is capable of interacting with a specificity conferring nucleic acid, and (b) specificity conferring nucleic acid (SCNA) comprising (i) a nucleotide sequence complementary to a region of the target nucleic acid flanking the target site, and (ii) a recognition region capable of specifically attaching to the linking domain of the polypeptide. The composition enables modifying a predetermined nucleic acid sequence target precisely, reliably and cost-effectively with high specificity and binding capabilities of molecular complex to the target nucleic acid through base-pairing of specificity-conferring nucleic acid and a target nucleic acid. The composition is less genotoxic, modular in their assembly, utilize single platform without customization, practical for independent use outside of specialized core-facilities, and has shorter development time frame and reduced costs.

CRISPR-Cas system and all its variants (also referred to herein as “CRISPR”)—Many bacteria and archea contain endogenous RNA-based adaptive immune systems that can degrade nucleic acids of invading phages and plasmids. These systems consist of clustered regularly interspaced short palindromic repeat (CRISPR) nucleotide sequences that produce RNA components and CRISPR associated (Cas) genes that encode protein components. The CRISPR RNAs (crRNAs) contain short stretches of homology to the DNA of specific viruses and plasmids and act as guides to direct Cas nucleases to degrade the complementary nucleic acids of the corresponding pathogen. Studies of the type II CRISPR/Cas system of Streptococcus pyogenes have shown that three components form an RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821).

It was further demonstrated that a synthetic chimeric guide RNA (sgRNA) composed of a fusion between crRNA and tracrRNA could direct Cas9 to cleave DNA targets that are complementary to the crRNA in vitro. It was also demonstrated that transient expression of Cas9 in conjunction with synthetic sgRNAs can be used to produce targeted double-stranded breaks (DSBs) in a variety of different species (Cho et al., 2013; Cong et al., 2013; DiCarlo et al., 2013; Hwang et al., 2013a,b; Jinek et al., 2013; Mali et al., 2013).

The CRISPR/Cas system for genome editing contains two distinct components: a sgRNA and an endonuclease e.g. Cas9.

The sgRNA (also referred to herein as short guide RNA (sgRNA)) is typically a 20-nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base-pairing between the sgRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break (DSB). Just as with ZFNs and TALENs, the double-stranded breaks (DSBs) produced by CRISPR/Cas can undergo homologous recombination or NHEJ and are susceptible to specific sequence modification during DNA repair.

The Cas9 nuclease has two functional domains: RuvC and HNH, each cutting a different DNA strand. When both of these domains are active, the Cas9 causes double strand breaks (DSBs) in the genomic DNA.

A significant advantage of CRISPR/Cas is that the high efficiency of this system is coupled with the ability to easily create synthetic sgRNAs. This creates a system that can be readily modified to target modifications at different genomic sites and/or to target different modifications at the same site. Additionally, protocols have been established which enable simultaneous targeting of multiple genes. The majority of cells carrying the mutation present biallelic mutations in the targeted genes.

However, apparent flexibility in the base-pairing interactions between the sgRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is mostly repaired by single strand break repair mechanism involving proteins such as but not only, PARP (sensor) and XRCC1/LIG III complex (ligation). If a single strand break (SSB) is generated by topoisomerase I poisons or by drugs that trap PARP1 on naturally occurring SSBs then these could persist and when the cell enters into S-phase and the replication fork encounter such SSBs they will become single ended DSBs which can only be repaired by HR. However, two proximal, opposite strand nicks introduced by a Cas9 nickase are treated as a double-strand break, in what is often referred to as a ‘double nick’ CRISPR system. A double-nick, which is basically non-parallel DSB, can be repaired like other DSBs by HR or NHEJ depending on the desired effect on the gene target and the presence of a donor sequence and the cell cycle stage (HR is of much lower abundance and can only occur in S and G2 stages of the cell cycle). Thus, if specificity and reduced off-target effects are crucial, using the Cas9 nickase to create a double-nick by designing two sgRNAs with target sequences in close proximity and on opposite strands of the genomic DNA would decrease off-target effect as either sgRNA alone will result in nicks that are not likely to change the genomic DNA, even though these events are not impossible.

Modified versions of the Cas9 enzyme containing two inactive catalytic domains (dead Cas9, or dCas9) have no nuclease activity while still able to bind to DNA based on sgRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

Additional variants of Cas9 which may be used by some embodiments of the invention include, but are not limited to, CasX and Cpf1. CasX enzymes comprise a distinct family of RNA-guided genome editors which are smaller in size compared to Cas9 and are found in bacteria (which is typically not found in humans), hence, are less likely to provoke the immune system/response in a human. Also, CasX utilizes a different PAM motif compared to Cas9 and therefore can be used to target sequences in which Cas9 PAM motifs are not found [see Liu J J et al., Nature. (2019) 566(7743):218-223.]. Cpf1, also referred to as Cas12a, is especially advantageous for editing AT rich regions in which Cas9 PAMs (NGG) are much less abundant [see Li T et al., Biotechnol Adv. (2019) 37(1):21-27; Murugan K et al., Mol Cell. (2017) 68(1):15-25].

According to another embodiment, the CRISPR system may be fused with various effector domains, such as DNA cleavage domains. The DNA cleavage domain can be obtained from any endonuclease or exonuclease. Non-limiting examples of endonucleases from which a DNA cleavage domain can be derived include, but are not limited to, restriction endonucleases and homing endonucleases (see, for example, New England Biolabs Catalog or Belfort et al. (1997) Nucleic Acids Res.). In exemplary embodiments, the cleavage domain of the CRISPR system is a Fokl endonuclease domain or a modified Fokl endonuclease domain. In addition, the use of Homing Endonucleases (HE) is another alternative. Hes are small proteins (<300 amino acids) found in bacteria, archaea, and in unicellular eukaryotes. A distinguishing characteristic of Hes is that they recognize relatively long sequences (14-40 bp) compared to other site-specific endonucleases such as restriction enzymes (4-8 bp). Hes have been historically categorized by small conserved amino acid motifs. At least five such families have been identified: LAGLIDADG; GIY-YIG; HNH; His-Cys Box and PD-(D/E)×K, which are related to Ed×HD enzymes and are considered by some as a separate family. At a structural level, the HNH and His-Cys Box share a common fold (designated Opa-metal) as do the PD-(D/E)×K and Ed×HD enzymes. The catalytic and DNA recognition strategies for each of the families vary and lend themselves to different degrees to engineering for a variety of applications. See e.g. Methods Mol Biol. (2014) 1123:1-26. Exemplary Homing Endonucleases which may be used according to some embodiments of the invention include, without being limited to, I-CreI, I-TevI, I-HmuI, I-PpoI and I-Ssp68031.

Modified versions of CRISPR, e.g. dead CRISPR (dCRISPR-endonuclease), may also be utilized for CRISPR transcription inhibition (CRISPRi) or CRISPR transcription activation (CRISPRa) see e.g. Kampmann M., ACS Chem Biol. (2018) 13(2):406-416; La Russa M F and Qi L S., Mol Cell Biol. (2015) 35(22):3800-9].

Other versions of CRISPR which may be used according to some embodiments of the invention include genome editing using components from CRISPR systems together with other enzymes to directly install point mutations into cellular DNA or RNA.

Thus, according to one embodiment, the editing agent is DNA or RNA editing agent.

According to one embodiment, the DNA or RNA editing agent elicits base editing.

The term “base editing” as used herein refers to installing point mutations into cellular DNA or RNA without making double-stranded or single-stranded DNA breaks.

In base editing, DNA base editors typically comprise fusions between a catalytically impaired Cas nuclease and a base modification enzyme that operates on single-stranded DNA (ssDNA). Upon binding to its target DNA locus, base pairing between the gRNA and the target DNA strand leads to displacement of a small segment of single-stranded DNA in an ‘R loop’. DNA bases within this ssDNA bubble are modified by the base-editing enzyme (e.g. deaminase enzyme). To improve efficiency in eukaryotic cells, the catalytically disabled nuclease also generates a nick in the non-edited DNA strand, inducing cells to repair the non-edited strand using the edited strand as a template.

Two classes of DNA base editor have been described: cytosine base editors (CBEs) convert a C-G base pair into a T-A base pair, and adenine base editors (ABEs) convert an A-T base pair into a G-C base pair. Collectively, CBEs and ABEs can mediate all four possible transition mutations (C to T, A to G, T to C and G to A). Similarly in RNA, targeted adenosine conversion to inosine utilizes both antisense and Cas13-guided RNA-targeting methods.

According to one embodiment, the DNA or RNA editing agent comprises a catalytically inactive endonuclease (e.g. CRISPR-dCas).

According to one embodiment, the catalytically inactive endonuclease is an inactive Cas9 (e.g. dCas9).

According to one embodiment, the catalytically inactive endonuclease is an inactive Cas13 (e.g. dCas13).

According to one embodiment, the DNA or RNA editing agent comprises an enzyme which is capable of epigenetic editing (i.e. providing chemical changes to the DNA, the RNA or the histone proteins).

Exemplary enzymes include, but are not limited to, DNA methyltransferases, methylases, acetyltransferases. More specifically, exemplary enzymes include e.g. DNA (cytosine-5)-methyltransferase 3A (DNMT3a), Histone acetyltransferase p300, Ten-eleven translocation methylcytosine dioxygenase 1 (TET1), Lysine (K)-specific demethylase 1A (LSD1) and Calcium and integrin binding protein 1 (CIB1).

In addition to the catalytically disabled nuclease, the DNA or RNA editing agents of the invention may also comprise a nucleobase deaminase enzyme and/or a DNA glycosylase inhibitor.

According to a specific embodiment, the DNA or RNA editing agents comprise BE1 (APOBEC1-XTEN-dCas9), BE2 (APOBEC1-XTEN-dCas9-UGI) or BE3 (APOBEC-XTEN-dCas9(A840H)-UGI), along with sgRNA. APOBEC1 is a deaminase full length or catalytically active fragment, XTEN is a protein linker, UGI is uracil DNA glycosylase inhibitor to prevent the subsequent U:G mismatch from being repaired back to a C:G base pair and dCas9 (A840H) is a nickase in which the dCas9 was reverted to restore the catalytic activity of the HNH domain which nicks only the non-edited strand, simulating newly synthesized DNA and leading to the desired U:A product.

Additional enzymes which can be used for base editing according to some embodiments of the invention are specified in Rees and Liu, Nature Reviews Genetics (2018) 19:770-788, incorporated herein by reference in its entirety.

There are a number of publicly available tools available to help choose and/or design target sequences as well as lists of bioinformatically determined unique sgRNAs for different genes in different species such as, but not limited to, the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

In order to use the CRISPR system, both sgRNA and a Cas endonuclease (e.g. Cas9, Cpf1, CasX) should be expressed or present (e.g., as a ribonucleoprotein complex) in a target cell. The insertion vector can contain both cassettes on a single plasmid or the cassettes are expressed from two separate plasmids. CRISPR plasmids are commercially available such as the px330 plasmid from Addgene (75 Sidney St, Suite 550A⋅Cambridge, Mass. 02139). Use of clustered regularly interspaced short palindromic repeats (CRISPR)-associated (Cas)-guide RNA technology and a Cas endonuclease for modifying plant genomes are also at least disclosed by Svitashev et al., 2015, Plant Physiology, 169 (2): 931-945; Kumar and Jain, 2015, J Exp Bot 66: 47-57; and in U.S. Patent Application Publication No. 20150082478, which is specifically incorporated herein by reference in its entirety. Cas endonucleases that can be used to effect DNA editing with sgRNA include, but are not limited to, Cas9, Cpf1, CasX (Zetsche et al., 2015, Cell. 163(3):759-71), C2c1, C2c2, and C2c3 (Shmakov et al., Mol Cell. 2015 Nov. 5; 60(3):385-97).

“Hit and run” or “in-out”—involves a two-step recombination procedure. In the first step, an insertion-type vector containing a dual positive/negative selectable marker cassette is used to introduce the desired sequence alteration. The insertion vector contains a single continuous region of homology to the targeted locus and is modified to carry the mutation of interest. This targeting construct is linearized with a restriction enzyme at a one site within the region of homology, introduced into the cells, and positive selection is performed to isolate homologous recombination mediated events. The DNA carrying the homologous sequence can be provided as a plasmid, single or double stranded oligo. These homologous recombinants contain a local duplication that is separated by intervening vector sequence, including the selection cassette. In the second step, targeted clones are subjected to negative selection to identify cells that have lost the selection cassette via intra-chromosomal recombination between the duplicated sequences. The local recombination event removes the duplication and, depending on the site of recombination, the allele either retains the introduced mutation or reverts to wild type. The end result is the introduction of the desired modification without the retention of any exogenous sequences.

The “double-replacement” or “tag and exchange” strategy—involves a two-step selection procedure similar to the hit and run approach, but requires the use of two different targeting constructs. In the first step, a standard targeting vector with 3′ and 5′ homology arms is used to insert a dual positive/negative selectable cassette near the location where the mutation is to be introduced. After the system components have been introduced to the cell and positive selection applied, HR mediated events could be identified. Next, a second targeting vector that contains a region of homology with the desired mutation is introduced into targeted clones, and negative selection is applied to remove the selection cassette and introduce the mutation. The final allele contains the desired mutation while eliminating unwanted exogenous sequences.

According to a specific embodiment, the DNA editing agent comprises a DNA targeting module (e.g., gRNA).

According to a specific embodiment, the DNA editing agent does not comprise an endonuclease.

According to a specific embodiment, the DNA editing agent comprises an endonuclease.

According to a specific embodiment, the DNA editing agent comprises a catalytically inactive endonuclease.

According to a specific embodiment, the DNA editing agent comprises a nuclease (e.g. an endonuclease) and a DNA targeting module (e.g., sgRNA).

According to a specific embodiment, the DNA editing agent is CRISPR/endonuclease.

According to a specific embodiment, the DNA editing agent is CRISPR/Cas, e.g. sgRNA and Cas9 or a sgRNA and dCas9.

According to a specific embodiment, the DNA editing agent is a CRISPR/Cas9 as disclosed, for example, in WO 2019/058255, incorporated herein in it's entirety by reference.

According to a specific embodiment, the DNA or RNA editing agent elicits base editing.

According to a specific embodiment, the DNA or RNA editing agent comprises an enzyme for epigenetic editing.

According to a specific embodiment, the DNA editing agent is TALEN.

According to a specific embodiment, the DNA editing agent is ZFN.

According to a specific embodiment, the DNA editing agent is meganuclease.

According to one embodiment, the DNA editing agent is linked to a reporter for monitoring expression in a cell (e.g. eukaryotic cell).

According to one embodiment, the reporter is a fluorescent reporter protein.

The term “a fluorescent protein” refers to a polypeptide that emits fluorescence and is typically detectable by flow cytometry, microscopy or any fluorescent imaging system, therefore can be used as a basis for selection of cells expressing such a protein.

Examples of fluorescent proteins that can be used as reporters are, without being limited to, the Green Fluorescent Protein (GFP), the Blue Fluorescent Protein (BFP) and the red fluorescent proteins (e.g. dsRed, mCherry, RFP). A non-limiting list of fluorescent or other reporters includes proteins detectable by luminescence (e.g. luciferase) or colorimetric assay (e.g. GUS). According to a specific embodiment, the fluorescent reporter is a red fluorescent protein (e.g. dsRed, mCherry, RFP) or GFP.

A review of new classes of fluorescent proteins and applications can be found in Trends in Biochemical Sciences [Rodriguez, Erik A.; Campbell. Robert E.; Lin, John Y.; Lin, Michael Z; Miyawaki, Atsushi; Palmer, Amy E.; Shu, Xiaokun; Zhang, Jin; Thien, Roger Y. “The Growing and Glowing Toolbox of Fluorescent and Photoactive Proteins”. Trends in Biochemical Sciences. Doi:10.1016/j.tibs.2016.09.010].

According to another embodiment, the reporter is an endogenous gene of a plant. An exemplary reporter is the phytoene desaturase gene (PDS3) which encodes one of the important enzymes in the carotenoid biosynthesis pathway. Its silencing produces an albino/bleached phenotype. Accordingly, plants with reduced expression of PDS3 exhibit reduced chlorophyll levels, up to complete albino and dwarfism. Additional genes which can be used in accordance with the present teachings include, but are not limited to, genes which take part in crop protection.

According to another embodiment, the reporter is an antibiotic selection marker. Examples of antibiotic selection markers that can be used as reporters are, without being limited to, neomycin phosphotransferase II (nptII) and hygromycin phosphotransferase (hpt). Additional marker genes which can be used in accordance with the present teachings include, but are not limited to, gentamycin acetyltransferase (accC3) resistance and bleomycin and phleomycin resistance genes.

It will be appreciated that the enzyme NPTII inactivates by phosphorylation a number of aminoglycoside antibiotics such as kanamycin, neomycin, geneticin (or G418) and paromomycin. Of these, kanamycin, neomycin and paromomycin are used in a diverse range of plant species, and G418 is routinely used for selection of transformed mammalian cells.

According to another embodiment, the reporter is a toxic selection marker. An exemplary toxic selection marker that can be used as a reporter is, without being limited to, allyl alcohol selection using the Alcohol dehydrogenase (ADH1) gene. ADH1, comprising a group of dehydrogenase enzymes which catalyse the interconversion between alcohols and aldehydes or ketones with the concomitant reduction of NAD+ or NADP+, breaks down alcoholic toxic substances within tissues. Plants harbouring reduced ADH1 expression exhibit increase tolerance to allyl alcohol. Accordingly, plants with reduced ADH1 are resistant to the toxic effect of allyl alcohol.

Regardless of the DNA editing agent used, the method of the invention is employed such that the gene encoding the aberrantly processed (e.g. non-processable), transcribable RNA silencing molecule is modified by at least one of a deletion, an insertion or a point mutation.

According to one embodiment, the modification is in a structured region of the RNA silencing molecule.

According to one embodiment, the modification is in a stem region of the RNA silencing molecule.

According to one embodiment, the modification is in a loop region of the RNA silencing molecule.

According to one embodiment, the modification is in a stem region and a loop region of the RNA silencing molecule.

According to one embodiment, the modification is in a non-structured region of the RNA silencing molecule.

According to one embodiment, the modification is in a stem region and a loop region and in non-structured region of the RNA silencing molecule.

According to one embodiment, the modification of the nucleic acid sequence of the transcribable nucleic acid sequences encoding the aberrantly processed RNA molecules exhibiting the predetermined sequence homology range is affected at nucleic acids other than those corresponding to the binding site to the first target RNA (e.g., a natural target RNA), e.g. nucleic acids other than those encoding the mature sequence of the RNAi capable of binding a natural target.

According to one embodiment, the modification imparts processability of the RNA silencing molecule into small RNAs that are engaged with RISC.

According to a specific embodiment, the modification comprises a modification of about 1-500 nucleotides, about 1-250 nucleotides, about 1-150 nucleotides, about 1-100 nucleotides, about 1-50 nucleotides, about 1-25 nucleotides, about 1-10 nucleotides, about 10-250 nucleotides, about 10-200 nucleotides, about 10-150 nucleotides, about 10-100 nucleotides, about 10-50 nucleotides, about 1-50 nucleotides, about 1-10 nucleotides, about 50-150 nucleotides, about 50-100 nucleotides or about 100-200 nucleotides (as compared to the aberrantly processed, transcribable RNA silencing molecule).

According to one embodiment, the modification comprises a modification of at most 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 60, 70, 80, 90, 100, 110, 120, 130, 140, 150, 160, 170, 180, 190, 200, 250, 300, 350, 400, 450 or at most 500 nucleotides (as compared to the aberrantly processed, transcribable RNA silencing molecule).

According to one embodiment, the modification can be in a consecutive nucleic acid sequence (e.g. at least 5, 10, 20, 30, 40, 50, 100, 150, 200, 300, 400, 500 bases).

According to one embodiment, the modification can be in a non-consecutive manner, e.g. throughout a 20, 50, 100, 150, 200, 500, 1000, 2000, 5000 nucleic acid sequence.

According to a specific embodiment, the modification comprises a modification of at most 200 nucleotides.

According to a specific embodiment, the modification comprises a modification of at most 150 nucleotides.

According to a specific embodiment, the modification comprises a modification of at most 100 nucleotides.

According to a specific embodiment, the modification comprises a modification of at most 50 nucleotides.

According to a specific embodiment, the modification comprises a modification of at most 25 nucleotides.

According to a specific embodiment, the modification comprises a modification of at most 24 nucleotides.

According to a specific embodiment, the modification comprises a modification of at most 23 nucleotides.

According to a specific embodiment, the modification comprises a modification of at most 22 nucleotides.

According to a specific embodiment, the modification comprises a modification of at most 21 nucleotides.

According to a specific embodiment, the modification comprises a modification of at most 20 nucleotides.

According to a specific embodiment, the modification comprises a modification of at most 15 nucleotides.

According to a specific embodiment, the modification comprises a modification of at most 10 nucleotides.

According to a specific embodiment, the modification comprises a modification of at most 5 nucleotides.

According to one embodiment, the modification is such that the recognition/cut site/PAM motif of the RNA silencing molecule is modified to abolish the original PAM recognition site.

According to a specific embodiment, the modification is in at least 1, 2, 3, 4, 5, 6, 7, 8, 9, 10 or more nucleic acids in a PAM motif.

According to one embodiment, the modification comprises an insertion.

According to a specific embodiment, the insertion comprises an insertion of about 1-500 nucleotides, about 1-250 nucleotides, about 1-150 nucleotides, about 1-100 nucleotides, about 1-50 nucleotides, about 1-25 nucleotides, about 1-10 nucleotides, about 10-250 nucleotides, about 10-200 nucleotides, about 10-150 nucleotides, about 10-100 nucleotides, about 10-50 nucleotides, about 1-50 nucleotides, about 1-10 nucleotides, about 50-150 nucleotides, about 50-100 nucleotides or about 100-200 nucleotides (as compared to the aberrantly processed, transcribable RNA silencing molecule).

According to one embodiment, the insertion comprises an insertion of at most 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 60, 70, 80, 90, 100, 110, 120, 130, 140, 150, 160, 170, 180, 190, 200, 250, 300, 350, 400 or at most 500 nucleotides (as compared to the aberrantly processed, transcribable RNA silencing molecule).

According to a specific embodiment, the insertion comprises an insertion of at most 200 nucleotides.

According to a specific embodiment, the insertion comprises an insertion of at most 150 nucleotides.

According to a specific embodiment, the insertion comprises an insertion of at most 100 nucleotides.

According to a specific embodiment, the insertion comprises an insertion of at most 50 nucleotides.

According to a specific embodiment, the insertion comprises an insertion of at most 25 nucleotides.

According to a specific embodiment, the insertion comprises an insertion of at most 24 nucleotides.

According to a specific embodiment, the insertion comprises an insertion of at most 23 nucleotides.

According to a specific embodiment, the insertion comprises an insertion of at most 22 nucleotides.

According to a specific embodiment, the insertion comprises an insertion of at most 21 nucleotides.

According to a specific embodiment, the insertion comprises an insertion of at most 20 nucleotides.

According to a specific embodiment, the insertion comprises an insertion of at most 15 nucleotides.

According to a specific embodiment, the insertion comprises an insertion of at most 10 nucleotides.

According to a specific embodiment, the insertion comprises an insertion of at most 5 nucleotides.

According to one embodiment, the modification comprises a deletion.

According to a specific embodiment, the deletion comprises a deletion of about 1-500 nucleotides, about 1-250 nucleotides, about 1-150 nucleotides, about 1-100 nucleotides, about 1-50 nucleotides, about 1-25 nucleotides, about 1-10 nucleotides, about 10-250 nucleotides, about 10-200 nucleotides, about 10-150 nucleotides, about 10-100 nucleotides, about 10-50 nucleotides, about 1-50 nucleotides, about 1-10 nucleotides, about 50-150 nucleotides, about 50-100 nucleotides or about 100-200 nucleotides (as compared to the aberrantly processed, transcribable RNA silencing molecule).

According to one embodiment, the deletion comprises a deletion of at most 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 60, 70, 80, 90, 100, 110, 120, 130, 140, 150, 160, 170, 180, 190, 200, 250, 300, 350, 400, 450 or at most 500 nucleotides (as compared to the aberrantly processed, transcribable RNA silencing molecule).

According to a specific embodiment, the deletion comprises a deletion of at most 200 nucleotides.

According to a specific embodiment, the deletion comprises a deletion of at most 150 nucleotides.

According to a specific embodiment, the deletion comprises a deletion of at most 100 nucleotides.

According to a specific embodiment, the deletion comprises a deletion of at most 50 nucleotides.

According to a specific embodiment, the deletion comprises a deletion of at most 25 nucleotides.

According to a specific embodiment, the deletion comprises a deletion of at most 24 nucleotides.

According to a specific embodiment, the deletion comprises a deletion of at most 23 nucleotides.

According to a specific embodiment, the deletion comprises a deletion of at most 22 nucleotides.

According to a specific embodiment, the deletion comprises a deletion of at most 21 nucleotides.

According to a specific embodiment, the deletion comprises a deletion of at most 20 nucleotides.

According to a specific embodiment, the deletion comprises a deletion of at most 15 nucleotides.

According to a specific embodiment, the deletion comprises a deletion of at most 10 nucleotides.

According to a specific embodiment, the deletion comprises a deletion of at most 5 nucleotides.

According to one embodiment, the modification comprises a point mutation.

According to a specific embodiment, the point mutation comprises a point mutation of about 1-500 nucleotides, about 1-250 nucleotides, about 1-150 nucleotides, about 1-100 nucleotides, about 1-50 nucleotides, about 1-25 nucleotides, about 1-10 nucleotides, about 10-250 nucleotides, about 10-200 nucleotides, about 10-150 nucleotides, about 10-100 nucleotides, about 10-50 nucleotides, about 1-50 nucleotides, about 1-10 nucleotides, about 50-150 nucleotides, about 50-100 nucleotides or about 100-200 nucleotides (as compared to the aberrantly processed, transcribable RNA silencing molecule).

According to one embodiment, the point mutation comprises a point mutation in at most 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 60, 70, 80, 90, 100, 110, 120, 130, 140, 150, 160, 170, 180, 190, 200, 250, 300, 350, 400, 450 or at most 500 nucleotides (as compared to the aberrantly processed, transcribable RNA silencing molecule).

According to a specific embodiment, the point mutation comprises a point mutation in at most 200 nucleotides.

According to a specific embodiment, the point mutation comprises a point mutation in at most 150 nucleotides.

According to a specific embodiment, the point mutation comprises a point mutation in at most 100 nucleotides.

According to a specific embodiment, the point mutation comprises a point mutation in at most 50 nucleotides.

According to a specific embodiment, the point mutation comprises a point mutation in at most 25 nucleotides.

According to a specific embodiment, the point mutation comprises a point mutation in at most 24 nucleotides.

According to a specific embodiment, the point mutation comprises a point mutation in at most 23 nucleotides.

According to a specific embodiment, the point mutation comprises a point mutation in at most 22 nucleotides.

According to a specific embodiment, the point mutation comprises a point mutation in at most 21 nucleotides.

According to a specific embodiment, the point mutation comprises a point mutation in at most 20 nucleotides.

According to a specific embodiment, the point mutation comprises a point mutation in at most 15 nucleotides.

According to a specific embodiment, the point mutation comprises a point mutation in at most 10 nucleotides.

According to a specific embodiment, the point mutation comprises a point mutation in at most 5 nucleotides.

According to one embodiment, the modification comprises a combination of any of a deletion, an insertion and/or a point mutation.

According to one embodiment, the modification comprises nucleotide replacement (e.g. nucleotide swapping).

According to a specific embodiment, the swapping comprises swapping of about 1-500 nucleotides, 1-450 nucleotides, 1-400 nucleotides, 1-350 nucleotides, 1-300 nucleotides, 1-250 nucleotides, 1-200 nucleotides, 1-150 nucleotides, 1-100 nucleotides, 1-90 nucleotides, 1-80 nucleotides, 1-70 nucleotides, 1-60 nucleotides, 1-50 nucleotides, 1-40 nucleotides, 1-30 nucleotides, 1-20 nucleotides, 1-10 nucleotides, 10-100 nucleotides, 10-90 nucleotides, 10-80 nucleotides, 10-70 nucleotides, 10-60 nucleotides, 10-50 nucleotides, 10-40 nucleotides, 10-30 nucleotides, 10-20 nucleotides, 10-15 nucleotides, 20-30 nucleotides, 20-50 nucleotides, 20-70 nucleotides, 30-40 nucleotides, 30-50 nucleotides, 30-70 nucleotides, 40-50 nucleotides, 40-80 nucleotides, 50-60 nucleotides, 50-70 nucleotides, 50-90 nucleotides, 60-70 nucleotides, 60-80 nucleotides, 70-80 nucleotides, 70-90 nucleotides, 80-90 nucleotides, 90-100 nucleotides, 100-110 nucleotides, 100-120 nucleotides, 100-130 nucleotides, 100-140 nucleotides, 100-150 nucleotides, 100-160 nucleotides, 100-170 nucleotides, 100-180 nucleotides, 100-190 nucleotides, 100-200 nucleotides, 110-120 nucleotides, 120-130 nucleotides, 130-140 nucleotides, 140-150 nucleotides, 160-170 nucleotides, 180-190 nucleotides, 190-200 nucleotides, 200-250 nucleotides, 250-300 nucleotides, 300-350 nucleotides, 350-400 nucleotides, 400-450 nucleotides, or about 450-500 nucleotides (as compared to the aberrantly processed, transcribable RNA silencing molecule).

According to one embodiment, the nucleotide swap comprises a nucleotide replacement in at most 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 60, 70, 80, 90, 100, 110, 120, 130, 140, 150, 160, 170, 180, 190, 200, 250, 300, 350, 400, 450 or at most 500 nucleotides (as compared to the aberrantly processed, transcribable RNA silencing molecule).

According to a specific embodiment, the nucleotide swapping comprises a nucleotide replacement in at most 200 nucleotides.

According to a specific embodiment, the nucleotide swapping comprises a nucleotide replacement in at most 150 nucleotides.

According to a specific embodiment, the nucleotide swapping comprises a nucleotide replacement in at most 100 nucleotides.

According to a specific embodiment, the nucleotide swapping comprises a nucleotide replacement in at most 50 nucleotides.

According to a specific embodiment, the nucleotide swapping comprises a nucleotide replacement in at most 25 nucleotides.

According to a specific embodiment, the nucleotide swapping comprises a nucleotide replacement in at most 24 nucleotides.

According to a specific embodiment, the nucleotide swapping comprises a nucleotide replacement in at most 23 nucleotides.

According to a specific embodiment, the nucleotide swapping comprises a nucleotide replacement in at most 22 nucleotides.

According to a specific embodiment, the nucleotide swapping comprises a nucleotide replacement in at most 21 nucleotides.

According to a specific embodiment, the nucleotide swapping comprises a nucleotide replacement in at most 20 nucleotides.

According to a specific embodiment, the nucleotide swapping comprises a nucleotide replacement in at most 15 nucleotides.

According to a specific embodiment, the nucleotide swapping comprises a nucleotide replacement in at most 10 nucleotides.

According to a specific embodiment, the nucleotide swapping comprises a nucleotide replacement in at most 5 nucleotides.

According to one embodiment, when the modification is an insertion or swapping, donor oligonucleotides are utilized (as discussed below).

According to one embodiment, any one or combination of the above described modifications can be carried out in order to impart processability of the RNA molecules into small RNAs that are engaged with RISC.

According to a specific embodiment, a deletion and insertion modification (e.g. swapping) is affected by gene editing (e.g. using the CRISPR/Cas9 technology) in combination with donor oligonucleotides (as discussed below), such that processability and silencing activity of the dysfunctional RNA silencing molecule is obtained. Such methods are disclosed, for example, in WO 2019/058255, incorporated herein in its entirety by reference.

According to a one embodiment, the RNA molecule is endogenous (naturally occurring, e.g. native) to the cell. It will be appreciated that the RNA molecule can also be exogenous to the cell (i.e. externally added and which is not naturally occurring in the cell).

According to some embodiments, the RNA molecule comprises an intrinsic translational inhibition activity.

According to some embodiments, the RNA molecule comprises an intrinsic RNA interference (RNAi) activity.

According to a specific embodiment, a precursor nucleic acid sequence of an RNA silencing molecule (i.e. RNAi molecule, e.g. miRNA, siRNA, piRNA, shRNA, etc.) is modified to preserve originality of structure and to be recognized and processed by cellular RNAi processing and executing factors.

According to a specific embodiment, a precursor nucleic acid sequence of a dysfunctional RNA silencing molecule (i.e. miRNA, rRNA, tRNA, lncRNA, snoRNA, etc.) is modified to be recognized and processed by cellular RNAi processing and executing factors.

According to a specific embodiment, imparting processability into small RNAs that are engaged with RISC is effected by restoring the structure of the dysfunctional RNA silencing molecule (e.g. at least 95%, at least 96%, at least 97%, at least 98%, at least 99%, or 100% of the structure of the corresponding homologous RNA silencing molecule processed into a RISC-engaged RNA molecule (e.g. wild-type precursor)), e.g. when the secondary structure of the dysfunctional RNA silencing molecule is translated to a linear string form and is compared to a string form of a secondary structure of the homologous RNA silencing molecule processed into a RISC-engaged RNA molecule (e.g. wild-type precursor). Any method known in the art can be used to translate a secondary structure to a series of strings which can be compared with another series of strings, such as but not limited to RNAfold.

According to a specific embodiment, a nucleic acid sequence of a dysfunctional RNA silencing molecule (i.e. tasiRNA etc.) is modified to bind factors and/or oligonucleotides (e.g. miRNA) which enable silencing activity and/or processing into a silencing RNA. In a non-limiting example, the dysfunctional RNA silencing molecule is homologous to a trans-activating RNA (tasiRNA) molecule but cannot bind an amplifier RNA molecule and thus is not processable to silencing small RNA. Accordingly, such an RNA silencing molecule is modified to bind factors (e.g. an amplifier) which enable silencing activity.

According to some embodiments, the RNA-like molecule (e.g. miRNA-like) does not comprise an intrinsic translational inhibition activity or an intrinsic RNAi activity (i.e. the RNA-like molecule does not have an intrinsic RNA silencing activity).

According to specific embodiments, when the cell is a cell of Arabidopsis (A. thaliana), the aberrantly processed, transcribable nucleic acid sequences encoding the RNA molecules exhibiting the predetermined sequence homology range include those listed in Table 2, herein below.

According to specific embodiments, when the cell is a cell of a Caenorhabditis elegans (C. elegans), the aberrantly processed, transcribable nucleic acid sequences encoding the RNA molecules exhibiting the predetermined sequence homology range include those listed in Table 3, herein below.

According to specific embodiments, when the cell is a cell of a human (H. sapiens), the aberrantly processed, transcribable nucleic acid sequences encoding the RNA molecules exhibiting the predetermined sequence homology range include those listed in Table 4, herein below.

According to one embodiment, the modification imparts processability of the RNA silencing molecule into small RNAs that bind a first target RNA.

According to an embodiment of the invention, the RNA molecule is specific to a first target RNA (e.g., a natural target RNA) and does not cross inhibit or silence a target RNA of interest unless designed to do so (as discussed below) exhibiting 100% or less global homology to the target gene, e.g., less than 99%, 98%, 97%, 96%, 95%, 94%, 93%, 92%, 91%, 90%, 89%, 88%, 87%, 86%, 85%, 84%, 83%, 82%, 81% global homology to the target gene; as determined at the RNA or protein level by RT-PCR, Western blot, Immunohistochemistry and/or flow cytometry, sequencing or any other detection methods.

According to one embodiment, the method further comprises modifying the specificity of the RNA molecule having the silencing activity in a cell (e.g. the RNA molecules imparted with a silencing activity), the method comprising introducing into the cell a DNA editing agent which redirects a silencing specificity of the RNA molecule towards a target RNA of interest, the target RNA of interest being distinct from the first target RNA, thereby modifying the specificity of the RNA molecule having the silencing activity in the cell.

As used herein, the term “redirects a silencing specificity” refers to reprogramming the original specificity of the RNA silencing molecule towards a non-natural target of the RNA silencing molecule (also referred to herein as “redirection” of silencing activity). Accordingly, the original specificity of the RNA silencing molecule is destroyed (i.e. loss of function) and the new specificity is towards an RNA target distinct of the natural target (i.e. RNA of interest), i.e., gain of function.

As used herein, the term “first target RNA” refers to an RNA sequence naturally bound by an RNA silencing molecule. Thus, the first target RNA is considered by the skilled artisan as a substrate for the RNA silencing molecule (e.g. which is to be silenced by that RNA silencing molecule).

According to some embodiments, when referring to an RNAi-like molecule (e.g. miRNA-like molecule), the first target RNA refers to the RNA sequence which would have been targeted by that RNAi-like molecule had is been processed like a canonical homolog of such RNAi-like molecule (e.g. the first target RNA is the RNA sequence which corresponds to the sequence that would have been the mature miRNA sequence of a miRNA-like molecule).

As used herein, the term “target RNA of interest” refers to an RNA sequence (coding or non-coding) to be silenced by the designed RNA silencing molecule.

As used herein, the phrase “silencing a target gene” refer to the absence or observable reduction in the level of protein and/or mRNA product from the target gene. Thus, silencing of a target gene can be by 5%, 10%, 20%, 30%, 40%, 50%, 60%, 70%, 80%, 90%, 95% or 100% as compared to a target gene not targeted by the designed RNA silencing molecule of the invention.

According to one embodiment, modifying the nucleic acid sequence of the transcribable nucleic acid sequences encoding the aberrantly processed RNA molecules exhibiting the predetermined sequence homology range imparts processability into small RNAs that are engaged with RISC and are complementary to a target an RNA of interest.

According to one embodiment, modifying the nucleic acid sequence of the transcribable nucleic acid sequences imparts a structure of the aberrantly processed RNA molecules, which results in processing of the RNA molecules into small RNAs that are engaged with RISC and target an RNA of interest.

The consequences of silencing can be confirmed by examination of the outward properties of a eukaryotic cell or organism (e.g. plant cell or whole plant), or by biochemical techniques (as discussed below).

It will be appreciated that the designed RNA silencing molecule of some embodiments of the invention can have some off-target specificity effect/s provided that it does not affect the growth, differentiation or function of the eukaryotic cell or organism, e.g. it does not affect an agriculturally valuable trait (e.g., biomass, yield, growth, etc.) of a plant.

According to one embodiment, the target RNA of interest is endogenous to the eukaryotic cell.

Exemplary endogenous target RNA of interest in animal cells (e.g. mammalian cells) include, but are not limited to, a product of a gene associated with cancer and/or apoptosis. Exemplary target genes associated with cancer include, but are not limited to, p53, BAX, PUMA, NOXA and FAS genes as discussed in detail herein below.

Exemplary endogenous target RNA of interest in a plant cell include, but are not limited to, a product of a gene conferring sensitivity to stress, to infection, to herbicides, or a product of a gene related to plant growth rate, crop yield, as further discussed herein below.

According to one embodiment, the target RNA of interest is exogenous to the eukaryotic cell e.g. plant cell (also referred to herein as heterologous). In such a case, the target RNA of interest is a product of a gene that is not naturally part of the eukaryotic cell genome (e.g. plant genome).

Exemplary exogenous target RNAs in animal cells (e.g. mammalian cells) include, but are not limited to, products of a gene associated with an infectious disease, such as a gene of a pathogen (e.g. an insect, a virus, a bacteria, a fungi, a nematode), as further discussed herein below.

Exemplary exogenous target RNA of interest in a plant cell include, but are not limited to, a product of a gene of a plant pathogen such as, but not limited to, an insect, a virus, a bacteria, a fungi, a nematode, as further discussed herein below.

An exogenous target RNA (coding or non-coding) may comprise a nucleic acid sequence which shares sequence identity with an endogenous RNA sequence (e.g. may be partially homologous to an endogenous nucleic acid sequence) of the eukaryotic organism (e.g. plant).

The specific binding of an RNA silencing molecule with a target RNA can be determined by computational algorithms (such as BLAST) and verified by methods including e.g. Northern blot, In Situ hybridization. QuantiGene Plex Assay etc.

By use of the term “complementarity” or “complementary” is meant that the RNA silencing molecule (or at least a portion of it that is present in the processed small RNA form, or at least one strand of a double-stranded polynucleotide or portion thereof, or a portion of a single strand polynucleotide) hybridizes under physiological conditions to the target RNA, or a fragment thereof, to effect regulation or function or suppression of the target gene. For example, in some embodiments, an RNA silencing molecule has 100 percent sequence identity or at least about 30, 40, 45, 50, 55, 60, 65, 70, 75, 80, 83, 84, 85, 86, 87, 88, 89, 90, 91, 92, 93, 94, 95, 96, 97, 98, or 99 percent sequence identity when compared to a sequence of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 70, 80, 90, 100, 150, 200, 300, 400, 500 or more contiguous nucleotides in the target RNA (or family members of a given target gene).

As used herein, an RNA silencing molecule, or it's processed small RNA forms, are said to exhibit “complete complementarity” when every nucleotide of one of the sequences read 5′ to 3′ is complementary to every nucleotide of the other sequence when read 3′ to 5′. A nucleotide sequence that is completely complementary to a reference nucleotide sequence will exhibit a sequence identical to the reverse complement sequence of the reference nucleotide sequence.

Methods for determining sequence complementarity are well known in the art and include, but not limited to, bioinformatics tools which are well known in the art (e.g. BLAST, multiple sequence alignment).

According to one embodiment, if the RNA silencing molecule is or processed into a siRNA, the complementarity is in the range of 90-100% (e.g. 100%) to its target sequence.

According to one embodiment, if the RNA silencing molecule is or processed into a miRNA or piRNA the complementarity is in the range of 33-100% to its target sequence.

According to one embodiment, if the RNA silencing molecule is a miRNA, the seed sequence complementarity (i.e. nucleotides 2-8 from the 5′) is in the range of 85-100% (e.g. 100%) to its target sequence.

According to one embodiment, the RNA silencing molecule is designed so as to comprise at least about 33%, 40%, 45%, 50%, 60%, 70%, 80%, 85%, 90%, 91%, 92%, 93%, 94%, 95%, 96%, 97%, 98%, 99% or even 100% complementarity towards the sequence of the target RNA of interest.

According to a specific embodiment, the RNA silencing molecule is designed so as to comprise a minimum of 33% complementarity towards the target RNA of interest (e.g. 85-100% seed match).

According to a specific embodiment, the RNA silencing molecule is designed so as to comprise a minimum of 40% complementarity towards the target RNA of interest.

According to a specific embodiment, the RNA silencing molecule is designed so as to comprise a minimum of 50% complementarity towards the target RNA of interest.

According to a specific embodiment, the RNA silencing molecule is designed so as to comprise a minimum of 60% complementarity towards the target RNA of interest.

According to a specific embodiment, the RNA silencing molecule is designed so as to comprise a minimum of 70% complementarity towards the target RNA of interest.

According to a specific embodiment, the RNA silencing molecule is designed so as to comprise a minimum of 80% complementarity towards the target RNA of interest.

According to a specific embodiment, the RNA silencing molecule is designed so as to comprise a minimum of 90% complementarity towards the target RNA of interest.

According to a specific embodiment, the RNA silencing molecule is designed so as to comprise a minimum of 95% complementarity towards the target RNA of interest.

According to a specific embodiment, the RNA silencing molecule is designed so as to comprise a minimum of 96% complementarity towards the target RNA of interest.

According to a specific embodiment, the RNA silencing molecule is designed so as to comprise a minimum of 97% complementarity towards the target RNA of interest.

According to a specific embodiment, the RNA silencing molecule is designed so as to comprise a minimum of 98% complementarity towards the target RNA of interest.

According to a specific embodiment, the RNA silencing molecule is designed so as to comprise a minimum of 99% complementarity towards the target RNA of interest.

According to a specific embodiment, the RNA silencing molecule is designed so as to comprise 100% complementarity towards the target RNA of interest.

Any of the above described DNA editing agents can be used to modify the specificity of the RNA molecule having the silencing activity.

According to one embodiment, the RNA silencing molecule is modified in the guide strand (silencing strand) as to comprise about 50-100% complementarity to the target RNA of interest.

According to one embodiment, the RNA silencing molecule is modified in the passenger strand (the complementary strand) as to comprise about 50-100% complementarity to the target RNA of interest.

According to one embodiment, the RNA silencing molecule is modified such that the seed sequence (e.g. for miRNA nucleotides 2-8 from the 5′ terminal) is complimentary to the target sequence.

According to one embodiment, modifying the nucleic acid sequence so as to impart processability into small RNAs, is carried out prior to modifying the specificity of the RNA silencing molecule.

According to one embodiment, modifying the nucleic acid sequence so as to impart processability into small RNAs, is carried out concomitantly with modifying the specificity of the RNA silencing molecule.

According to one embodiment, modifying the specificity of the RNA silencing molecule is carried out without impairing processability.

Accordingly, when the RNA silencing molecule contains a non-essential structure (i.e. a secondary structure of the RNA silencing molecule which does not play a role in its proper biogenesis and/or function) or is purely dsRNA (i.e. the RNA silencing molecule having a perfect or almost perfect dsRNA), a few modifications (e.g. 20-30 nucleotides, e.g. 1-10 nucleotides, e.g. 5 nucleotides) are introduced in order to impart processability and optionally modify the specificity of the RNA silencing molecule.

According to another embodiment, when the RNA silencing molecule has an essential structure (i.e. the proper biogenesis and/or activity of the RNA silencing molecule is dependent on its secondary structure), larger modifications (e.g. 1-500 nucleotides, 10-250 nucleotides, 50-150 nucleotides, more than 30 nucleotides and not exceeding 200 nucleotides, 30-200 nucleotides, 35-200 nucleotides, 35-150 nucleotides, 35-100 nucleotides) are introduced in order to impart processability and optionally modify the specificity of the RNA silencing molecule.

According to one embodiment, the gene encoding the RNA silencing molecule is modified by swapping a sequence of an endogenous RNA silencing molecule (e.g. miRNA) with an RNA silencing sequence of choice (e.g. siRNA).

According to one embodiment, the guide strand of the RNA silencing molecule, such as miRNA precursors (pri/pre-miRNAs) or siRNA precursors (dsRNA), is modified to preserve originality of structure and keep the same base pairing profile.

According to one embodiment, the passenger strand of the RNA silencing molecule, such as miRNA precursors (pri/pre-miRNAs) or siRNA precursors (dsRNA), is modified to preserve originality of structure and keep the same base pairing profile.

It will be appreciated that additional mutations can be introduced by additional events of editing (i.e., concomitantly or sequentially).

The DNA editing agent of the invention may be introduced into cells (e.g. eukaryotic cells) using DNA delivery methods (e.g. by expression vectors) or using DNA-free methods.

According to one embodiment, the sgRNA (or any other DNA recognition module used, dependent on the DNA editing system that is used) can be provided as RNA to the cell.

Thus, it will be appreciated that the present techniques relate to introducing the DNA editing agent using transient DNA or DNA-free methods such as RNA transfection (e.g. mRNA+sgRNA transfection), or Ribonucleoprotein (RNP) transfection (e.g. protein-RNA complex transfection, e.g. Cas9/gRNA ribonucleoprotein (RNP) complex transfection).

For example, Cas9 can be introduced as a DNA expression plasmid, in vitro transcript (i.e. RNA), or as a recombinant protein bound to the RNA portion in a ribonucleoprotein particle (RNP). sgRNA, for example, can be delivered either as a DNA plasmid or as an in vitro transcript (i.e. RNA).

Any method known in the art for RNA or RNP transfection can be used in accordance with the present teachings, such as, but not limited to microinjection [as described by Cho et al., “Heritable gene knockout in Caenorhabditis elegans by direct injection of Cas9-sgRNA ribonucleoproteins,” Genetics (2013) 195:1177-1180, incorporated herein by reference], electroporation [as described by Kim et al., “Highly efficient RNA-guided genome editing in human cells via delivery of purified Cas9 ribonucleoproteins” Genome Res. (2014) 24:1012-1019, incorporated herein by reference], or lipid-mediated transfection e.g. using liposomes [as described by Zuris et al., “Cationic lipid-mediated delivery of proteins enables efficient protein-based genome editing in vitro and in vivo” Nat Biotechnol. (2014) doi: 10.1038/nbt.3081, incorporated herein by reference]. Additional methods of RNA transfection are described in U.S. Patent Application No. 20160289675, incorporated herein by reference in its entirety.

One advantage of RNA transfection methods of the invention is that RNA transfection is essentially transient and vector-free. An RNA transgene can be delivered to a cell and expressed therein, as a minimal expressing cassette without the need for any additional sequences (e.g. viral sequences).

According to one embodiment, for expression of exogenous DNA editing agents of the invention in cells, a polynucleotide sequence encoding the DNA editing agent is ligated into a nucleic acid construct suitable for cell expression. Such a nucleic acid construct includes a promoter sequence for directing transcription of the polynucleotide sequence in the cell in a constitutive or inducible manner.

The nucleic acid construct (also referred to herein as an “expression vector”) of some embodiments of the invention includes additional sequences which render this vector suitable for replication and integration in eukaryotes (e.g., shuttle vectors). In addition, typical cloning vectors may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of some embodiments of the invention is active in the specific cell population transformed. Examples of cell type-specific and/or tissue-specific promoters include promoters such as albumin that is liver specific [Pinkert et al., (1987) Genes Dev. 1:268-277], lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166).

For expression in a plant cell, the plant promoter employed can be a constitutive promoter, a tissue specific promoter, an inducible promoter, a chimeric promoter or a developmentally regulated promoter.

Examples of preferred promoters useful for the methods of some embodiments of the invention (in plant cells) are presented in Table I, II, III and IV.

TABLE I
Exemplary constitutive promoters for use in the performance
of some embodiments of the invention in plant cells

| | Expression | |
|---|---|---|
| Gene Source | Pattern | Reference |
| Actin | constitutive | McElroy et al, Plant Cell, 2: |
| | | 163-171, 1990 |
| CAMV 35S | constitutive | Odell et al, Nature, 313: 810-812, |
| | | 1985 |
| CaMV 19S | constitutive | Nilsson et al., Physiol. Plant 100: |
| | | 456-462, 1997 |
| GOS2 | constitutive | de Pater et al, Plant J Nov; 2(6): |
| | | 837-44, 1992 |
| ubiquitin | constitutive | Christensen et al, Plant Mol. Biol. |
| | | 18: 675-689, 1992 |
| Rice cyclophilin | constitutive | Bucholz et al, Plant Mol Biol. |
| | | 25(5): 837-43, 1994 |
| Maize H3 histone | constitutive | Lepetit et al, Mol. Gen. Genet. |
| | | 231: 276-285, 1992 |
| Actin 2 | constitutive | An et al, Plant J. 10(1); 107121, |
| | | 1996 |
| CVMV (Cassava Vein | constitutive | Lawrenson et al, Gen Biol 16: |
| Mosaic Virus | | 258, 2015 |
| U6 (AtU626; TaU6) | constitutive | Lawrenson et al, Gen Biol 16: |
| | | 258, 2015 |

TABLE II
Exemplary seed-preferred promoters for use in the performance
of some embodiments of the invention in plant cells

| | Expression | |
|---|---|---|
| Gene Source | Pattern | Reference |
| Seed specific genes | seed | Simon, et al., Plant Mol. Biol. 5. 191, 1985; |
| | | Scofield, et al., J. Biol. Chem. 262: 12202, 1987; |
| | | Baszczynski, et al., Plant Mol. Biol. 14: 633, 1990. |
| Brazil Nut albumin | seed | Pearson' et al., Plant Mol. Biol. 18: 235-245, 1992. |
| Legumin | seed | Ellis, et al. Plant Mol. Biol. 10: 203-214, 1988 |
| Glutelin (rice) | seed | Takaiwa, et al., Mol. Gen. Genet. 208: 15-22, 1986; |
| | | Takaiwa, et al., FEBS Letts. 221: 43-47, 1987 |
| Zein | seed | Matzke et al Plant Mol Biol, 143). 323-32 1990 |
| napA | seed | Stalberg, et al, Planta 199: 515-519, 1996 |
| wheat LMW and | endosperm | Mol Gen Genet 216: 81-90, 1989; NAR 17: |
| HMW, glutenin-1 | | 461-2, |
| Wheat SPA | seed | Albanietal, Plant Cell, 9: 171-184, 1997 |
| wheat a, b and g gliadins | endosperm | EMBO3: 1409-15, 1984 |
| Barley ltrl promoter | endosperm | |
| barley B1, C, D hordein | endosperm | Theor Appl Gen 98: 1253-62, 1999; Plant J 4: |
| | | 343-55, 1993; Mol Gen Genet 250: 750-60, 1996 |
| Barley DOF | endosperm | Mena et al. The Plant Journal, 116(1): 53-62, 1998 |
| Biz2 | endosperm | EP99106056.7 |
| Synthetic promoter | endosperm | Vicente-Carbajosa et al., Plant J. 13: 629-640, 1998 |
| rice prolamin NRP33 | endosperm | Wu et al, Plant Cell Physiology 39(8) 885-889, 1998 |
| rice -globulin Glb-1 | endosperm | Wu et al, Plant Cell Physiology 398) 885-889, 1998 |
| rice OSHI | emryo | Sato et al, Proc. Nati. Acad. Sci. USA, 93: 8117-8122 |
| rice alpha-globulin | endosperm | Nakase et al. Plant Mol. Biol. 33: 513-S22, 1997 |
| REB/OHP-1 | | |
| rice ADP-glucose PP | endosperm | Trans Res 6: 157-68, 1997 |
| maize ESR gene family | endosperm | Plant J 12: 235-46, 1997 |
| sorgum gamma- kafirin | endosperm | PMB 32: 1029-35, 1996 |
| KNOX | emryo | Postma-Haarsma ef al, Plant Mol. Biol. 39: 257-71, 1999 |
| rice oleosin | Embryo and aleuton | Wu et at, J. Biochem., 123: 386, 1998 |
| sunflower oleosin | Seed (embryo and | Cummins, et al., Plant Mol. Biol. 19: 873-876, 1992 |
| | dry seed) | |

TABLE III
Exemplary flower-specific promoters for use in
the performance of the invention in plant cells

| | Expression | |
|---|---|---|
| Gene Source | Pattern | Reference |
| AtPRP4 | flowers | www(dot)salus(dot) |
| | | medium(dot)edu/m mg/70inaliz/html |
| chalene synthase | flowers | Van der Meer, et al., Plant Mol. Biol. |
| (chsA) | | 15: 95-109, 1990. |
| LAT52 | anther | Twell et al Mol. Gen Genet. 217: 240- |
| | | 245 (1989) |
| apetala- 3 | flowers | |

TABLE IV
Alternative rice promoters for use in the
performance of the invention in plant cells

| PRO # | Gene | Expression |
|---|---|---|
| PR00001 | Metallothionein Mte | transfer layer of |
| | | embryo + calli |
| PR00005 | putative beta-amylase | transfer layer of embryo |
| PR00009 | Putative cellulose synthase | Weak in roots |
| PR00012 | lipase (putative) | |
| PR00014 | Transferase (putative) | |
| PR00016 | peptidyl prolyl cis-trans | |
| | isomerase (putative) | |
| PR00019 | unknown | |
| PR00020 | prp protein (putative) | |
| PR00029 | noduline (putative) | |
| PR00058 | Proteinase inhibitor Rgpi9 | seed |
| PR00061 | beta expansine EXPB9 | Weak in young flowers |
| PR00063 | Structural protein | young tissues + |
| | | calli + embryo |
| PR00069 | xylosidase (putative) | |
| PR00075 | Prolamine 10 Kda | strong in endosperm |
| PR00076 | allergen RA2 | strong in endosperm |
| PR00077 | prolamine RP7 | strong in endosperm |
| PR00078 | CBP80 | |
| PR00079 | starch branching enzyme I | |
| PR00080 | Metallothioneine-like ML2 | transfer layer of |
| | | embryo + calli |
| PR00081 | putative caffeoyl- CoA | shoot |
| | 3-0 methyltransferase | |
| PR00087 | prolamine RM9 | strong in endosperm |
| PR00090 | prolamine RP6 | strong in endosperm |
| PR00091 | prolamine RP5 | strong in endosperm |
| PR00092 | allergen RA5 | |
| PR00095 | putative | embryo |
| | methionine aminopeptidase | |
| PR00098 | ras-related GTP binding protein | |
| PR00104 | beta expansine EXPB1 | |
| PR00105 | Glycine rich protein | |
| PR00108 | metallothionein like | |
| | protein (putative) | |
| PR00110 | RCc3 strong root | |
| PR00111 | uclacyanin 3-like protein | weak discrimination |
| | | center/shoot meristem |
| PR00116 | 26S proteasome regulatory | very weak meristem |
| | particle non-ATPase subunit 11 | specific |
| PR00117 | putative 40S ribosomal protein | weak in endosperm |
| PR00122 | chlorophyll a/lo-binding | very weak in shoot |
| | protein precursor (Cab27) | |
| PR00123 | putative | Strong leaves |
| | protochlorophyllide reductase | |
| PR00126 | metallothionein RiCMT | strong discrimination |
| | | center shoot meristem |
| PR00129 | GOS2 | Strong constitutive |
| PR00131 | GOS9 | |
| PR00133 | chitinase Cht-3 | very weak meristem |
| | | specific |
| PR00135 | alpha- globulin | Strong in endosperm |
| PR00136 | alanine aminotransferase | Weak in endosperm |
| PR00138 | Cyclin A2 | |
| PR00139 | Cyclin D2 | |
| PR00140 | Cyclin D3 | |
| PR00141 | Cyclophyllin 2 | Shoot and seed |
| PR00146 | sucrose synthase SS1 (barley) | medium constitutive |
| PR00147 | trypsin inhibitor ITR1 (barley) | weak in endosperm |
| PR00149 | ubiquitine 2 with intron | strong constitutive |
| PR00151 | WSI18 | Embryo and stress |
| PR00156 | HVA22 homologue (putative) | |
| PR00157 | EL2 | |
| PR00169 | aquaporine | medium constitutive in |
| | | young plants |
| PR00170 | High mobility group protein | Strong constitutive |
| PR00171 | reversibly glycosylated | weak constitutive |
| | protein RGP1 | |
| PR00173 | cytosolic MDH | shoot |
| PR00175 | RAB21 | Embryo and stress |
| PR00176 | CDPK7 | |
| PR00177 | Cdc2-1 | very weak in meristem |
| PR00197 | sucrose synthase 3 | |
| PRO0198 | OsVP1 | |
| PRO0200 | OSH1 | very weak in young |
| | | plant meristem |
| PRO0208 | putative chlorophyllase | |
| PRO0210 | OsNRT1 | |
| PRO0211 | EXP3 | |
| PRO0216 | phosphate transporter OjPT1 | |
| PRO0218 | oleosin 18 kd | aleurone + embryo |
| PRO0219 | ubiquitine 2 without intron | |
| PRO0220 | RFL | |
| PRO0221 | maize UBI delta intron | not detected |
| PRO0223 | glutelin-1 | |
| PRO0224 | fragment of prolamin | |
| | RP6 promoter | |
| PRO0225 | 4xABRE | |
| PRO0226 | glutelin OSGLUA3 | |
| PRO0227 | BLZ-2 short (barley) | |
| PR00228 | BLZ-2 long (barley) | |

The inducible promoter is a promoter induced in a specific plant tissue, by a developmental stage or by a specific stimuli such as stress conditions comprising, for example, light, temperature, chemicals, drought, high salinity, osmotic shock, oxidant conditions or in case of pathogenicity and include, without being limited to, the light-inducible promoter derived from the pea rbcS gene, the promoter from the alfalfa rbcS gene, the promoters DRE, MYC and MYB active in drought; the promoters INT, INPS, prxEa, Ha hsp17.7G4 and RD21 active in high salinity and osmotic stress, and the promoters hsr203J and str246C active in pathogenic stress.

According to one embodiment the promoter is a pathogen-inducible promoter. These promoters direct the expression of genes in plants following infection with a pathogen such as bacteria, fungi, viruses, nematodes and insects. Such promoters include those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J. Plant Pathol 89:245-254; Uknes et al. (1992) Plant Cell 4:645-656: and Van Loon (1985) Plant Mol. Virol. 4:111-116.

According to one embodiment, when more than one promoter is used in the expression vector, the promoters are identical (e.g., all identical, at least two identical).

According to one embodiment, when more than one promoter is used in the expression vector, the promoters are different (e.g., at least two are different, all are different).

According to one embodiment, the promoter in the expression vector for expression in a plant cell includes, but is not limited to, CaMV 35S, 2× CaMV 35S, CaMV 19S, ubiquitin, AtU626 or TaU6.

According to a specific embodiment, the promoter in the expression vector for expression in a plant cell comprises a 35S promoter.

According to a specific embodiment, the promoter in the expression vector for expression in a plant cell comprises a U6 promoter.

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the invention include those derived from SV40.

According to a specific embodiment, the expression vector for expression in a plant cell comprises a termination sequence, such as but not limited to, a G7 termination sequence, an AtuNos termination sequence or a CaMV-35S terminator sequence.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of some embodiments of the invention can further include additional polynucleotide sequences that allow, for example, the translation of several proteins from a single mRNA such as an internal ribosome entry site (IRES) and sequences for genomic integration of the promoter-chimeric polypeptide.

It will be appreciated that the individual elements comprised in the expression vector can be arranged in a variety of configurations. For example, enhancer elements, promoters and the like, and even the polynucleotide sequence(s) encoding a DNA editing agent can be arranged in a “head-to-tail” configuration, may be present as an inverted complement, or in a complementary configuration, as an anti-parallel strand. While such variety of configuration is more likely to occur with non-coding elements of the expression vector, alternative configurations of the coding sequence within the expression vector are also envisioned.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Stratagene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 late promoter, metallothionein promoter, murine mammary tumor virus promoter, Rous sarcoma virus promoter, 75inalized75 promoter, or other promoters shown effective for expression in eukaryotic cells.

Viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by some embodiments of the invention will depend on the cell type transformed. The ability to select suitable vectors according to the cell type transformed is well within the capabilities of the ordinary skilled artisan and as such no general description of selection consideration is provided herein. For example, bone marrow cells can be targeted using the human T cell leukemia virus type I (HTLV-I) and kidney cells may be targeted using the heterologous promoter present in the baculovirus Autographa californica nucleopolyhedrovirus (AcMNPV) as described in Liang C Y et al., 2004 (Arch Virol. 149: 51-60).

Recombinant viral vectors are useful for in vivo expression of DNA editing agents since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This contrasts with vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

According to one embodiment the nucleic acid construct for expression in a plant cell is a binary vector. Examples for binary vectors are pBIN19, pBI101, pBinAR, pGPTV, pCAMBIA, pBIB-HYG, pBecks, pGreen or pPZP (Hajukiewicz, P. et al., Plant Mol. Biol. 25, 989 (1994), and Hellens et al, Trends in Plant Science 5, 446 (2000)).

Examples of other vectors to be used in other methods of DNA delivery in a plant cell (e.g. transfection, electroporation, bombardment, viral inoculation as discussed below) are: pGE-sgRNA (Zhang et al. Nat. Comms. 2016 7:12697), pJIT163-Ubi-Cas9 (Wang et al. Nat. Biotechnol 2004 32, 947-951), pICH47742::2x35S-5′UTR-hCas9(STOP)-NOST (Belhan et al. Plant Methods 2013 11; 9(1):39), pAHC25 (Christensen, A. H. & P. H. Quail, 1996. Ubiquitin promoter-based vectors for high-level expression of selectable and/or screenable marker genes in monocotyledonous plants. Transgenic Research 5: 213-218), pHBT-sGFP(S65T)-NOS (Sheen et al. Protein phosphatase activity is required for light-inducible gene expression in maize, EMBO J. 12 (9), 3497-3505 (1993).

According to one embodiment, in order to express a functional DNA editing agent, in cases where the cleaving module (nuclease) is not an integral part of the DNA recognition unit, the expression vector may encode the cleaving module as well as the DNA recognition unit (e.g. sgRNA in the case of CRISPR/Cas).

Alternatively, the cleaving module (nuclease) and the DNA recognition unit (e.g. sgRNA) may be cloned into separate expression vectors. In such a case, at least two different expression vectors must be transformed into the same eukaryotic cell.

Alternatively, when a nuclease is not utilized (i.e. not administered from an exogenous source to the cell), the DNA recognition unit (e.g. sgRNA) may be cloned and expressed using a single expression vector.

According to one embodiment, the DNA editing agent comprises a nucleic acid agent encoding at least one DNA recognition unit (e.g. sgRNA) operatively linked to a cis-acting regulatory element active in eukaryotic cells (e.g., promoter).

According to one embodiment, the nuclease (e.g. endonuclease) and the DNA recognition unit (e.g. sgRNA) are encoded from the same expression vector. Such a vector may comprise a single cis-acting regulatory element active in eukaryotic cells (e.g., promoter) for expression of both the nuclease and the DNA recognition unit. Alternatively, the nuclease and the DNA recognition unit may each be operably linked to a cis-acting regulatory element active in eukaryotic cells (e.g., promoter).

According to one embodiment, the nuclease (e.g. endonuclease) and the DNA recognition unit (e.g. sgRNA) are encoded from different expression vectors whereby each is operably linked to a cis-acting regulatory element active in eukaryotic cells (e.g., promoter).

According to one embodiment, the method of some embodiments of the invention does not comprise introducing into the cell donor oligonucleotides.

According to one embodiment, the method of some embodiments of the invention further comprises introducing into the cell donor oligonucleotides.

According to one embodiment, when the modification is an insertion, the method further comprises introducing into the cell donor oligonucleotides.

According to one embodiment, when the modification is a deletion, the method further comprises introducing into the cell donor oligonucleotides.

According to one embodiment, when the modification is a deletion and insertion (e.g. swapping), the method further comprises introducing into the cell donor oligonucleotides.

According to one embodiment, when the modification is a point mutation, the method further comprises introducing into the cell donor oligonucleotides.

As used herein, the term “donor oligonucleotides” or “donor oligos” refers to exogenous nucleotides, i.e. externally introduced into the cell to generate a precise change in the genome.

According to one embodiment, the donor oligonucleotides are synthetic.

According to one embodiment, the donor oligos are RNA oligos.

According to one embodiment, the donor oligos are DNA oligos.

According to one embodiment, the donor oligos are synthetic oligos.

According to one embodiment, the donor oligonucleotides comprise single-stranded donor oligonucleotides (ssODN).

According to one embodiment, the donor oligonucleotides comprise double-stranded donor oligonucleotides (dsODN).

According to one embodiment, the donor oligonucleotides comprise double-stranded DNA (dsDNA).

According to one embodiment, the donor oligonucleotides comprise double-stranded DNA-RNA duplex (DNA-RNA duplex).

According to one embodiment, the donor oligonucleotides comprise double-stranded DNA-RNA hybrid

According to one embodiment, the donor oligonucleotides comprise single-stranded DNA-RNA hybrid.

According to one embodiment, the donor oligonucleotides comprise single-stranded DNA (ssDNA).

According to one embodiment, the donor oligonucleotides comprise double-stranded RNA (dsRNA).

According to one embodiment, the donor oligonucleotides comprise single-stranded RNA (ssRNA).

According to one embodiment, the donor oligonucleotides comprise the DNA or RNA sequence for swapping (as discussed above).

According to one embodiment, the donor oligonucleotides are provided in a non-expressed vector format or oligo.

According to one embodiment, the donor oligonucleotides comprise a DNA donor plasmid (e.g. circular or linearized plasmid).

According to one embodiment, the donor oligonucleotides comprise about 50-5000, about 100-5000, about 250-5000, about 500-5000, about 750-5000, about 1000-5000, about 1500-5000, about 2000-5000, about 2500-5000, about 3000-5000, about 4000-5000, about 50-4000, about 100-4000, about 250-4000, about 500-4000, about 750-4000, about 1000-4000, about 1500-4000, about 2000-4000, about 2500-4000, about 3000-4000, about 50-3000, about 100-3000, about 250-3000, about 500-3000, about 750-3000, about 1000-3000, about 1500-3000, about 2000-3000, about 50-2000, about 100-2000, about 250-2000, about 500-2000, about 750-2000, about 1000-2000, about 1500-2000, about 50-1000, about 100-1000, about 250-1000, about 500-1000, about 750-1000, about 50-750, about 150-750, about 250-750, about 500-750, about 50-500, about 150-500, about 200-500, about 250-500, about 350-500, about 50-250, about 150-250, or about 200-250 nucleotides of single- or double-stranded DNA as well as chimeric DNA-RNA hybrid.

According to a specific embodiment, the donor oligonucleotides comprising the ssODN (e.g. ssDNA or ssRNA) comprise about 200-500 nucleotides.

According to a specific embodiment, the donor oligonucleotides comprising the dsODN (e.g. dsDNA or dsRNA) comprise about 250-5000 nucleotides.

Exemplary donor DNAs and sgRNAs which can be used according to some embodiments of the invention are described in Tables 1A and 1B herein below.

According to one embodiment, for gene swapping of an endogenous RNA silencing molecule (e.g. miRNA) with an RNA silencing sequence of choice (e.g. siRNA), the expression vector, ssODN (e.g. ssDNA or ssRNA) or dsODN (e.g. dsDNA or dsRNA) does not have to be expressed in a cell and could serve as a non-expressing template. According to a specific embodiment, in such a case only the DNA editing agent (e.g. Cas9/sgRNA modules) need to be expressed if provided in a DNA form.

According to some embodiments, for gene editing of an endogenous RNA silencing molecule without the use of a nuclease, the DNA editing agent (e.g., gRNA) may be introduced into the eukaryotic cell with or without (e.g. oligonucleotide donor DNA or RNA, as discussed herein).

According to one embodiment, introducing into the cell donor oligonucleotides is effected using any of the methods described above (e.g. using the expression vectors or RNP transfection).

According to one embodiment, the sgRNA and the DNA donor oligonucleotides are co-introduced into the cell (e.g. eukaryotic cell). It will be appreciated that any additional factors (e.g. nuclease) may be co-introduced therewith.

According to one embodiment, the sgRNA and the DNA donor oligonucleotides are co-introduced into the plant cell (e.g. via bombardment). It will be appreciated that any additional factors (e.g. nuclease) may be co-introduced therewith.

According to one embodiment, the sgRNA is introduced into the cell prior to the DNA donor oligonucleotides (e.g. within a few minutes or a few hours). It will be appreciated that any additional factors (e.g. nuclease) may be introduced prior to, concomitantly with, or following the sgRNA or the DNA donor oligonucleotides.

According to one embodiment, the sgRNA is introduced into the cell subsequent to the DNA donor oligonucleotides (e.g. within a few minutes or a few hours). It will be appreciated that any additional factors (e.g. nuclease) may be introduced prior to, concomitantly with, or following the sgRNA or the DNA donor oligonucleotides.

According to one embodiment, there is provided a composition comprising at least one sgRNA and DNA donor oligonucleotides for genome editing.

According to one embodiment, there is provided a composition comprising at least one sgRNA, a nuclease (e.g. endonuclease) and DNA donor oligonucleotides for genome editing.

According to one embodiment, the at least one sgRNA is operatively linked to a plant expressible promoter.

The DNA editing agents and optionally the donor oligos of some embodiments of the invention can be administered to a single cell, to a group of cells (e.g. plant cells, primary cells or cell lines as discussed above) or to an organism (e.g. plant, mammal, bird, fish, and insect, as discussed above).

Various methods can be used to introduce the expression vector or donor oligos of some embodiments of the invention into eukaryotic cells (e.g. stem cells or plant cells). Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation, microinjection, microparticle bombardment, infection with recombinant viral vectors. In addition, see U.S. Pat. Nos. 5,464,764 and 5,487,992 for positive-negative selection methods.

Thus, the delivery of nucleic acids may be introduced into a cell in embodiments of the invention by any method known to those of skill in the art, including, for example and without limitation: by transformation of protoplasts (See, e.g., U.S. Pat. No. 5,508,184); by desiccation/inhibition-mediated DNA uptake (See, e.g., Potrykus et al. (1985) Mol. Gen. Genet. 199:183-8); by electroporation (See, e.g., U.S. Pat. No. 5,384,253); by agitation with silicon carbide fibers (See, e.g., U.S. Pat. Nos. 5,302,523 and 5,464,765); by Agrobacterium-mediated transformation (See, e.g., U.S. Pat. Nos. 5,563,055, 5,591,616, 5,693,512, 5,824,877, 5,981,840, and 6,384,301); by acceleration of DNA-coated particles (See, e.g., U.S. Pat. Nos. 5,015,580, 5,550,318, 5,538,880, 6,160,208, 6,399,861, and 6,403,865) and by Nanoparticles, nanocarriers and cell penetrating peptides (WO201126644A2; WO2009046384A1; WO2008148223A1) in the methods to deliver DNA, RNA, Peptides and/or proteins or combinations of nucleic acids and peptides into cells.

Other methods of transfection include the use of transfection reagents (e.g. Lipofectin, ThermoFisher), dendrimers (Kukowska-Latallo, J. F. et al., 1996, Proc. Natl. Acad. Sci. USA93, 4897-902), cell penetrating peptides (Mae et al., 2005, Internalisation of cell-penetrating peptides into tobacco protoplasts, Biochimica et Biophysica Acta 1669(2):101-7) or polyamines (Zhang and Vinogradov, 2010, Short biodegradable polyamines for gene delivery and transfection of brain capillary endothelial cells, J Control Release, 143(3):359-366).

According to a specific embodiment, for introducing DNA into cells (e.g. plant cells e.g. protoplasts) the method comprises polyethylene glycol (PEG)-mediated DNA uptake. For further details see Karesch et al. (1991) Plant Cell Rep. 9:575-578; Mathur et al. (1995) Plant Cell Rep. 14:221-226; Negrutiu et al. (1987) Plant Cell Mol. Biol. 8:363-373.

Introduction of nucleic acids to cells (e.g. eukaryotic cells) by viral infection offers several advantages over other methods such as lipofection and electroporation, since higher transfection efficiency can be obtained due to the infectious nature of viruses.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. For gene therapy, the preferred constructs are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of some embodiments of the invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers. Other than containing the necessary elements for the transcription and translation of the inserted coding sequence, the expression construct of some embodiments of the invention can also include sequences engineered to enhance stability, production, purification, yield or toxicity of the expressed peptide.

According to a specific embodiment, a bombardment method is used to introduce foreign genes into eukaryotic cells (e.g. non-plant cells, e.g. animal cells, e.g. mammalian cells). According to one embodiment, the method is transient. Bombardment of eukaryotic cells (e.g. mammalian cells) is also taught by Uchida M et al., Biochim Biophys Acta. (2009) 1790(8):754-64, incorporated herein by reference.

According to one embodiment, plant cells may be transformed stably or transiently with the nucleic acid constructs of some embodiments of the invention. In stable transformation, the nucleic acid molecule of some embodiments of the invention is integrated into the plant genome and as such it represents a stable and inherited trait. In transient transformation, the nucleic acid molecule is expressed by the cell transformed but it is not integrated into the genome and as such it represents a transient trait.

There are various methods of introducing foreign genes into both monocotyledonous and dicotyledonous plants (Potrykus, I., Annu. Rev. Plant. Physiol., Plant. Mol. Biol. (1991) 42:205-225: Shimamoto et al., Nature (1989) 338:274-276).

The principle methods of causing stable integration of exogenous DNA into plant genomic DNA include two main approaches:

(i) Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S. and Arntzen, C. J., Butterworth Publishers, Boston, Mass. (1989) p. 93-112.

(ii) direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563, McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; glass fibers or silicon carbide whisker transformation of cell cultures, embryos or callus tissue, U.S. Pat. No. 5,464,765 or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. Horsch et al. in Plant Molecular Biology Manual A5, Kluwer Academic Publishers, Dordrecht (1988) p. 1-9. A supplementary approach employs the Agrobacterium delivery system in combination with vacuum infiltration. The Agrobacterium system is especially viable in the creation of transgenic dicotyledonous plants.

According to one embodiment, an Agrobacterium-free expression method is used to introduce foreign genes into plant cells. According to one embodiment, the Agrobacterium-free expression method is transient. According to a specific embodiment, a bombardment method is used to introduce foreign genes into plant cells. According to another specific embodiment, bombardment of a plant root is used to introduce foreign genes into plant cells. An exemplary bombardment method which can be used in accordance with some embodiments of the invention is discussed in the examples section which follows.

Furthermore, various cloning kits or gene synthesis can be used according to the teachings of some embodiments of the invention.

Following stable transformation plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. Therefore, it is preferred that the transformed plant be regenerated by micropropagation which provides a rapid, consistent reproduction of the genetically identical transformed plants.

Micropropagation is a process of growing new generation plants from a single piece of tissue that has been excised from a selected parent plant or cultivar. This process permits the mass reproduction of plants having the desired trait. The new generated plants are genetically identical to, and have all of the characteristics of, the original plant. Micropropagation (or cloning) allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment.

Although stable transformation is presently preferred, transient transformation of leaf cells, meristematic cells or the whole plant is also envisaged by some embodiments of the invention.

Transient transformation can be effected by any of the direct DNA transfer methods described above or by viral infection using modified plant viruses.

Viruses that have been shown to be useful for the transformation of plant hosts include CaMV, TMV, TRV and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

Construction of plant RNA viruses for the introduction and expression of non-viral exogenous nucleic acid sequences in plants is demonstrated by the above references as well as by Dawson, W. O. et al., Virology (1989) 172:285-292; Takamatsu et al. EMBO J. (1987) 6:307-311; French et al. Science (1986) 231:1294-1297; and Takamatsu et al. FEBS Letters (1990) 269:73-76.

When the virus is a DNA virus, suitable modifications can be made to the virus itself. Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of this DNA will produce the coat protein which will encapsulate the viral DNA. If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

Construction of plant RNA viruses for the introduction and expression in plants of non-viral exogenous nucleic acid sequences such as those included in the construct of some embodiments of the invention is demonstrated by the above references as well as in U.S. Pat. No. 5,316,931.

In one embodiment, a plant viral nucleic acid is provided in which the native coat protein coding sequence has been deleted from a viral nucleic acid, a non-native plant viral coat protein coding sequence and a non-native promoter, preferably the subgenomic promoter of the non-native coat protein coding sequence, capable of expression in the plant host, packaging of the recombinant plant viral nucleic acid, and ensuring a systemic infection of the host by the recombinant plant viral nucleic acid, has been inserted. Alternatively, the coat protein gene may be inactivated by insertion of the non-native nucleic acid sequence within it, such that a protein is produced. The recombinant plant viral nucleic acid may contain one or more additional non-native subgenomic promoters. Each non-native subgenomic promoter is capable of transcribing or expressing adjacent genes or nucleic acid sequences in the plant host and incapable of recombination with each other and with native subgenomic promoters. Non-native (foreign) nucleic acid sequences may be inserted adjacent the native plant viral subgenomic promoter or the native and a non-native plant viral subgenomic promoters if more than one nucleic acid sequence is included. The non-native nucleic acid sequences are transcribed or expressed in the host plant under control of the subgenomic promoter to produce the desired products.

In a second embodiment, a recombinant plant viral nucleic acid is provided as in the first embodiment except that the native coat protein coding sequence is placed adjacent one of the non-native coat protein subgenomic promoters instead of a non-native coat protein coding sequence.

In a third embodiment, a recombinant plant viral nucleic acid is provided in which the native coat protein gene is adjacent its subgenomic promoter and one or more non-native subgenomic promoters have been inserted into the viral nucleic acid. The inserted non-native subgenomic promoters are capable of transcribing or expressing adjacent genes in a plant host and are incapable of recombination with each other and with native subgenomic promoters. Non-native nucleic acid sequences may be inserted adjacent the non-native subgenomic plant viral promoters such that the sequences are transcribed or expressed in the host plant under control of the subgenomic promoters to produce the desired product.

In a fourth embodiment, a recombinant plant viral nucleic acid is provided as in the third embodiment except that the native coat protein coding sequence is replaced by a non-native coat protein coding sequence.

The viral vectors are encapsidated by the coat proteins encoded by the recombinant plant viral nucleic acid to produce a recombinant plant virus. The recombinant plant viral nucleic acid or recombinant plant virus is used to infect appropriate host plants. The recombinant plant viral nucleic acid is capable of replication in the host, systemic spread in the host, and transcription or expression of foreign gene(s) (isolated nucleic acid) in the host to produce the desired protein.

In addition to the above, the nucleic acid molecule of some embodiments of the invention can also be introduced into a chloroplast genome thereby enabling chloroplast expression.

A technique for introducing exogenous nucleic acid sequences to the genome of the chloroplasts is known. This technique involves the following procedures. First, plant cells are chemically treated so as to reduce the number of chloroplasts per cell to about one. Then, the exogenous nucleic acid is introduced via particle bombardment into the cells with the aim of introducing at least one exogenous nucleic acid molecule into the chloroplasts. The exogenous nucleic acid is selected such that it is integratable into the chloroplast's genome via homologous recombination which is readily effected by enzymes inherent to the chloroplast. To this end, the exogenous nucleic acid includes, in addition to a gene of interest, at least one nucleic acid stretch which is derived from the chloroplast's genome. In addition, the exogenous nucleic acid includes a selectable marker, which serves by sequential selection procedures to ascertain that all or substantially all of the copies of the chloroplast genomes following such selection will include the exogenous nucleic acid. Further details relating to this technique are found in U.S. Pat. Nos. 4,945,050; and 5,693,507 which are incorporated herein by reference. A polypeptide can thus be produced by the protein expression system of the chloroplast and become integrated into the chloroplast's inner membrane.

Regardless of the transformation/infection method employed, the present teachings further select transformed cells comprising a genome editing event.

According to a specific embodiment, selection is carried out such that only cells comprising a successful accurate modification (e.g. swapping, insertion, deletion, point mutation) in the specific locus are selected. Accordingly, cells comprising any event that includes a modification (e.g. an insertion, deletion, point mutation) in an unintended locus are not selected.

According to one embodiment, selection of modified cells can be performed at the phenotypic level, by detection of a molecular event, by detection of a fluorescent reporter, or by growth in the presence of selection (e.g., antibiotic or other selection marker such as resistance to a drug i.e. Nutlin3 in the case of TP53 silencing).

According to one embodiment, selection of modified cells is performed by analyzing the biogenesis and occurrence of the newly edited RNA silencing molecule (e.g. the presence of novel edited miRNA, siRNAs, piRNAs, tasiRNAs, etc).

According to one embodiment, selection of modified cells is performed by analyzing the silencing activity and/or specificity of the RNA silencing molecule, or it's processed small RNA forms, towards a target RNA of interest by validating at least one eukaryotic cell or organism phenotype of the organism that encode the target RNA of interest e.g. cell size, growth rate/inhibition, cell shape, cell membrane integrity, tumor size, tumor shape, a pigmentation of an organism, a size of an organism, infection parameters in an organism (such as viral load or bacterial load) or inflammation parameters in an organism (such as fever or redness), plant leaf coloring, e.g. partial or complete loss of chlorophyll in leaves and other organs (bleaching), presence/absence of necrotic patterns, flower coloring, fruit traits (such as shelf life, firmness and flavor), growth rate, plant size (e.g. dwarfism), crop yield, biotic stress resistance (e.g. disease resistance, nematode mortality, beetle's egg laying rate, or other resistant phenotypes associated with any of bacteria, viruses, fungi, parasites, insects, weeds, and cultivated or native plants), crop yield, metabolic profile, fruit trait, biotic stress resistance, abiotic stress resistance (e.g. heat/cold resistance, drought resistance, salt resistance, resistance to allyl alcohol, or resistant to lack of nutrients e.g. Phosphorus (P)).

According to one embodiment, the silencing specificity of the RNA silencing molecule is determined genotypically, e.g. by expression of a gene or lack of expression.

According to one embodiment, the silencing specificity of the RNA silencing molecule is determined phenotypically.

According to one embodiment, a phenotype of the eukaryotic cell or organism is determined prior to a genotype.

According to one embodiment, a genotype of the eukaryotic cell or organism is determined prior to a phenotype.

According to one embodiment, selection of modified cells is performed by analyzing the silencing activity and/or specificity of RNA silencing molecule towards a target RNA of interest by measuring an RNA level of the target RNA of interest. This can be effected using any method known in the art, e.g. by Northern blotting, Nuclease Protection Assays, In Situ hybridization, quantitative RT-PCR or immunoblotting.

According to one embodiment, selection of modified cells is performed by analyzing eukaryotic cells or clones comprising the DNA editing event also referred to herein as “mutation” or “edit”, dependent on the type of editing sought e.g., insertion, deletion, insertion-deletion (Indel), inversion, substitution and combinations thereof.

Methods for detecting sequence alteration are well known in the art and include, but not limited to, DNA and RNA sequencing (e.g., next generation sequencing), electrophoresis, an enzyme-based mismatch detection assay and a hybridization assay such as PCR, RT-PCR, Rnase protection, in-situ hybridization, primer extension, Southern blot, Northern Blot and dot blot analysis. Various methods used for detection of single nucleotide polymorphisms (SNPs) can also be used, such as PCR based T7 endonuclease, Heteroduplex and Sanger sequencing, or PCR followed by restriction digest to detect appearance or disappearance of unique restriction site/s.

Another method of validating the presence of a DNA editing event e.g., Indels comprises a mismatch cleavage assay that makes use of a structure selective enzyme (e.g. endonuclease) that recognizes and cleaves mismatched DNA.

According to one embodiment, selection of transformed cells is effected by flow cytometry (FACS) selecting transformed cells exhibiting fluorescence emitted by the fluorescent reporter. Following FACS sorting, positively selected pools of transformed eukaryotic cells, displaying the fluorescent marker are collected and an aliquot can be used for testing the DNA editing event as discussed above.

In cases where antibiotic selection marker was used, following transformation eukaryotic cell are cultivated in the presence of selection (e.g., antibiotic), e.g. in a cell culture or until the plant cells develop into colonies i.e., clones and micro-calli. A portion of the cells of the cell culture or of the calli are then analyzed (validated) for the DNA editing event, as discussed above.

According to one embodiment of the invention, the method further comprises validating in the transformed cells complementarity of the endogenous RNA silencing molecule towards the target RNA of interest.

As mentioned above, following modification of the gene encoding the RNA silencing molecule, the RNA silencing molecule comprises at least about 30%, 33%, 40%, 50%, 60%, 70%, 80%, 85%, 90%, 91%, 92%, 93%, 94%, 95%, 96%, 97%, 98%, 99% or even 100%0 complementarity towards the sequence of the target RNA of interest.

The specific binding of designed RNA silencing molecule, or it's processed small RNA forms, with a target RNA of interest can be determined by any method known in the art, such as by computational algorithms (e.g. BLAST) and verified by methods including e.g. Northern blot, In Situ hybridization, QuantiGene Plex Assay etc.

It will be appreciated that positive eukaryotic cells or clones (e.g. plant cell clones) can be homozygous or heterozygous for the DNA editing event. In case of a heterozygous cell, the cell (e.g., when diploid plant cell) may comprise a copy of a modified gene and a copy of a non-modified gene of the RNA silencing molecule. The skilled artisan will select the cells for further culturing/regeneration according to the intended use.

According to one embodiment, when a transient method is desired, eukaryotic cells or clones (e.g. plant cell clones) exhibiting the presence of a DNA editing event as desired are further analyzed and selected for the presence of the DNA editing agent, namely, loss of DNA sequences encoding for the DNA editing agent. This can be done, for example, by analyzing the loss of expression of the DNA editing agent (e.g., at the mRNA, protein) e.g., by fluorescent detection of GFP or q-PCR, HPLC.

According to one embodiment, when a transient method is desired, the eukaryotic cells or clones (e.g. plant cell clones) may be analyzed for the presence of the nucleic acid construct as described herein or portions thereof e.g., nucleic acid sequence encoding the DNA editing agent. This can be affirmed by fluorescent microscopy, q-PCR, FACS, and or any other method such as Southern blot, PCR, sequencing, HPLC).

Positive eukaryotic cell clones may be stored (e.g., cryopreserved).

Alternatively, eukaryotic cells may be further cultured and maintained, for example, in an undifferentiated state for extended periods of time or may be induced to differentiate into other cell types, tissues, organs or organisms as required.

According to one embodiment, when the eukaryotic organism is a plant, the plant is crossed in order to obtain a plant devoid of the DNA editing agent (e.g. of the endonuclease), as discussed below.

Alternatively, plant cells (e.g., protoplasts) may be regenerated into whole plants first by growing into a group of plant cells that develops into a callus and then by regeneration of shoots (callogenesis) from the callus using plant tissue culture methods. Growth of protoplasts into callus and regeneration of shoots requires the proper balance of plant growth regulators in the tissue culture medium that must be customized for each species of plant.

Protoplasts may also be used for plant breeding, using a technique called protoplast fusion. Protoplasts from different species are induced to fuse by using an electric field or a solution of polyethylene glycol. This technique may be used to generate somatic hybrids in tissue culture.

Methods of protoplast regeneration are well known in the art. Several factors affect the isolation, culture, and regeneration of protoplasts, namely the genotype, the donor tissue and its pre-treatment, the enzyme treatment for protoplast isolation, the method of protoplast culture, the culture, the culture medium, and the physical environment. For a thorough review see Maheshwari et al. 1986 Differentiation of Protoplasts and of Transformed Plant Cells: 3-36. Springer-Verlag, Berlin.

The regenerated plants can be subjected to further breeding and selection as the skilled artisan sees fit.

Thus, embodiments of the invention further relate to plants, plant cells and processed product of plants comprising the RNA silencing molecule capable of silencing a target RNA of interest generated according to the present teachings.

According to one aspect of the invention, there is provided a method of producing a plant with reduced expression of a target gene, the method comprising: (a) breeding the plant of some embodiments of the invention, and (b) selecting for progeny plants that have reduced expression of the target RNA of interest, or progeny that comprises a silencing specificity in the RNA molecule towards the target RNA of interest, and which do not comprise the DNA editing agent, thereby producing the plant with reduced expression of a target gene.

According to one aspect of the invention, there is provided a method of producing a plant comprising an RNA molecule having a silencing activity towards a target RNA of interest, the method comprising:

(a) breeding the plant of some embodiments of the invention; and

(b) selecting for progeny plants that comprise the RNA molecule having the silencing activity towards the target RNA of interest, or progeny that comprise a silencing specificity in the RNA molecule towards the target RNA of interest, and which do not comprise the DNA editing agent, thereby producing a plant comprising an RNA molecule having a silencing activity towards a target RNA of interest.

According to one aspect of the invention, there is provided a method producing a plant or plant cell of some embodiments of the invention, comprising growing the plant or plant cell under conditions which allow propagation.

The term “plant” as used herein encompasses whole plants, a grafted plant, ancestors and progeny of the plants and plant parts, including seeds, shoots, stems, roots (including tubers), rootstock, scion, and plant cells, tissues and organs. The plant may be in any form including suspension cultures, embryos, meristematic regions, callus tissue, leaves, gametophytes, sporophytes, pollen, and microspores. Plants that may be useful in the methods of the invention include all plants which belong to the superfamily Viridiplantee, in particular monocotyledonous and dicotyledonous plants including a fodder or forage legume, ornamental plant, food crop, tree, or shrub selected from the list comprising Acacia spp., Acer spp., Actinidia spp., Aesculus spp., Agathis australis, Albizia amara, Alsophila tricolor, Andropogon spp., Arachis spp, Areca catechu, Astelia fragrans, Astragalus cicer, Baikiaea plurijuga, Betula spp., Brassica spp., Bruguiera gymnorrhiza, Burkea 90inalize, Butea frondosa, Cadaba 90inalize, Calliandra spp, Camellia sinensis, Cannabaceae, Cannabis indica, Cannabis, Cannabis saliva, Hemp, industrial Hemp, Capsicum spp., Cassia spp., Centroema pubescens, Chacoomeles spp., Cinnamomum cassia, Coffea arabica, Colophospermum mopane, Coronillia varia, Cotoneaster 90inalize, Crataegus spp., Cucumis spp., Cupressus spp., Cyathea dealbata, Cydonia oblonga, Cryptomeria japonica, Cymbopogon spp., Cynthea dealbata, Cydonia oblonga, Dalbergia monetaria, Davallia 90inalized90, Desmodium spp., Dicksonia squarosa, Dibeteropogon amplectens, Dioclea spp, Dolichos spp., Dorycnium rectum, Echinochloa pyramidalis, Ehraffia spp., Eleusine coracana, Eragrestis spp., Erythrina spp., Eucalypfus spp., Euclea schimperi, Eulalia vi/losa, Pagopyrum spp., Feijoa sellowlana, Fragaria spp., Flemingia spp, Freycinetia banksli, Geranium thunbergii, GinAgo biloba, Glycine javanica, Gliricidia spp, Gossypium hirsutum, Grevillea spp., Guibourtia coleosperma, Hedysarum spp., Hemaffhia altissima, Heteropogon contoffus, Hordeum vulgare, Hyparrhenia rufa, Hypericum erectum, Hypeffhelia dissolute, Indigo incamata, Iris spp., Leptarrhena pyrolifolia, Lespediza spp., Lettuca spp., Leucaena leucocephala, Loudetia simplex, Lotonus bainesli, Lotus spp., Macrotyloma axillare, Malus spp., Manihot esculenta, Medicago saliva, Metasequoia glyptostroboides, Musa sapientum, banana, Nicotianum spp., Onobrychis spp., Ornithopus spp., Oryza spp., Peltophorum africanum, Pennisetum spp., Persea gratissima, Petunia spp., Phaseolus spp., Phoenix canariensis, Phormium cookianum, Photinia spp., Picea glauca, Pinus spp., Pisum sativam, Podocarpus totara, Pogonarthria fleckii, Pogonaffhria squarrosa, Populus spp., Prosopis cineraria, Pseudotsuga menziesii, Pterolobium stellatum, Pyrus communis, Quercus spp., Rhaphiolepsis 90inalized, Rhopalostylis sapida, Rhus natalensis, Ribes grossularia, Ribes spp., Robinia pseudoacacia, Rosa spp., Rubus spp., Salix spp., Schyzachyrium sanguineum, Sciadopitys vefficillata, Sequoia sempervirens, Sequoiadendron giganteum, Sorghum bicolor, Spinacia spp., Sporobolus fimbriatus, Stiburus alopecuroides, Stylosanthos humilis, Tadehagi spp, Taxodium distichum, Themeda triandra, Trifolium spp., Triticum spp., Tsuga heterophylla, Vaccinium spp., Vicia spp., Vitis vinifera, Watsonia pyramidata, Zantedeschia aethiopica, Zea mays, amaranth, artichoke, asparagus, broccoli, Brussels sprouts, cabbage, canola, carrot, cauliflower, celery, collard greens, flax, kale, lentil, oilseed rape, okra, onion, potato, rice, soybean, straw, sugar beet, sugar cane, sunflower, tomato, squash tea, trees. Alternatively algae and other non-Viridiplantae can be used for the methods of some embodiments of the invention.

According to a specific embodiment, the plant is a crop, a flower or a tree.

According to a specific embodiment, the plant is a woody plant species e.g., Actinidia chinensis (Actinidiaceae), Manihot esculenta (Euphorbiaceae), Firiodendron tulipifera (Magnoliaceae), Populus (Salicaceae), Santalum album (Santalaceae), Ulmus (Ulmaceae) and different species of the Rosaceae (Malus, Prunus, Pyrus) and the Rutaceae (Citrus, Microcitrus), Gymnospermae e.g., Picea glauca and Pinus taeda, forest trees (e.g., Betulaceae, Fagaceae, Gymnospermae and tropical tree species), fruit trees, shrubs or herbs, e.g., (banana, cocoa, coconut, coffee, date, grape and tea) and oil palm.

According to a specific embodiment, the plant is of a tropical crop e.g., coffee, macadamia, banana, pineapple, taro, papaya, mango, barley, beans, cassava, chickpea, cocoa (chocolate), cowpea, maize (corn), millet, rice, sorghum, sugarcane, sweet potato, tobacco, taro, tea, yam.

“Grain,” “seed,” or “bean,” refers to a flowering plant's unit of reproduction, capable of developing into another such plant. As used herein, the terms are used synonymously and interchangeably.

According to a specific embodiment, the plant is a plant cell e.g., plant cell in an embryonic cell suspension.

According to a specific embodiment, the plant comprises a plant cell generated by the method of some embodiments of the invention.

According to one embodiment, breeding comprises crossing or selfing.

The term “crossing” as used herein refers to the fertilization of female plants (or gametes) by male plants (or gametes). The term “gamete” refers to the haploid reproductive cell (egg or sperm) produced in plants by mitosis from a gametophyte and involved in sexual reproduction, during which two gametes of opposite sex fuse to form a diploid zygote. The term generally includes reference to a pollen (including the sperm cell) and an ovule (including the ovum). “crossing” therefore generally refers to the fertilization of ovules of one individual with pollen from another individual, whereas “selfing” refers to the fertilization of ovules of an individual with pollen from the same individual. Crossing is widely used in plant breeding and results in a mix of genomic information between the two plants crossed one chromosome from the mother and one chromosome from the father. This will result in a new combination of genetically inherited traits.

As mentioned above, the plant may be crossed in order to obtain a plant devoid of undesired factors e.g. DNA editing agent (e.g. endonuclease).

According to some embodiments of the invention, the plant is non-transgenic.

According to some embodiments of the invention, the plant is a transgenic plant.

According to one embodiment, the plant is non-genetically modified (non-GMO) plant.

According to one embodiment, the plant is a genetically modified (GMO) plant.

According to one embodiment, there is provided a seed of the plant generated according to the method of some embodiments of the invention.

According to one embodiment, there is provided a method of generating a plant with increased stress tolerance, increased yield, increased growth rate or increased yield quality, the method comprising: (a) breeding the plant of some embodiments of the invention, and (b) selecting for progeny plants that have increased stress tolerance, increased yield, increased growth rate or increased yield quality.

The phrase “stress tolerance” as used herein refers to the ability of a plant to endure a biotic or abiotic stress without suffering a substantial alteration in metabolism, growth, productivity and/or viability.

The phrase “abiotic stress” as used herein refers to the exposure of a plant, plant cell, or the like, to a non-living (“abiotic”) physical or chemical agent that has an adverse effect on metabolism, growth, development, propagation, or survival of the plant (collectively, “growth”). An abiotic stress can be imposed on a plant due, for example, to an environmental factor such as water (e.g., flooding, drought, or dehydration), anaerobic conditions (e.g., a lower level of oxygen or high level of CO₂), abnormal osmotic conditions (e.g. osmotic stress), salinity, or temperature (e.g., hot/heat, cold, freezing, or frost), an exposure to pollutants (e.g. heavy metal toxicity), anaerobiosis, nutrient deficiency (e.g., nitrogen deficiency or limited nitrogen), atmospheric pollution or UV irradiation.

The phrase “biotic stress” as used herein refers to the exposure of a plant, plant cell, or the like, to a living (“biotic”) organism that has an adverse effect on metabolism, growth, development, propagation, or survival of the plant (collectively, “growth”). Biotic stress can be caused by, for example, bacteria, viruses, fungi, parasites, beneficial and harmful insects, weeds, and cultivated or native plants.

The phrase “yield” or “plant yield” as used herein refers to increased plant growth (growth rate), increased crop growth, increased biomass, and/or increased plant product production (including grain, fruit, seeds, etc.).

According to one embodiment, in order to generate a plant with increased stress tolerance, increased yield, increased growth rate or increased yield quality, the RNA silencing molecule is designed to target an RNA of interest being of a gene of the plant conferring sensitivity to stress, decreased yield, decreased growth rate or decreased yield quality.

According to one embodiment, exemplary susceptibility plant genes to be targeted (e.g. knocked out) include, but are not limited to, the susceptibility S-genes, such as those residing at genetic loci known as MLO (Mildew Locus O).

According to one embodiment, the plants generated by the present method comprise increased stress tolerance, increased yield, increased yield quality, increased growth rate, by at least about 10%, 20%, 30%, 40%, 50%, 60%, 70%, 80%, 90%, 95% or 100% as compared to plants not generated by the present methods.

Any method known in the art for assessing increased stress tolerance may be used in accordance with the present invention. Exemplary methods of assessing increased stress tolerance include, but are not limited to, downregulation of PagSAP1 in poplar for increased salt stress tolerance as described in Yoon, SK., Bae, E K., Lee, H. et al. Trees (2018) 32: 823. www(dot)doi(dot)org/10.1007/s00468-018-1675-2), and increased drought tolerance in tomato by downregulation of SlbZIP38 (Pan Y et al. Genes 2017, 8, 402; doi:10.3390/genes8120402, incorporated herein by reference.

Any method known in the art for assessing increased yield may be used in accordance with the present invention. Exemplary methods of assessing increased yield include, but are not limited to, reduced DST expression in rice as described in Ar-Rafi Md. Faisal, et al, AJPS>Vol. 8 No. 9, August 2017 DOI: 10.4236/ajps.2017.89149; and downregulation of BnFTA in canola resulted in increased yield as described in Wang Y et al., Mol Plant. 2009 January; 2(1): 191-200.doi: 10.1093/mp/ssn088), both incorporated herein by reference.

Any method known in the art for assessing increased growth rate may be used in accordance with the present invention. Exemplary methods of assessing increased growth rate include, but are not limited to, reduced expression of BIG BROTHER in Arabidopsis or GA2-OXIDASE results in enhance growth and biomass as described in Marcelo de Freitas Lima et al. Biotechnology Research and Innovation(2017)1, 14-25, incorporated herein by reference.

Any method known in the art for assessing increased yield quality may be used in accordance with the present invention. Exemplary methods of assessing increased yield quality include, but are not limited to, down regulation of OsCKX2 in rice results in production of more tillers, more grains, and the grains were heavier as described in Yeh S_Y et al. Rice (N Y). 2015; 8: 36; and reduce OMT levels in many plants, which result in altered lignin accumulation, increase the digestibility of the material for industry purposes as described in Verma S R and Dwivedi U N, South African Journal of Botany Volume 91, March 2014, Pages 107-125, both incorporated herein by reference.

According to one embodiment, the method further enables generation of a plant comprising increased sweetness, increased sugar content, increased flavor, improved ripening control, increased water stress tolerance, increased heat stress tolerance, and increased salt tolerance. One of skill in the art will know how to utilize the methods described herein to choose target RNA sequences for modification.

According to one embodiment, there is provided a method of generating a pathogen or pest tolerant or resistant plant, the method comprising: (a) breeding the plant of some embodiments of the invention, and (b) selecting for progeny plants that are pathogen or pest tolerant or resistant.

According to one embodiment, the target RNA of interest is of a gene of the plant conferring sensitivity to a pathogen or a pest.

According to one embodiment, the target RNA of interest is of a gene of a pathogen.

According to one embodiment, the target RNA of interest is of a gene of a pest.

As used herein the term “pathogen” refers to an organism that negatively affect plants by colonizing, damaging, attacking, or infecting them. Thus, pathogen may affect the growth, development, reproduction, harvest or yield of a plant. This includes organisms that spread disease and/or damage the host and/or compete for host nutrients. Plant pathogens include, but are not limited to, fungi, oomycetes, bacteria, viruses, viroids, virus-like organisms, phytoplasmas, protozoa, nematodes, insects and parasitic plants.

Non-limiting examples of pathogens include, but are not limited to, Roundheaded Borer such as long horned borers; psyllids such as red gum lerp psyllids (Glycaspis brimblecombei), blue gum psyllid, spotted gum lerp psyllids, lemon gum lep psyllids; tortoise beetles; snout beetles: leaf beetles; honey fungus; Thaumastocoris peregrimss; sessile gall wasps (Cynipidae) such as Leptocybe invasa, Ophelimus maskelli and Selitrichodes globules; Foliage-feeding caterpillars such as Omnivorous looper and Orange tortrix; Glassy-winged sharpshooter; and Whiteflies such as Giant whitefly. Other non-limiting examples of pathogens include Aphids such as Chaitophorus spp., Cloudywinged cottonwood and Periphyllus spp.; Armored scales such as Oystershell scale and San Jose scale; Carpenterworm; Clearwing moth borers such as American hornet moth and Western poplar clearwing; Flatheaded borers such as Bronze birch borer and Bronze poplar borer; Foliage-feeding caterpillars such as Fall webworm, Fruit-tree leafroller, Redhumped caterpillar, Satin moth caterpillar, Spiny elm caterpillar, Tent caterpillar, Tussock moths and Western tiger swallowtail; Foliage miners such as Poplar shield bearer; Gall and blister mites such as Cottonwood gall mite; Gall aphids such as Poplar petiolegall aphid; Glassy-winged sharpshooter; Leaf beetles and flea beetles; Mealybugs; Poplar and willow borer; Roundheaded borers; Sawflies; Soft scales such as Black scale, Brown soft scale, Cottony maple scale and European fruit lecanium; Treehoppers such as Buffalo treehopper; and True bugs such as Lace bugs and Lygus bugs.

Other non-limiting examples of viral plant pathogens include, but are not limited to Species: Pea early-browning virus (PEBV), Genus: Tobravirus. Species: Pepper ringspot virus (PepRSV), Genus: Tobravirus. Species: Watermelon mosaic virus (WMV), Genus: Potyvirus and other viruses from the Potyvirus Genus. Species: Tobacco mosaic virus Genus (TMV), Tobamovirus and other viruses from the Tobamovirus Genus. Species: Potato virus X Genus (PVX), Potexvirus and other viruses from the Potexvirus Genus. Thus the present teachings envisage targeting of RNA as well as DNA viruses (e.g. Gemini virus or Bigeminivirus). Geminiviridae viruses which may be targeted include, but are not limited to, Abutilon mosaic bigeminivirus, Ageratum yellow vein bigeminivirus, Bean calico mosaic bigeminivirus, Bean golden mosaic bigeminivirus, Bhendi yellow vein mosaic bigeminivirus, Cassava African mosaic bigeminivirus, Cassava Indian mosaic bigeminivirus, Chino del 95inali bigeminivirus, Cotton leaf crumple bigeminivirus, Cotton leaf curl bigeminivirus, Croton yellow vein mosaic bigeminivirus, Dolichos yellow mosaic bigeminivirus, Euphorbia mosaic bigeminivirus, Horsegram yellow mosaic bigeminivirus, Jatropha mosaic bigeminivirus, Lima bean golden mosaic bigeminivirus, Melon leaf curl bigeminivirus, Mung bean yellow mosaic bigeminivirus, Okra leaf-curl bigeminivirus, Pepper hausteco bigeminivirus, Pepper Texas bigeminivirus, Potato yellow mosaic bigeminivirus, Rhynchosia mosaic bigeminivirus, Serrano golden mosaic bigeminivirus, Squash leaf curl bigeminivirus, Tobacco leaf curl bigeminivirus, Tomato Australian leafcurl bigeminivirus, Tomato golden mosaic bigeminivirus, Tomato Indian leafcurl bigeminivirus, Tomato leaf crumple bigeminivirus, Tomato mottle bigeminivirus, Tomato yellow leaf curl bigeminivirus, Tomato yellow mosaic bigeminivirus, Watermelon chlorotic stunt bigeminivirus and Watermelon curly mottle bigeminivirus.

As used herein the term “pest” refers to an organism which directly or indirectly harms the plant. A direct effect includes, for example, feeding on the plant leaves. Indirect effect includes, for example, transmission of a disease agent (e.g. a virus, bacteria, etc.) to the plant. In the latter case the pest serves as a vector for pathogen transmission.

According to one embodiment, the pest is an invertebrate organism.

Exemplary pests include, but are not limited to, insects, nematodes, snails, slugs, spiders, caterpillars, scorpions, mites, ticks, fungi, and the like.

Insect pests include, but are not limited to, insects selected from the orders Coleoptera (e.g. beetles), Diptera (e.g. flies, mosquitoes), Hymenoptera (e.g. sawflies, wasps, bees, and ants), Lepidoptera (e.g. butterflies and moths), Mallophaga (e.g. lice, e.g. chewing lice, biting lice and bird lice), Hemiptera (e.g. true bugs), Homoptera including suborders Sternorrhyncha (e.g. aphids, whiteflies, and scale insects), Auchenorrhyncha (e.g. cicadas, leafhoppers, treehoppers, planthoppers, and spittlebugs), and Coleorrhyncha (e.g. moss bugs and beetle bugs), Orthroptera (e.g. grasshoppers, locusts and crickets, including katydids and wetas), Thysanoptera (e.g. Thrips), Dermaptera (e.g. Earwigs), Isoptera (e.g. Termites), Anoplura (e.g. Sucking lice), Siphonaptera (e.g. Flea), Trichoptera (e.g. caddisflies), etc.

Insect pests of the invention include, but are not limited to, Maize: Ostrinia nubilalis, European corn borer; Agrotis ipsilon, black cutworm; Helicoverpa zea, corn earworm; Spodoptera frugiperda, fall armyworm; Diatraea grandiosella, southwestern corn borer; Elasmopalpus lignosellus, lesser cornstalk borer; Diatraea saccharalis, sugarcane borer; Diabrotica virgifera, western corn rootworm; Diabrotica longicornis barberi, northern corn rootworm; Diabrotica undecimpunctata howardi, southern corn rootworm; Melanotus spp., wireworms; Cyclocephala borealis, northern masked chafer (white grub); Cyclocephala 96inalized96, southern masked chafer (white grub); Popillia japonica, Japanese beetle; Chaetocnema pulicaria, corn flea beetle; Sphenophorus maidis, maize billbug; Rhopalosiphum maidis, corn leaf aphid; Anuraphis maidiradicis, corn root aphid; Blissus leucopterus leucopterus, chinch bug; Melanoplus femurrubrum, redlegged grasshopper; Melanoplus sanguinipes, migratory grasshopper; Hylemya platura, seedcorn maggot; Agromyza parvicornis, corn blot leafminer; Anaphothrips obscrurus, grass thrips; Solenopsis milesta, thief ant; Tetranychus urticae, twospotted spider mite; Sorghum: Chilo partellus, sorghum borer; Spodoptera frugiperda, fall armyworm; Helicoverpa zea, corn earworm; Elasmopalpus lignosellus, lesser cornstalk borer; Feltia 96inalized9696n, granulate cutworm; Phyllophaga 96inaliz, white grub; Eleodes, Conoderus, and Aeolus spp., wireworms; Oulema melanopus, cereal leaf beetle; Chaetocnema pulicaria, corn flea beetle; Sphenophorus maidis, maize billbug; Rhopalosiphum maidis; corn leaf aphid; Sipha flava, yellow sugarcane aphid; Blissus leucopterus leucopterus, chinch bug; Contarinia sorghicola, sorghum midge, Tetranychus cinnabarinus, carmine spider mite; Tetranychus urticae, twospotted spider mite; Wheat: Pseudaletia unipunctata, army worm; Spodoptera frugiperda, fall armyworm; Elasmopalpus lignosellus, lesser cornstalk borer; Agrotis orthogonia, western cutworm; Elasmopalpus lignosellus, lesser cornstalk borer; Oulema melanopus, cereal leaf beetle; Hypera 96inalize, clover leaf weevil; Diabrotica undecimpunctata howardi, southern corn rootworm; Russian wheat aphid; Schizaphis graminum, greenbug; Macrosiphum avenae, English grain aphid; Melanoplus femurrubrum, redlegged grasshopper; Melanoplus differentialis, differential grasshopper; Melanoplus sanguinipes, migratory grasshopper; Mayetiola destructor, Hessian fly; Sitodiplosis mosellana, wheat midge, Meromyza 97inalized, wheat stem maggot; Hylemya coarctate, wheat bulb fly; Frankliniella fusca, tobacco thrips; Cephus cinctus, wheat stem sawfly; Aceria tulipae, wheat curl mite; Sunflower: Suleima helianthana, sunflower bud moth: Homoeosoma electellum, sunflower moth; zygogramma exclamationis, sunflower beetle; Bothyrus gibbosus, carrot beetle; Neolasioptera murtfeldtiana, sunflower seed midge; Cotton: Heliothis virescens, cotton budworm; Helicoverpa zea, cotton bollworm; Spodoptera exigua, beet armyworm; Pectinophora gossypiella, pink bollworm; Anthonomus grandis, boll weevil; Aphis gossypii, cotton aphid; Pseudatomoscelis seriatus, cotton fleahopper; Trialeurodes abutilonea, bandedwinged whitefly; Lygus lineolaris, tarnished plant bug; Melanoplus femurrubrum, redlegged grasshopper; Melanoplus differentialis, differential grasshopper; Thrips tabaci, onion thrips; Franklinkiella fusca, tobacco thrips; Tetranychus cinnabarinus, carmine spider mite; Tetranychus urticae, twospotted spider mite; Rice: Diatraea saccharalis, sugarcane borer; Spodoptera frugiperda, fall armyworm; Helicoverpa zea, corn earworm, Colaspis brunnea, grape colaspis; Lissorhoptrus oryzophilus, rice water weevil; Sitophilus oryzae, rice weevil; Nephotettix nigropictus, rice leafhopper; Blissus leucopterus leucopterus, chinch bug; Acrosternum hilare, green stink bug; Soybean: Pseudoplusia 97inalize, soybean looper; Anticarsia gemmatalis, velvetbean caterpillar; Plathypena scabs, green cloverworm, Ostrinia nubilalis, European corn borer; Agrotis ipsilon, black cutworm; Spodoptera exigua, beet armyworm; Heliothis virescens, cotton budworm; Helicoverpa zea, cotton bollworm; Epilachna varivestis, Mexican bean beetle; Myzus persicae, green peach aphid; Empoasca fabae, potato leafhopper; Acrosternum hilare, green stink bug; Melanoplus femurrubrum, redlegged grasshopper; Melanoplus differentialis, differential grasshopper; Hylemya platura, seedcorn maggot; Sericothrips variabilis, soybean thrips; Thrips tabaci, onion thrips; Tetranychus turkestani, strawberry spider mite; Tetranychus urticae, twospotted spider mite: Barley: Ostrinia nubilalis, European corn borer; Agrotis ipsilon, black cutworm; Schizaphis graminum, greenbug; Blissus leucopterus leucopterus, chinch bug; Acrosternum hilare, green stink bug; Euschistus servus, brown stink bug; Delia platura, seedcorn maggot; Mayetiola destructor, Hessian fly; Petrobia latens, brown wheat mite; Oil Seed Rape: Brevicoryne brassicae, cabbage aphid; Phyllotreta cruciferae, Flea beetle, Mamestra configurata, Bertha armyworm; Plutella xylostella, Diamond-back moth; Delia ssp., Root maggots. According to one embodiment, the pathogen is a nematode.

Exemplary nematodes include, but are not limited to, the burrowing nematode (Radopholus similis), Caenorhabditis elegans, Radopholus arabocoffeae, Pratylentchus coffeae, root-knot nematode (Meloidogyne spp.), cyst nematode (Heterodera and Globodera spp.), root lesion nematode (Pratylenchus spp.), the stem nematode (Ditylenchus dipsaci), the pine wilt nematode (Bursaphelenchus xylophilus), the reniform nematode (Rotylenchulus reniformis), Xiphinema index, Nacobbus aberrans and Aphelenchoides besseyi.

According to one embodiment, the pathogen is a fungus. Exemplary fungi include, but are not limited to, Fusarium oxysporum, Leptosphaeria maculans (Phoma lingam), Sclerotinia sclerotiorum, Pyricularia grisea, Gibberella fujikuroi (Fusarium moniliforme), Magnaporthe oryzae, Botrytis cinereal, Puccinia spp., Fusarium graminearum, Blumeria graminis, Mycosphaerella graminicola, Colletotrichum spp., Ustilago maydis, Melampsora lini, Phakopsora pachyrhizi and Rhizoctonia solani.

According to a specific embodiment, the pest is an ant, a bee, a wasp, a caterpillar, a beetle, a snail, a slug, a nematode, a bug, a fly, a whitefly, a mosquito, a grasshopper, an earwig, an aphid, a scale, a thrip, a spider, a mite, a psyllid, and a scorpion.

According to one embodiment, in order to generate a pathogen or pest resistant or tolerant plant, the RNA silencing molecule is designed to target an RNA of interest being of a gene of the plant conferring sensitivity to a pathogen or the pest.

Preferably, silencing of the pathogen or pest gene results in the suppression, control, and/or killing of the pathogen or pest which results in limiting the damage that the pathogen or pest causes to the plant. Controlling a pest includes, but is not limited to, killing the pest, inhibiting development of the pest, altering fertility or growth of the pest in such a manner that the pest provides less damage to the plant, decreasing the number of offspring produced, producing less fit pests, producing pests more susceptible to predator attack, or deterring the pests from eating the plant.

According to one embodiment, an exemplary plant gene to be targeted includes, but is not limited to, the gene eIF4E which confers sensitivity to viral infection in cucumber.

According to one embodiment, in order to generate a pathogen resistant or tolerant plant, the RNA silencing molecule is designed to target an RNA of interest being of a gene of the pathogen.

Determination of the plant or pathogen target genes may be achieved using any method known in the art such as by routine bioinformatics analysis.

According to one embodiment, the nematode pathogen gene comprises the Radopholus similis genes Calreticulin13 (CRT) or collagen 5 (col-5).

According to one embodiment, the fungi pathogen gene comprises the Fusarium oxysporum genes FOW2, FRP1, and OPR.

According to one embodiment, the pathogen gene includes, for example, vacuolar ATPase (vATPase), dvssj1 and dvssj2, α-tubulin and snf7.

According to a specific embodiment, when the plant is a Brassica napus (rapeseed), the target RNA of interest includes, but is not limited to, a gene of Leptosphaeria maculans (Phoma lingam) (causing e.g. Phoma stem canker) (e.g. as set forth in GenBank Accession No: AM933613.1); a gene of Flea beetle (Phyllotreta vittula or Chrysomelidae, e.g. as set forth in GenBank Accession No: KT959245.1); or a gene of by Sclerotinia sclerotiorum (causing e.g. Sclerotinia stem rot) (e.g. as set forth in GenBank Accession No: NW_001820833.1).

According to a specific embodiment, when the plant is a Citrus x sinensis (Orange), the target RNA of interest includes, but is not limited to, a gene of Citrus Canker (CCK) (e.g. as set forth in GenBank Accession No: AE008925); a gene of Candidatus Liberibacter spp. (causing e.g. Citrus greening disease) (e.g. as set forth in GenBank Accession No: CP001677.5); or a gene of Armillaria root rot (e.g. as set forth in GenBank Accession No: KY389267.1).

According to a specific embodiment, when the plant is a Elaeis guineensis (Oil palm), the target RNA of interest includes, but is not limited to, a gene of Ganoderma spp. (causing e.g. Basal stem rot (BSR) also known as Ganoderma butt rot) (e.g. as set forth in GenBank Accession No: U56128.1), a gene of Nettle Caterpillar or a gene of any one of Fusarium spp., Phytophthora spp., Pythium spp., Rhizoctonia solani (causing e.g. Root rot).

According to a specific embodiment, when the plant is a Fragaria vesca (Wild strawberry), the target RNA of interest includes, but is not limited to, a gene of Verticillium dahlia (causing e.g. Verticillium Wilt) (e.g. as set forth in GenBank Accession No: DS572713.1); or a gene of Fusarium oxysporum f. sp. fragariae (causing e.g. Fusarium wilt) (e.g. as set forth in GenBank Accession No: KR855868.1);

According to a specific embodiment, when the plant is a Glycine max (Soybean), the target RNA of interest includes, but is not limited to, a gene of P. pachyrhizi (causing e.g. Soybean rust, also known as Asian rust) (e.g. as set forth in GenBank Accession No: DQ026061.1); a gene of Soybean Aphid (e.g. as set forth in GenBank Accession No: KJ451424.1); a gene of Soybean Dwarf Virus (SbDV) (e.g. as set forth in GenBank Accession No: NC_003056.1); or a gene of Green Stink Bug (Acrosternum hilare) (e.g. as set forth in GenBank Accession No: NW_020110722.1).

According to a specific embodiment, when the plant is a Gossypium raimondii (Cotton), the target RNA of interest includes, but is not limited to, a gene of Fusarium oxysporum f. sp. vasinfectum (causing e.g. Fusarium wilt) (e.g. as set forth in GenBank Accession No: JN416614.1); a gene of Soybean Aphid (e.g. as set forth in GenBank Accession No: KJ451424.1); or a gene of Pink bollworm (Pectinophora gossypiella) (e.g. as set forth in GenBank Accession No: KU550964.1).

According to a specific embodiment, when the plant is a Oryza sativa (Rice), the target RNA of interest includes, but is not limited to, a gene of Pyricularia grisea (causing e.g. Rice Blast) (e.g. as set forth in GenBank Accession No: AF027979.1); a gene of Gibberella fujikuroi (Fusarium moniliforme) (causing e.g. Bakanae Disease) (e.g. as set forth in GenBank Accession No: AY862192.1); or a gene of a Stem borer, e.g. Scirpophaga incertulas Walker—Yellow Stem Borer, S. innota Walker—White Stem Borer, Chilo suppressalis Walker—Striped Stem Borer, Sesa-mia inferens Walker—Pink Stem Borer (e.g. as set forth in GenBank Accession No: KF290773.1).

According to a specific embodiment, when the plant is a Solanum lycopersicum (Tomato), the target RNA of interest includes, but is not limited to, a gene of Phytophthora infestans (causing e.g. Late blight) (e.g. as set forth in GenBank Accession No: AY855210.1); a gene of a whitefly Bemisia tabaci (e.g. Gennadius, e.g. as set forth in GenBank Accession No: KX390870.1); or a gene of Tomato yellow leaf curl geminivirus (TYLCV) (e.g. as set forth in GenBank Accession No: LN846610.1).

According to a specific embodiment, when the plant is a Solanum tuberosum (Potato), the target RNA of interest includes, but is not limited to, a gene of Phytophthora infestans (causing e.g. Late Blight) (e.g., as set forth in GenBank Accession No: AY050538.3); a gene of Erwinia spp. (causing e.g. Blackleg and Soft Rot) (e.g. as set forth in GenBank Accession No: CP001654.1); or a gene of Cyst Nematodes (e.g. Globodera pallida and G. rostochiensis) (e.g. as set forth in GenBank Accession No: KF963519.1).

According to a specific embodiment, when the plant is a Theobroma cacao (Cacao), the target RNA of interest includes, but is not limited to, a gene of a gene of basidiomycete Moniliophthora roreri (causing e.g. Frosty Pod Rot) (e.g. as set forth in GenBank Accession No: LATX01001521.1); a gene of Moniliophthora perniciosa (causing e.g. Witches' Broom disease); or a gene of Mirids e.g. Distantiella 100inalized and Sahlbergella singularis, Helopeltis spp, Monalonion specie.

According to a specific embodiment, when the plant is a Vitis vinifera (Grape or Grapevine), the target RNA of interest includes, but is not limited to, a gene of closterovirus GVA (causing e.g. Rugose wood disease) (e.g. as set forth in GenBank Accession No: AF007415.2); a gene of Grapevine leafroll virus (e.g. as set forth in GenBank Accession No: FJ436234.1); a gene of Grapevine fanleaf degeneration disease virus (GFLV) (e.g. as set forth in GenBank Accession No: NC_003203.1); or a gene of Grapevine fleck disease (GFkV) (e.g. as set forth in GenBank Accession No: NC_003347.1).

According to a specific embodiment, when the plant is a Zea mays (Maize also referred to as corn), the target RNA of interest includes, but is not limited to, a gene of a Fall Armyworm (e.g. Spodoptera frugiperda) (e.g. as set forth in GenBank Accession No: AJ488181.3); a gene of European corn borer (e.g. as set forth in GenBank Accession No: GU329524.1); or a gene of Northern and western corn rootworms (e.g. as set forth in GenBank Accession No: NM_001039403.1).

According to a specific embodiment, when the plant is a sugarcane, the target RNA of interest includes, but is not limited to, a gene of an Internode Borer (e.g. Chilo Saccharifagus Indicus), a gene of a Xanthomonas Albileneans (causing e.g. Leaf Scald) or a gene of a Sugarcane Yellow Leaf Virus (SCYLV).

According to a specific embodiment, when the plant is a wheat, the target RNA of interest includes, but is not limited to, a gene of a Puccinia striiformis (causing e.g. stripe rust) or a gene of an Aphid.

According to a specific embodiment, when the plant is a barley, the target RNA of interest includes, but is not limited to, a gene of a Puccinia hordei (causing e.g. Leaf rust), a gene of Puccinia striiformis f. sp. Hordei (causing e.g. stripe rust), or a gene of an Aphid.

According to a specific embodiment, when the plant is a sunflower, the target RNA of interest includes, but is not limited to, a gene of a Puccinia helianthi (causing e.g. Rust disease); a gene of Boerema macdonaldii (causing e.g. Phoma black stem); a gene of a Seed weevil (e.g. red and gray), e.g. Smicronyx fulvus (red); Smicronyx sordidus (gray); or a gene of Sclerotinia sclerotiorum (causing e.g. Sclerotinia stalk and head rot disease).

According to a specific embodiment, when the plant is a rubber plant, the target RNA of interest includes, but is not limited to, a gene of a Microcyclus ulei (causing e.g. South American leaf blight (SALB)); a gene of Rigidoporus microporus (causing e.g. White root disease); a gene of Ganoderma pseudoferreum (causing e.g. Red root disease).

According to a specific embodiment, when the plant is an apple plant, the target RNA of interest includes, but is not limited to, a gene of Neonectria ditissima (causing e.g. Apple Canker), a gene of Podosphaera leucotricha (causing e.g. Apple Powdery Mildew), or a gene of Venturia inaequalis (causing e.g. Apple Scab).

According to one embodiment, the plants generated by the present method are more resistant or tolerant to pathogens by at least about 10%, 20%, 30%, 40%, 50%, 60%, 70%, 80%, 90%, 95% or 100% as compared to plants not generated by the present methods (i.e. as compared to wild type plants).

Any method known in the art for assessing tolerance or resistance to a pathogen of a plant may be used in accordance with the present invention. Exemplary methods include, but are not limited to, reducing MYB46 expression in Arabidopsis which results in enhanced resistance to Botrytis cinerea as described in Ramírez V1, García-Andrade J, Vera P., Plant Signal Behav. 2011 June; 6(6):911-3. Epub 2011 Jun. 1; or downregulation of HCT in alfalfa promotes activation of defense response in the plant as described in Gallego-Giraldo L. et al. New Phytologist (2011) 190: 627-639 doi: 10.1111/j.1469-8137.2010.03621.x), both incorporated herein by reference.

According to one embodiment, there is provided a method of generating a herbicide resistant plant, the method comprising: (a) breeding the plant of some embodiments of the invention, and (b) selecting for progeny plants that are herbicide resistant.

According to one embodiment, the herbicides target pathways that reside within plastids (e.g. within the chloroplast).

Thus to generate herbicide resistant plants, the RNA silencing molecule is designed to target an RNA of interest including, but not limited to, the chloroplast gene psbA (which codes for the photosynthetic quinone-binding membrane protein QB, the target of the herbicide atrazine) and the gene for EPSP synthase (a nuclear protein, however, its overexpression or accumulation in the chloroplast enables plant resistance to the herbicide glyphosate as it increases the rate of transcription of EPSPs as well as by a reduced turnover of the enzyme).

According to one embodiment, the plants generated by the present method are more resistant to herbicides by at least about 10%, 20%, 30%, 40%, 50%, 60%, 70%, 80%, 90%, 95% or 100% as compared to plants not generated by the present methods.

According to one embodiment, there is provided a plant generated according to the method of some embodiments of the invention.

According to one embodiment, there is provided a genetically modified cell comprising a genome comprising a polynucleotide sequence encoding an RNA molecule having a nucleic acid sequence alteration which results in processing of the RNA molecules into small RNAs that are engaged with RISC, the processing being absent from a wild type cell of the same origin devoid of the nucleic acid sequence alteration.

According to one aspect of the invention, there is provided a method of treating a disease in a subject in need thereof, the method comprising generating an RNA molecule having a silencing activity and/or specificity according to the method of some embodiments of the invention, wherein the RNA molecule comprises a silencing activity towards a transcript of a gene associated with an onset or progression of the disease, thereby treating the subject.

According to one aspect of the invention, there is provided an RNA molecule having a silencing activity and/or specificity generated according to the method of some embodiments of the invention, for treating a disease in a subject in need thereof, wherein the RNA molecule comprises a silencing activity towards a transcript of a gene associated with an onset or progression of the disease, thereby treating the subject.

According to one embodiment the disease is an infectious disease, a monogenic recessive disorder, an autoimmune disease and a cancerous disease.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease, disorder or condition from occurring in a subject who may be at risk for the disease, but has not yet been diagnosed as having the disease.

As used herein, the term “subject” or “subject in need thereof” includes animals, including mammals, preferably human beings, at any age or gender which suffer from the pathology. Preferably, this term encompasses individuals who are at risk to develop the pathology.

According to one embodiment, the disease is derived from a virus, a fungus, a bacteria, a trypanosoma or a protozoan parasites (e.g. Plasmodium).

The term “infectious diseases” as used herein refers to any of chronic infectious diseases, subacute infectious diseases, acute infectious diseases, viral diseases, bacterial diseases, protozoan diseases, parasitic diseases, fungal diseases, mycoplasma diseases and prion diseases.

According to one embodiment, in order to treat an infectious disease in a subject, the RNA silencing molecule is designed to target an RNA of interest associated with onset or progression of the infectious disease.

According to one embodiment, the gene associated with the onset or progression of the disease comprises a gene of a pathogen, as discussed below.

According to one embodiment, the gene associated with the onset or progression of the disease comprises a gene of the subject, as discussed below.

According to one embodiment, the target RNA of interest comprises a product of a gene of the eukaryotic cell conferring resistance to the pathogen (e.g. virus, bacteria, fungi, etc.). Exemplary genes include, but are not limited to, CyPA- (Cyclophilins (CyPs)), Cyclophilin A (e.g. for Hepatitis C virus infection), CD81, scavenger receptor class B type I (SR-BI), ubiquitin specific peptidase 18 (USP18), phosphatidylinositol 4-kinase III alpha (PI4K-IIIα) (e.g. for HSV infection) and CCR5- (e.g. for HIV infection). According to one embodiment, the target RNA of interest comprises a product of a gene of the pathogen.

According to one embodiment, the virus is an arbovirus (e.g. Vesicular stomatitis Indiana virus—VSV). According to one embodiment, the target RNA of interest comprises a product of a VSV gene, e.g. G protein (G), large protein (L), phosphoprotein, matrix protein (M) or nucleoprotein.

According to one embodiment, the target RNA of interest includes but is not limited to gag and/or vif genes (i.e. conserved sequences in HIV-1); P protein (i.e. an essential subunit of the viral RNA-dependent RNA polymerase in RSV); P mRNA (i.e. in PIV); core, NS3, NS4B and NS5B (i.e. in HCV); VAMP-associated protein (hVAP-A), La antigen and polypyrimidine tract binding protein (PTB) (i.e. for HCV).

According to a specific embodiment, when the organism is a human, the target RNA of interest includes, but is not limited to, a gene of a pathogen causing Malaria; a gene of HIV virus (e.g. as set forth in GenBank Accession No: NC_001802.1); a gene of HCV virus (e.g. as set forth in GenBank Accession No: NC_004102.1); and a gene of Parasitic worms (e.g. as set forth in GenBank Accession No: XM_003371604.1).

According to a specific embodiment, when the organism is a human, the target RNA of interest includes, but is not limited to, a gene related to a cancerous disease (e.g. Homo sapiens mRNA for bcr/abl e8a2 fusion protein, as set forth in GenBank Accession No: AB069693.1) or a gene related to a myelodysplastic syndrome (MDS) and to vascular diseases (e.g. Human heparin-binding vascular endothelial growth factor (VEGF) mRNA, as set forth in GenBank Accession No: M32977.1)

According to a specific embodiment, when the organism is a cattle, the target RNA of interest includes, but is not limited to, a gene of Infectious bovine rhinotracheitis virus (e.g. as set forth in GenBank Accession No: AJ004801.1), a type 1 bovine herpesvirus (BHV1), causing e.g. BRD (Bovine Respiratory Disease complex); a gene of Bluetongue disease (BTV virus) (e.g. as set forth in GenBank Accession No: KP821170.1); a gene of Bovine Virus Diarrhhoea (BVD) (e.g. as set forth in GenBank Accession No: NC_001461.1); a gene of picornavirus (e.g. as set forth in GenBank Accession No: NC_004004.1), causing e.g. Foot & Mouth disease; a gene of Parainfluenza virus type 3 (PI3) (e.g. as set forth in GenBank Accession No: NC_028362.1), causing e.g. BRD; a gene of Mycobacterium bovis (M. bovis) (e.g. as set forth in GenBank Accession No: NC_037343.1), causing e.g. Bovine Tuberculosis (bTB).

According to a specific embodiment, when the organism is a sheep, the target RNA of interest includes, but is not limited to, a gene of a pathogen causing Tapeworms disease (E. granulosus life cycle, Echinococcus granulosus, Taenia ovis, Taenia hydatigena, Moniezia species) (e.g. as set forth in GenBank Accession No: AJ012663.1); a gene of a pathogen causing Flatworms disease (Fasciola hepatica, Fasciola gigantica, Fascioloides magna, Dicrocoelium dendriticum, Schistosoma bovis) (e.g. as set forth in GenBank Accession No: AY644459.1); a gene of a pathogen causing Bluetongue disease (BTV virus, e.g. as set forth in GenBank Accession No: KP821170.1); and a gene of a pathogen causing Roundworms disease (Parasitic bronchitis, also known as ““hoose””, Elaeophora schneideri, Haemonchus contortus, Trichostrongylus species, Teladorsagia circumcincta, Cooperia species, Nematodirus species, Dictyocaulus 105inalize, Protostrongylus refescens, Muellerius capillaris, Oesophagostomum species, Neostrongylus linearis, Chabertia ovina, Trichuris ovis) (e.g. as set forth in GenBank Accession No: NC_003283.11).

According to a specific embodiment, when the organism is a pig, the target RNA of interest includes, but is not limited to, a gene of African swine fever virus (ASFV) (causing e.g. African Swine Fever) (e.g. as set forth in GenBank Accession No: NC_001659.2); a gene of Classical swine fever virus (causing e.g. Classical Swine Fever) (e.g. as set forth in GenBank Accession No: NC_002657.1); and a gene of a picornavirus (causing e.g. Foot & Mouth disease) (e.g. as set forth in GenBank Accession No: NC_004004.1).

According to a specific embodiment, when the organism is a chicken, the target RNA of interest includes, but is not limited to, a gene of Bird flu (or Avian influenza), a gene of a variant of avian paramyxovirus 1 (APMV-1) (causing e.g. Newcastle disease), or a gene of a pathogen causing Marek's disease.

According to a specific embodiment, when the organism is a tadpole shrimp, the target RNA of interest includes, but is not limited to, a gene of White Spot Syndrome Virus (WSSV), a gene of Yellow Head Virus (YHV), or a gene of Taura Syndrome Virus (TSV).

According to a specific embodiment, when the organism is a salmon, the target RNA of interest includes, but is not limited to, a gene of Infectious Salmon Anaemia (ISA), a gene of Infectious Hematopoietic Necrosis (IHN), a gene of Sea lice (e.g. ectoparasitic copepods of the genera Lepeophtheirus and Caligus).

Assessing the efficacy of treatment may be carried out using any method known in the art, such as by assessing the subject's physical well-being, by blood tests, by assessing viral/bacterial load, etc.

As used herein, the term “monogenic recessive disorder” refers to a disease or condition caused as a result of a single defective gene on the autosomes.

According to one embodiment, the monogenic recessive disorder is a result of a spontaneous or hereditary mutation.

According to one embodiment, the monogenic recessive disorder is autosomal dominant, autosomal recessive or X-linked recessive.

Exemplary monogenic recessive disorders include, but are not limited to, severe combined immunodeficiency (SCID), hemophilia, enzyme deficiencies, Parkinson's Disease, Wiskott-Aldrich syndrome, Cystic Fibrosis, Phenylketonuria, Friedrich's Ataxia, Duchenne Muscular Dystrophy, Hunter disease, Aicardi Syndrome, Klinefelter's Syndrome, Leber's hereditary optic neuropathy (LHON).

According to one embodiment, in order to treat a monogenic recessive disorder in a subject, the RNA silencing molecule is designed to target an RNA of interest associated with the monogenic recessive disorder.

According to one embodiment, when the disorder is Parkinson's disease the target RNA of interest comprises a product of a SNCA (PARK1=4), LRRK2 (PARK8), Parkin (PARK2), PINK1 (PARK6), DJ-1 (PARK7), or ATP13A2 (PARK9) gene.

According to one embodiment, when the disorder is hemophilia or von Willebrand disease the target RNA of interest comprises, for example, a product of an anti-thrombin gene, of coagulation factor VIII gene or of factor IX gene.

Assessing the efficacy of treatment may be carried out using any method known in the art, such as by assessing the subject's physical well-being, by blood tests, bone marrow aspirate, etc.

Non-limiting examples of autoimmune diseases include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), Wegener's granulomatosis, Takayasu's arteritis, Kawasaki syndrome (Praprotnik S. et al., Wien Klin Wochenschr 2000 Aug. 25; 112 (15-16):660), anti-factor VIII autoimmune disease (Lacroix-Desmazes S. et al., Semin Thromb Hemost. 2000; 26 (2):157), necrotizing small vessel vasculitis, microscopic polyangiitis, Churg and Strauss syndrome, pauci-immune focal necrotizing and crescentic glomerulonephritis (Noel L H. Ann Med Interne (Paris). 2000 May; 151 (3):178), antiphospholipid syndrome (Flamholz R. et al., J Clin Apheresis 1999; 14 (4):171), antibody-induced heart failure (Wallukat G. et al., Am J Cardiol. 1999 Jun. 17; 83 (12A):75H), thrombocytopenic purpura (Moccia F. Ann Ital Med Int. 1999 April-June; 14 (2):114; Semple J W. Et al., Blood 1996 May 15; 87 (10):4245), autoimmune hemolytic anemia (Efremov D G. Et al., Leuk Lymphoma 1998 January; 28 (3-4):285; Sallah S. et al., Ann Hematol 1997 March; 74 (3):139), cardiac autoimmunity in Chagas' disease (Cunha-Neto E. et al., J Clin Invest 1996 Oct. 15:98 (8):1709) and anti-helper T lymphocyte autoimmunity (Caporossi A P. Et al., Viral Immunol 1998; 11 (1):9).

Examples of autoimmune rheumatoid diseases include, but are not limited to rheumatoid arthritis (Krenn V. et al., Histol Histopathol 2000 July; 15 (3):791; Tisch R, McDevitt H O. Proc Natl Acad Sci units S A 1994 Jan. 18; 91 (2):437) and ankylosing spondylitis (Jan Voswinkel et al., Arthritis Res 2001; 3 (3): 189).

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. Diseases include, but are not limited to autoimmune diseases of the pancreas, Type 1 diabetes (Castano L. and Eisenbarth G S. Ann. Rev. Immunol. 8:647; Zimmet P. Diabetes Res Clin Pract 1996 October; 34 Suppl:S125), autoimmune thyroid diseases, Graves' disease (Orgiazzi J. Endocrinol Metab Clin North Am 2000 June; 29 (2):339; Sakata S. et al., Mol Cell Endocrinol 1993 March; 92 (1):77), spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), idiopathic myxedema (Mitsuma T. Nippon Rinsho. 1999 August; 57 (8):1759), ovarian autoimmunity (Garza K M. et al., J Reprod Immunol 1998 February; 37 (2):87), autoimmune anti-sperm infertility (Diekman A B. Et al., Am J Reprod Immunol. 2000 March; 43 (3):134), autoimmune prostatitis (Alexander R B. E F al., Urology 1997 December; 50 (6):893) and Type I autoimmune polyglandular syndrome (Hara T. et al., Blood. 1991 Mar. 1; 77 (5):1127).

Examples of autoimmune gastrointestinal diseases include, but are not limited to, chronic inflammatory intestinal diseases (Garcia Herola A. et al., Gastroenterol Hepatol. 2000 January; 23 (1):16), celiac disease (Landau Y E. And Shoenfeld Y. Harefuah 2000 Jan. 16; 138 (2):122), colitis, ileitis and Crohn's disease.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis (Franco A. et al., Clin Immunol Immunopathol 1990 March; 54 (3):382), primary biliary cirrhosis (Jones D E. Clin Sci (Colch) 1996 November; 91 (5):551; Strassburg C P. Et al., Eur J Gastroenterol Hepatol. 1999 June; 11 (6):595) and autoimmune hepatitis (Manns M P. J Hepatol 2000 August; 33 (2):326).

Examples of autoimmune neurological diseases include, but are not limited to, multiple sclerosis (Cross A H. E al., J Neuroimmunol 2001 Jan. 1; 12 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83; Oshima M. et al., Eur J Immunol 1990 December; 20 (12):2563), neuropathies, motor neuropathies (Kornberg A J. J Clin Neurosci. 2000 May; 7 (3):191); Guillain-Barre syndrome and autoimmune neuropathies (Kusunoki S. Am J Med Sci. 2000 April; 319 (4):234), myasthenia, Lambert-Eaton myasthenic syndrome (Takamori M. Am J Med Sci. 2000 April; 319 (4):204); paraneoplastic neurological diseases, cerebellar atrophy, paraneoplastic cerebellar atrophy and stiff-man syndrome (Hiemstra H S. Et al., Proc Natl Acad Sci units S A 2001 Mar. 27; 98 (7):3988); non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies (Antoine J C. And Honnorat J. Rev Neurol (Paris) 2000 January; 156 (1):23); dysimmune neuropathies (Nobile-Orazio E. et al., Electroencephalogr Clin Neurophysiol Suppl 1999; 50:419); acquired neuromyotonia, arthrogryposis multiplex 108inalized108 (Vincent A. et al., Ann N Y Acad Sci. 1998 May 13; 841:482), neuritis, optic neuritis (Soderstrom M. et al., J Neurol Neurosurg Psychiatry 1994 May; 57 (5):544) and neurodegenerative diseases.

Examples of autoimmune muscular diseases include, but are not limited to, myositis, autoimmune myositis and primary Sjogren's syndrome (Feist E. et al., Int Arch Allergy Immunol 2000 September; 123 (1):92) and smooth muscle autoimmune disease (Zauli D. et al., Biomed Pharmacother 1999 June; 53 (5-6):234).

Examples of autoimmune nephric diseases include, but are not limited to, nephritis and autoimmune interstitial nephritis (Kelly C J. J Am Soc Nephrol 1990 August; 1 (2):140).

Examples of autoimmune diseases related to reproduction include, but are not limited to, repeated fetal loss (Tincani A. et al., Lupus 1998:7 Suppl 2:S107-9).

Examples of autoimmune connective tissue diseases include, but are not limited to, ear diseases, autoimmune ear diseases (Yoo T J. Et al., Cell Immunol 1994 August; 157 (1):249) and autoimmune diseases of the inner ear (Gloddek B. et al., Ann N Y Acad Sci 1997 Dec. 29; 830:266).

Examples of autoimmune systemic diseases include, but are not limited to, systemic lupus erythematosus (Erikson J. et al., Immunol Res 1998; 17 (1-2):49) and systemic sclerosis (Renaudineau Y. et al., Clin Diagn Lab Immunol. 1999 March; 6 (2):156); Chan O T. Et al., Immunol Rev 1999 June:169:107).

According to one embodiment, the autoimmune disease comprises systemic lupus erythematosus (SLE).

According to one embodiment, in order to treat an autoimmune disease in a subject, the RNA silencing molecule is designed to target an RNA of interest associated with the autoimmune disease.

According to one embodiment, when the disease is lupus, the target RNA of interest comprises an antinuclear antibody (ANA) such as that pathologically produced by B cells.

Assessing the efficacy of treatment may be carried out using any method known in the art, such as by assessing the subject's physical well-being, by blood tests, bone marrow aspirate, etc.

Non-limiting examples of cancers which can be treated by the method of some embodiments of the invention can be any solid or non-solid cancer and/or cancer metastasis or precancer, including, but is not limiting to, tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer, sporadic; breast cancer, susceptibility to breast cancer, type 4 breast cancer, breast cancer-1, breast cancer-3; breast-ovarian cancer), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B cell, Burkitt, cutaneous T cell, histiocytic, lymphoblastic, T cell, thymic), gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcinosarcoma, choriocarcinoma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypernephroma, insulinoma, islet tumor, keratoma, leiomyoblastoma, leiomyosarcoma, leukemia (e.g., acute lymphatic, acute lymphoblastic, acute lymphoblastic pre-B cell, acute lymphoblastic T cell leukemia, acute—megakaryoblastic, monocytic, acute myelogenous, acute myeloid, acute myeloid with eosinophilia, B cell, basophilic, chronic myeloid, chronic, B cell, eosinophilic, Friend, granulocytic or myelocytic, hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia), lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

According to one embodiment, the cancer which can be treated by the method of some embodiments of the invention comprises a hematologic malignancy. An exemplary hematologic malignancy comprises one which involves malignant fusion of the ABL tyrosine kinase to different other chromosomes generating what is termed BCR-ABL which in turn resulting in malignant fusion protein. Accordingly, targeting the fusion point in the mRNA may silence only the fusion mRNA for down-regulation while the normal proteins, essential for the cell, will be, spared.

According to one embodiment, in order to treat a cancerous disease in a subject, the RNA silencing molecule is designed to target an RNA of interest associated with the cancerous disease.

According to one embodiment, the target RNA of interest comprises a product of an oncogene (e.g. mutated oncogene).

According to one embodiment, the target RNA of interest restores the function of a tumor suppressor.

According to one embodiment, the target RNA of interest comprises a product of a RAS, MCL-1 or MYC gene.

According to one embodiment, the target RNA of interest comprises a product of a BCL-2 family of apoptosis-related genes.

Exemplary target genes include, but are not limited to, mutant dominant negative TP53, Bcl-x, IAPs, Flip, Faim3 and SMS1.

According to one embodiment, when the cancer is melanoma, the target RNA of interest comprises BRAF. Several forms of BRAF mutations are contemplated herein, including e.g. V600E, V600K, V600D, V600G, and V600R.

According to one embodiment, the method is affected by targeting RNA silencing molecules in healthy immune cells, such as white blood cells e.g. T cells, B cells or NK cells (e.g. from a patient or from a cell donor) to a target an RNA of interest such that the immune cells are capable of killing (directly or indirectly) malignant cells (e.g. cells of a hematological malignancy).

According to one embodiment, the method is affected by targeting RNA silencing molecules to silence proteins (i.e. target RNA of interest) that are manipulated by cancer factors (i.e. in order to suppress immune responses from recognizing the malignancy), such that the cancer can be recognized and eradicated by the native immune system.

Assessing the efficacy of treatment may be carried out using any method known in the art, such as by assessing the tumor growth or the number of neoplasms or metastases, e.g. by MRI, CT, PET-CT, by blood tests, ultrasound, x-ray, etc.

According to one aspect of the invention, there is provided a method of enhancing efficacy and/or specificity of a chemotherapeutic agent in a subject in need thereof, the method comprising generating an RNA molecule having a silencing activity and/or specificity according to the method of some embodiments of the invention, wherein the RNA molecule comprises a silencing activity towards a transcript of a gene associated with enhancement of efficacy and/or specificity of the chemotherapeutic agent.

As used herein, the term “chemotherapeutic agent” refer to an agent that reduces, prevents, mitigates, limits, and/or delays the growth of neoplasms or metastases, or kills neoplastic cells directly by necrosis or apoptosis of neoplasms or any other mechanism, or that can be otherwise used, in a pharmaceutically-effective amount, to reduce, prevent, mitigate, limit, and/or delay the growth of neoplasms or metastases in a subject with neoplastic disease (e.g. cancer).

Chemotherapeutic agents include, but are not limited to, fluoropyrimidines; pyrimidine nucleosides; purine nucleosides; anti-folates, platinum agents; anthracyclines/anthracenediones; epipodophyllotoxins; camptothecins (e.g., Karenitecin); hormones; hormonal complexes; antihormonals; enzymes, proteins, peptides and polyclonal and/or monoclonal antibodies; immunological agents; vinca alkaloids; taxanes; epothilones; antimicrotubule agents; alkylating agents; antimetabolites; topoisomerase inhibitors; antivirals; and various other cytotoxic and cytostatic agents.

According to a specific embodiment, the chemotherapeutic agent includes, but is not limited to, abarelix, aldesleukin, aldesleukin, alemtuzumab, alitretinoin, allopurinol, altretamine, amifostine, anastrozole, arsenic trioxide, asparaginase, azacitidine, bevacuzimab, bexarotene, bleomycin, bortezomib, busulfan, calusterone, capecitabine, carboplatin, carmustine, celecoxib, cetuximab, cisplatin, cladribine, clofarabine, cyclophosphamide, cytarabine, dacarbazine, dactinomycin, actinomycin D, Darbepoetin alfa, Darbepoetin alfa, daunorubicin liposomal, daunorubicin, decitabine, Denileukindiftitox, dexrazoxane, dexrazoxane, docetaxel, doxorubicin, dromostanolone propionate, Elliott's B Solution, epirubicin, Epoetin alfa, erlotinib, estramustine, etoposide, exemestane, Filgrastim, floxuridine, fludarabine, fluorouracil 5-FU, fulvestrant, gefitinib, gemcitabine, gemtuzumabozogamicin, goserelin acetate, histrelin acetate, hydroxyurea, IbritumomabTiuxetan, idarubicin, ifosfamide, imatinibmesylate, interferon alfa 2a, Interferon alfa-2b, irinotecan, lenalidomide, letrozole, leucovorin, Leuprolide Acetate, levamisole, lomustine, CCNU, meclorethamine, nitrogen mustard, megestrol acetate, melphalan, L-PAM, mercaptopurine 6-MP, mesna, methotrexate, mitomycin C, mitotane, mitoxantrone, nandrolonephenpropionate, nelarabine, Nofetumomab, Oprelvekin, Oprelvekin, oxaliplatin, paclitaxel, palifermin, pamidronate, pegademase, pegaspargase, Pegfilgrastim, pemetrexed disodium, pentostatin, pipobroman, plicamycinmithramycin, porfimer sodium, procarbazine, quinacrine, Rasburicase, Rituximab, sargramostim, sorafenib, streptozocin, sunitinib maleate, tamoxifen, temozolomide, teniposide VM-26, testolactone, thioguanine 6-TG, thiotepa, thiotepa, topotecan, toremifene, Tositumomab, Trastuzumab, tretinoin ATRA, Uracil Mustard, valrubicin, vinblastine, vinorelbine, zoledronate and zoledronic acid.

According to one embodiment, the effect of the chemotherapeutic agent is enhanced by about 5%, 10%, 20%, 30%, 40%, 50%, 60%, 70%, 80%, 90%, 95%, 99% or by 100% as compared to the effect of a chemotherapeutic agent in a subject not treated by the DNA editing agent designed to confer a silencing activity and/or specificity of an RNA silencing molecule towards a target RNA of interest.

Assessing the efficacy and/or specificity of a chemotherapeutic agent may be carried out using any method known in the art, such as by assessing the tumor growth or the number of neoplasms or metastases, e.g. by MRI, CT, PET-CT, by blood tests, ultrasound, x-ray, etc.

According to one embodiment, the method is affected by targeting RNA silencing molecules in healthy immune cells, such as white blood cells e.g. T cells, B cells or NK cells (e.g. from a patient or from a cell donor) to target an RNA of interest such that the immune cells are capable of decreasing resistance of the cancer to chemotherapy.

According to one embodiment, the method is affected by targeting RNA silencing molecules in healthy immune cells, such as white blood cells e.g. T cells, B cells or NK cells (e.g. from a patient or from a cell donor) to target an RNA of interest such that the immune cells are resistant to chemotherapy.

According to one embodiment, in order to enhance efficacy and/or specificity of a chemotherapeutic agent in a subject, the RNA silencing molecule is designed to target an RNA of interest associated with suppression of efficacy and/or specificity of the chemotherapeutic agent.

According to one embodiment, the target RNA of interest comprises a product of a drug-metabolising enzyme gene (e.g. cytochrome P450 [CYP] 2C8, CYP2C9, CYP2C19, CYP2D6, CYP3A4, CYP3A5, dihydropyrimidine dehydrogenase, uridine diphosphate glucuronosyltransferase [UGT] 1A1, glutathione S-transferase, sulfotransferase [SULT] 1A1, N-acetyltransferase [NAT], thiopurine methyltransferase [TPMT]) and drug transporters (P-glycoprotein [multidrug resistance 1], multidrug resistance protein 2 [MRP2], breast cancer resistance protein [BCRP]).

According to one embodiment, the target RNA of interest comprises an anti-apoptotic gene. Exemplary target genes include, but are not limited to, Bcl-2 family members, e.g. Bcl-x, IAPs, Flip, Faim3 and SMS1.

According to one aspect of the invention, there is provided a method of inducing cell apoptosis in a subject in need thereof, the method comprising generating an RNA molecule having a silencing activity and/or specificity according to the method of some embodiments of the invention, wherein the RNA molecule comprises a silencing activity towards a transcript of a gene associated with apoptosis, thereby inducing cell apoptosis in the subject.

The term “cell apoptosis” as used herein refers to the cell process of programmed cell death. Apoptosis characterized by distinct morphologic alterations in the cytoplasm and nucleus, chromatin cleavage at regularly spaced sites, and endonucleolytic cleavage of genomic DNA at internucleosomal sites. These changes include blebbing, cell shrinkage, nuclear fragmentation, chromatin condensation, and chromosomal DNA fragmentation.

According to one embodiment, cell apoptosis is enhanced by about 5%, 10%, 20%, 30%, 40%, 50%, 60%, 70%, 80%, 90%, 95%, 99% or by 100% as compared to cell apoptosis in a subject not treated by the DNA editing agent conferring a silencing activity and/or specificity of an RNA silencing molecule towards a target RNA of interest.

Assessing cell apoptosis may be carried out using any method known in the art, e.g. cell proliferation assay, FACS analysis etc.

According to one embodiment, in order to induce cell apoptosis in a subject, the RNA silencing molecule is designed to target an RNA of interest associated with the apoptosis.

According to one embodiment, the target RNA of interest comprises a product of a BCL-2 family of apoptosis-related genes.

According to one embodiment, the target RNA of interest comprises an anti-apoptotic gene. Exemplary genes include, but are not limited to, mutant dominant negative TP53, Bcl-x, IAPs, Flip, Faim3 and SMS1.

According to one aspect of the invention, there is provided a method of generating a eukaryotic non-human organism, wherein at least some of the cells of the eukaryotic non-human organism comprise a genome comprising a polynucleotide sequence encoding an RNA molecule having a nucleic acid sequence alteration which results in processing of the RNA molecules into small RNAs that are engaged with RISC, the processing being absent from a wild type cell of the same origin devoid of the nucleic acid sequence alteration.

The DNA editing agents, RNA editing agents and optionally the donor oligos of some embodiments of the invention (or expression vectors or RNP complex comprising same) can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the DNA editing agents and optionally the donor oligos accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinylpyrrolidone, agar, or alginic acid or a salt thereof such as sodium alginate.

Dragee cores are provided with suitable coatings. For this purpose, concentrated sugar solutions may be used which may optionally contain gum 116inali, talc, polyvinylpyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (e.g. DNA editing agent) effective to prevent, alleviate or ameliorate symptoms of a disorder (e.g., cancer or infectious disease) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Animal models for cancerous diseases are described e.g. in Yee et al., Cancer Growth Metastasis. (2015) 8(Suppl 1): 115-118. Animal models for infectious diseases are described e.g. in Shevach, Current Protocols in Immunology, Published Online: 1 Apr. 2011, DOI: 10.1002/0471142735.im1900s93.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide the active ingredient at a sufficient amount to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

Additionally, there is provided:

According to some embodiments, silencing activity of a silencing RNA, as used herein, is mediated by the silencing RNA being processed into RNA that can bind the RNA-induced silencing complex (RISC). According to some embodiments, the identified genes are homologous to genes encoding silencing RNA molecules whose silencing activity and/or processing into small silencing RNA is dependent on their secondary structure, and which encode for RNA molecules that are processed into RNA that can bind RNA-induced silencing complex (RISC).

The present invention is further based in part on the development of a method which enables imparting silencing activity to RNA molecules encoded by the identified genes. According to some embodiments, the identified genes further include identified gene elements which encode for RNA molecules that are homologous to silencing RNA molecules. In non-limiting examples, such gene elements may be a region encoding for an intron or a UTR of an RNA molecule.

According to some embodiments, imparting the silencing activity comprises introducing nucleotide changes into the identified genes, such that RNA encoded by them is processed into a RISC-binding RNA. According to some embodiments, the nucleotide changes enable altering the secondary structure of an RNA encoded by the identified gene such that it corresponds to the secondary structure of a homolgous canonical RNA (which is processable to a RISC-binding RNA). According to some embodiments, a mature sequence of an RNA molecule encoded by an identified gene refers to a sequence which corresponds in sequence location to the mature sequence in the corresponding homologous canonical silencing RNA.

According to some embodiments, the imparted silencing activity is towards a sequence corresponding to the mature sequence of the silencing-dysfunctional RNA encoded by the identified gene (also referred to herein as “reactivation” of silencing activity). According to other embodiments, the imparted silencing activity is towards a target gene of choice, such that the mature sequence of the silencing-dysfunctional RNA is altered (also referred to herein as “redirection” of silencing activity), wherein the other target gene can be endogenous or exogenous to the cell in which silencing is imparted. Without wishing to be bound by theory or mechanism, reactivation of silencing activity is performed, according to some embodiments, by introducing nucleotide changes into an identified gene, such that it encodes an RNA molecule having a secondary structure that is substantially equivalent to that of a homologous RNA molecule processable to a silencing RNA with silencing activity (while maintaining the targeting specificity of the mature sequence within the previously silencing-dysfunctional RNA). According to some embodiments, this change in secondary structure enables the RNA encoded by the identified gene to be processed to silencing RNA which can binds RISC. According to some embodiments, introducing nucleotide changes is through gene editing (e.g. using the CRISPR/Cas9 technology), potentially in combination with introduction of a template, as disclosed, for example, in WO 2019/058255, incorporated herein by reference.

According to some embodiments, the term “identified gene” further includes gene elements, such as, but not limited to, an exon, an intron or a UTR (i.e. the identified sequences which encode RNA homologous to an RNA processable to a silencing molecule might not be stand-alone genes).

According to some embodiments, an RNA molecule processable to RNA that has a silencing activity is processed into an RNA molecule which has a silencing activity mediated by engaging RISC. According to some embodiments, an RNA molecule which has a silencing activity is an RNA molecule which is able to engage with RNA-induced silencing complex (RISC).

According to some embodiments, an RNA molecule whose silencing activity and/or processing into small silencing RNA is dependent on the RNA molecule's secondary structure is a microRNA (miRNA) molecule.

According to one embodiment, an RNA molecule which has a secondary structure that enables it to be processed into an RNA having a silencing activity is selected from the group consisting of: microRNA (miRNA), short-hairpin RNA (shRNA), small nuclear RNA (snRNA or URNA), small nucleolar RNA (snoRNA), Small Cajal body RNA (scaRNA), transfer RNA (tRNA), ribosomal RNA (rRNA), repeat-derived RNA, autonomous and non-autonomous transposable and retro-transposable element-derived RNA, autonomous and non-autonomous transposable and retro-transposable element RNA and long non-coding RNA (lncRNA).

According to one aspect of the present invention, provided herein is a method of introducing silencing activity to a first RNA molecule in a cell (also referred to herein as “the method of introducing silencing activity”), the method comprising:

(a) selecting a first nucleic acid sequence within the cell, wherein:
i. the first nucleic acid sequence is transcribed into the first RNA molecule within the cell;
ii. the sequence of the first RNA molecule has a partial homology to the sequence of a second RNA molecule, excluding sequence identity; wherein the second RNA molecule is processable to a third RNA molecule having a silencing activity; and wherein the second RNA molecule is encoded by a second nucleic acid sequence in the cell; and
iii. the first RNA molecule is not processable, or is processable differently than the second RNA molecule (i.e. non-canonical processing), such that the first RNA molecule is not processed to an RNA molecule having a silencing activity of the same nature as the third RNA molecule;
   (b) modifying the first nucleic acid sequence such that it encodes a modified first RNA molecule, the modified first RNA molecule being processable to a fourth RNA in the same way that the second RNA molecule is processable to the third RNA molecule, such that the fourth RNA molecule has a silencing activity of the same nature as the third RNA molecule, thereby introducing a silencing activity to the first RNA molecule.

According to some embodiments, the second nucleic acid sequence is a gene encoding a microRNA (miRNA) molecule. According to some embodiments, the second RNA molecule is a precursor for miRNA.

According to some embodiments, a first RNA molecule which is processable differently than the second RNA molecule does not undergo canonical processing with respect to the second RNA molecule.

According to some embodiments, the first RNA molecule does not have a silencing activity as it does not have a secondary structure which enables it to have a silencing activity. According to some embodiments, the first RNA molecule is not processable to an RNA silencing molecule having silencing activity corresponding to that of the third RNA molecule, because the secondary structure of the first RNA molecule does not render it processable to an RNA molecule that has such silencing activity. In a non-limiting example, the first RNA molecule is homologous to a second RNA molecule which is a micro-RNA precursor, but the first RNA molecule does not have a secondary structure enabling it to be processed to a micro RNA having silencing activity.

According to some embodiments, the first RNA molecule has a secondary structure different than of the second RNA molecule and thus the first RNA molecule is processable, but is processable differently than the second RNA molecule, resulting in the first RNA molecule not being processed to an RNA molecule having a silencing activity corresponding to that of the third RNA molecule. In a non-limiting example, the second RNA molecule is a precursor of a microRNA but the secondary structure of the first RNA molecule is different than that of the second RNA molecule, and thus the first RNA molecule is not proceaable to a small RNA which has a silencing activity corresponding to that of a micro RNA.

According to some embodiments, modifying the first nucleic acid sequence comprises modifying the sequence such that the modified first RNA molecule has a secondary structure that enables it to be processed into the fourth RNA molecule that has a silencing activity.

According to some embodiments, modifying the first nucleic acid sequence comprises modifying the sequence such that the modified first RNA molecule has essentially the same secondary structure as that of the second RNA molecule, optionally a secondary structure which is at least 95%, 96%, 97%, 98%, 99%, 99.5%, 99.9% or 100% identical to the secondary structure of the second RNA molecule, preferably at least 99%, 99.5%, 99.9% or 100% identical to the secondary structure of the second RNA molecule. Each possibility represents a separate embodiment of the present invention.

According to some embodiments, the secondary structure is at least 95%, 96%, 97%, 98%, 99%, 99.5%, 99.9% or 100% identical to the secondary structure of the second RNA molecule (e.g. when the secondary structure of the first RNA molecule is translated to a linear string form and is compared to a string form of a secondary structure of the second RNA molecule). Any method known in the art can be used to translate a secondary structure to a series of strings which can be compared with another series of strings, such as but not limited to RNAfold.

According to some embodiments, the second RNA molecule has a secondary structure which enables it to be processed into the third RNA molecule having a silencing activity; and modifying the first nucleic acid sequence comprises modifying the sequence such that the modified first RNA molecule has substantially the same secondary structure as that of the second RNA molecule.

According to some embodiments, (i) the second RNA molecule has a secondary structure which enables it to be processed into the third RNA molecule having a silencing activity; (ii) modifying the first nucleic acid sequence comprises modifying the sequence such that the modified first RNA molecule has substantially the same secondary structure as that of the second RNA molecule; and (iii) modifying the first nucleic acid sequence excludes modifying those nucleotides which correspond in location to those of the third RNA molecule, thus resulting in a modified first RNA molecule which is processable to a fourth RNA molecule having a silencing activity. This embodiment describes “reactivation” of silencing activity within the first RNA molecule, without directing it to a target of choice. According to other embodiments, (i) the second RNA molecule has a secondary structure which enables it to be processed into the third RNA molecule having a silencing activity; (ii) modifying the first nucleic acid sequence comprises modifying the sequence such that the modified first RNA molecule has substantially the same secondary structure as that of the second RNA molecule; and (iii) modifying the first nucleic acid sequence includes modifying the nucleotides which correspond in location to those of the third RNA molecule, such that the fourth RNA molecule has a silencing activity towards a target of choice. This embodiment describes “redirection” of silencing activity within the first RNA molecule, directing it to a target of choice, which may be endogenous or exogenous.

According to some embodiments, the method of introducing silencing activity further comprises predicting the secondary structure of the first RNA molecule and second RNA molecule based on their nucleotide sequences. According to some embodiments, the method of introducing silencing activity further comprises determining the nucleotide changes required for changing the secondary structure of the first RNA to be essentially identical to that of the secondary RNA.

According to some embodiments, modifying the first nucleic acid sequence comprises modifying the sequence such that the modified first RNA molecule is processable to a fourth RNA molecule which has a silencing activity which is mediated by engaging RISC.

According to some embodiments, the sequence of the first RNA molecule has a partial homology to the sequence of the second RNA molecule such that there is at least a partial homology between the sequence encoding the third RNA molecule and the sequence in the corresponding location within the first RNA molecule, excluding complete identity.

According to one embodiment, the first nucleic acid molecule is a gene from H. sapiens, wherein the gene is selected from the group consisting of the genes having the sequences set forth in any of SEQ ID Nos. 352 to 392.

As used herein the term “about” refers to +10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

It is understood that any Sequence Identification Number (SEQ ID NO) disclosed in the instant application can refer to either a DNA sequence or an RNA sequence, depending on the context where that SEQ ID NO is mentioned, even if that SEQ ID NO is expressed only in a DNA sequence format or an RNA sequence format. For example, SEQ ID NO: 1 is expressed in a DNA sequence format (e.g., reciting T for thymine), but it can refer to either a DNA sequence that corresponds to a nucleic acid sequence, or the RNA sequence of an RNA molecule nucleic acid sequence. Similarly, though some sequences are expressed in an RNA sequence format (e.g., reciting U for uracil), depending on the actual type of molecule being described, it can refer to either the sequence of an RNA molecule comprising a dsRNA, or the sequence of a DNA molecule that corresponds to the RNA sequence shown. In any event, both DNA and RNA molecules having the sequences disclosed with any substitutes are envisioned.

### EXAMPLES

Reference is now made to the following examples, which together with the above descriptions, illustrate the invention in a non-limiting fashion.

Generally, the nomenclature used herein and the laboratory procedures utilized in the present invention include molecular, biochemical, microbiological, microscopy and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8^{th} Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Experimental Procedures

### Design to Impart and Redirect Silencing Activity of Non-Coding RNA

Stage A: Identification of miRNA-Like Precursors

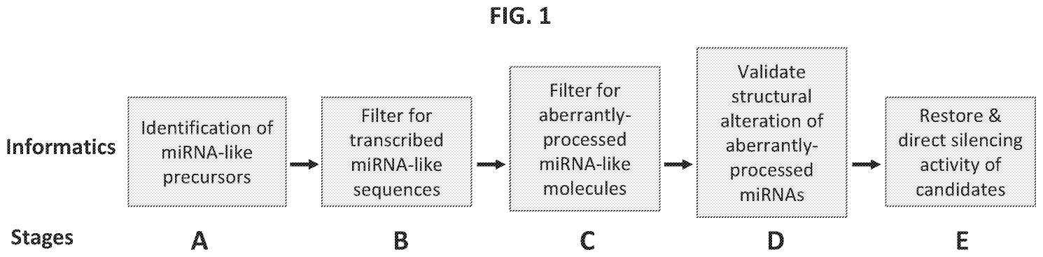

As illustrated in FIG. 1 step (A), the scheme starts with identification of sequences that relate, but are not identical, to non-coding RNA (ncRNA) precursors, e.g. miRNA-like precursors, as follows:
Sequences derived from known miRNAs of various host species, e.g. Arabidopsis (A. thaliana), Human (H. sapiens) and Caenorhabditis elegans (C. elegans), were used in order to find potential miRNA-like precursors in these organisms.
Briefly, a Blast search using the functional miRNA precursors and/or mature miRNA sequences of a certain organism was performed against the corresponding host genome, thus identifying precursor sequences that are similar but not identical (i.e. miRNA-like sequences) to the functional miRNAs. Search parameters are further detailed below under “construction of candidate sets”.
Out of the identified miRNA-like sequences, it was determined whether each sequence originates from a protein-coding gene or a non-coding gene.
As detailed below, the initial list of candidate genes encoding miRNA-like precursors was further filtered according to expression data to identify ncRNA precursors which can serve as basis for reactivation (and possibly redirection) of silencing activity.
   Stage B: Filter for Transcribed miRNA-Like Molecules

Next, as illustrated in FIG. 1 step (B), the scheme continues with filtering for transcribed ncRNA-like molecules. e.g. miRNA-like molecules, as follows:
To avoid detection of similar functional miRNA precursors, a stringent search against the dysfunctional precursors was performed in several, publicly available sRNAseq datasets.
A total of 142 publicly available sRNAseq samples were utilized for sensitive expression detection (when expression is non-ubiquitous).
A total of 142 small RNA-seq sequencing samples were extracted from publicly available resources. The H. sapiens datasets included seven samples from the liver, 18 blood samples, 34 brain samples, 24 lung samples and 3 bladder samples. All human samples used in the analysis were from healthy individuals. C. elegans samples were derived from several developmental stages—embryos (24 samples), young adults (9 samples), L4 (6 samples) and 3 samples from mixed stages. The samples of A. thaliana were derived from various parts of the plant—root (5 samples), shoot (2 samples), leaf (3 samples) and seedlings (7 samples).
To detect and trim specific sequencing primers, a QC analysis was performed for each sRNAseq sample using fastqc. The adapter sequence of each sample was identified and trimmed using cutadapt (M. Martin. Cutadapt removes adapter sequences from high-throughput sequencing reads. EMBnet.journal 17(1):10-12, May 11).
All sRNAseq samples were aligned with no mismatches to the genome of the corresponding species and the output bam alignments were then sorted to detect non-processed miRNA-like molecules.
   Stage C: Filter for Non-Processed miRNA-Like Molecules

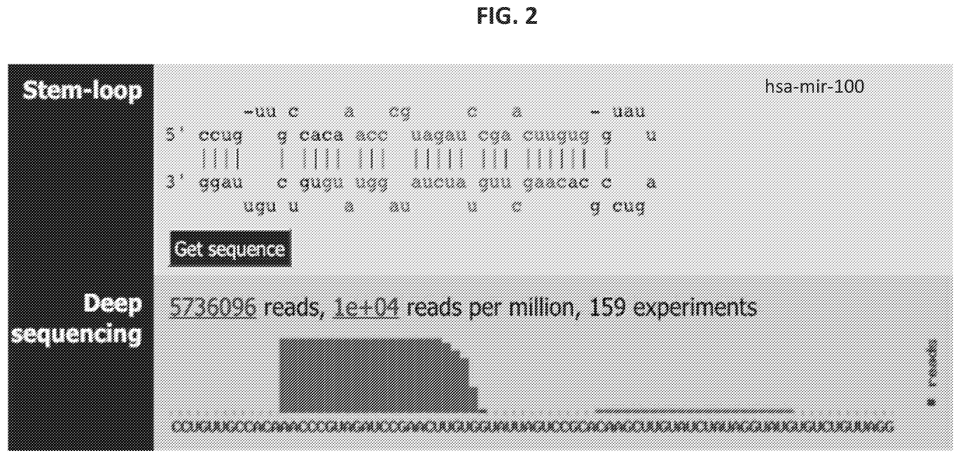

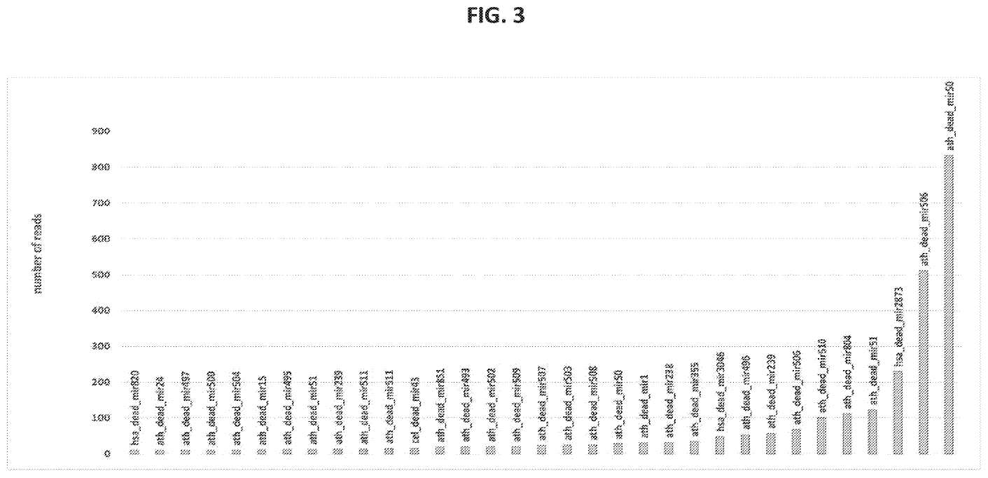

Next, as illustrated in FIG. 1 step (C), and as further discussed below under “detection of expressed candidates” and “detection of expressed non-processed candidates”, the scheme continues with filtering for non-processed ncRNA, e.g. miRNA-like molecules, such that only ncRNAs which are expressed but not processed like their wild-type counterpart are selected. Briefly, the filtering process is as follows:
To avoid detection of candidate genes in the tested genomes which give rise to short RNAs with a silencing functionality corresponding to that of their wild-type homologs (e.g. miRNA precursors), a stringent search of the candidate genes against small RNAs (19-24 nt) was performed on the aforementioned sRNAseq samples (only with complete match between sRNAs and candidate genes). The sRNAs were 19-24 nt as these are the lengths of mature silencing RNAs processed from precursors such as miRNA.
Typically, miRNA processing generates two types of small RNAs which make the mature miRNA sequence: the guide strand and the passenger strand. As illustrated in FIG. 2, one strand of a mature miRNA is typically more abundant when examining sRNA-seq data (in FIG. 2 the guide sequence of human miR-100), while the other strand is typically degraded in the cell and thus of low or undetectable levels.
Thus, miRNA-like precursors that are not processed into mature miRNAs were selected by filtering out candidate ncRNAs in the examined genomes (in this example miRNA-like molecules), which are processed like their homologous counterparts that have a canonical silencing activity.
To do so, several sRNAseq datasets were utilized for sensitive detection of the expression patterns of the ncRNA homologs (when expression is non-ubiquitous, i.e. not expressed in all tissues).
   Stage D: Validate Structural Alteration of Non-Processed miRNAs

Next, as illustrated in FIG. 1 step (D), the scheme continues with validation of structural alteration of non-processed ncRNA from Stage C, e.g. miRNAs, as follows:
The secondary RNA structure of the miRNA precursor and the identified non-processed ncRNAs was predicted based on their nucleotide sequence.
Comparative structural analysis was performed between that of the functional precursors and the precursors of the non-processed miRNA-like molecules (i.e. dysfunctional miRNA) of the same length.
Candidate miRNA-like precursors which were identified in Stage C as expressed but not processed, and which further showed an altered structure from canonical miRNA structure were selected.
Of note, this validation step is relevant only when trying to identify homologs of ncRNAs whose silencing activity is affected by their secondary structure, e.g. miRNAs.

### Stage E: Restore the Structure and Direct Silencing Activity of Candidates

Next, as illustrated in FIG. 1 step (E), the scheme continues with restoring and potentially redirecting the silencing activity of the identified ncRNA towards a target of choice. In order to do so, the nucleotide changes in the ncRNA sequence which are required to restore its silencing activity were determined. For a ncRNA which was found via homology to a silencing molecule whose silencing activity is at least partly dependent on its secondary structure (e.g. a miRNA), the required nucleotide changes for restoration and/or redirection of silencing activity comprised those needed for restoring the secondary structure of the ncRNA such that it corresponds to that of the homologous silencing molecule.

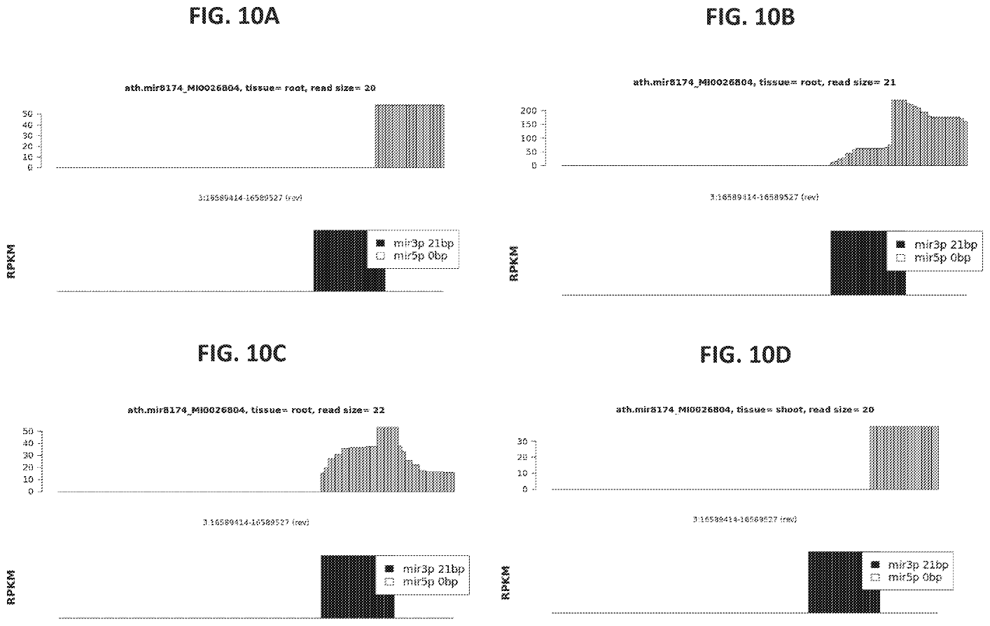

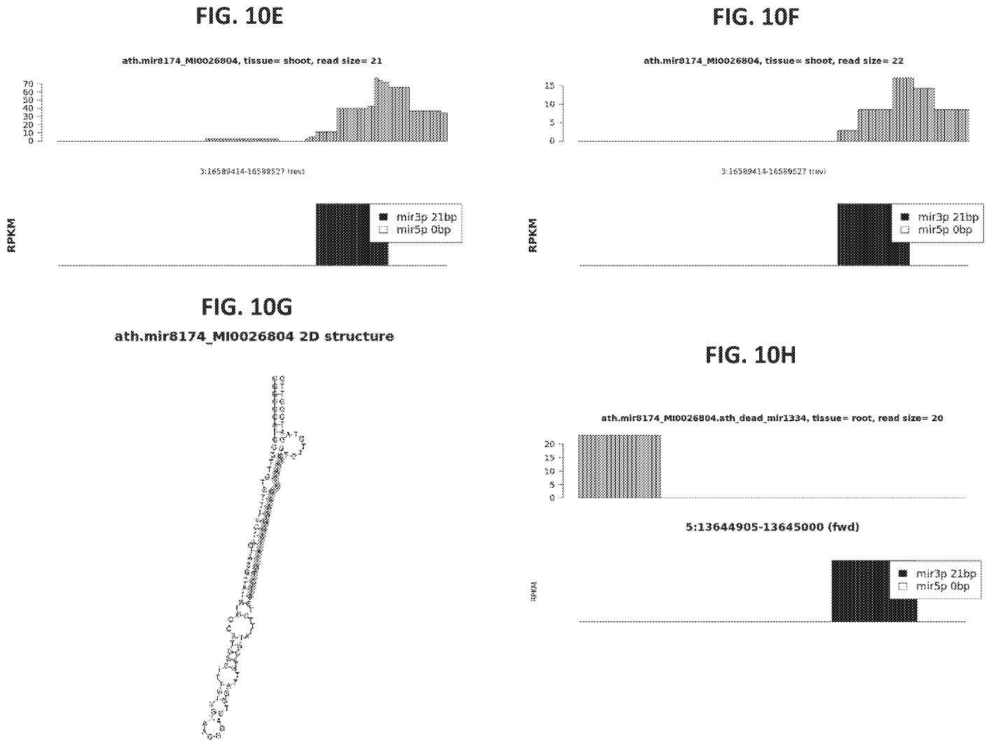

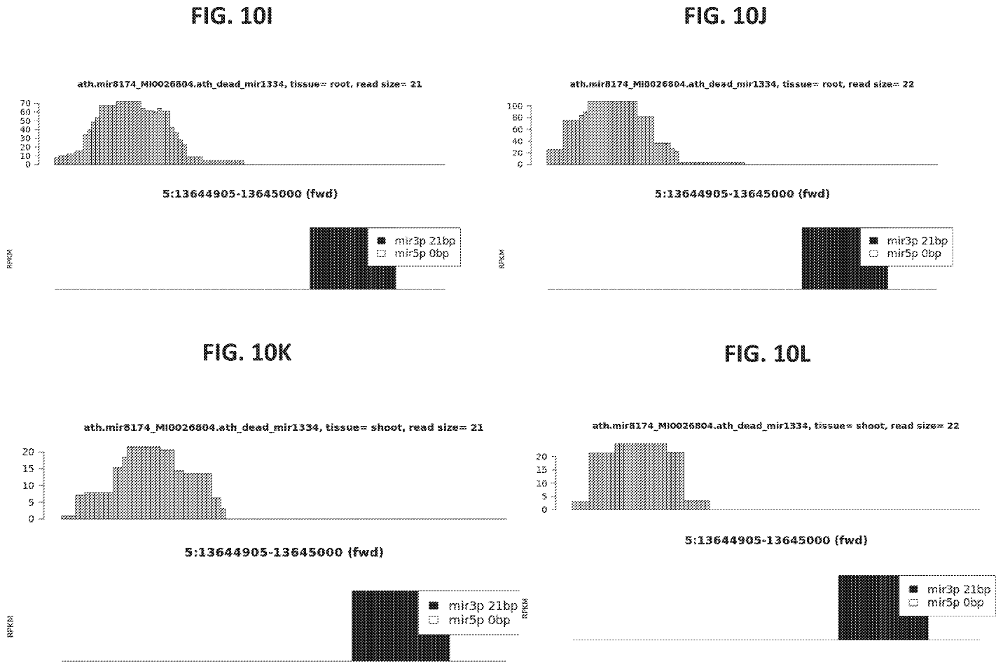

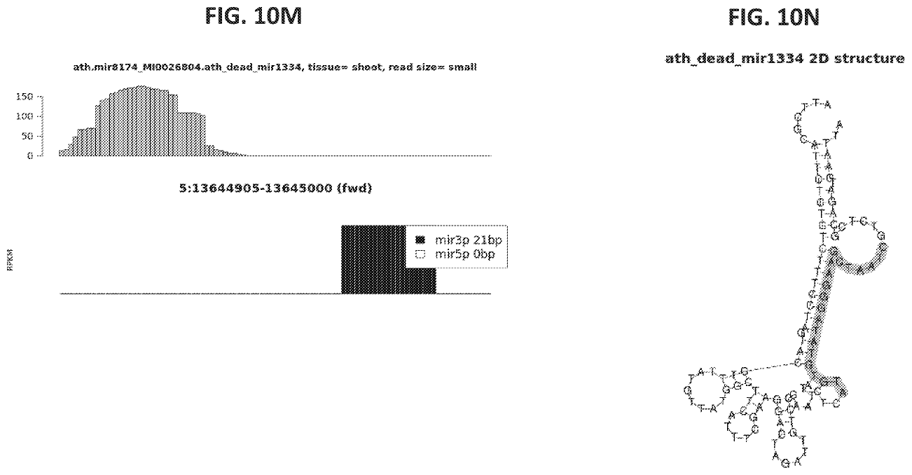

Nucleotide changes required for restoration and/or redirection of silencing activity can be introduced, for example, my Genome Editing methods. Specifically, Genome Editing induced Gene Silencing (GEiGS), as described in WO 2019/058255 (incorporated herein by reference), and as exemplified herein below, can be used to introduce the necessary changes. This can be done by cutting the gene encoding the ncRNA at a desired location (e.g. using the CRISPR/Cas9 technology) and introducing the nucleotide changes by providing a DNA donor carrying them via Homologous DNA Repair (HDR). In short this can be performed on the filtered candidate as follows:
The structure of a dysfunctional miRNA-like precursor molecule expressed by a candidate gene is predicted based on its sequence (see for example the predicted structures of miRNA-like genes identified in Arabidopsis thaliana in FIGS. 10A-N, 11A-J and 12A-I.
The changes in the sequence of the candidate miRNA-like RNA molecule which are necessary to bring its secondary structure to match that of the corresponding functional miRNA (and thus introduce a silencing activity into it) are determined. This can be done computationally by iteratively testing different combinations of nucleotide changes. Of note, the changed nucleotides excluded the nucleotides in positions that correspond to the location of the mature miRNA in the corresponding functional miRNA molecule.
In order to direct the silencing specificity of the re-activated miRNA molecule towards an RNA of interest, additional necessary changes in the sequence of the identified miRNA-like RNA molecule are determined. These changes are in the location corresponding to that of the mature miRNA in the corresponding functional miRNA (as discussed below). These changes introduce a sequence of a potent miRNA/siRNA against the target of interest.
In order to introduce the necessary nucleotide changes to restore the secondary structure of the miRNA-like molecule and redirect it to silence a target gene of choice, Genome Editing induced Gene Silencing (GEiGS) can be used. As described above, this can be achieved by introducing the Cas9 machinery, a sgRNA targeting the gene encoding the miRNA-like gene and a donor DNA into cells. The donor DNA includes the sequence of the miRNA-like gene with the desired changes to reactive it and direct it to a target of choice. As described in WO 2019/058255 (incorporated herein by reference), and as exemplified herein below, this enables introducing the desired changes through use of HDR.
Tables 1A-B below list designs of donor DNAs and sgRNAs which can be used with GeiGS, as described above, to introduce silencing activity into miRNA-like genes Dead_mir859 and Dead_mir1334 (which have been identified in Arabidopsis thaliana) and redirect them to target the PDS3 gene in Arabidopsis thaliana. As demonstrated in Example 2 herein below, re-activation and re-direction of silencing activity was achieved by using miRNAs corresponding to those obtainable by using these donor DNAs and sgRNAs.

TABLE 1A
Designs of donor DNAs and sgRNAs which can be used with GEiGS

| | | | SEQ |
|---|---|---|---|
| Dead_mir859 | | | ID NO: |
| Wt miRNA | Wt-miRNA | ath-miR405a | |
| | Wt sequence | TCAAAATGGGTAACCCAACCCAACCCAACTCATAATCAAATGAGT | 1 |
| | | TTATGATTAAATGAGTTATGGGTTGACCCAACTCATTTTGTTAAA | |
| | | TGAGTTGGGTCTAACCCATAACTCATTTCATTTGATGGGTTGAGT | |
| | | TGTTAAATGGGTTAACCATTTA | |
| | Mature sequence | ATGAGTTGGGTCTAACCCATAACT | 2 |
| | Target analysed | AGTTATGGGTTAGACCCAACTCAT | 3 |
| Dead | ID | ath_dead_miR859 | |
| miRNA | Wt sequence | TCAAAATGGGTAATCCAACTCAACTCAACTCATAATCAAATGAGT | 4 |
| (DmiR) | | TTAGGATTAAATGAGTTATGGGTTGACCCAACTCATTTTGTTAAA | |
| | | TGGGTTCGGTCAACCCATAACTCAATTAATTTGATGGATTGAGTT | |
| | | GGTAAATGAGTTAACCCATTTA | |
| | Mature sequence | ATGGGTTCGGTCAACCCATAACTC | 5 |
| | Target analysed | GAGTTATGGGTTGACCGAACCCAT | 6 |
| Reactivated | Sequence | CCAGATTGGATTGCCTCACACCACACACGACTCAATTCACTAAGA | 7 |
| (RmiR) | | CGAGGATTAAATTGGGTTATGGGTGACCGAACTCATTTTGCCAAa | |
| | | tgggttcggtcaacccataactcAATTTTGGTGAAGGTCGTGGGT | |
| | | GGAAAAGGAGGCAACCCAGTCA | |
| | Mature sequence | ATGGGTTCGGTCAACCCATAACTC | 8 |
| | Target analysed | GAGTTATGGGTTGACCGAACCCAT | 9 |
| Redirected | Sequence | TCAAATTGGGTAAACTACCCCAACATCTCTCAAAATCCAAGGTTG | 10 |
| (Anti | | TTAGGACCAAATGTGGTTTGTGGACAGAGTTTTCATTTTGCTAAa | |
| PDS-PDSmiR) | | tgaaaattttgatttacgaattgCATTATCTTGGGTGAGGGAGGT | |
| | | TGCAAATTAGTTTAGCCAGTTA | |
| | Mature sequence | ATGAAAATTTTGATTTACGAATTG | 11 |
| | Target analysed | CAATTCGTAAATCAAAATTTTAAT | 12 |
| | (PDS3-At4g14210) | | |
| | sgRNA | ATTAATTTGATGGATTGAGTTGG | 13 |
| | DONOR (1.2 kb) | GTCAAAATATGTCAAAATTCATGCGTCAAACTCAACTCAACTCAA | 14 |
| | | CCCATGAACCCTAATGAGTTAAAAATTTGGACTCAAATGGGTTGA | |
| | | TGAGTCAAATGAGTTATTGAGTCAATTGGTTTGATGAGTAAAATG | |
| | | AGTTGGGTTGTAATGATTAATGGTTTCAATGGTTTACCCAATTAA | |
| | | CTCATCAAGTTTTGTAAAATTGAACTAAACCAACTAAAATCTTTA | |
| | | AACCAATGCCAATTTAAGTTTAACCAACATATCTAAACCAATTTA | |
| | | ATAAAATCAATATTTTTCCAAATTTCTTAAATATACAAGCGATAA | |
| | | AATTGAGAAAAAGTAAACTCGTAATTTTTCCACCAAAAAACATAA | |
| | | ACCCGTGATTTTCCCGCCAAAACCGTAAACCCGTGATTTTCCCGC | |
| | | CCAAAACGTAAACCCTTGATTTTTCCGCCCAAAACGTAAATATCC | |
| | | TAAGTTTGATGATAATGAATTAATAATTATTATTTATTATTTTTT | |
| | | ATAATAATAATTAATTAAATTATTACTTAACTGGCTAAACTAATT | |
| | | TGCAACCTCCCTCACCCAAGATAATGcaattcgtaaatcaaaatt | |
| | | ttcatTTAGCAAAATGAAAACTCTGTCCACAAACCACATTTGGTC | |
| | | CTAACAACCTTGGATTTTGAGAGATGTTGGGGTAGTTTACCCAAT | |
| | | TTGACACCCCTAATGACAATATGAGTTTAAAGTTCATTAGTTCAT | |
| | | ATGTATGACAATATAAGTTTATATGAACTAACAAAAATAAATACT | |
| | | TTAAGATCATAGTAATAAATACGTGAATATCATAATAATATAGAA | |
| | | AAATCGTATATATATATACATAGACCTCAAATGCAACAAAAATAC | |
| | | TAAAGAAAAACTTTTATCAAATTACGTGATAAATAAATAATTGTT | |
| | | CTTTTATCAAAATTACTAAAAACAATTCATTCCTTCTTCTTATTT | |
| | | TTTTTAATAATACTATAATAACTAGGATACGACACAGCAGGTTAA | |
| | | ATATTTTATTTATTTTTCTTTTTTATAAACGAAATTTATTGTTTA | |
| | | TTGTTATTTGTGTTTATTAATAATTATCTATAAAACTGTGTATAT | |
| | | TTTTATTGAGTCGTACTTATGATATTAGTAAGTCTAATAGGTTAT | |
| | | TTTATCTTTTAGGATTTGACTCGTGCTAGACCACACCACGTGATA | |
| | | ATTTTTACTTTTAGTGTTTTTAGATTAATG | |

TABLE 1B
Designs of donor DNAs and sgRNAs which can be used with GEiGS

| | | | SEQ |
|---|---|---|---|
| Dead_mir1334 | | | ID NO: |
| Wt miRNA | Wt-miRNA | ath-miR8174 | |
| | Wt sequence | CGGCCCATCCGTTGTCTTTCCTGGTACGCATGTGCCATGGCTTTCT | 15 |
| | | CGTAAGGGACTGGATTGTCCGTATTTCTCATGTGTATAGGGAAGCT | |
| | | AATCGTCTTGTAGATGGGTTG | |
| | Mature sequence | ATGTGTATAGGGAAGCTAATC | 16 |
| | Target analysed | GATTAGCTTCCCTATACACAT | 17 |
| Dead | ID | ath_dead_miR1334 | |
| miRNA | Wt sequence | ATTCGCATTCTCTGTCTTTCCTAGTACGTTTATGTTATGGCTTCAT | 18 |
| (DmiR) | | TTCGAAGGACTAGATTGTCCGAATTACTCATGTGTATAGGGAAGCT | |
| | | AATCGTCTCGCAGATGAATTA | |
| | Mature sequence | ATGTGTATAGGGAAGCTAATC | 19 |
| | Target analysed | GATTAGCTTCCCTATACACAT | 20 |
| Reactivated | Sequence | TCACGCATTCGTTGACTTCCCTAGTACGCATATTGAACTGCTGTAA | 21 |
| (RmiR) | | GGTGAAGGACGTTAATGTACCAAAAACTTatgtgtatagggaagct | |
| | | aatcGTCCCGCAGATGTGTGA | |
| | Mature sequence | ATGTGTATAGGGAAGCTAATC | 22 |
| | Target analysed | GATTAGCTTCCCTATACACAT | 23 |
| Redirected | Sequence | ATGTGCATCGCAGTGATTGGTGTGTTATATGACTAAAAGTCTTTAT | 24 |
| (Anti | | CGCGAAGGGCTATATCGACCTAGGTACTTtatatgaacattaataa | |
| PDS-PDSmiR) | | ctggCCCCCCCAGATGCATGT | |
| | Mature sequence | TATATGAACATTAATAACTGG | 25 |
| | Target | CCAGTTATTAATGTTCATATA | 26 |
| | analysed | | |
| | (PDS3-At4g14210) | | |
| | sgRNA | ATGTTATGGCTTCATTTCGAAGG | 27 |
| | DONOR (1.2 kb) | GTTATATGTGTTCTTTACACAATCATTGCTTGAATGGGTATACAGT | 28 |
| | | AATTTGGGAGAACAAGAACTTGTCGGAGGTTATCCGTGGGCTACTT | |
| | | TATTCGCTTTGGCACCATGGTGGGGTTGGAAACGGCGCTGCAGAAA | |
| | | TGTGTTTGGGGAGAATAGGAAATGTCGAGATAGAGTTCGTTTCCTA | |
| | | AAGGATTCAGCGAAAGAGGTGGTGGAGGCTCACTCGCTGCTTGGGA | |
| | | GTAATCGAGGTAATGTAACTAGGGTGGAGAGACAAATAGCATGAGT | |
| | | TCCGCCAGGAGATGGTTGGCTGAAGTTAAACACGGATGGCGCATCA | |
| | | CGTGGAAATCCGGGTTTAGCAATAGCTGGTGGTGTTTTACGGGATA | |
| | | ATGAGGGTATTTGGTGTGGTGGTTTTGCGGGAATCTCGGAGTTTGT | |
| | | TCGGCTCCTTTAGTTAAGTTATGAGGTGTGTATTACGGGCTTTTCA | |
| | | TAGCTTGGGAGAAAAAGGCTACGCGGGTGTAGCTGGAAGTGGATTC | |
| | | AGATATGGTGGTGGGTTTTCTTAAAACATGGATTAGCGATGTGCAT | |
| | | CGCAGTGATTGGTGTGTTATATGACTAAAAGTCTTTATCGCGAAGG | |
| | | GCTATATCGACCTAGGTACTTtatatgaacattaataactggCCCC | |
| | | CCCAGATGCATGTGCAAACCATGCTTTTTTGTTACCTTTGGGGTTT | |
| | | CATAGTTTTCCCCTTAGGCCTGATTTTGCTACTTCGATTATTTTTG | |
| | | AGGATGCTAGTAGTGCTACGCGCCCACGGAATGTTCGTGTGTAATT | |
| | | TTTTTATTTTGTTTTTTAATAATATGGGAGACTAGTCTCCCTCATT | |
| | | CTAAAAAAAATAAAAAATTATAATTATATAAAATAGATATAAAATT | |
| | | ATTAATTACATAATAATACACACAAAAAATGAATATCAAGAAAAAT | |
| | | CTCTCTCTCTCTAAATCAAAATCAAATGAGAGAAGAGAGGCGATAC | |
| | | GACGAACGATTGCATCTCTTCGATTCCTACGGCTGTCTCTCGCTCG | |
| | | CCGAGAGTTTTCTTCGCCAGTTTCCGGCGGTTACTTCAGGGATGAA | |
| | | TAACGGTAGAACGGTTGTGGACCCCATAACTGCTTCTCAACCAAAC | |
| | | CTATTTATACCCTGCGCATGTCTCTGTTCTCGTTGGGTTGATCAGA | |
| | | GTGAAAGTACACAAATTCCTTTGTTCATATTGACAATGGCAGATAA | |
| | | TCTC | |

Genomes, Genomic Annotations and miRNA Sequences

The list of all known precursor miRNA sequences and their corresponding mature guide and passenger sequences for H. sapiens, C. elegans and A. thaliana were downloaded from miRBase (version 22) [The microRNA Registry. Griffiths-Jones S. Nucleic Acids Res (2004) 32:D109-D111]. Next, the corresponding genomes of each species and annotation files were obtained. For C. elegans, the ensemble genome (release-95) was downloaded. For H. sapiens, GRCh38.p12 (version 29) was downloaded from genecode. The genome of A. thaliana was downloaded from TAIR (version 10).

### Construction of Candidate Sets

As described above (for Stage A), the precursor and/or mature sequences of known miRNAs were used to perform a blast search against the corresponding genome of each species in order to identify the initial list of candidate genes encoding miRNA-like molecules, the expression pattern of which will be further examined. For each candidate, its sequence was extracted based on its genomic coordinates and the known miRNA(s) to which it mapped was recorded according to the blast search. Based on the alignment of the candidate to its corresponding known miRNA and the location of its guide and passenger sequences, the putative guide and passenger sequences of the candidate were extracted and marked as to whether they were aberrantly processed relative to the guide and passenger sequences of its corresponding known miRNA. In addition, using the genomic annotation file, it was determined whether the candidate is located within an intronic or exonic region.

List of candidate genes in A. thaliana, C. elegans and H. sapiens were generated as follows. According to some embodiments, an initial candidate gene, which is suitable for Stage A above, and for which sRNA expression should be determined, should have at least the following predetermined homology parameters to an existing ncRNA (e.g. a miRNA):
1. The initial candidate gene encodes an RNA molecule which is identified through a blast search using default parameters (www(dot)Arabidopsis(dot)org/Blast/BLASToptions(dot)jsp) with respect to a corresponding ncRNA (e.g. miRNA); and
2. The initial candidate gene comprises a sequence which covers at least 50% of a mature miRNA sequence of a wild-type miRNA from the same organism. According to some embodiments this sequence is of 19-24 nt, possibly 19-21 nt.
   A. thaliana

The precursor sequences of known A. thaliana miRNAs from miRbase were used to perform a blast search against the genome of A. thaliana using default parameters (www(dot)arabidopsis(dot)org/Blast/BLASToptions(dot)jsp). Genomic regions that intersected with genomic coordinates of known miRNA genes were discarded. The resulting set of initial candidates comprised 795 distinct genomic locations. Each candidate was named according to the miRbase miRNA it matched in the blast search. For example, the miRNA-like molecule that was identified based on ath-mir-8174 was named ath_dead_mir1334. Accordingly, the full name of the miRNA-like molecule was named: ath-mir-8174-MI0026804.ath_dead_mir1334.

Next, the fasta sequence of each candidate was obtained and, based on the alignment of the candidate to its corresponding WT miRNA (and the location of the WT miRNA mature guide and/or passenger sequences), the sequences of the candidate which correspond in their location to the mature miRNA were identified (also referred to herein as the “mature” sequence of the candidate). In addition, using the corresponding genomic annotation file, it was determined whether the candidate is located within an intronic or exonic region.

Table 2, below, provides a list of A. thaliana candidates that have been found as described above.

TABLE 2
list of A. thaliana candidates

| | | mut_seq | | mut_5p | | 5p_ | | 5p_ | mut_3p | | 3p_ | | 3p_ |
|---|---|---|---|---|---|---|---|---|---|---|---|---|---|
| | chr:start- | (SEQ ID | 5p_ | (SEQ | 5p_ | cover- | 5p_ | muta- | (DEQ | 3p_ | cover- | 3p_ | muta- |
| dead_mir_id | end(strand) | NO) | length | ID NO) | length | age | % id | tions | ID NO) | length | age | % id | tions |
| ath_dead_mir1224 | 5:11953932- | 65 | 78 | none | 0 | 0 | 0 | 0 | 123 | 20 | 100 | 0.38 | 13 |
| | 11954009(−) | | | | | | | | | | | | |
| ath_dead_mir1235 | 5:11961192- | 66 | 81 | none | 0 | 0 | 0 | 0 | 124 | 71 | 100 | 1 | 0 |
| | 11961272(−) | | | | | | | | | | | | |
| ath_dead_mr1264 | 5:12052914- | 67 | 83 | none | 0 | 0 | 0 | 0 | 125 | 21 | 100 | 1 | 0 |
| | 12052996(−) | | | | | | | | | | | | |
| ath_dead_mir1264 | 5:12052914- | 68 | 83 | none | 0 | 0 | 0 | 0 | 126 | 21 | 100 | 1 | 0 |
| | 12052996(−) | | | | | | | | | | | | |
| allt_dead _mir134 | 1:17710617- | 69 | 151 | none | 0 | 0 | 0 | 0 | 127 | 23 | 100 | 0.46 | 13 |
| | 17710767(−) | | | | | | | | | | | | |
| ath_dead_mir1387 | 5:20594526- | 70 | 76 | none | 0 | 0 | 0 | 0 | 128 | 22 | 100 | 0.92 | 2 |
| | 20594601(−) | | | | | | | | | | | | |
| ath_dead_mir1388 | 5:20627868- | 71 | 76 | none | 0 | 0 | 0 | 0 | 129 | 22 | 100 | 0.92 | 2 |
| | 20627943(−) | | | | | | | | | | | | |
| ath_dead_mir1419 | 5:6460872- | 72 | 324 | none | 0 | 0 | 0 | 0 | 130 | 21 | 95.24 | 1 | 0 |
| | 6461195(+) | | | | | | | | | | | | |
| ath_dead_mir148 | 1:19268620- | 73 | 182 | 165 | 20 | 100 | 0.95 | 1 | none | 0 | 0 | 0 | 0 |
| | 19268801(−) | | | | | | | | | | | | |
| ath_dead_mir169 | 1:22579562- | 74 | 83 | none | 0 | 0 | 0 | 0 | 131 | 70 | 0 | 0 | 0 |
| | 22579644(−) | | | | | | | | | | | | |
| ath_dead_mir189 | 1:24908498- | 75 | 78 | none | 0 | 0 | 0 | 0 | 132 | 22 | 100 | 0.92 | 2 |
| | 24908575(+) | | | | | | | | | | | | |
| ath_dead_mir231 | 1:8276509- | 76 | 157 | none | 0 | 0 | 0 | 0 | 133 | 23 | 95 | 0.83 | 5 |
| | 8276665(+) | | | | | | | | | | | | |
| ath_dead_mir30 | 1:13151181- | 77 | 195 | 166 | 20 | 100 | 0.95 | 1 | none | 0 | 0 | 0 | 0 |
| | 13151375(−) | | | | | | | | | | | | |
| ath_dead_mir31 | 1:13151183- | 78 | 230 | 167 | 20 | 100 | 0.95 | 1 | none | 0 | 0 | 0 | 0 |
| | 13151412(−) | | | | | | | | | | | | |
| ath_dea_mir363 | 2:4947743- | 79 | 261 | 168 | 20 | 94.74 | 0.95 | 1 | none | 0 | 0 | 0 | 0 |
| | 4948003(+) | | | | | | | | | | | | |
| ath_dead_mir371 | 2:5056548- | 80 | 242 | 169 | 20 | 92.86 | 0.7 | 6 | none | 0 | 0 | 0 | 0 |
| | 5056789(+) | | | | | | | | | | | | |
| ath_dead_mir375 | 2:5056789- | 81 | 84 | 170 | 20 | 89.47 | 0.95 | 1 | none | 0 | 0 | 0 | 0 |
| | 5056872(−) | | | | | | | | | | | | |
| ath_dead_mir430 | 3:10414059- | 82 | 81 | none | 0 | 0 | 0 | 0 | 134 | 22 | 100 | 0.92 | 2 |
| | 10414139(−) | | | | | | | | | | | | |
| ath_dead_mir4 | 1:11287559- | 83 | 78 | none | 0 | 0 | 0 | 0 | 135 | 22 | 95.45 | 0.92 | 2 |
| | 11287636(+) | | | | | | | | | | | | |
| ath_dead _mir500 | 3:15681719- | 84 | | none | 0 | 0 | 0 | 0 | 136 | 22 | 100 | 0.92 | 2 |
| | 15681802(+) | | | | | | | | | | | | |
| ath_dead_mir511 | 3:16353222- | 85 | 85 | none | 0 | 0 | 0 | 0 | 137 | 23 | 95 | 0.83 | 5 |
| | 16353306(−) | | | | | | | | | | | | |
| ath_dead_mir718 | 4:3360153- | 86 | 84 | none | 0 | 0 | 0 | 0 | 138 | 22 | 100 | 0.92 | 2 |
| | 3360236(+) | | | | | | | | | | | | |
| ath_dead_mir741 | 4:3809888- | 87 | 204 | 171 | 20 | 100 | 0.95 | 1 | none | 0 | 0 | 0 | 0 |
| | 3810091(−) | | | | | | | | | | | | |
| ath_dead_mir742 | 4:3809888- | 88 | 212 | 172 | 20 | 100 | 0.95 | 1 | none | 0 | 0 | 0 | 0 |
| | 3810099 (−) | | | | | | | | | | | | |
| ath_dead_mir835 | 4:4549055- | 89 | 188 | 173 | 20 | 95 | 1 | 0 | none | 0 | 0 | 0 | 0 |
| | 4549242(−) | | | | | | | | | | | | |
| ath_dead_mir90 | 1:15729928- | 90 | 204 | 174 | 20 | 100 | 0.95 | 1 | none | 0 | 0 | 0 | 0 |
| | 15730131(−) | | | | | | | | | | | | |
| ath_dead_mir919 | 5:10681297- | 91 | 85 | none | 0 | 0 | 0 | 0 | 139 | 22 | 100 | 0.92 | 1 |
| | 10681381(−) | | | | | | | | | | | | |
| ath_dead_mir91 | 1:15729930- | 92 | 228 | 175 | 20 | 100 | 0.95 | 1 | none | 0 | 0 | 0 | 0 |
| | 15730157(−) | | | | | | | | | | | | |
| ath_dead_mir983 | 5:11682841- | 93 | 82 | none | 0 | 0 | 0 | 0 | 140 | 21 | 0 | 0 | 0 |
| | 11682922(−) | | | | | | | | | | | | |
| ath_dead_mir983 | 5:11682841- | 94 | 82 | none | 0 | 0 | 0 | 0 | 141 | 21 | 100 | 0.67 | 7 |
| | 11682922(+) | | | | | | | | | | | | |
| ath_dead_mir990 | 5:11755186- | 95 | 81 | none | 0 | 0 | 0 | 0 | 142 | 21 | 89.47 | 0.9 | 1 |
| | 11755266(−) | | | | | | | | | | | | |
| ath_dead_mir990 | 5:11755186- | 96 | 81 | none | 0 | 0 | 0 | 0 | 143 | 21 | 89.47 | 0.9 | 2 |
| | 11755266(−) | | | | | | | | | | | | |
| ath_dead_mir123 | 1:16613364- | 97 | 157 | none | 0 | 0 | 0 | 0 | 144 | 23 | 95 | 0.83 | 5 |
| | 16613520(+) | | | | | | | | | | | | |
| ath_dead_mir1267 | 5:12054516- | 98 | 80 | none | 0 | 0 | 0 | 0 | 145 | 20 | 95 | 0.95 | |
| | 12054595(−) | | | | | | | | | | | | |
| ath_dead_mir126 | 1:16737861- | 99 | 84 | none | 0 | 0 | 0 | 0 | 146 | 22 | 90.91 | 0.92 | 2 |
| | 16737944(−) | | | | | | | | | | | | |
| ath dead_mir1272 | 5:12055937- | 100 | 80 | none | 0 | 0 | 0 | 0 | 147 | 20 | 95 | 0.95 | 1 |
| | 12056016(−) | | | | | | | | | | | | |
| ath_dead_mir1289 | 5:12061448- | 101 | 81 | none | 0 | 0 | 0 | 0 | 148 | 21 | 100 | 0.62 | 8 |
| | 12061528(−) | | | | | | | | | | | | |
| ath_ dead_ rnir1382 | 5:19495975- | 102 | 85 | none | 0 | 0 | 0 | 0 | 149 | 23 | 95.83 | 1 | 1 |
| | 19496059(−) | | | | | | | | | | | | |
| ath_dead_mir1434 | 5:744992- | 103 | 155 | none | 0 | 0 | 0 | 0 | 150 | 23 | 100 | 0.46 | 13 |
| | 745146(−) | | | | | | | | | | | | |
| ath_dead_rnirl73 | 1:23299203- | 104 | 446 | 176 | 21 | 100 | 0.33 | 14 | none | 0 | 0 | 0 | 0 |
| | 23299648(−) | | | | | | | | | | | | |
| ath_dead_mir178 | 1:23419542- | 105 | 446 | 177 | 21 | 100 | 0.33 | 14 | none | 0 | 0 | 0 | 0 |
| | 23419987(−) | | | | | | | | | | | | |
| ath_ dead_mirl79 | 1:23489801- | 106 | 409 | 178 | 22 | 100 | 0.33 | 14 | none | 0 | 0 | 0 | 0 |
| | 23490209(−) | | | | | | | | | | | | |
| ath_dead_mirl80 | 1:23507472- | 107 | 443 | 179 | 22 | 100 | 0.33 | 14 | none | 0 | 0 | 0 | 0 |
| | 23507914(−) | | | | | | | | | | | | |
| ath_dead_mir225 | 1:7725358- | 108 | 78 | none | 0 | 0 | 0 | 0 | 151 | 22 | 100 | 0.79 | 5 |
| | 7725435(−) | | | | | | | | | | | | |
| ath_dead_mir269 | 2:15566967- | 109 | 75 | none | 0 | 0 | 0 | 0 | 152 | 22 | 100 | 1 | 0 |
| | 15567041(−) | | | | | | | | | | | | |
| ath_ dead_mir269 | 2:15566967- | 110 | 75 | none | 0 | 0 | 0 | 0 | 153 | 22 | 100 | 1 | 0 |
| | 15567041(−) | | | | | | | | | | | | |
| ath_dead_mir269 | 2:15566967- | 111 | 75 | none | 0 | 0 | 0 | 154 | 22 | 100 | 1 | 0 | |
| | 15567041(−) | | | | | | | | | | | | |
| ath_dead_mire269 | 2:15566967- | 112 | 75 | none | 0 | 0 | 0 | 0 | 155 | 22 | 100 | 1 | 0 |
| | 15567041(−) | | | | | | | | | | | | |
| ath_dead_mir269 | 2:15566976- | 113 | 75 | none | 0 | 0 | 0 | 0 | 156 | 22 | 100 | 1 | 0 |
| | 15567041(−) | | | | | | | | | | | | |
| ath_dead_mir269 | 2:15566967- | 114 | 75 | none | 0 | 0 | 0 | 0 | 157 | 22 | 100 | 1 | 0 |
| | 15567014(−) | | | | | | | | | | | | |
| ath_dead_mir404 | 2:6733086- | 115 | 78 | none | 0 | 0 | 0 | 0 | 158 | 22 | 95.45 | 0.92 | 2 |
| | 6733163(−) | | | | | | | | | | | | |
| ath_dead_mir498 | 3:15371881- | 116 | 84 | none | 0 | 0 | 0 | 0 | 159 | 22 | 94.74 | 0.79 | 5 |
| | 15371964(−) | | | | | | | | | | | | |
| ath_dead_mir547 | 3:18243841- | 117 | 85 | none | 0 | 0 | 0 | 0 | 160 | 22 | 100 | 0.79 | 5 |
| | 18243925(−) | | | | | | | | | | | | |
| ath_dead_mir548 | 3:18244457- | 118 | 75 | none | 0 | 0 | 0 | 0 | 161 | 25 | 100 | 0.46 | 13 |
| | 18244531(−) | | | | | | | | | | | | |
| ath_dead_mir859 | 4:5279033- | 119 | 157 | none | 0 | 0 | 0 | 0 | 162 | 23 | 100 | 0.46 | 13 |
| | 5279189(+) | | | | | | | | | | | | |
| ath_dead_mir913 | 5:10458714- | 120 | 157 | 180 | 21 | 94.74 | 0.9 | 2 | none | 0 | 0 | 0 | 0 |
| | 10458870(−) | | | | | | | | | | | | |
| ath_dead_mir991 | 5:11755719- | 121 | 82 | none | 0 | 0 | 0 | 0 | 163 | 21 | 100 | 0.9 | 2 |
| | 11755800(−) | | | | | | | | | | | | |
| ath_dead_mir991 | 5:11755719- | 122 | 82 | none | 0 | 0 | 0 | 0 | 164 | 21 | 100 | 0.9 | 2 |
| | 11755800(−) | | | | | | | | | | | | |

| | | wt_seq | | wt_5p | | wt_3p | |
|---|---|---|---|---|---|---|---|
| | | (SEQ ID | wt_ | (SEQ ID | 5p_ | (SEQ ID | 3p_ |
| WT_mire_id | chr:start-end(strand) | NO) | length | NO) | length | NO) | length |
| MIR5643a\|MI0019216 | 5:11667796-11667(+) | 181 | 79 | none | 0 | 239 | 21 |
| MIR5643b\|MI0019256 | 5:11757139-11757222(−) | 182 | 81 | none | 0 | 240 | 21 |
| MIR5643a\|MI0019216 | 5:11667796-11667879(+) | 183 | 83 | none | 0 | 241 | 21 |
| MIR5643b\|MI0019256 | 5:11757139-11757222(−) | 184 | 83 | none | 0 | 242 | 21 |
| MIR405a\|MI0001074 | 2:9634956-9635113(−) | 185 | 152 | none | 0 | 743 | 74 |
| MIR5653\|MI0019236 | 1:19026914-19027000(−) | 186 | 78 | none | 0 | 244 | 24 |
| MIR5653\|MI0019236 | 1:19026914-19027000(−) | 187 | 78 | none | 0 | 245 | 24 |
| MIR5635a\|MI0019207 | 5:6926004-6926446(+) | 188 | 324 | none | 0 | 246 | 21 |
| MIR5645b\|MI0019221 | 4:4889420-4889914(+) | 189 | 182 | 281 | 20 | none | 0 |
| MIR846\|MI0005402 | 1:22577374-22577733(+) | 190 | 83 | 282 | 21 | 247 | 21 |
| MIR5653\|MI0019236 | 1:19026914-19027000(−) | 191 | 78 | none | 0 | 248 | 24 |
| MIR405a\|MI0001074 | 2:9634956-9635113(−) | 192 | 157 | none | 0 | 249 | 24 |
| MIR645e\|MI0019257 | 4:5321226-5321643(+) | 193 | 186 | 283 | 20 | none | 0 |
| MIR5645b\|MI0019221 | 4:4889420-4889914(+) | 194 | 221 | 284 | 20 | none | 0 |
| MIR5645d\|M10019244 | 1:16116571-16117041(+) | 195 | 251 | 285 | 20 | none | 0 |
| MIR5645e\|MI0019257 | 4:5321226-5321643(+) | 196 | 242 | 286 | 20 | none | 0 |
| MIR5645d\|MI0019244 | 1:16116571-16117041(+) | 197 | 84 | 287 | 20 | none | 0 |
| MIR5653\|MI0019236 | 1:19026914-19027000(−) | 198 | 81 | none | 0 | 250 | 24 |
| MIR5653\|MI0019236 | 1:19026914-19027000(−) | 199 | 78 | none | 0 | 251 | 24 |
| MIR5653\|MI0019236 | 1:19026914-19027000(−) | 200 | 84 | none | 0 | 252 | 24 |
| MIR405d\|MI0001077 | 4:2789655-2789744(−) | 201 | 86 | none | 0 | 253 | 24 |
| MIR5653\|MI0019236 | 1:19026914-19027000(−) | 202 | 84 | none | 0 | 254 | 24 |
| MIR5645d\|MI0019244 | 1:16116571-16117041(+) | 203 | 194 | 288 | 20 | none | 0 |
| MIR5645a\|MI0019220 | 3:17418775-17419220(+) | 204 | 202 | 289 | 20 | none | 0 |
| MIR5645d\|MI0019244 | 1:16116571-16117041(+) | 205 | 188 | 290 | 20 | none | 0 |
| MIR5645d\|MI0019244 | 1:16116571-16117041(+) | 206 | 194 | 291 | 20 | none | 0 |
| MIR5653\|MI0019236 | 1:19026914-19027000(−) | 207 | 84 | none | 0 | 755 | 24 |
| MIR5645b\|MI0019221 | 4:4889420-4889914(+) | 208 | 218 | 292 | 20 | none | 0 |
| MIR5643a\|MI0019216 | 5:11667796-11667879(+) | 209 | 82 | none | 0 | 256 | 21 |
| MIR5643b\|MI0019256 | 5:11757139-11757222(−) | 210 | 82 | none | 0 | 257 | 21 |
| MIR5643a\|MI0019216 | 5:11667796-11667879(+) | 211 | 81 | none | 0 | 258 | 21 |
| MIR5643b\|MI0019256 | 5:11757139-11757222(−) | 212 | 81 | none | 0 | 259 | 21 |
| MIR405a\|MI0001074 | 2:9634956-9635113(−) | 213 | 157 | none | 0 | 260 | 24 |
| MIR5643a\|MI0019216 | 5:11667796-11667879(+) | 214 | 80 | none | 0 | 261 | 21 |
| MIR5653\|MI0019236 | 1:19026914-19027000(−) | 215 | 84 | none | 0 | 262 | 24 |
| MIR5643a\|MI0019216 | 5:11667796-11667879(+) | 216 | 80 | none | 0 | 263 | 21 |
| MIR5643b\|MI0019256 | 5:11757139-11757222(−) | 217 | 82 | none | 0 | 264 | 21 |
| MIR405d\|MI0001077 | 4:2789655-2789741(−) | 218 | 86 | none | 0 | 265 | 24 |
| MIR405a\|MI0001074 | 2:9634956-9635113(−) | 219 | 155 | none | 0 | 266 | 24 |
| MIR5652\|MI0019235 | 1:23412988-23413436(−) | 220 | 443 | 293 | 21 | none | 0 |
| MIR5652\|MI0019235 | 1:23412988-23413436(−) | 221 | 443 | 294 | 21 | none | 0 |
| MIR5652\|MI0019235 | 1:23412988-23413436(−) | 222 | 409 | 295 | 21 | none | 0 |
| MIR5652\|MI10019235 | 1:23412988-23413436(−) | 223 | 443 | 296 | 21 | none | 0 |
| MIR5653\|MI0019236 | 1:19026914-19027000(−) | 224 | 78 | none | 0 | 267 | 24 |
| MIR8167a\|MI0026795 | 2:8894931-8895006(+) | 225 | 75 | none | 0 | 268 | 22 |
| MIR8167b\|MI0026796 | 3:17469945-17470020(−) | 226 | 75 | none | 0 | 269 | 22 |
| MIR8167c\|MI0026797 | 3:18843648-18843723(−) | 227 | 75 | none | 0 | 270 | 22 |
| MIR8167d\|MI0031739 | 5:7057156-7057231(+) | 228 | 75 | none | 0 | 271 | 22 |
| MIR8167e\|MI0031740 | 5:23431702-23431777(−) | 229 | 75 | none | 0 | 272 | 22 |
| MIR8167f\|MI0031741 | 5:24002238-24002313(−) | 230 | 75 | none | 0 | 273 | 22 |
| MIR5653\|MI0019236 | 1:19026914-19027000(−) | 231 | 78 | none | 0 | 274 | 24 |
| MIR5653\|MI0019236 | 1:19026914-19027000(−) | 232 | 84 | none | 0 | 275 | 24 |
| MIR5653\|MI0019236 | 1:19026914-19027000(−) | 233 | 84 | none | 0 | 276 | 24 |
| MIR405a\|MI0001074 | 2:9634956-9635113(−) | 234 | 73 | none | 0 | 277 | 24 |
| MIR4050a\|MI0001074 | 2:9634956-9635113(−) | 235 | 157 | none | 0 | 278 | 24 |
| MIR5651\|MI0019233 | 3:17178446-17178608(+) | 236 | 155 | 297 | 21 | none | 0 |
| MIR5643a\|MI0019216 | 5:11667796-11667879(+) | 237 | 82 | none | 0 | 779 | 21 |
| MIR5643b\|MI0019256 | 5:11757139-11757222(−) | 238 | 82 | none | 0 | 280 | 21 |

C. elegans

The mature guide and/or passenger sequences of known C. elegans miRNA's from miRbase were used to perform a blast search against the genome of C. elegans (13,971 matches) from which all known miRNA's (13,522 matches) were removed. For each location that matched at least 70% of a mature miRNA sequence (potentially a ‘guide’ or a ‘passanger’ strand), it was checked whether a complementary sequence maped to the genome within a distance that was no more than 20% longer or shorter that the distance between the guide and passenger sequences in the wild-type (WT) miRNA. 385 pairs were found that matched the aforementioned criteria and the genes comprising these pairs were deemed candidates. The fasta sequences of the candidate sequences comprising the 385 found pairs (the length of the fasta sequences corresponding to the length of the wild-type miRNA homologous to each candidate) were then extracted, their genomic location recorded and based on the corresponding genomic annotation file, it was determined whether the candidate is located within an intronic or exonic region.

Table 3, below, provides a list of C. elegans candidates that have been found as described above.

TABLE 3
List of C. elegans candidates

| | | mut_seq | | mut_5p | | 5p_ | | 5p_ |
|---|---|---|---|---|---|---|---|---|
| | | (SEQ ID | mut_ | (SEQ ID | 5p_ | cover- | 5p_ | muta- |
| dead_mir_id | chr:start-end(strand) | NO) | length | NO) | length | age | % id | tions |
| cel_dead_mir219 | I:1048824-1048940(−) | 298 | 116 | 316 | 24 | 79.17 | 100 | 5 |
| cel_dead_mir537 | X:16566649- | 299 | 66 | 317 | 23 | 100 | 100 | 0 |
| | 16566715(+) | | | | | | | |
| cel_dead_mir291 | II:6778742-6778897(+) | 300 | 155 | 318 | 23 | 100 | 100 | 0 |
| cel_dead_mir204 | I:1931479-1931601(+) | 301 | 122 | 319 | 24 | 75 | 100 | 6 |
| cel_dead_mir188 | I:11872678- | 302 | 122 | 320 | 24 | 91.67 | 100 | 2 |
| | 11872800(+) | | | | | | | |
| cel_dead_mir481 | V:18041465- | 303 | 163 | 321 | 23 | 100 | 95.65 | 1 |
| | 18041628(+) | | | | | | | |
| cel_dead_mir513 | V:2662770- | 304 | 126 | 322 | 24 | 75 | 100 | 6 |
| | 2662896(−) | | | | | | | |
| cel_dead_mir400 | III:2160054- | 305 | 112 | 323 | 24 | 79.17 | 100 | 5 |
| | 2160166(−) | | | | | | | |
| cel_dead_mir363 | III:12613971- | 306 | 123 | 324 | 24 | 75 | 100 | 6 |
| | 12614094(+) | | | | | | | |

| | mut_3p | | | | |
|---|---|---|---|---|---|
| | (SEQ ID | | 3p_ | 3p_ | 3p_ |
| dead_mir_id | NO) | 3p_length | coverage | % id | mutations |
| cel_dead_mir219 | 307 | 23 | 73.91 | 100 | 6 |
| cel_dead_mir537 | 308 | 24 | 70.83 | 100 | 7 |
| cel_dead_mir291 | 309 | 22 | 100 | 95.45 | 1 |
| cel_dead_mir204 | 310 | 23 | 73.91 | 100 | 6 |
| cel_dead_mir188 | 311 | 23 | 73.91 | 94.12 | 7 |
| cel_dead_mir481 | 312 | 23 | 73.91 | 94.12 | 7 |
| cel_dead_mir513 | 313 | 23 | 73.91 | 100 | 6 |
| cel_dead_mir400 | 314 | 23 | 73.91 | 100 | 6 |
| cel_dead_mir363 | 315 | 23 | 73.91 | 94.12 | 7 |

| | | |
|---|---|---|
| wt_seq | wt_5p | wt_3p |

| | | | | | |
|---|---|---|---|---|---|
| (SEQ | wt_ | (SEQ | 5p_ | (SEQ | 3p_ |

| Wt_mir_id | chrstart-end(strand) | ID NO) | length | ID NO) | length | ID NO) | length |
|---|---|---|---|---|---|---|---|
| cel.mir5545_MI0019066 | I:11885595-11885705(+) | 325 | 110 | 343 | 24 | 334 | 23 |
| cel.mir8196b_MI0026837 | X:14324405-14324470(−) | 326 | 65 | 344 | 23 | 335 | 24 |
| cel.mir4805_MI0017535 | II:1061647-106 I 741(+) | 327 | 94 | 345 | 23 | 336 | 22 |
| cel.mir5545_MI0019066 | I:11885595-11885705(+) | 328 | 110 | 346 | 24 | 337 | 23 |
| cel.mir5545_MI0019066 | I:11885595-11885705(+) | 329 | 110 | 347 | 24 | 338 | 23 |
| cel.mir5552_MI0019073 | V:18036731-18036841(+) | 330 | 110 | 348 | 23 | 339 | 23 |
| cel.mir5545_MI0019066 | I:11885595-11885705(+) | 331 | 110 | 349 | 24 | 340 | 23 |
| cel.mir5545_MI0019066 | I:11885595-11885705(+) | 332 | 110 | 350 | 24 | 341 | 23 |
| cel.mir5545_MI0019066 | I:11885595-11885705(+) | 333 | 110 | 351 | 24 | 342 | 23 |

H. sapiens

To generate the initial list of candidates, the list of all known human miRNA precursors from miRbase were blasted against the human genome. This resulted in a list of 85,399 candidate locations from which all the known miRNAs and cases that mapped to uncharacterized genomic regions were removed, and 73,340 initial candidates were left. Next, the mature guide and passenger sequences of all known human miRNA's were mapped to the human genome. If a mature sequence mapped to any of the locations in the initial candidates list with at least 50% sequence similarity, it was deemed a candidate. The final candidates list consisted of 5406 candidates. Next, the sequence of each candidate was extended to match the length of the WT miRNA to which it initially matched, such that the location of the mature miRNA in the WT miRNA corresponded to the location of the identified sequence in the candidate. Finally, the fasta sequences of each of the final candidates were extracted and the positions of their mature sequences(s) were marked based on the position of the mature sequences in the miRbase miRNA according to which they were initially derived. In addition, using the corresponding genomic annotation file, it was determined whether the candidate is located within an intronic or exonic region.

Table 4 provides a list of H. sapiens candidates that have been found as described above.

TABLE 4
List of H. sapiens candidates

| | | mut_seq | | | | |
|---|---|---|---|---|---|---|
| | | (SEQ ID) | mut_ | precursor_ | precursor_ | precursor_ |
| dead_mire_id | chr:start-end(strand) | NO) | length | coverage | pid | mutations |
| hsa_dead_mir54124 | 19:53702736-53702820(+) | 352 | 85 | 100 | 84.52 | 13 |
| hsa_dead_mir71535 | 14:26172166-26172250(+) | 353 | 97 | 96.91 | 84.52 | 13 |
| hsa_dead_mir54066 | 19:53707443-53707544(+) | 354 | 101 | 100 | 88.23 | 12 |
| hsa_dead_mir54013 | 19:53702737-53702818(+) | 355 | 81 | 100 | 90.12 | 8 |
| hsa_dead_mir54736 | 10:119005798-119005865(−) | 356 | 70 | 98.57 | 82.61 | 12 |
| hsa_dead_mir54158 | 19:53702736-53702820(+) | 357 | 87 | 98.85 | 84.52 | 13 |
| hsa_dead_mir54175 | 1:212824339-212824410(+) | 358 | 83 | 85.54 | 95.78 | 3 |
| hsa_dead_mir54878 | 13:99216012-99216051(+) | 359 | 70 | 55.71 | 97.44 | 1 |
| hsa_dead_mir54042 | 19:53702747-53702807(+) | 360 | 61 | 98.36 | 90 | 6 |
| hsa_dead_mir54572 | 21:8208011-8208126(+) | 361 | 115 | 100 | 92.17 | 9 |
| hsa_dead_mir54678 | 2:239069702-239069796(−) | 362 | 98 | 95.92 | 95.75 | 4 |
| hsa_dead_mir54174 | 8:80301189-80301272(+) | 363 | 83 | 100 | 92.77 | 6 |
| hsa_dead_mir54172 | 6:116258709-116258792(−) | 364 | 83 | 100 | 98.8 | 1 |
| hsa_dead_mir54573 | 21:8391058-8391173(+) | 365 | 115 | 100 | 92.17 | 9 |
| hsa_dead_mir50078 | 13:107724634-107724670(−) | 366 | 116 | 46.55 | 97.22 | 1 |
| hsa_dead_mir54115 | 19:53702736-53702820(+) | 367 | 87 | 98.85 | 86.91 | 11 |
| hsa_dead_mir54701 | 20:30488755-30488845(−) | 368 | 93 | 100 | 92.22 | 7 |
| hsa_dead_mir54024 | 19:53702736-53702820(+) | 369 | 87 | 98.85 | 89.29 | 9 |
| hsa_dead_mir53999 | 19:53702741-53702820(+) | 370 | 87 | 98.85 | 87.34 | 10 |
| hsa_dead_mir54975 | 21:8252690-8252858(+) | 371 | 180 | 100 | 97.66 | 4 |
| hsa_dead_mir54979 | 21:8252690-8252858(+) | 372 | 180 | 100 | 97.66 | 4 |
| hsa_dead_mir54822 | 3:67680989-67681021(+) | 373 | 70 | 45.71 | 96.88 | 1 |
| hsa_dead_mir54041 | 19:53761159-53761209(+) | 374 | 61 | 100 | 96 | 2 |
| hsa_dead_mir54025 | 19:53686468-53686555(+) | 375 | 87 | 100 | 87.36 | 11 |
| hsa_dead_mir53996 | 19:53698384-53698471(+) | 376 | 87 | 100 | 87.36 | 11 |
| hsa_dead_mir54027 | 19:53762343-53762430(+) | 377 | 87 | 100 | 86.21 | 12 |
| hsa_dead_mir59305 | 3:195699410-195699449(+) | 378 | 88 | 45.45 | 97.44 | 1 |
| hsa_dead_mir54125 | 19:53731005-53731090(+) | 379 | 85 | 100 | 94.12 | 5 |
| hsa_dead_mir54576 | 21:8986604-8986652(+) | 380 | 115 | 100 | 97.92 | 1 |
| hsa_dead_mir54040 | 19:53756767-53756817(+) | 381 | 61 | 100 | 96 | 2 |
| hsa_dead_mir51151 | 2:36435593-36435676(+) | 382 | 118 | 80.51 | 88.09 | 10 |
| hsa_dead_mir54053 | 19:53729838-53729924(+) | 383 | 85 | 100 | 87.21 | 11 |
| hsa_dead_mir53992 | 19:53752396-53752482(+) | 384 | 87 | 100 | 91.86 | 7 |
| hsa_dead_mir54074 | 19:53748635-53748722(+) | 385 | 87 | 100 | 95.4 | 4 |
| hsa_dead_mir54091 | 19:53695209-53695295(+) | 386 | 87 | 98.85 | 88.37 | 10 |
| hsa_dead_mir73320 | X:147189681-147189810(−) | 387 | 129 | 100 | 96.9 | 4 |
| hsa_dead_mir73323 | X:147189682-147189809(−) | 388 | 127 | 100 | 95.28 | 6 |
| hsa_dead_mir54155 | 19:53751210-53751297(−) | 389 | 87 | 100 | 90.81 | 8 |
| hsa_dead_mir54071 | 19:53758230-53758317(+) | 390 | 87 | 100 | 87.36 | 11 |
| hsa_dead_mir54020 | 19:53729837-53729925(+) | 391 | 87 | 100 | 89.77 | 9 |
| hsa_dead_mir54068 | 19:53756732-53756835(+) | 392 | 101 | 100 | 87.5 | 13 |

| | mut_5p | | | | | | | | | 3p_ |
|---|---|---|---|---|---|---|---|---|---|---|
| | (SEQ ID | 5p_ | 5p_ | | 5p_ | mut_3p | | 3p_ | | muta- |
| dead_mir_id | NO) | length | coverage | 5p_% id | mutations | (SEQ ID NO) | 3p_length | coverage | 3p_% id | tions |
| hsa_dead_mir54124 | 405 | 22 | 81.82 | 100 | 0 | none | N/A | N/A | N/A | NIA |
| hsa_dead_mir71535 | 406 | 22 | 100 | 100 | 0 | none | N/A | N/A | N/A | N/A |
| hsa_dead_mir54066 | 407 | 23 | 95.65 | 95.45 | 0 | none | N/A | N/A | N/A | N/A |
| hsa_dead_mir54013 | 408 | 22 | 81.82 | 100 | 0 | none | N/A | N/A | N/A | N/A |
| hsa_dead_mir54736 | 409 | 22 | 100 | 100 | 0 | none | N/A | N/A | N/A | N/A |
| hsa_dead_mir54158 | 410 | 21 | 66.67 | 100 | 0 | none | N/A | N/A | N/A | N/A |
| hsa_dead_mir54175 | 411 | 24 | 100 | 100 | 0 | none | N/A | N/A | N/A | N/A |
| hsa_dead_mir54878 | 412 | 22 | 100 | 100 | 0 | none | N/A | N/A | N/A | N/A |
| hsa_dead_mir54042 | 413 | 21 | 66.67 | 100 | 0 | none | N/A | N/A | N/A | N/A |
| hsa_dead_mir54572 | none | N/A | N/A | N/A | N/A | 393 | 22 | 100 | 100 | 0 |
| hsa_dead_mir54678 | 414 | 22 | 81.82 | 100 | 0 | none | N/A | N/A | N/A | N/A |
| hsa_dead_mir54174 | 415 | 24 | 100 | 100 | 0 | none | N/A | N/A | N/A | N/A |
| hsa_dead_mir54172 | 416 | 24 | 100 | 100 | 0 | 394 | 23 | 100 | 95.65 | 0 |
| hsa_dead_mir54573 | none | N/A | N/A | N/A | N/A | 395 | 22 | 100 | 100 | 0 |
| hsa_dead_mir50078 | 417 | 21 | 100 | 100 | 0 | none | N/A | N/A | N/A | NIA |
| hsa_dead_mir54115 | 418 | 22 | 81.82 | 100 | 0 | none | N/A | N/A | N/A | N/A |
| hsa detid mir54701 | 419 | 22 | 90.91 | 90 | 0 | none | N/A | N/A | N/A | N/A |
| hsa_dead_mir54024 | 420 | 22 | 81.82 | 100 | 0 | none | N/A | N/A | N/A | N/A |
| hsa_dead_mir53999 | 421 | 22 | 81.82 | 100 | 0 | none | N/A | N/A | N/A | N/A |
| hsa_dead_mir54975 | 422 | 21 | 80.95 | 100 | 0 | none | N/A | N/A | N/A | N/A |
| hsa_dead_mir54979 | 423 | 21 | 80.95 | 100 | 0 | none | N/A | N/A | N/A | N/A |
| hsa_dead_mir54822 | 424 | 22 | 100 | 100 | 0 | none | N/A | N/A | N/A | N/A |
| hsa_dead_mir54041 | none | N/A | N/A | N/A | N/A | 396 | 21 | 100 | 95.24 | 0 |
| hsa_dead_mir54025 | 425 | 22 | 100 | 100 | 0 | none | N/A | N/A | N/A | N/A |
| hsa_dead_mir53996 | 426 | 22 | 100 | 100 | 0 | none | N/A | N/A | N/A | N/A |
| hsa_dead_mir54027 | 427 | 22 | 100 | 95.45 | 0 | none | NIA | N/A | N/A | N/A |
| hsa_dead_mir59305 | 428 | 22 | 100 | 100 | 0 | none | N/A | N/A | N/A | N/A |
| hsa_dead_mir54125 | 429 | 20 | 100 | 100 | 0 | none | N/A | N/A | N/A | NIA |
| hsa_dead_mir54576 | none | N/A | N/A | N/A | N/A | 397 | 27 | 100 | 100 | 0 |
| hsa_dead_mir54040 | none | N/A | N/A | N/A | N/A | 398 | 21 | 100 | 95.24 | 0 |
| hsa_dead_mir51151 | 430 | 22 | 100 | 95.45 | 0 | none | N/A | N/A | N/A | N/A |
| hsa_dead_mir54053 | 43 | 22 | 100 | 95.45 | 0 | 399 | 22 | 54.55 | 100 | 0 |
| hsa_dead_mir53992 | 432 | 22 | 100 | 100 | 0 | 400 | 22 | 86.36 | 94.74 | 0 |
| hsa_dead_mir54074 | none | N/A | N/A | N/A | N/A | 401 | 22 | 100 | 100 | 0 |
| hsa_dead_mir54091 | 433 | 22 | 100 | 95.45 | 0 | none | N/A | N/A | N/A | N/A |
| hsa_dead_mir73320 | none | NIA | N/A | N/A | N/A | 402 | 23 | 95.65 | 100 | 0 |
| hsa_dead_mir73323 | none | N/A | N/A | N/A | N/A | 403 | 23 | 95.65 | 100 | 0 |
| hsa_dead_mir54155 | 434 | 21 | 100 | 95.24 | 0 | none | N/A | N/A | N/A | N/A |
| hsa_dead_mir54071 | 435 | 72 | 95.45 | 90.48 | 0 | 404 | 71 | 76.19 | 100 | 0 |
| hsa_dead_mir54020 | 436 | 22 | 100 | 100 | 0 | none | NIA | N/A | N/A | NIA |
| hsa_dead_mir54068 | 437 | 23 | 56.52 | 100 | 0 | none | N/A | N/A | N/A | NIA |

| | | wt_seq | | wt_5p | | wt_3p | |
|---|---|---|---|---|---|---|---|
| | | DEQ ID | | SEQ ID | | SEQ ID | |
| WT_mir_id | chr:start-end(strand) | NO) | wt_length | NO) | 5p_length | NO) | 3p_length |
| hsa-mir-519a-1_MI0003178 | 19:53752396-53752481(+) | 438 | 85 | none | N/A | none | N/A |
| hsa-mir-548d-1_MI0003668 | 8:123348033-123348130(−) | 439 | 97 | none | N/A | none | N/A |
| hsa-mir-518c_MI0003159 | 19:53708734-53708835(+) | 440 | 101 | 499 | 23 | 479 | 23 |
| hsa-mir-519b_MI0003151 | 19:53695212-53695293(+) | 441 | 81 | 500 | 22 | 480 | 72 |
| hsa-mir-548o-2_MI0016746 | 20:38516562-38516632(+) | 442 | 70 | none | N/A | none | N/A |
| hsa-mir-519a-2_MI0003182 | 19.53762343-53762430(+) | 443 | 87 | 501 | 21 | none | N/A |
| hsa-mir-10394_MI0033418 | 19.58393363-58393446(+) | 444 | 83 | 502 | 24 | 481 | 23 |
| hsa-mir-548o-2_MI0016746 | 20:38516562-38516632(+) | 445 | 70 | none | N/A | none | N/A |
| hsa-mir-520b_MI0003155 | 19:53701226-53701287(+) | 446 | 61 | 503 | 21 | 482 | 21 |
| hsa-mir-663b_MI0006336 | 2:132256965-132257080(−) | 447 | 115 | 504 | 22 | none | N/A |
| hsa-mir--4440_MI0016783 | 2:239068816-239068914(−) | 448 | 98 | 505 | 22 | none | N/A |
| hsa-mir-10394_MI0033418 | 19:58393363-58393446(+) | 449 | 83 | 506 | 24 | 483 | 23 |
| hsa-mir-10394_MI0033418 | 19:58393363-58393446(+) | 450 | 83 | 507 | 24 | 484 | 23 |
| hsa-mir-663b_MI0006336 | 2:132256965-132257080(−) | 451 | 115 | 508 | 22 | none | N/A |
| hsa-mir-1273h_MI0025512 | 16:24203115-24203231(+) | 452 | 116 | 509 | 21 | 485 | 22 |
| hsa-mir-522_MI0003177 | 19:53751210-53751297(+) | 453 | 87 | 510 | 72 | 486 | 22 |
| hsa-mir-663a_ MI0003672 | 20:26208185-26208278(−) | 454 | 93 | 511 | 22 | none | N/A |
| hsa-mir-523_MI0003153 | 19:53698384-53698471(+) | 455 | 87 | 512 | 22 | 487 | 23 |
| hsa-mir-519c_ MI0003148 | 19:53686468-53686555(+) | 456 | 87 | 513 | 22 | 488 | 72 |
| hsa-mir-3648-1_MI0016048 | 21:8208472-8208652(+) | 457 | 180 | none | N/A | none | N/A |
| hsa-mir-3648-2_MI0031512 | 21:8986998-8987178(+) | 458 | 180 | none | N/A | none | N/A |
| hsa-mir-548o-2_MI0016746 | 20:38516562-38516632(+) | 459 | 70 | none | N/A | none | N/A |
| hsa-mir-520b_MI0003155 | 19:53701226-53701287(+) | 460 | 61 | 514 | 21 | 489 | 21 |
| hsa-mir--523_MI0003153 | 19:53698384-53698471(+) | 461 | 87 | 515 | 22 | 490 | 23 |
| hsa-mir--519c_MI0003148 | 19:53686468-53686555(+) | 462 | 87 | 516 | 22 | 491 | 22 |
| hsa-mir-523_MI0003153 | 19:53698384-53698471(+) | 463 | 87 | 517 | 22 | 492 | 23 |
| hsa-mir-548ai_MI0016813 | 6:99124608-99124696(+) | 464 | 88 | 518 | 22 | none | N/A |
| hsa-mir-5272.410003179 | 19:53754017-53754102(+) | 465 | 85 | 519 | 20 | none | N/A |
| hsa-mir-663b_MI0006336 | 2:132256965-132257080(−) | 466 | 115 | 520 | 72 | none | N/A |
| hsa-mir--520b_MI0003155 | 19:53701226-53701287(+) | 467 | 61 | 521 | 21 | 493 | 21 |
| hsa-mir-548h-3_MI0006413 | 17:13543528-13543646(−) | 468 | 118 | none | N/A | none | N/A |
| hsa-mir-526a-1_MI0003157 | 19:53706251-53706336(+) | 469 | 85 | none | N/A | none | N/A |
| hsa-mir-519c_ MI0003148 | 19:53686468-53686555(+) | 470 | 87 | 572 | 22 | 494 | 72 |
| hsa-mir-521-2_MI0003163 | 19:53716593-53716680(+) | 4711 | 87 | none | N/A | none | N/A |
| hsa-mir-518d_MI0003171 | 19:53734876-53734963(+) | 472 | 87 | 523 | 22 | 495 | 21 |
| hsa-mir-513a-1_MI0003191 | X:147213462-147213594(−) | 473 | 129 | none | N/A | none | N/A |
| hsa-mir-513a-2_MI0003192 | X:147225825-147225952(−) | 474 | 127 | none | N/A | none | N/A |
| hsa-mir-519a-2_MI0003182 | 19:53762343-53762430(+) | 475 | 87 | 524 | 21 | none | N/A |
| hsa-mir-524_MI0003160 | 19:53711001-53711088(+) | 476 | 87 | 525 | 22 | 496 | 21 |
| hsa-mir-523_MI0003153 | 19:53698384-53698471(+) | 477 | 87 | 526 | 22 | 497 | 23 |
| hsa-mir-518c_MI0003159 | 19:53708734-53708835(+) | 478 | 101 | 527 | 23 | 498 | 23 |

### Detection of Expressed Candidates

To identify expressed candidates, the IntersectBed software was used, in a stranded manner, to determine the overlap between the genomic coordinates of each candidate with all of the small RNAseq samples from the relevant organism and recorded the number of small RNA reads that matched each genomic location within each candidate gene. Raw read counts were then normalized to RPKM (Reads Per Kilobase Million) using the following formula:

$$R P K {M}_{i} = \frac{{X}_{i}}{( \frac{{l}_{i}}{{10}^{3}} ) ( \frac{N}{{10}^{6}} )}$$

$$where {X}_{i} = number of reads mapping to gene i , {l}_{i} = length of gene i and N = total number of mapped reads$$

Candidates for which there were at least 10 reads on the same genomic location were considered expressed. The expression of each corresponding WT miRNAs was also determined in the exact same manner.

To identify expressed candidates, the number of small RNA-seq reads with a length of 19-24 bp and ≥19 bp that perfectly matched the genomic position of the candidates or the corresponding known WT miRNA was recorded. Once all the small RNA-seq samples were mapped to all of the candidates and their corresponding known miRNAs, their coverage plot, along each of their genomic positions, was generated and analysed. As discussed above, only expressed candidates were selected following this analysis.

### Detection of Expressed Non-Processed Candidates

Typically, using the analysis described above, miRNAs that are processed in a canonical fashion have at least one, if not two, peaks of small RNA reads that match the length of the mature guide and/or passenger sequences (typically 21-22 bp long), thus, in order to identify non-processed miRNAs, the small RNA expression plots of each candidate was inspected and it was determined whether they display an expression pattern similar to that of a canonical miRNA (which means that they are processed as a silencing miRNA and thus may not be used for silencing reactivation/redirection) or whether they are non-processed (namely, display an expression pattern different than that of a wild-type miRNA).

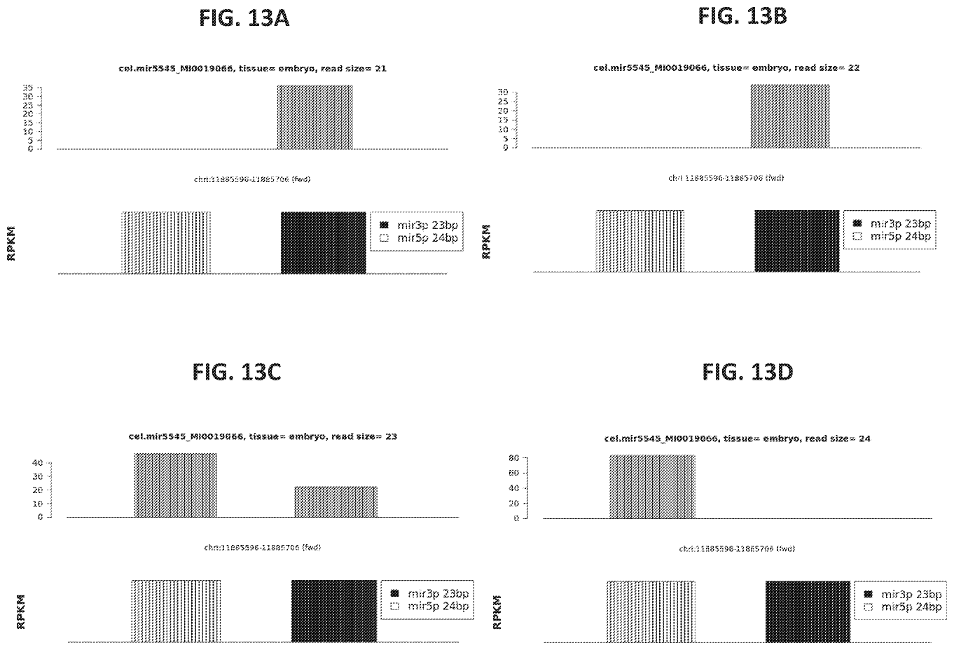

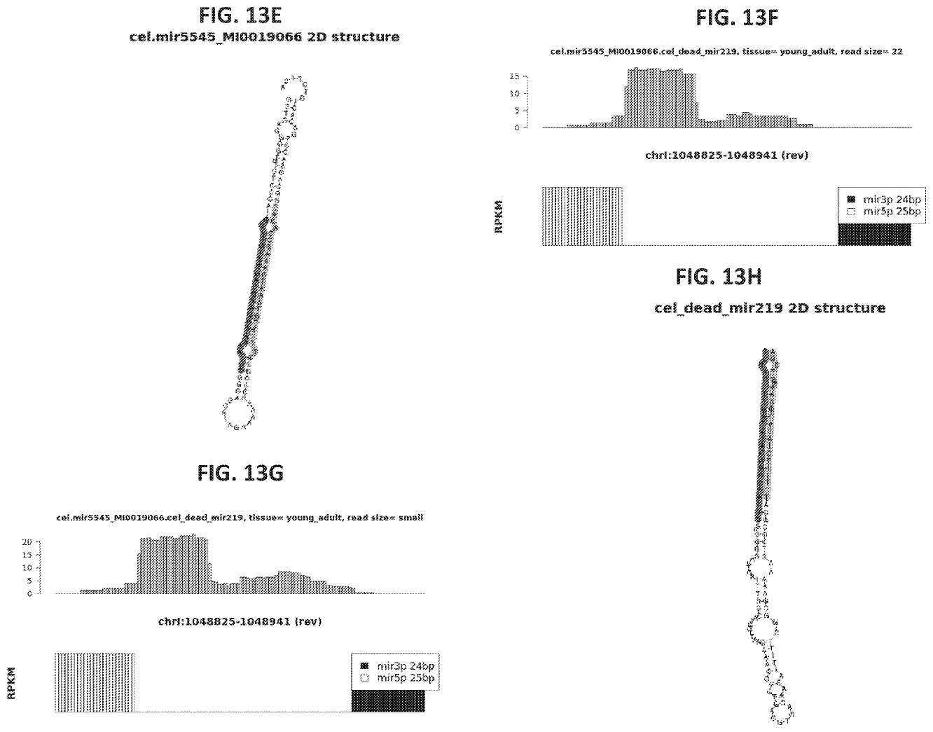

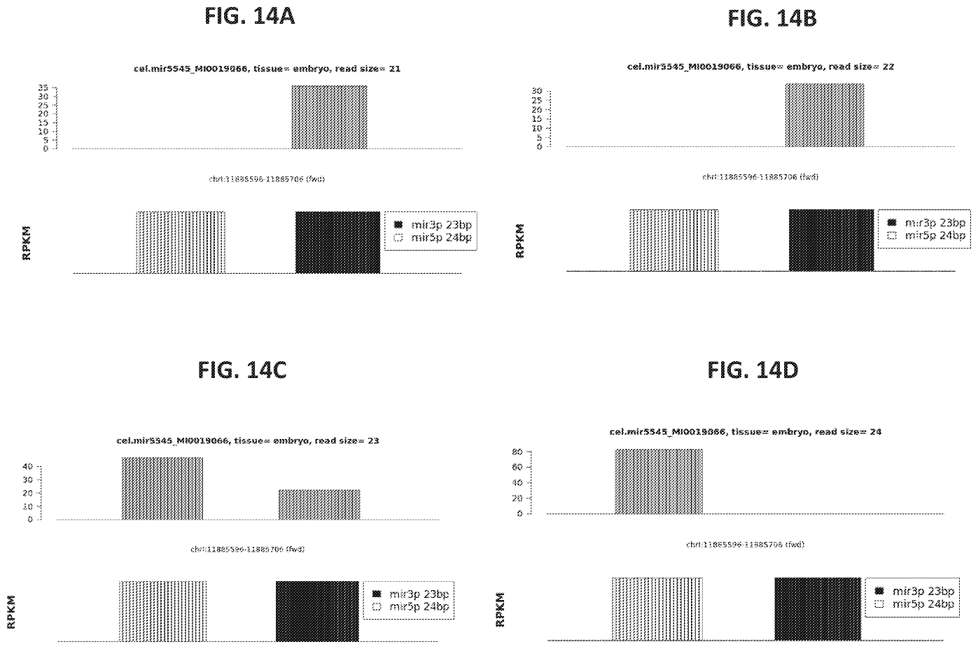

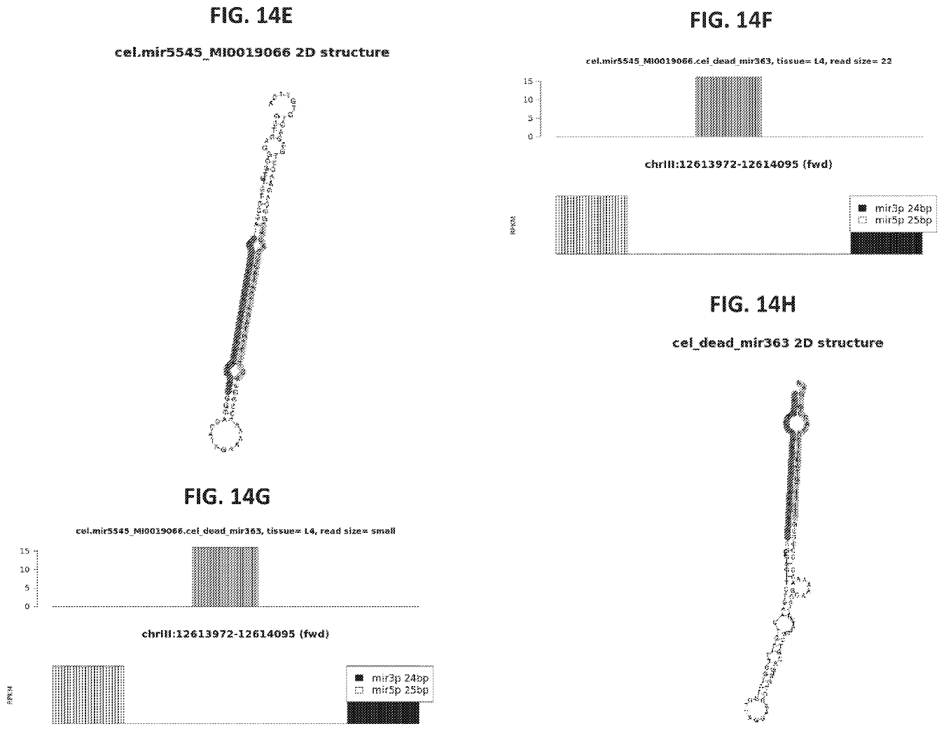

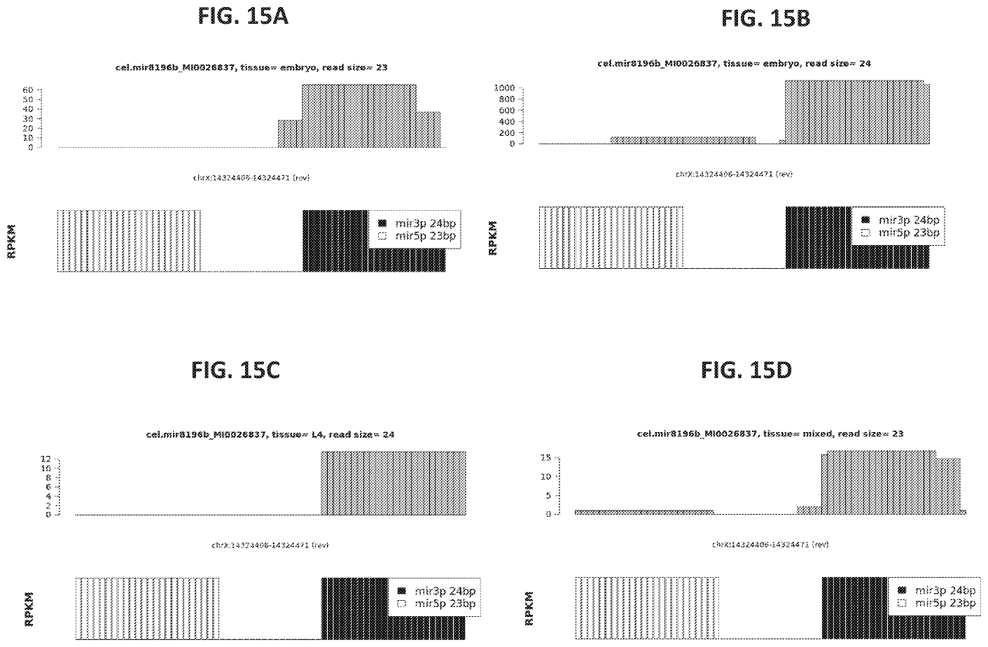

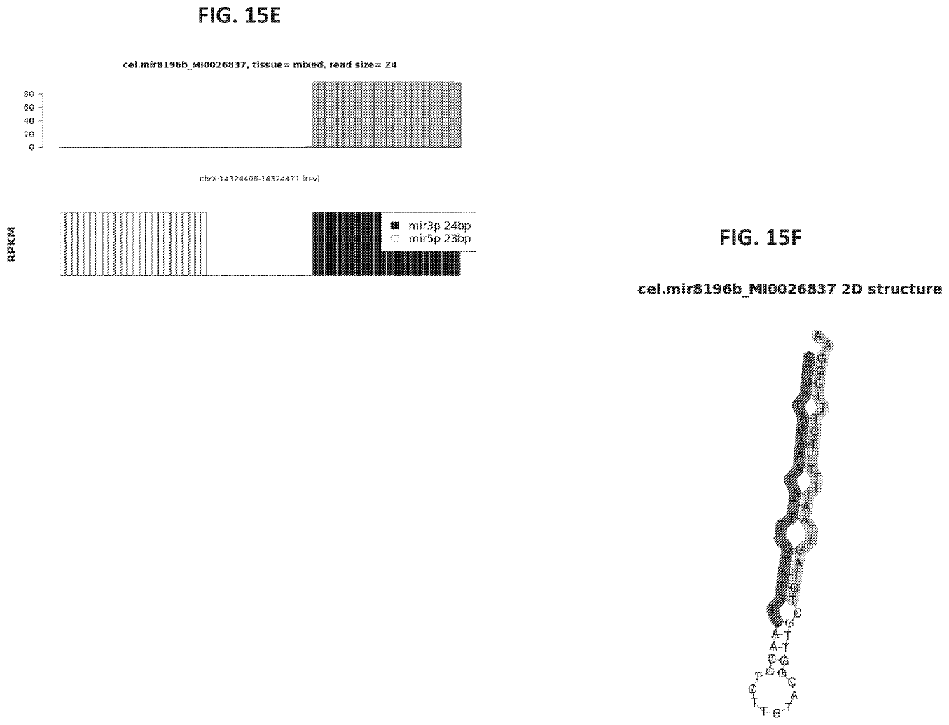

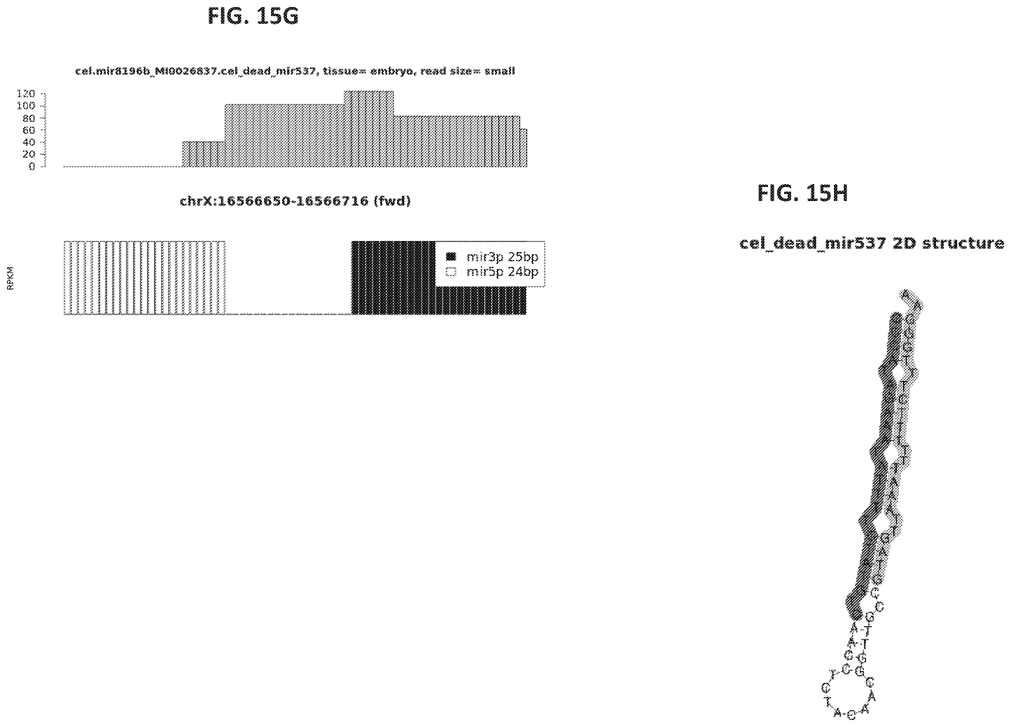

FIGS. 13A-H, for example, show the sRNA expression of wild-type miRNA cel-mir-5545 (MI0019066) and one if its corresponding miRNA-like genes, cel_dead_mir219. FIG. 13A displays the small RNA seq expression plot of cel-mir-5545 in embryos for reads that are 21 bp long. The x-axis presents the genomic location of the precursor sequence (chrI, between posions 11885596 and 11885706 on the forward strand) in a 5′ to 3′ orientation and the y-axis denotes the expression values in RPKM. The lower plot marks the positions of the mature miRNA sequences as defined according to miRbase. The 3′ miRNA is marked in black bars along the x-axis positions that mark the 3p mature miRNA and the 5′ miRNA is marked in white bars along the x-axis positions that mark the 5p mature miRNA. The legend in the lower plot indicates the length of the mature miRNAs according to miRbase. A processed miRNA shows an expression pattern in which the location of expressed small RNAs is aligned with the positions of the mature miRNAs. By looking at the locations of the mature miRNAs and the positions of the miRNAs in FIGS. 13C and 13D, it can be determined that cel-mir-5545 undergoes processing. In a similar manner, FIGS. 13F and 13G depict the expression of small RNA for mir-like cel_dead_mir219 along the genomic location of its putative precursor sequence. The black and white bars represent the locations of its mature miRNAs and the upper plot shows that the expression pattern is not located in the positions of the mature miRNAs but rather along the central part of the mir-like precursor sequence. Thus, clearly indicating that cel_dead_mir219 is expressed but not processed like its corresponding wild-type miRNA.

FIGS. 10A-N demonstrate the distribution of small RNAs of various sizes from shoot and root tissues against the A. thaliana miRNA-like candidate gene ath_dead_mir1334 (encoding a miRNA-like molecule that has been identified as described above, FIGS. 10H-M) and its corresponding wild-type miRNA, ath-mir-8174 (FIGS. 10A-F). As can be seen, while the plots for the wild-type miRNA show that small RNA expression (upper graph in each plot) correspond with the genomic location of the miRNA's mature sequence (lower graph in each plot), the sRNAs corresponding to ath_dead_mir1334 do not intersect with the genomic location in which its “mature” sequence would have been. Analysis of RNA secondary structure predicted on the basis of sequence shows that while the precursor of the wild-type miRNA folds like a canonical miRNA (FIG. 10G), as known from the art, the RNA from the miRNA-like gene does not (FIG. 10N), further confirming that it does not have a silencing activity corresponding to that of its wild-type counterpart. The guide strand of the mature miRNA is highlighted in grey in FIG. 10G, and the corresponding sequence in the RNA “precursor” of the miRNA-like candidate is highlighted in FIG. 10N.

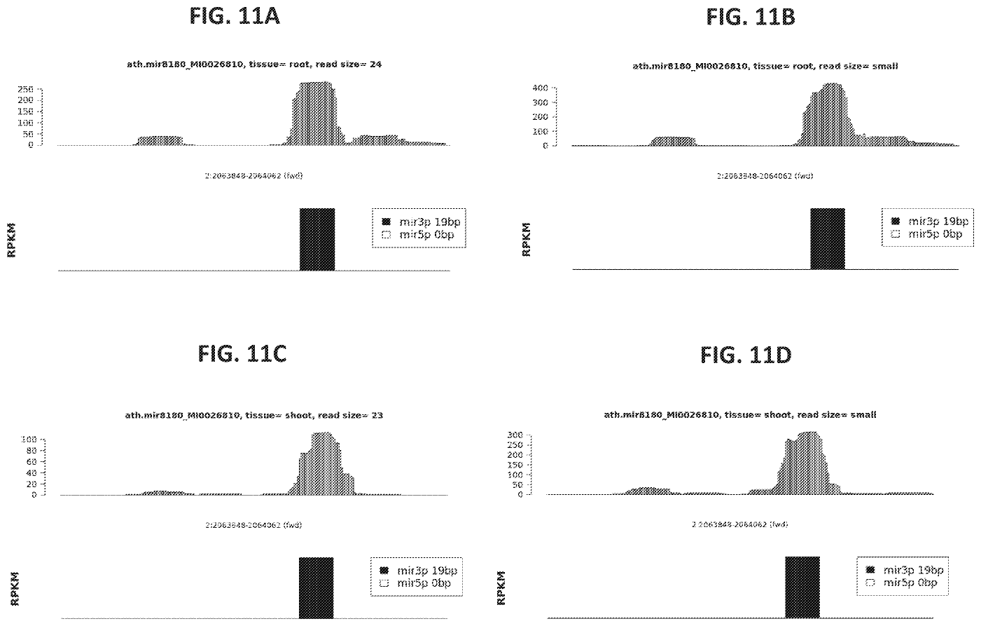

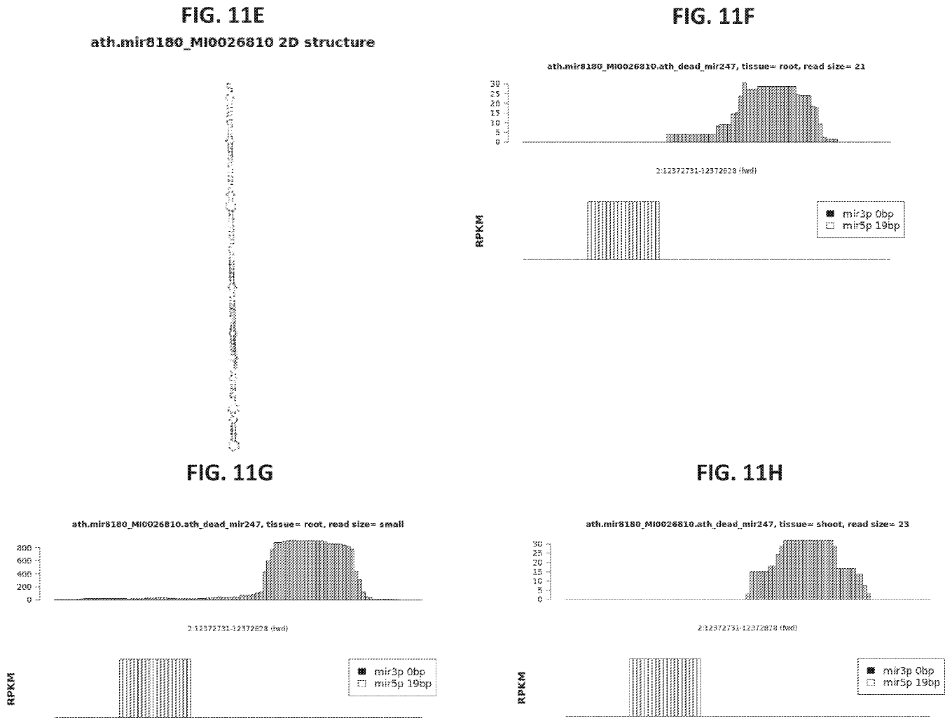

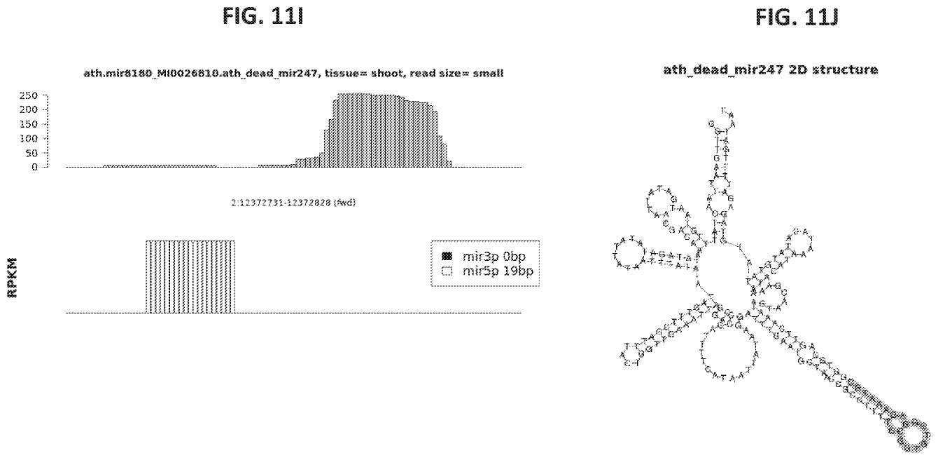

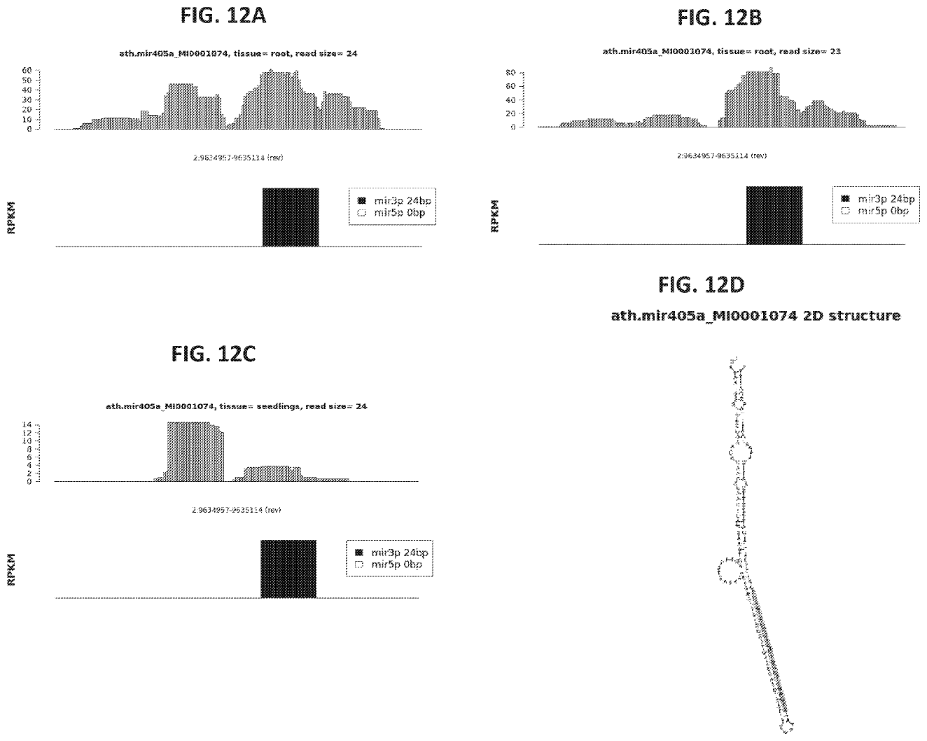

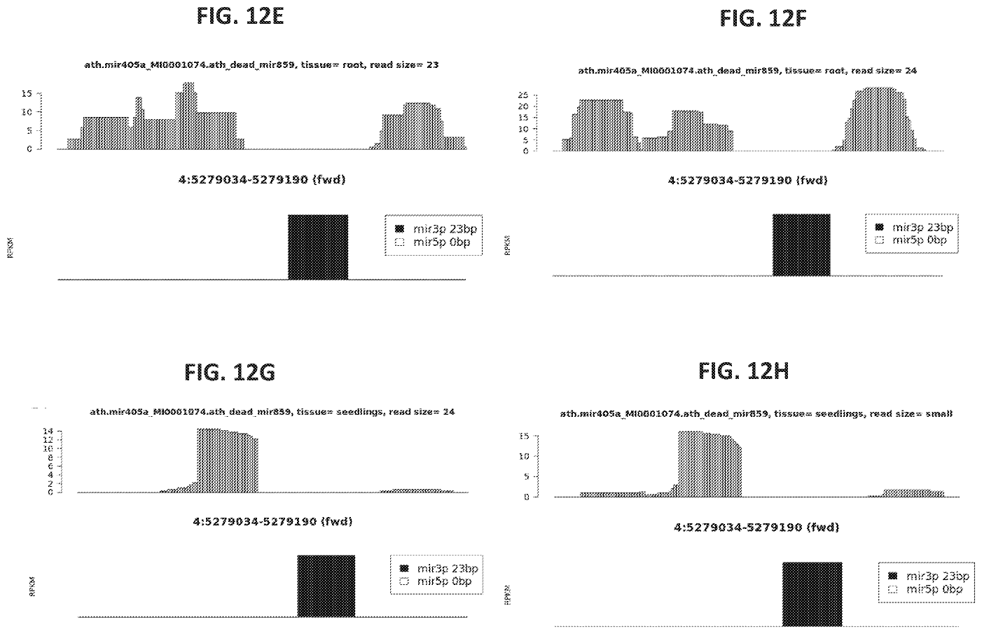

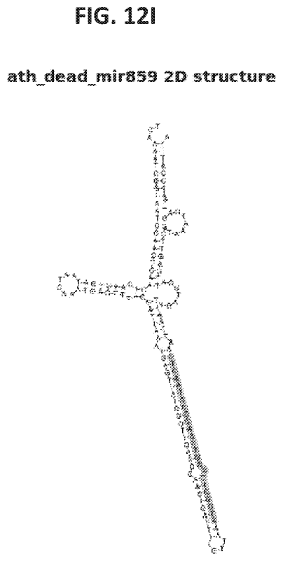

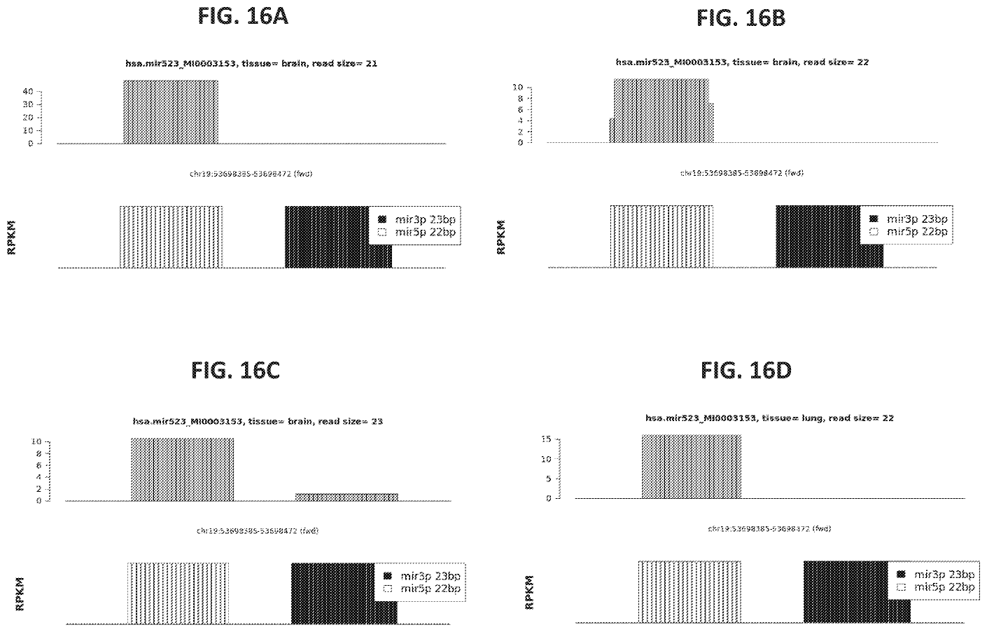

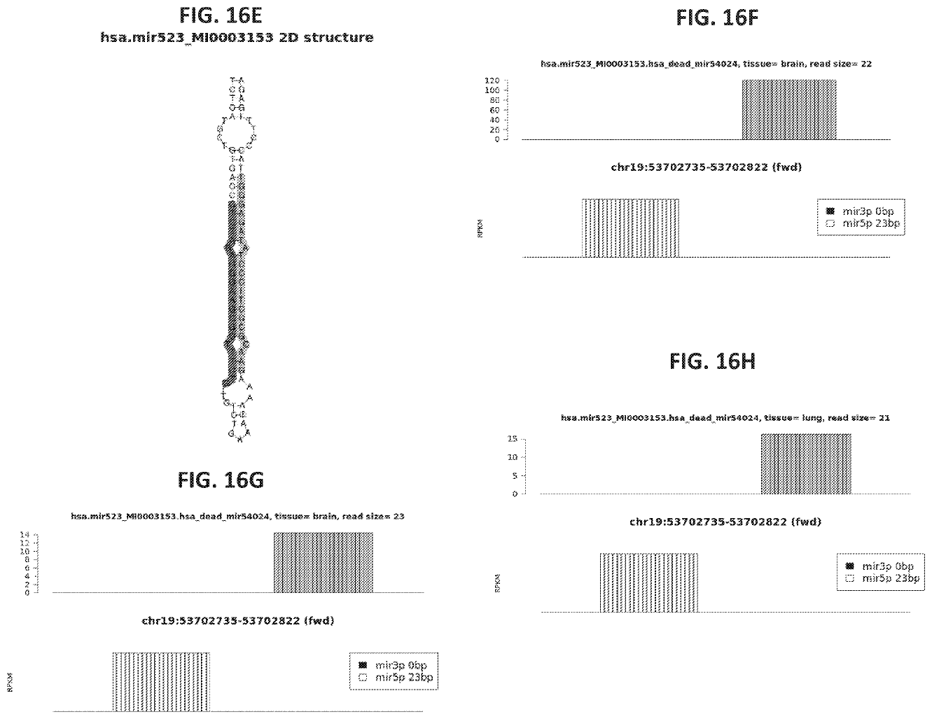

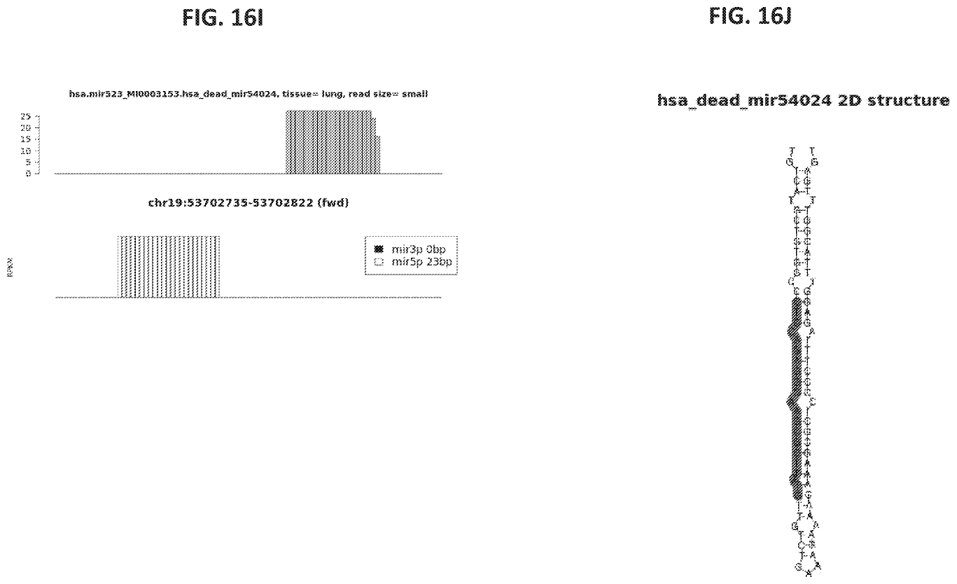

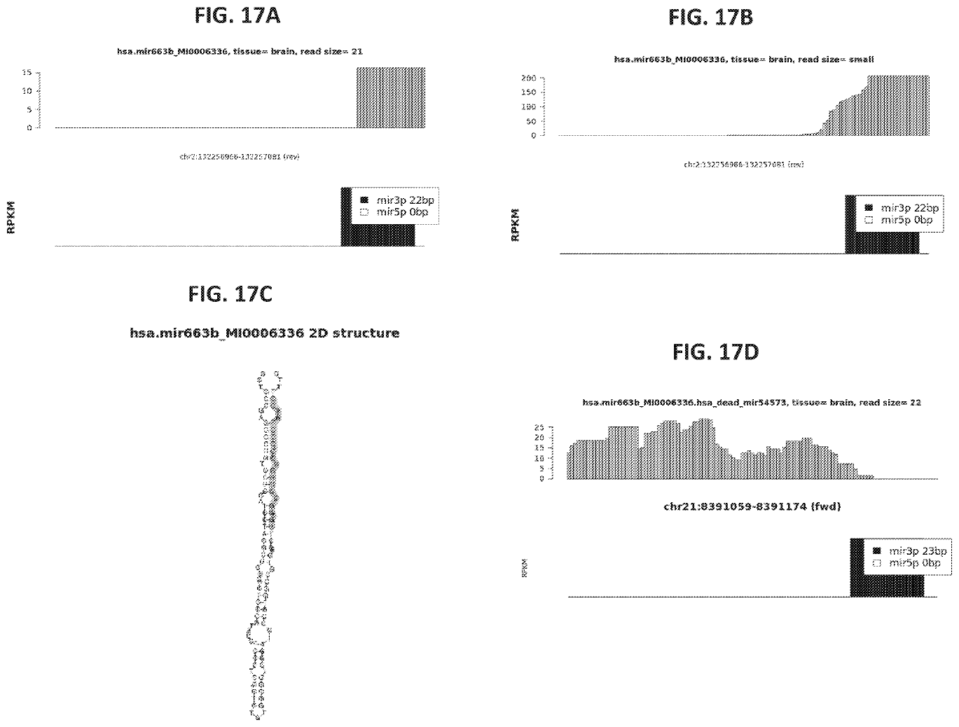

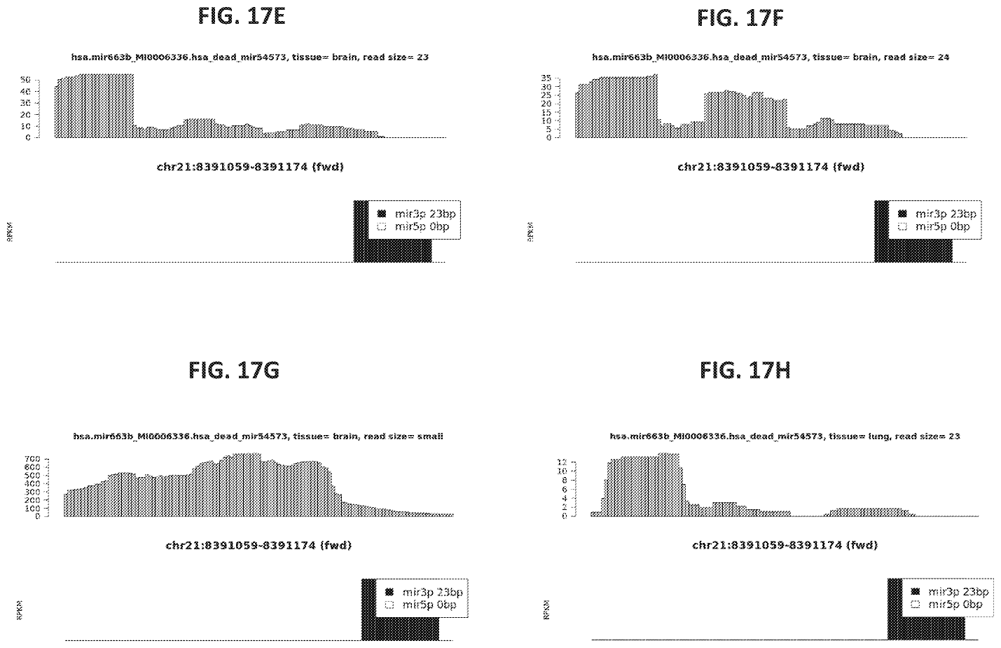

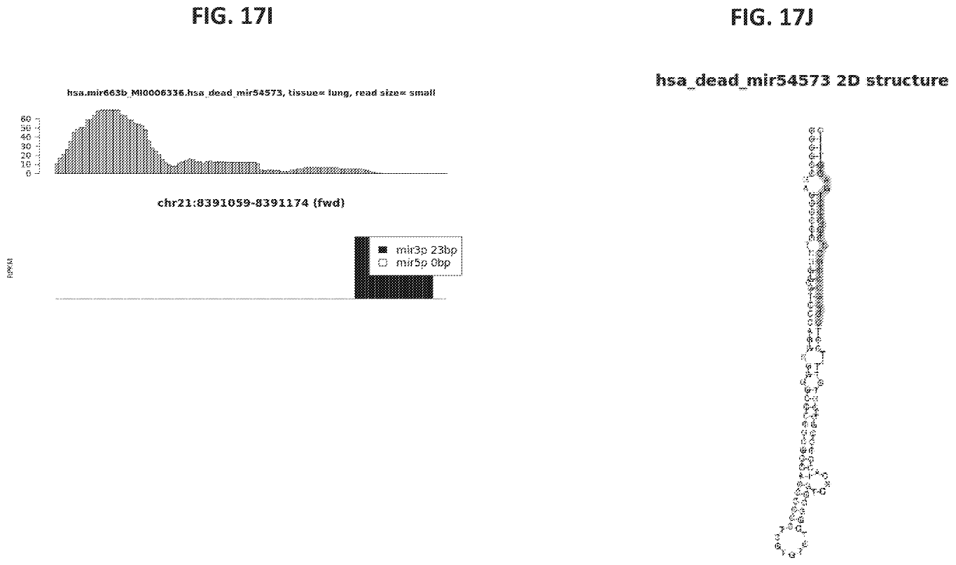

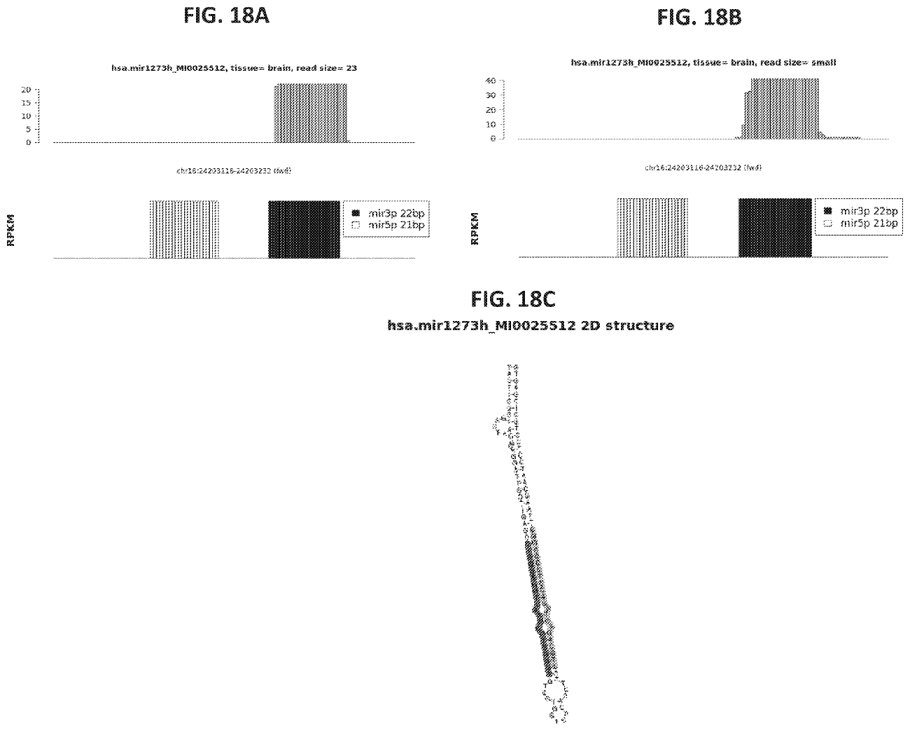

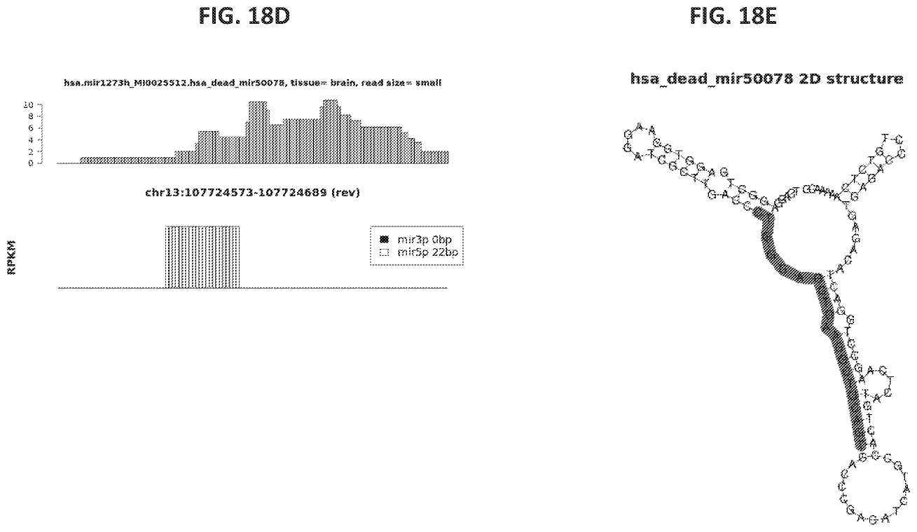

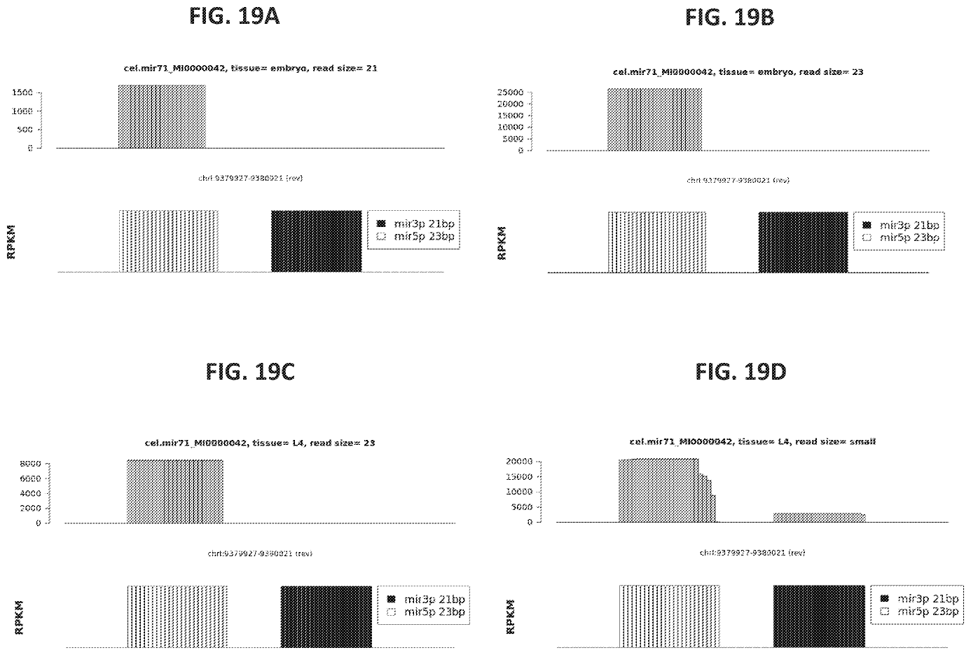

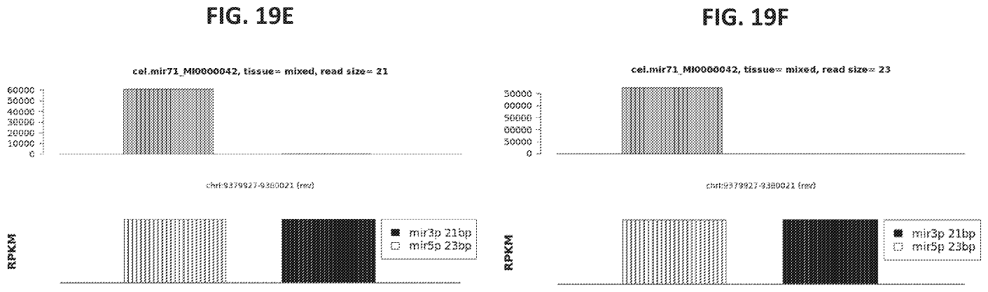

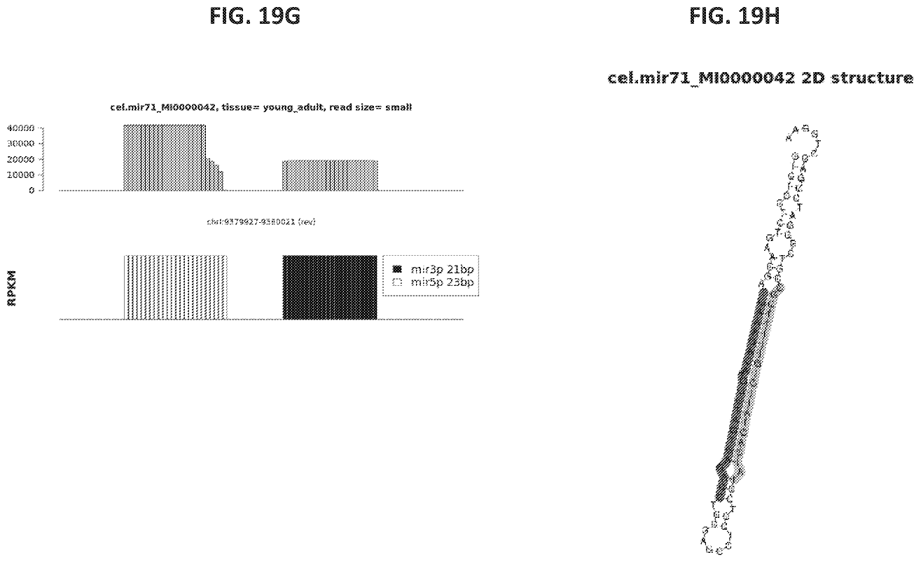

FIGS. 11A-J and 12A-I present a similar analysis for other miRNA-like genes from A. thaliana, FIGS. 13A-H, 14A-H and 15A-H from C. elegans and FIGS. 16A-J, 17A-J and 18A-E from H. sapiens demonstrating that the miRNA-like genes are expressed but not processed like their counterpart wild-type miRNAs. FIGS. 19A-G present the expression analysis of a canonical wild-type miRNAs from C. elegans and FIG. 19H shows the predicted RNA secondary structure of the wild-type miRNA cel-mir-71.

siRNA Design

Target-specific siRNAs are designed by publically available siRNA-designers such as ThermoFisher Scientific's “BLOCK-iT™ RNAi Designer” and Invivogen's “Find siRNA sequences”.

sgRNAs Design

As described above, silencing activity of an identified candidate gene (encoding a ncRNA which is expressed but not processed like a corresponding wild-type silencing molecule), such as a gene encoding a miRNA-like molecule, can be reactivated (and possibly redirected) by introducing nucleotide changes to the gene sequence. The required nucleotide changes can be introduced using the GEiGS technology. In order to do so, an endonuclease such as Cas9 is introduced into a cell together with a donor DNA molecule encoding the relevant sequence of the candidate gene with desired nucleotide changes. The Cas9 endonuclease will cut the sequence of the candidate gene in the cell based on the sequence of a sgRNA which is further introduced to the cells. sgRNAs are designed to target endogenous candidate genes encoding miRNA-like molecules using the publically available sgRNA designer, as previously described in Park et al., Bioinformatics, (2015) 31(24): 4014-4016. Two sgRNAs are designed for each cassette, and a single sgRNA is expressed per cell, to initiate gene swapping with the introduced donor DNA. sgRNAs correspond to the pre-miRNA-like sequence that is intended to be modified post swapping.

To maximize the chance of efficient sgRNA choice, two or more different publicly available algorithms (CRISPER Design: www(dot)crispr(dot)mit(dot)edu:8079/and CHOPCHOP: www(dot)chopchop(dot)cbu(dot)uib(dot)no/) are used and the top scoring sgRNA from each algorithm is selected.

Swapping ssDNA Oligo Design

To design the DNA donor to be used with GEiGS, as described above, a GEiGS-oligo is first designed. A 400 nt ssDNA (sizing between 100-1000 bp) oligo is designed based on the genomic DNA sequence of the miRNA-like candidate gene. The pre-miRNA-like sequence of the target gene is located in the center of the donor oligo (including the desired nucleotide changes to reactivate/redirect silencing activity), and the mature-like miRNA sequence of the candidate gene is replaced with a double-stranded siRNA sequence against a target of choice, such that the guide (silencing) siRNA strand is kept 70-100% complementary to the target (additional nucleotide changes along the pre-miRNA-like sequence of the target gene might be introduced so as to effect modification to reactive or redirect silencing specificity, as described herein). The sequence of the passenger siRNA strand is modified to preserve the original miRNA structure, keeping the same base pairing profile.

### Swapping Plasmid DNA Design

A 4000 bp (range between 200-4000 bp) dsDNA fragment is designed based on the genomic DNA sequence of the miRNA gene. The GEiGS-oligo, as described above, is located in the center of the dsDNA fragment. The fragment is cloned into a standard vector (e.g. Bluescript comprising or not comprising a fluorescence marker) and transfected into the cells with the Cas9 system components.

Possible Target Genes for Redirected ncRNAs

The above described ncRNA are modified into siRNA targeting, for example:
Arabidopsis host: TuMV, Luciferase (target and control)
Human host: HIV, Luciferase (target and control)
C. elegans host: UNC-22, Luciferase (target and control)

(as discussed in Table 5, below)

TABLE 5
Target Genes

| | Query sequence | SEQ |
|---|---|---|
| Gene name | ID | ID NO |
| Arabidopsis TuMV | AF169561.2 | 47 |
| Arabidopsis | AP018660.1 (Publically | 48 |
| Luciferase | available sequence from | |
| | Gateway | |
| | vector R4L1pMpGWB435) | |
| Human | AFQ33819.3 | 49 |
| HIV | | |
| Human | FJ376737.1 (commercially | 50 |
| Luciferase | available sequences from | |
| | PmirGLO Dual-Luciferase | |
| | miRNA Target Expression | |
| | Vector (Promega, USA) | |
| C. elegans | NC_003282.8 | 51 |
| UNC-22 | | |
| C. elegans | FJ376737.1 (commercially | 52 |
| Luciferase | available sequences from | |
| | PmirGLO Dual-Luciferase | |
| | miRNA Target Expression | |
| | Vector (Promega, USA) | |

### Computational Pipeline to Generate GEiGS Templates

The computational GEiGS pipeline applies biological metadata and enables an automatic generation of GEiGS DNA templates that are used to minimally edit non-coding RNA genes (e.g. miRNA genes), leading to a new gain of function. i.e. redirection of their silencing capacity to target sequence of interest.

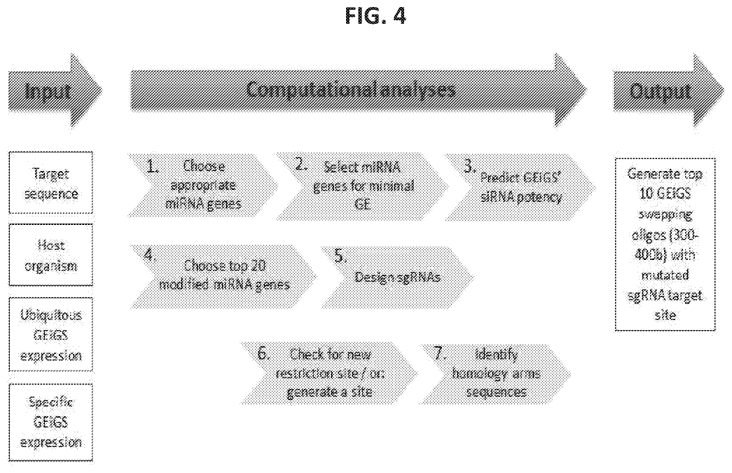

As illustrated in FIG. 4, the pipeline starts with filling and submitting input; a) target sequence to be silenced by GEiGS; b) the host organism to be gene edited and to express the GEiGS; c) one can choose whether the GEiGS would be expressed ubiquitously or not. If specific GEiGS expression is required, one can choose from a few options (expression specific to a certain tissue, developmental stage, stress, heat/cold shock etc).

When all the required input is submitted, the computational process begins with searching among miRNA (or other non-coding RNAs) datasets (e.g. small RNA sequencing, microarray etc.) and filtering only relevant miRNAs that match the input criteria. Next, the selected mature miRNA sequences are aligned against the target sequence and miRNA with the highest complementary levels are filtered. These naturally target-complementary mature miRNA sequences are then modified to perfectly match the target's sequence. Then, the modified mature miRNA sequences are run through an algorithm that predicts siRNA potency and the top 20 with the highest silencing score are filtered. These final modified miRNA genes are then used to generate 200-500 nt ssDNA or 250-5000 nt dsDNA sequences as follows:

200-500 nt ssDNA oligos and 250-5000 nt dsDNA fragments are designed based on the genomic DNA sequence that flanks the modified miRNA. The pre-miRNA sequence is located in the center of the oligo. The modified miRNA's guide strand (silencing) sequence is 100% complementary to the target. However, the sequence of the modified passenger miRNA strand is further modified to preserve the original (unmodified) miRNA structure, keeping the same base pairing profile.

Next, differential sgRNAs are designed to specifically target the original unmodified miRNA gene, and not the modified swapping version. Finally, comparative restriction enzyme site analysis is performed between the modified and the original miRNA gene and differential restriction sites are summarized.

Therefore, the pipeline output includes:

a) 200-500 nt ssDNA oligo or 250-5000 nt dsDNA fragment sequence with minimally modified miRNA

b) 2-3 differential sgRNAs that target specifically the original miRNA gene and not the modified

c) List of differential restriction enzyme sites among the modified and original miRNA gene.

Sequences
Target Oligos (used in the “Luc-sensor vector”):
1. Dead/Reactivated_859-
SEQ ID Nos: 6 and 9
GAGTTATGGGTTGACCGAACCCAT
2. Dead/Reactivated_1334-
SEQ ID Nos: 20 and 23
GATTAGCTTCCCTATACACAT
3. WT_Active_405a-
SEQ ID NO: 3
AGTTATGGGTTAGACCCAACTCAT
4. WT_Active_8174-
SEQ ID NO: 17
GATTAGCTTCCCTATACACAT
5. Redirected_859-
SEQ ID NO: 12
CAATTCGTAAATCAAAATTTTAAT
6. Redirected_1334-
SEQ ID NO: 26
CCAGTTATTAATGTTCATATA
“GEiGS-Oligos” (used in the “GEiGS-oligo” vector)
1. miR405a_Active-
SEQ ID NO: 1
TCAAAATGGGTAACCCAACCCAACCCAACTCATAATCAAATGAGTTT
ATGATTAAATGAGTTATGGGTTGACCCAACTCATTTTGTTAAATGAG
TTGGGTCTAACCCATAACTCATTTCATTTGATGGGTTGAGTTGTTAA
ATGGGTTAACCATTTA
2. miR8174_Active-
SEQ ID NO: 15
CGGCCCATCCGTTGTCTTTCCTGGTACGCATGTGCCATGGCTTTCTC
GTAAGGGACTGGATTGTCCGTATTTCTCATGTGTATAGGGAAGCTAA
TCGTCTTGTAGATGGGTTG
3. miR859_Dead-
SEQ ID NO: 4
TCAAAATGGGTAATCCAACTCAACTCAACTCATAATCAAATGAGTTT
AGGATTAAATGAGTTATGGGTTGACCCAACTCATTTTGTTAAATGGG
TTCGGTCAACCCATAACTCAATTAATTTGATGGATTGAGTTGGTAAA
TGAGTTAACCCATTTA
4. miR1334_Dead-
SEQ ID NO: 18
ATTCGCATTCTCTGTCTTTCCTAGTACGTTTATGTTATGGCTTCATT
TCGAAGGACTAGATTGTCCGAATTACTCATGTGTATAGGGAAGCTAA
TCGTCTCGCAGATGAATTA
5. miR859_Reactivated-
SEQ ID NO: 7
CCAGATTGGATTGCCTCACACCACACACGACTCAATTCACTAAGACG
AGGATTAAATTGGGTTATGGGTGACCGAACTCATTTTGCCAAatggg
ttcggtcaacccataactcAATTTTGGTGAAGGTCGTGGGTGGAAAA
GGAGGCAACCCAGTCA
6. miR1334_Reactivated-
SEQ ID NO: 21
TCACGCATTCGTTGACTTCCCTAGTACGCATATTGAACTGCTGTAAG
GTGAAGGACGTTAATGTACCAAAAACTTatgtgtatagggaagctaa
tcGTCCCGCAGATGTGTGA
7. miR859_Redirected-
SEQ ID NO: 10
TCAAATTGGGTAAACTACCCCAACATCTCTCAAAATCCAAGGTTGTT
AGGACCAAATGTGGTTTGTGGACAGAGTTTTCATTTTGCTAAatgaa
aattttgatttacgaattgCATTATCTTGGGTGAGGGAGGTTGCAAA
TTAGTTTAGCCAGTTA
8. miR1334_Redirected-
SEQ ID NO: 24
ATGTGCATCGCAGTGATTGGTGTGTTATATGACTAAAAGTCTTTATC
GCGAAGGGCTATATCGACCTAGGTACTTtatatgaacattaataact
ggCCCCCCCAGATGCATGT

PCR for Amplification of miRNA Oligos

The miRNA oligos were amplified from synthetic template ordered from Genewiz in order to introduce compatible ends for in-fusion cloning. CloneAmp HiFi PCR Premix (Takara Bio) was used according to the manufacturer's instructions.

To add in Fw Oligo primer:
(SEQ ID NO: 29)
5′ AAACGAGCTCGCTAG
To add in Rev Target primer:
(SEQ ID NO: 30)
5′ GCAGGTCGACTCTAG

Each PCR reaction included a negative control with H2O (No template).

TABLE 6A
template for PCR
PCR Template

| | |
|---|---|
| A | Dead miRNAs |
| B | WT miRNAs |
| C | Redirected miRNAs |
| D | Reactivated miRNAs |

PCR products were loaded on 0.8% agarose gels and specific PCR bands were excised and purified using Monarch DNA Gel Extraction Kit (NEB) according to the manufacturer's instructions.

TABLE 6B
Combinations of PCR primers and templates used

| | | SEQ | | |
|---|---|---|---|---|
| Name | Sequence | ID NO: | Template | PCR |
| miR859_Dead_F | 5′-AAACGAGCTCGCTAGTCAAAATGGGT | 31 | A | 3 |
| | AATCCAACTCAACTCAACTCAT-3′ | | | |
| miR859_Dead_R | 5′-GCAGGTCGACTCTAGTAAATGGGTTA | 32 | | |
| | ACTCATTTACCAACTCAATCCATCAA-3′ | | | |
| miR1334_Dead_F | 5′-AAACGAGCTCGCTAGATTCGCATTCT | 33 | A | 4 |
| | CTGTCTTTCCTAGTACG-3′ | | | |
| miR1334_Dead_R | 5′-GCAGGTCGACTCTAGTAATTCATCTG | 34 | | |
| | CGAGACGATTAGCTTCCC-3′ | | | |
| miR405a_Active_F | 5′-AAACGAGCTCGCTAGTCAAAATGGGT | 35 | B | 5 |
| | AACCCAACCCAACCCAACT-3′ | | | |
| miR405a_Active_R | 5′-GCAGGTCGACTCTAGTAAATGGTTAA | 36 | | |
| | CCCATTTAACAACTCAACCCATCA-3′ | | | |
| miR8174_Active_F | 5′-AAACGAGCTCGCTAGCGGCCCATCCG | 37 | B | 7 |
| | TTGTCT-3′ | | | |
| miR8174_Active_R | 5′-GCAGGTCGACTCTAGCAACCCATCTA | 38 | | |
| | CAAGACGATTAGCT-3′ | | | |
| miR859_ | 5′-AAACGAGCTCGCTAGTCAAATTGGGT | 39 | C | 11 |
| Redirected_F | AAACTACCCCAACATCTCT-3′ | | | |
| miR859_ | 5′-GCAGGTCGACTCTAGTAACTGGCTAA | 40 | | |
| Redirected_R | ACTAATTTGCAACCTCCCT-3′ | | | |
| miR1334_ | 5′-AAACGAGCTCGCTAGATGTGCATCGC | 41 | C | 12 |
| Redirected_F | AGTGATTGGT-3′ | | | |
| miR1334_ | 5′-GCAGGTCGACTCTAGACATGCATCTG | 42 | | |
| Redirected_R | GGGGGG-3′ | | | |
| miR859_ | 5′-AAACGAGCTCGCTAGCCAGATTGGAT | 43 | D | 15 |
| Reactivated_F | TGCCTCACACC-3′ | | | |
| miR859_ | 5′-GCAGGTCGACTCTAGTGACTGGGTTG | 44 | | |
| Reactivated_R | CCTCCTTTTCC-3′ | | | |
| miR1334_ | 5′-AAACGAGCTCGCTAGTCACGCATTCG | 45 | D | 16 |
| Reactivated_F | TTGACTTCCCTAGT-3′ | | | |
| miR1334_ | 5′-GCAGGTCGACTCTAGTCACACATCTG | 46 | | |
| Reactivated_R | CGGGACgattag-3′ | | | |

Cloning of Annealed Targets into Multiple_Cloning_Site_Target

Luc-sensor vector was restriction enzyme digested with XmaI and HpaI.

TABLE 6C
Typical restriction reaction

| | Vol x1 (μl) |
|---|---|
| Cutsmart Buffer 10X (NEB) | 3.5 |
| Enzyme 1 | 1 |
| Enzyme 2 | 1 |
| Luc-sensor vector (1 μg/ul) | 2 |
| H20 | 27.5 |
| | Final volume: 35 |

Incubated at 37° C. for 4 hours.

The volume was run in a 0.8% agarose gel and the restricted band was purified using Monarch DNA Gel Extraction Kit (NEB) according to the manufacturer's instructions.

In Fusion Cloning of Annealed Target Oligos into Luc-Sensor Vector XmaI and HpaI Restricted

Annealed targets were cloned into the restricted MCS of Luc-sensor vector using In-Fusion HD Cloning Kit (Takara Bio) according to the manufacturer's instructions. Final plasmids were transformed intro Stellar Competent Cells (Takara Bio) according to the manufacturer's instructions and cells were plated for selection on LB Carbenicillin agar plates and incubated for overnight growth at 37° C. Cultures were started for 3 clones obtained from each reaction and plasmid DNA was extracted using QIAprep Spin Miniprep Kit (QIAGEN) according to the manufacturer's instructions. Confirmation of cloned DNA sequences was obtained by Sanger sequencing. Sequencing results were analysed using Snapgene software.

Necessary amount of vectors for transfection were obtained using QIAGEN Plasmid Plus Kits (QIAGEN) according to the manufacturer's instructions.

Cloning of GEiGS-Oligos into Multiple_Cloning_Site_GEiGS_Oligo

Luc-sensor vector was restriction enzyme digested with NheI and XbaI.

TABLE 6D
Typical restriction reaction

| | Vol x1 (μl) |
|---|---|
| Cutsmart Buffer 10X (NEB) | 3.5 |
| Enzyme 1 | 1 |
| Enzyme 2 | 1 |
| Luc-sensor vector (1 μg/ul) | 2 |
| H20 | 27.5 |
| | Final volume: 35 |

Incubated at 37° C. for 4 hours.

The volume was run in a 0.8% agarose gel and the restricted band was purified using Monarch DNA Gel Extraction Kit (NEB) according to the manufacturer's instructions.

In-Fusion Cloning of GEiGS-Oligos into GEiGS-Oligo Vector NheI and XbaI Restricted.

Purified PCR products were cloned into the restricted MCS of GEiGS-Oligo vector using In-Fusion HD Cloning Kit (Takara Bio) according to the manufacturer's instructions.

Final plasmids were transformed intro Stellar Competent Cells (Takara Bio) according to the manufacturer's instructions and cells were plated for selection on LB Carbenicillin agar plates and incubated for overnight growth at 37° C. Cultures were started for 3 clones obtained from each reaction and plasmid DNA was extracted using QIAprep Spin Miniprep Kit (QIAGEN) according to the manufacturer's instructions. Confirmation of cloned DNA sequences was obtained by Sanger sequencing. Sequencing results were analysed using Snapgene software.

Necessary amount of vectors for transfection were obtained using QIAGEN Plasmid Plus Kits (QIAGEN) according to the manufacturer's instructions.

### Protoplasts Isolation

Arabidopsis (Col-0 ecotype) protoplasts were isolated by incubating plant material (e.g. leaves, calli, cell suspensions) in a digestion solution (1% cellulase, 0.3% macerozyme, 0.4 M mannitol, 154 mM NaCl, 20 mM KCl, 20 mM MES pH 5.6, 10 mM CaCl₂)) for 4-24 hours at room temperature and gentle shaking. After digestion, remaining plant material was washed with W5 solution (154 mM NaCl, 125 mM CaCl2, 5 mM KCl, 2 mM MES pH5.6) and protoplasts suspension was filtered through a 40 μm strainer. After centrifugation at 80 g for 3 minutes at room temperature, protoplasts were resuspended in 2 ml W5 buffer and precipitated by gravity in ice. The final protoplast pellet was resuspended in 2 ml of MMg (0.4 M mannitol, 15 mM MgCl2, 4 mM MES pH 5.6) and protoplast concentration was determined using a hemocytometer.

### Protoplasts Viability was Estimated Using Trypan Blue Staining.

Polyethylene glycol (PEG)-mediated plasmid transfection PEG-transfection of protoplasts was effected using a modified version of the strategy reported by Wang [Wang et al., Scientia Horticulturae (2015) 191: p. 82-89]. Protoplasts were resuspended to a density of 2-5×10⁶ protoplasts/ml in MMg solution. 100-200 μl of protoplast suspension was added to a tube containing the plasmid. The plasmid:protoplast ratio greatly affects transformation efficiency therefore a range of plasmid concentrations in protoplast suspension, 5-300 μg/μl, were assayed. PEG solution (100-200 μl) was added to the mixture and incubated at 23° C. for various lengths of time ranging from 10-60 minutes. PEG4000 concentration was optimized, a range of 20-80% PEG4000 in 200-400 mM mannitol, 100-500 mM CaCl₂ solution was assayed. The protoplasts were then washed in W5 and centrifuged at 80 g for 3 minutes, prior resuspension in 1 ml W5 and incubated in the dark at 23° C. After incubation for 24-72 hours fluorescence was detected by microscopy.

PEG Transfection for Reactivation Experiments

Molar ratio Luc-sensor vector: GEiGS-Oligo vector was 1:4. Which translates into 5 μg Luc-sensor vector and approximately 20.5 μg GEiGS-Oligo vector per transfection.

TABLE 7A
PEG experimental conditions for reactivation

| Experimental | Target in Luc-sensor | Oligo in GEiGS-Oligo vector |
|---|---|---|
| Condition | vector (5 μg) | (approx. 20.5 μg) |
| 1 | WT_Active_405a | miR405a_Active |
| 2 | WT_Active_405a | EMPTY |
| 3 | WT_Active_8174 | miR8174_Active |
| 4 | WT_Active_8174 | EMPTY |
| 5 | Dead/Reactivated_859 | miR859_Dead |
| 6 | Dead/Reactivated_859 | miR859_Reactivated |
| 7 | Dead/Reactivated_859 | EMPTY |
| 8 | Dead/Reactivated_1334 | miR1334_Dead |
| 9 | Dead/Reactivated_1334 | EMPTY |
| 10 | — | — |

Transfections were done in independent triplicates for all experimental conditions.

PEG Transfection for Redirection Experiments

Molar ratio Luc-sensor vector: GEiGS-Oligo vector was 1:4. Which translates into 5 μg Luc-sensor vector and approximately 20.5 μg GEiGS-Oligo vector per transfection.

TABLE 7B
PEG experimental conditions for redirection

| Exp. | Target in Luc-sensor | Oligo in GEiGS-Oligo vector |
|---|---|---|
| Condition | vector (5 ug) | (approx. 20.5 ug) |
| 1 | Redirected_859 | miR859_Redirected |
| 2 | Redirected_859 | miR859_Reactivated |
| 3 | Redirected_1334 | miR1334_Redirected |
| 4 | Redirected_1334 | miR1334_Reactivated |
| 5 | — | — |

Transfections were done in independent triplicates for all experimental conditions.

### Bombardment and Plant Regeneration

### Arabidopsis Root Preparation

Chlorine gas sterilized Arabidopsis (cv. Col-O0) seeds are sown on MS minus sucrose plates and vernalised for three days in the dark at 4° C., followed by germination vertically at 25° C. in constant light. After two weeks, roots are excised into 1 cm root segments and placed on Callus Induction Media (CIM: ½ MS with B5 vitamins, 2% glucose, pH 5.7, 0.8% agar, 2 mg/l IAA, 0.5 mg/l 2,4-D, 0.05 mg/l kinetin) plates. Following six days incubation in the dark, at 25° C., the root segments are transferred onto filter paper discs and placed onto CIMM plates, (½ MS without vitamins, 2% glucose, 0.4 M mannitol, pH 5.7 and 0.8% agar) for 4-6 hours, in preparation for bombardment.

### Bombardment

Plasmid constructs are introduced into the root tissue via the PDS-1000/He Particle Delivery (Bio-Rad; PDS-1000/He System #1652257), several preparative steps, outlined below, are required for this procedure to be carried out.

### Gold Stock Preparation

40 mg of 0.6 μm gold (Bio-Rad; Cat: 1652262) is mixed with 1 ml of 100% ethanol, pulse centrifuged to pellet and the ethanol is removed. This wash procedure is repeated another two times.

Once washed, the pellet is resuspended in 1 ml of sterile distilled water and dispensed into 1.5 ml tubes of 50 μl aliquot working volumes.

### Bead Preparation

In short, the following is performed:

A single tube is sufficient gold to bombard 2 plates of Arabidopsis roots, (2 shots per plate), therefore each tube is distributed between 4 (1,100 psi) Biolistic Rupture disks (Bio-Rad; Cat: 1652329).

Bombardments requiring multiple plates of the same sample, tubes are combined and volumes of DNA and CaCl₂/spermidine mixture adjusted accordingly, in order to maintain sample consistency and minimize overall preparations.

The following protocol summarizes the process of preparing one tube of gold, these should be adjusted according to number of tubes of gold used.

All subsequent processes are carried out at 4° C. in an Eppendorf thermomixer.

Plasmid DNA samples are prepared, each tube comprising 11 μg of DNA added at a concentration of 1000 ng/μl

1) 493 μl ddH2O is added to 1 aliquot (7 μl) of spermidine (Sigma-Aldrich; S0266), giving a final concentration of 0.1 M spermidine. 1250 μl 2.5M CaCl₂) is added to the spermidine mixture, vortexed and placed on ice.

2) A tube of pre-prepared gold is placed into the thermomixer, and rotated at a speed of 1400 rpm.

3) 11 μl of DNA is added to the tube, vortexed, and placed back into the rotating thermomixer.

4) To bind, DNA/gold particles, 70 μl of spermidine CaCl₂ mixture is added to each tube (in the thermomixer).

5) The tubes are vigorously vortexed for 15-30 seconds and placed on ice for about 70-80 seconds.

6) The mixture is centrifuged for 1 minute at 7000 rpm, the supernatant is removed and placed on ice.

7) 500 μl 100% ethanol is added to each tube and the pellet is resuspended by pipetting and vortexed.

8) The tubes are centrifuged at 7000 rpm for 1 minute.

9) The supernatant is removed and the pellet resuspended in 50 μl 100% ethanol, and stored on ice.

### Macro Carrier Preparation

The following is performed in a laminar flow cabinet:

1) Macro carriers (Bio-Rad; 1652335), stopping screens (Bio-Rad; 1652336), and macro carrier disk holders are sterilized and dried.

2) Macro carriers are placed flatly into the macro carrier disk holders.

3) DNA coated gold mixture is vortexed and spread (5 μl) onto the center of each Biolistic Rupture disk.

Ethanol is allowed to evaporate.

### PDS-1000 (Helium Particle Delivery System)

In short, the following is performed:

The regulator valve of the helium bottle is adjusted to at least 1300 psi incoming pressure. Vacuum is created by pressing vac/vent/hold switch and holding the fire switch for 3 seconds. This ensured helium is bled into the pipework.

1100 psi rupture disks are placed into isopropanol and mixed to remove static.

1) One rupture disk is placed into the disk retaining cap.

2) Microcarrier launch assembly is constructed (with a stopping screen and a gold containing microcarrier).

3) Petri dish Arabidopsis root callus is placed 6 cm below the launch assembly.

4) Vacuum pressure is set to 27 inches of Hg (mercury) and helium valve is opened (at approximately 1100 psi).

5) Vacuum is released; microcarrier launch assembly and the rupture disk retaining cap are removed.

6) Bombardment on the same tissue (i.e. each plate is bombarded 2 times).

7) Bombarded roots are subsequently placed on CIM plates, in the dark, at 25° C., for additional 24 hours.

### Co-Bombardments

When bombarding GEiGS plasmids combinations, 5 μg (1000 ng/μl) of the sgRNA plasmid is mixed with 8.5 μg (1000 ng/μl) swap plasmid (e.g. DONOR) and 11 μl of this mixture is added to the sample. If bombarding with more GEiGS plasmids at the same time, the concentration ratio of sgRNA plasmids to swap plasmids (e.g. DONOR) used is 1:1.7 and 11 μg (1000 ng/μl) of this mixture is added to the sample. If co-bombarding with plasmids not associated with GEiGS swapping, equal ratios are mixed and 11 μg (1000 ng/μl) of the mixture is added to each sample.

### Protoplast Microscopy

A Leica DM6000 fluorescence microscope was used for visualising fluorescent protein (FP and FP2) fluorescence 48 hours post transfection for qualitatively assessing transfection efficiency.

### Luciferase Assay on Transfected Protoplasts and Cell Analysis

24-72 hours after plasmid delivery, cells were collected and resuspended in D-PBS media. Half of the solution was used for analysis of luciferase activity, and half was analyzed for small RNA sequencing. Analysis of Dual luciferase assay was carried out using Dual-Glo® Luciferase Assay System (Promega, USA) according to the manufacturer's instructions. Total RNA was extracted with Total RNA Purification Kit (Norgene Biotek Corp., Canada), according to manufacturer's instructions. Small RNA sequencing was carried out for the identification of the desired mature small RNA in these samples.

### Plant Regeneration

For shoot regeneration, a modified protocol from Valvekens et al. [Valvekens, D. et al., Proc Natl Acad Sci USA (1988) 85(15): 5536-5540] is carried out. Bombarded roots are placed on Shoot Induction Media (SIM) plates, which included ½ MS with B5 vitamins, 2% glucose, pH 5.7, 0.8% agar, 5 mg/l 2 iP, 0.15 mg/l IAA. Plates are left in 16 hours light at 25° C.—8 hours dark at 23° C. cycles. After 10 days, plates are transferred to MS plates with 3% sucrose, 0.8% agar for a week, then transferred to fresh similar plates. Once plants regenerated, they are excised from the roots and placed on MS plates with 3% sucrose, 0.8% agar, until analyzed.

### Genotyping

Tissue samples are treated, and amplicons amplified in accordance with the manufacturer's recommendations using Phire Plant Direct PCR Kit (Thermo Scientific; F-130WH). Oligos used for these amplifications are designed to amplify the genomic region spanning from a region in the modified sequence of the GEiGS system, to outside of the region used as HDR template, to distinguish from DNA incorporation. Different modifications in the modified loci are identified through different digestion patterns of the amplicons, given by specifically chosen restriction enzymes.

### DNA and RNA Isolation

Samples are harvested into liquid nitrogen and stored in −80° C. until processed. Grinding of tissue is carried out in tubes placed in dry ice, using plastic Tissue Grinder Pestles (Axygen, US). Isolation of DNA and total RNA from ground tissue is carried out using RNA/DNA Purification kit (cat. 48700; Norgen Biotek Corp., Canada), according to manufacturer's instructions. In the case of low 260/230 ratio (<1.6), of the RNA fraction, isolated RNA is precipitated overnight in −20° C., with 1 μl glycogen (cat. 10814010; Invitrogen, US) 10% V/V sodium acetate, 3 M pH 5.5 (cat. AM9740, Invitrogen, US) and 3 times the volume of ethanol. The solution is centrifuged for 30 minutes in maximum speed, at 4° C. This is followed by two washes with 70% ethanol, air-drying for 15 minutes and resuspending in Nuclease-free water (cat. 10977035; Invitrogen, US).

Reverse Transcription (RT) and Quantitative Real-Time PCR (qRT-PCR)

One microgram of isolated total RNA is treated with DNase I according to manufacturer's manual (AMPD1; Sigma-Aldrich, US). The sample is reverse transcribed, following the instructor's manual of High-Capacity cDNA Reverse Transcription Kit (cat 4368814; Applied Biosystems, US).

For gene expression, Quantitative Real Time PCR (qRT-PCR) analysis is carried out on CFX96 Touch™ Real-Time PCR Detection System (BioRad, US) and SYBR® Green JumpStart™ Taq ReadyMix™ (S4438, Sigma-Aldrich, US), according to manufacturer's' protocols, and analysed with Bio-RadCFX manager program (version 3.1).

### Protein Sample Preparation

For protein analysis, proteins are extracted with the following protocol:

1. Wash the cells on the plate with 1×PBS
2. Drain/aspirate any access PBS from the plate
3. Lyse the cells on plate at room temperature (RT) using lysis buffer (150 μl per 6 cm dish, e.g. Lysis buffer: 50 mM Tris-Hcl Ph 7.5, 2% SDS, 20 mM NEM (N-Ethylmaleimide), protease inhibitor cocktail (cOmplete™ Protease Inhibitor Cocktail 1 tablet Roche into 50 ml of lysis buffer), Phosphatase inhibitor cocktails (SIGMA) using both 1:100).
4. Collect the lysate into an Eppendorf tube.
5. Boil the sample for 5 min at 95° C. to reduce viscosity
6. Measure protein concentration usingQuantiPro™ BCA Assay Kit, QPBCA (Sigma Aldrich, USA) according to manufacturer's protocol.
7. Equalise all samples (same volume and same concentration with lysis buffer)

### Protein Electrophoresis and Transfer

1. Add SDS loading buffer (x1—50 mM Tris-Cl (pH 6.8), 2% (w/v) SDS (sodium dodecyl sulfate; electrophoresis grade), 0.1% (w/v) bromophenol blue, 100 mM β-mercaptoethanol).
2. Boil the samples for 5 min at 95° C.
3. Load the samples and a protein ladder on the appropriate precast SDS-PAGE gel (NuPAGE™ 4-12% Bis-Tris Protein Gels; TheromFisher, USA).
4. Run the SDS-PAGE gel using running buffer (NuPAGE MOPS SDS Running Buffer; ThermoFisher, USA).
5. Disassemble the gel cassette and prepare the transfer cassette
6. Pre-wet nitrocellulose membrane, filter paper and pads in transfer buffer. Place pads and 2 layers of filter paper in cassette (on the black site (−), protein transfer from − to +).
7. Place gel on the filter paper and carefully smoothen out.
8. Place nitrocellulose membrane on gel, using a glass rod to carefully roll out air bubbles.
9. Place two layers of filter paper on top of nitrocellulose membrane, followed by pre-wetted pad before closing the cassette.
10. Run the blot for 1 hour, 100V. Put into ice box to keep temperature down.
11. Stain membrane with 1× Ponceau solution (0.1% in 3% Acetic acid) for 1-3 minutes to visualize the protein bands. Take a picture. Remove Ponceau solution (recycle solution for next use) and wash with 0.1M NaOH until Ponceau bands are vanished. Wash with DDW.

### Immunoblotting

1. Wash 3 times with PBS, for 5 minutes each.
2. Block the membrane for 1 hour in 20 ml 1×PBS+5% non-fat dry milk, in a small Tupperware dish on a shaker. Wash 3 times with PBS containing 0.05% TWEEN20 (5 μl/10 ml), for 5 minutes each.
3. Place the membrane in a falcon tube and add 50 ml of blocking solution (2.5 gr non-fat Milk powder in 50 ml PBS/0.05% Tween20). Incubate in room temp in gentle shaking for at least 30-60 min (e.g. over-night).
4. Primary biotinylated antibody (AB) incubation: Wash briefly the falcon with membrane with approximately 35 ml washing solution (25 ml Blocking solution To—250 ml PBS/0.05% Tween20). Discard the liquid.
5. Add 5 ml washing solution and the primary antibody biotin labelled (usual dilution 1:1000-5000) (Abcam, Cambridge, UK). Incubate in room temp for at least 1 h
6. Wash briefly the falcon with membrane with approximately 35 ml washing solution. Discard the liquid.
7. Add 35 ml washing solution and incubate for 10 min; repeat wash 3 times
8. Wash briefly the falcon with membrane with approximately 35 ml Phosphate-washing solution (1.25 gr Milk powder in 250 ml TBST (Tris buffered Saline with Tween 20 pH=8). Discard the liquid.
Add 35 ml phosphate-washing solution and incubate for 10 min; repeat this stage 3 times
9. Add 4 μl of Avidin-AP (Sigma-Aldrich, USA) to 4 ml of Phosphate-washing solution (1:1000 dilution) and incubate in room temp for at least 1 h.
10. Washing: Avidin-AP. Wash briefly the falcon with membrane with approximately 35 ml TBST. Discard the liquid. Add 35 ml TBST solution and incubate for 10 min; repeat wash 3 times.
11. Detection: Membrane development is carried out using Alkaline phosphatase substrate according to manufacturer's protocol (Sigma-Aldrich, USA).
Arabidopsis Protection from TuMV Infection and Disease

### Plant Material

Arabidopsis seeds, collected from plants harboring the desired GEiGS sequence, are chlorine gas sterilized and sown 1 seed/well in MS-S agar plates. Two weeks old seedlings are transferred to soil. Plants are grown in 24° C. under 16 hours light/8 hours dark cycles. Wild type non-modified (plants) are grown and treated in parallel, as control.

Plant Inoculation with TuMV and Analysis

Procedures for the inoculation and analysis of plants with TuMV vectors are carried out as previously described (Sardaru, P. et al., Molecular Plant Pathology (2018) 19: 1984-1994. doi:10.1111/mpp.12674). In short, four weeks old Arabidopsis seedlings are inoculated with TuMV as previously described [Sánchez, F. et al. (1998) Virus Research, 55(2): 207-219] or TuMV-GFP as previously described [Touriño, A., et al. (2008) Spanish Journal of Agricultural Research, 6(S1), p. 48] expressing viral vectors. Scoring of symptoms, in the case of TuMV, takes place 10-28 days post inoculation. Analysis of GFP signal, in the case of TuMV-GFP, takes place 7-14 days post inoculation.

In addition, 14 days post inoculation, new leaves growing above the inoculation site, are harvested, and total RNA is extracted using Total RNA Purification Kit (Norgene Biotek Corp., Canada), according to manufacturer's instructions. Small RNA analysis and RNA-seq is carried out for profiling of gene expression and small RNA expression on these samples.

Human Cells Protection from HIV Infection

### Cell Lines

HIV-1 susceptible human cell lines [Reil, H. et al., Virology (1994) 205(1): 371-375] are transfected using the Expi293 Expression System (Thermo Fisher, USA) with GEiGS constructs, according to manufacturer's instructions. HIV-1 titers are measured by qRT-PCR, Western blot. Integrated HIV-1 copy number analysis is performed using Southern-blot.

Knock-Down of Endogenous Gene in C. elegans
Transformation of C. elegans

Transformation of C. elegans is carried out as previously described [Germline transformation of Caenorhabditis elegans by injection. Methods Mol Biol. (2009) 518: 123-133. doi:10.1007/978-1-59745-202-1_10). Gene knockdowns are assessed by qRT_PCR, RNA-seq and small RNA-seq.

### Example 1A

### Genome Editing Induced Gene Silencing (GEiGS)

In order to design GEiGS oligos, template non-coding RNA molecules (precursors) that are processed and give raise to derivate small silencing RNA molecules (matures) are required. Two sources of precursors and their corresponding mature sequences were used for generating GEiGS oligos. For miRNAs, sequences were obtained from the miRBase database [Kozomara, A. and Griffiths-Jones, S., Nucleic Acids Res (2014) 42: D68,ÄiD73]. tasiRNA precursors and matures were obtained from the tasiRNAdb database [Zhang, C. et al, Bioinformatics (2014) 30: 1045,Äi1046].

Silencing targets were chosen in a variety of host organisms (data not shown). siRNAs were designed against these targets using the siRNArules software [Holen, T., RNA (2006) 12: 1620,Äi1625.]. Each of these siRNA molecules was used to replace the mature sequences present in each precursor, generating “naive” GEiGS oligos. The structure of these naive sequences was adjusted to approach the structure of the wild type precursor as much as possible using the ViennaRNA Package v2.6 [Lorenz, R. et al., ViennaRNA Package 2.0. Algorithms for Molecular Biology (2011) 6: 26]. After the structure adjustment, the number of sequences and secondary structure changes between the wild type and the modified oligo were calculated. These calculations are essential to identify potentially functional GEiGS oligos that require minimal sequence changes with respect to the wild type.

CRISPR/cas9 small guide RNAs (sgRNAs) against the wild type precursors were generated using the CasOT software [Xiao, A. et al., Bioinformatics (2014) 30: 1180,Äi1182]. sgRNAs were selected where the modifications applied to generate the GEiGS oligo affect the PAM region of the sgRNA, rendering it ineffective against the modified oligo.

### Example 1B

### Gene Silencing of Endogenous Plant Gene—PDS

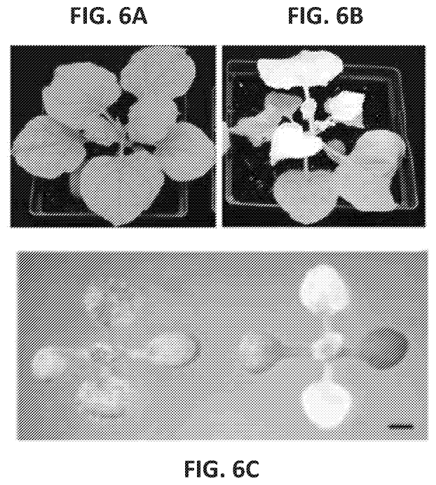

In order to establish a high-throughput screening for quantitative evaluation of endogenous gene silencing using Genome Editing Induced Gene Silencing (GEiGS), the present inventors considered several potential visual markers. The present inventors chose to focus on genes involved in pigment accumulation, such as those encoding for phytoene desaturase (PDS). Silencing of PDS causes photobleaching (FIG. 6B) which allows to use it as robust seedling screening after gene editing as proof-of-concept (POC). FIGS. 6A-C show a representative experiment with N. benthamiana and Arabidopsis plants silenced for PDS. Plants show the characteristic photobleaching phenotype observed in plants with diminished amounts of carotenoids.

In the POC experiment, choosing siRNAs was carried out as follows:

In order to initiate the RNAi machinery in Arabidopsis or Nicotiana benthamiana against the PDS gene using GEiGS application, there is a need to identify effective 21-24 bp siRNA targeting PDS. Two approaches are used in order to find active siRNA sequences: 1) screening the literature—since PDS silencing is a well-known assay in many plants, the present inventors are identifying well characterized short siRNA sequences in different plants that might be 100% match to the gene in Arabidopsis or Nicotiana benthamiana. 2) There are many public algorithms that are being used to predict which siRNA will be effective in initiating gene silencing to a given gene. Since the predictions of these algorithms are not 100%, the present inventors are using only sequences that are the outcome of at least two different algorithms.

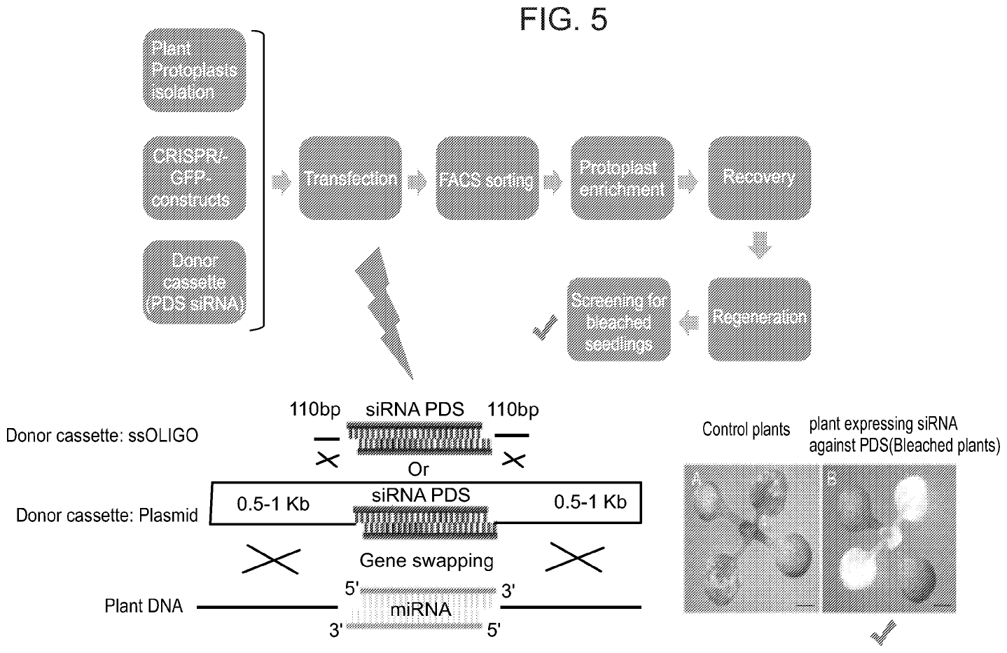

In order to use siRNA sequences that silence the PDS gene, the present inventors are swapping them with a known endogenous non-coding RNA gene sequence using the CRISPR/Cas9 system (e.g. changing a miRNA sequence, changing a long dsRNA sequence, creating antisense RNA, changing tRNA etc.). There are many databases of characterized non-coding RNAs e.g. miRNAs; the present inventors are choosing several known Arabidopsis or Nicotiana benthamiana endogenous non-coding RNAs e.g. miRNAs with different expression profiles (e.g. low constitutive expression, highly expressed, induced in stress etc.). For example, in order to swap the endogenous miRNA sequence with siRNA targeting PDS gene, the present inventors are using the HR approach (Homologous Recombination). Using HR, two options are contemplated: using a donor ssDNA oligo sequence of around 250-500 nt which includes, for example, the modified miRNA sequence in the middle or using plasmids carrying 1 Kb-4 Kb insert which comprises only minimal changes with respect to the miRNA surrounding in the plant genome except the 2×21 bp of the miRNA and the *miRNA that is changed to the siRNA of the PDS (500-2000 bp up and downstream the siRNA, as illustrated in FIG. 5). The transfection includes the following constructs: CRISPR:Cas9/GFP sensor to track and enrich for positive transformed cells, gRNAs that guides the Cas9 to produce a double stranded break (DSB) which is repaired by HR depending on the insertion vector/oligo. The insertion vector/oligo contains two continuous regions of homology surrounding the targeted locus that are replaced (i.e. miRNA) and is modified to carry the mutation of interest (i.e. siRNA). If plasmid is used, the targeting construct comprises or is free from restriction enzymes-recognition sites and is used as a template for homologous recombination ending with the replacement of the miRNA with the siRNA of choice. After transfection to protoplasts, FACS is used to enrich for Cas9/sgRNA-transfected events, protoplasts are regenerated to plants and bleached seedlings are screened and scored (see FIG. 5). As control, protoplasts are transfected with an oligo carrying a random non-PDS targeting sequence. The positive edited plants are expected to produce siRNA sequences targeting PDS and therefore PDS gene is silenced and seedling are seen as white compared to the control with no gRNA. It is important to note that after the swap, the edited miRNA will still be processed as miRNA because the original base-pairing profile is kept. However, the newly edited processed miRNA has a high complementary to the target (e.g. 100%), and therefore, in practice, the newly edited small RNA will act as siRNA.

### Example 1C

### Harboring Resistance of Arabidopsis Plants to TuMV Viral Infection

Changes in the Arabidopsis genome are designed to introduce silencing specificity in dysfunctional non-coding RNAs to target the Turnip Mosaic Virus (TuMV), or a random sequence (negative TuMV-silencing control). These sequences, together with extended homologous arms in the context of the genomic loci, are introduced in PUC57 vector, named DONOR. Guide RNAs are introduced in the CRISPR/CAS9 vector system, in order to generate DNA cleavage in the desired loci. The CRISPR/CAS9 vector system is co-introduced to the plants with the DONOR vectors via gene bombardment protocol, to introduce desired modifications through Homologous DNA Repair (HDR).

Arabidopsis seedlings with the desired changes in their genome are identified through genotyping, and inoculated with Agrobacterium harboring either TuMV or TuMV-GFP and scored for viral response.

### Example 2

### Functionality of the Reactivated and Redirected Plant Silencing RNA

In order to demonstrate that a miRNA-like non-coding RNA is able to gain a silencing activity when its silencing activity is reactivated or redirected, its biogenesis and activity was tested in a transient system within A. thaliana protoplasts.

The system used was aimed at comparing the silencing efficiency of a wild-type miRNA, a miRNA-like candidate molecule homologous to the wild-type miRNA and the miRNA-like molecule whose silencing activity has been reactivated (i.e. targeting a target sequence complementary to that of the guide strand present within the original miRNA-like molecule sequence) or reactivated and redirected (i.e. targeting another target sequence of choice). As described above, reactivation (or reactivation and redirection) of silencing activity in a candidate gene (encoding a ncRNA that is expressed but not processed like its corresponding wild-type silencing molecule) can be achieved using the GEiGS platform as described above and disclosed, for example, in WO 2019/058255. Using GEiGS for reactivation/redirection of silencing specificity, a DNA oligonucleotide termed “GEiGS-oligo” is designed. The sequence of the GEiGS-oligo comprises the sequence of the gene to be genetically edited, including the desired nucleotide changes (e.g. nucleotide changes required to redirect silencing specificity in the RNA encoded by that gene towards a target gene of choice). Next, a DNA oligonucleotide termed “GEiGS-donor” (also referred to herein as “donor”) is designed such that it comprises the “GEiGS-oligo” which is situated in between two sequences corresponding to sequences of the gene to be edited that flank the region targeted by the GEiGS-oligo. When a vector comprising the GEiGS-donor is introduced to a cell together with an endonuclease such as Cas9 and a sgRNA targeting the gene to be edited, the GEiGS-oligo is introduced into the genome of the cell (mediated by HDR), such that the edited gene now includes the desired changes (e.g. encodes a miRNA-like gene whose silencing activity has been reactivated and redirected).

The system used herein therefore also provided a comparative experimental assay to quantify the silencing efficiency of miRNA-like molecules whose silencing activity would be reactivated or redirected in a cell using a GEiGS-oligo encoding the necessary nucleotide changes for reactivation/redirection, as described above. Thus, a sequence within a vector termed “GEiGS-oligo vector”, which is used in the system described below to express precursors of wild-type miRNA or miRNA-like molecules, is also referred to as a “GEiGS-oligo” as such precursor sequences could be introduced to cells using a GEiGS-oligo, as described above.

In the transient system used, co-transfection of two plasmids was done into protoplasts:
i) The “Luc-sensor vector”—harbours a luciferase (LUC) coding reporter sequence fused to a MCS (Multiple Cloning Site) into which a target sequence to be silenced by a tested miRNA or miRNA-like (also referred to herein as the “GEiGS-sRNA target site”) was cloned. The vector also harbours an additional fluorescent protein (FP) marker used for normalization of the LUC signal (also referred to herein as “normalizer”).
ii) The “GEiGS-oligo vector”—harbours (1) the “GEiGS construct” (namely the miRNA precursor, miRNA-like precursor or reactivated/redirected miRNA-like precursor that could be generated by use of a GEiGS-oligo), which was cloned in a MCS; and (2) a different fluorescent protein (FP2) marker.

When the “GEiGS-Oligo” (miRNA/miRNA-like precursor) is processed by the RNAi machinery in the cells, a sRNA is generated. If that sRNA has a silencing activity and matches the target fused to the LUC transcript, that mRNA will be degraded, resulting in reduction in luciferase levels. If the sRNA does not match the target, no silencing will take place and there will be an accumulation of Luciferase which will result in a higher detectable signal. No silencing was expected for the fluorescent proteins (FP and FP2) regardless of the identity and silencing specificity of the sRNA. Qualitative transfection efficiency for both plasmids was visualised by fluorescent microscopy to detect the fluorescent proteins beginning at 2-days post-transfection.

To measure silencing of the target sequence which was cloned into the Luc-sensor vector as a result of expression of the miRNA/miRNA like precursor that was cloned into the GEiGS-oligo vector, the Luc-sensor and normaliser signals (luminescence and fluorescence, respectively) were measured 3-days post-transfection. The LUC/FP ratio was then calculated for different experimental conditions, as detailed below, and the silencing value was then calculated taking into account the activity of the treatment vs. the control treatment, using the same Luc-sensor vector.

As can be seen in Tables 1A and 1B, and further detailed below, the following combinations of target sequence (in the Luc-sensor vector) and tested miRNA/miRNA-like precursor (in the GEiGS-oligo vector) were examined:
1. A precursor sequence of a wild-type (canonical) miRNA (miR405a or miR8174), with a target sequence corresponding to its mature miRNA sequence. As a negative control, the same target vector was used with the second vector not expressing any miRNA precursor sequence.
2. A precursor sequence of a silencing-deficient miRNA-like molecule that is not processed as its corresponding canonical miRNA (Dead_miR859 or Dead_mir_1334), found as described above, with a target sequence corresponding to where its mature miRNA would have been located (according to alignment to its corresponding wild-type miRNA, miR405a or miR8174, respectively). As a negative control, the same target vector was used with the second vector not expressing any miRNA precursor sequence.
3. A precursor sequence of a reactivated (originally silencing-deficient) miRNA-like molecule (Dead_miR859), with a target sequence corresponding to its mature miRNA (the same one as in (2)). As a negative control, the same target vector was used with the second vector not expressing any miRNA precursor sequence.
4. A precursor sequence of a reactivated and redirected silencing-deficient miRNA-like molecule (Dead_miR859 or Dead_mir_1334), which has been reactivated and redirected to silence a target sequence from the AtPDS3 gene, with a target sequence from the AtPDS3 gene. As a negative control, the same target vector was used with the second vector expressing the reactivated silencing-deficient miRNA-like molecule (Dead_miR859 or Dead_mir_1334), which is not targeted against AtPDS3. The sequences of the GEiGS donor oligonucleotides and sgRNAs which can be used in cells in order to perform the redirection of genes encoding Dead_miR859 or Dead_mir_1334 towards AtPDS3, using the GEiGS gene-editing method, are presented in Tables 1A and 1B above.
5. Mock—Non-transfected cells.

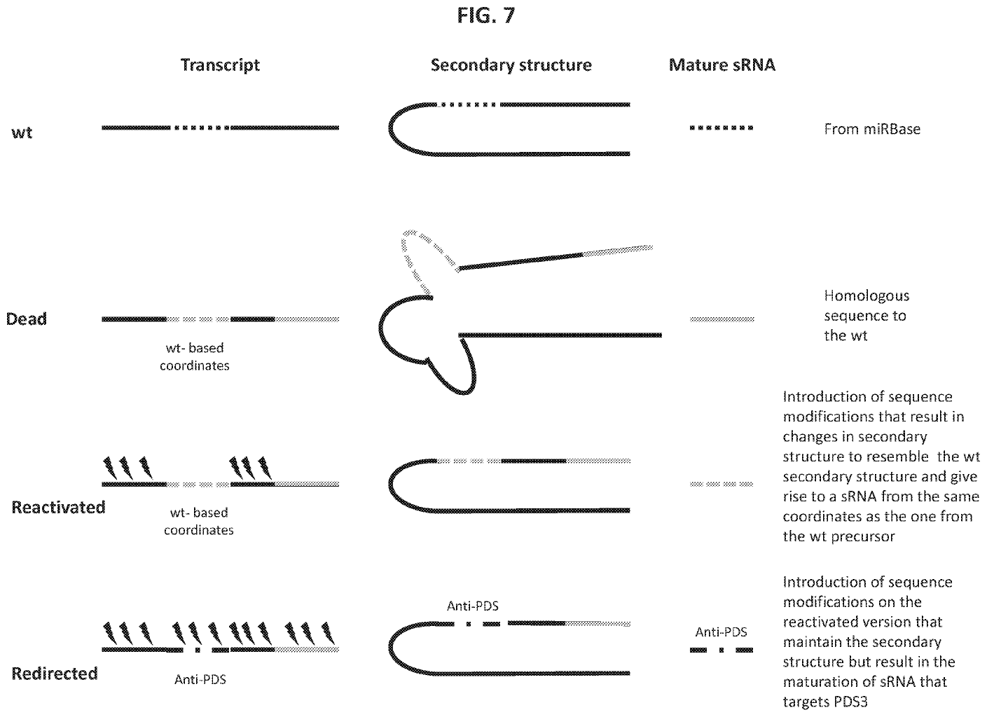

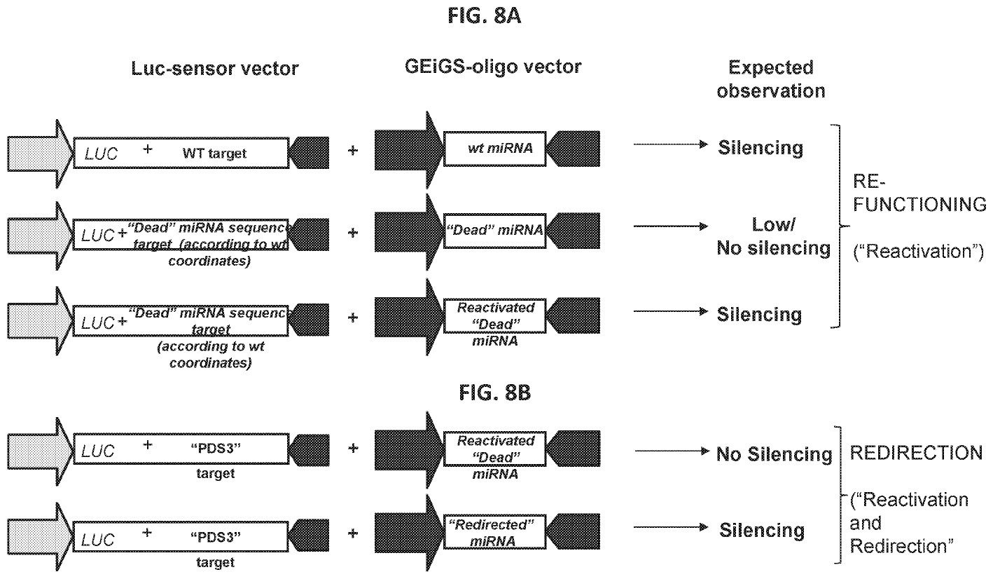

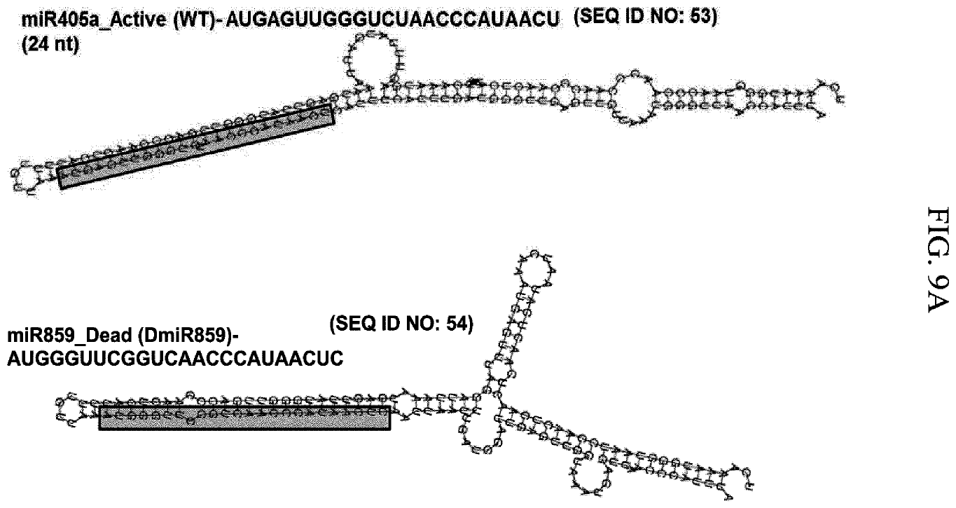

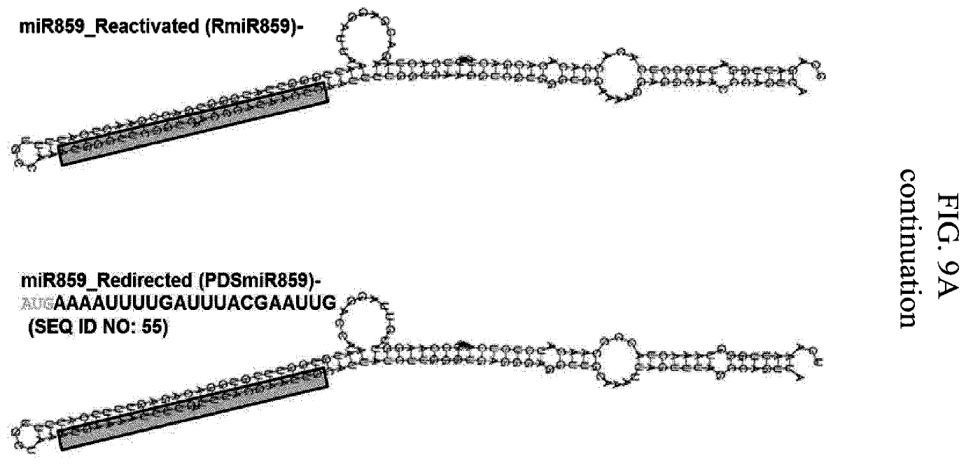

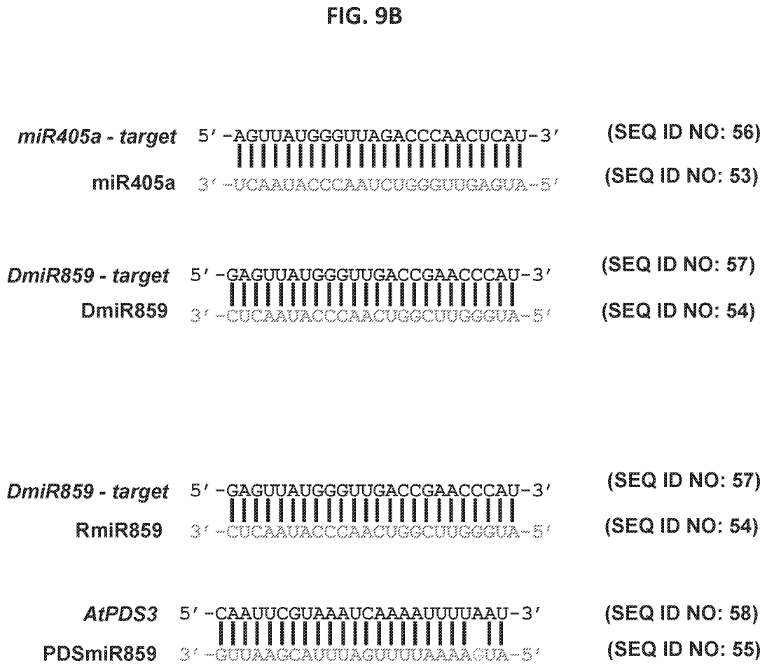

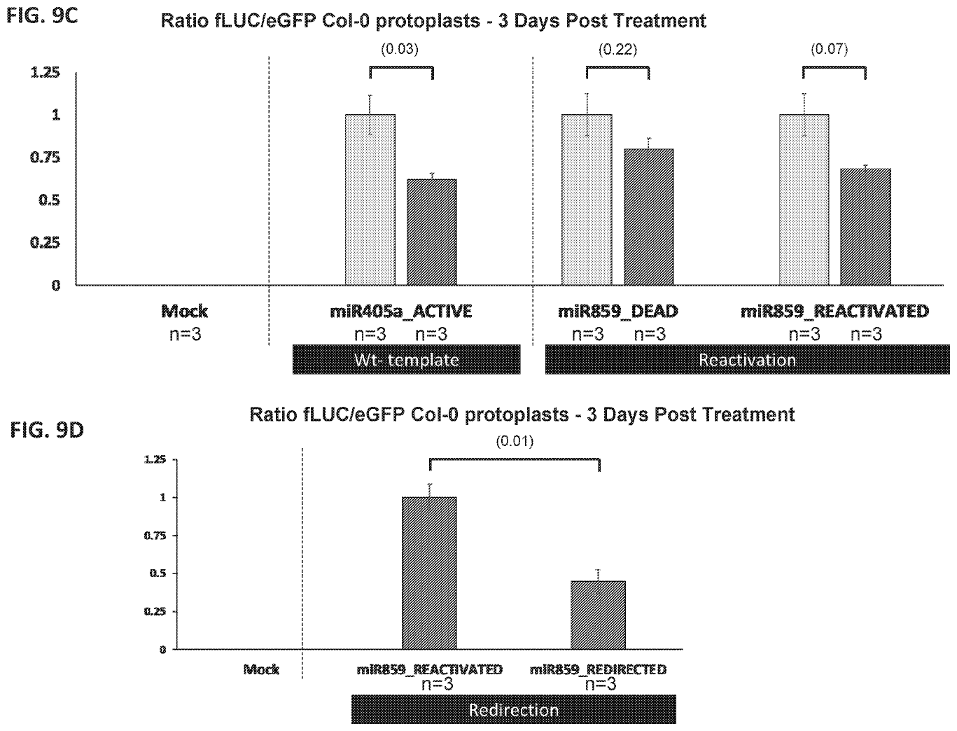

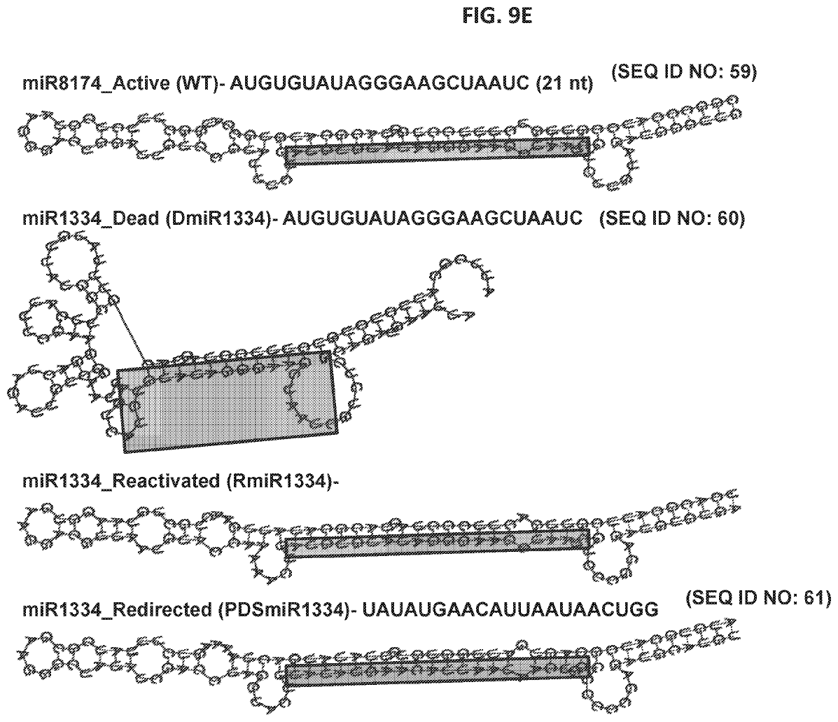

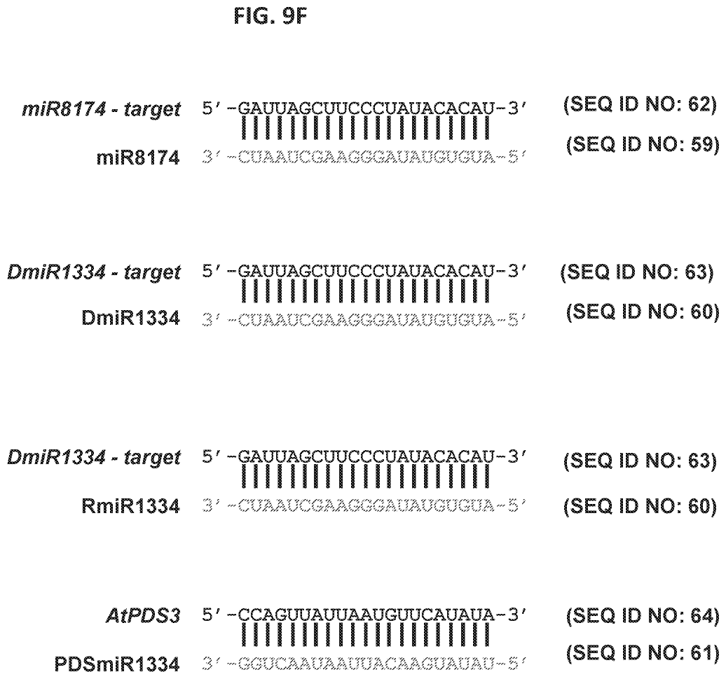

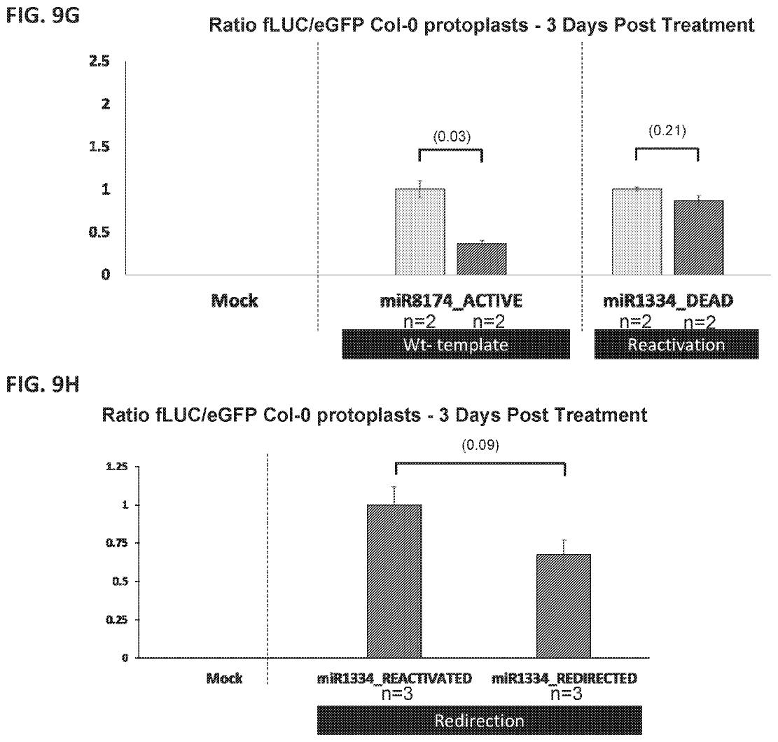

The above combinations, including the expected silencing results (as confirmed by the below results), are summarized in FIGS. 8A-B. The rationale for the changes of the miRNA-like transcripts from dead to reactivated or reactivated and redirected, as used in this example, is depicted schematically in FIG. 7. The predicted secondary structures of the tested miRNAs/miRNA-like molecules, as described above, are presented in FIGS. 9A-B and 9E-F.

The experimental procedures and results obtained using the above-described system are provided below:

### Protoplast Microscopy—Results

A good signal was detected for fluorescent proteins (FP and FP2) for all the transfected treatments, which indicated that a significant fraction of the protoplast cell population was successfully co-transfected with the Luc-sensor vector and the GEiGS-Oligo vector. No fluorescent protein signal (FP and FP2) was detected for the negative controls (Mock).

### Re-Functioning (Reactivation) of miRNA-Like Molecules—Results

For each treatment, Col-0 protoplasts were co-transfected with a Luc-sensor vector and a GEiGS-Oligo vector as described above and in FIGS. 8A-B.

Significant reduction in LUC/FP ratios was observed for wild-type miRNAs miR405a (FIG. 9C) and miR8174 (FIG. 9G), when comparing ratios in treatments with or without the precursors (dark grey vs light grey bars, respectively). Values were normalised to the control treatment in each assay so silencing is measured compared to control. According to these results, the potency of miR405a and miR8174 to silence their target sequences was 38% and 64%, respectively.

No significant reduction in LUC/FP ratios was observed for “Dead” miRNA-like precursors miR859 (FIG. 9C) and miR1334 (FIG. 9G), when comparing ratios for treatments with and without the precursors. This was as expected, as these miRNA-like precursors were not predicted to be processed to sRNAs having silencing activity.

Statistically significant reduction in the LUC/FP ratio was observed for reactivated miR859 (FIG. 9C), for treatments with and without the precursor. The silencing potency for reactivated miR859 was 32%.

### Redirection of Reactivated miRNAs—Results

For each treatment, Col-0 protoplasts were co-transfected with a Luc-sensor vector and a GEiGS-Oligo vector as described above and in FIGS. 8A-B.

When silencing of the AtPDS3 sequence was tested, a significant reduction in LUC/FP ratios was observed when comparing ratios for the miR859 and miR1334 reactivated and redirected against AtPDS3 versus the ratios for miR859 and miR1334 which were only reactivated (FIGS. 9D and 9H). The anti-PDS silencing potency for the reactivated and redirected miR859 and miR1334 was 55% and 33%, respectively. Length of expected sRNAs for miR859 and miR1334 was 24 nt and 21 nt, respectively. The observed silencing effect meant that the redirected oligos were properly processed and the mature sRNAs were able to target their respective new target sequence in the PDS3 gene.

### Example 3

### Functionality of the Reactivated Silencing RNA in Human Cells

To verify that a reactivated non-coding RNA is functional in humans, its biogenesis and activity is tested in a transient system, through the use of pmirGLO Dual-Luciferase miRNA Target Expression Vector kit (Promega, USA). The target sequence is introduced in the MCS downstream the fLUC sequence, according to the manufacturer's instructions. The tested GEiGS-oligo is cloned using the T-REx system (Thermo Fisher, USA) for transient over-expression. Human cell lines are transfected using the Expi293 Expression System (Thermo Fisher, USA). 24-72 hours after plasmid delivery, half of the cells are analyzed for their luciferase activity, and the other half is subjected to small RNA sequencing analysis. Dual luciferase assay is carried out using Dual-Glo® Luciferase Assay System (Promega, USA) according to the manufacturer's instructions. Total RNA is extracted with MirVana™ miRNA isolation kit (Thermo Fisher, USA), according to manufacturer's instructions. Small RNA sequencing analysis is carried out for the identification of the desired mature small RNA in these samples. Reactivated non-coding RNA that is functional down-regulates the LUC gene compared to control constructs that express dysfunctional non-coding RNA or reactivated non-coding RNA that is processed into non-LUC-specific siRNAs.

### Example 4

### Immunity to HIV-1 by Reactivated Silencing RNA in Human Cells

HIV-1 susceptible human cell lines [Reil, H. et al., Virology (1994) 205(1): 371-375] are transfected using the Expi293 Expression System (Thermo Fisher, USA) with GEiGS constructs, according to manufacturer's instructions. Single colonies are isolated and genotyped to identify successful GEiGS events and further maintained. Western blot analysis, for the quantification of the viral proteins p24 and gp120, as well as analysis of their transcription levels by qRT-PCR are used to monitor viral replication. Integrated HIV-1 copy number is assessed by southern blot.

### Example 5

### Functionality of the Reactivated Silencing RNA in C. elegans

To verify that a reactivated non-coding RNA is functional in C. elegans, its biogenesis and activity is tested in a stable gene marker system, through the use of a ubiquitously-expressed GFP marker. Nematodes with reactivated non-coding RNA are generated to target the GFP transgene sequence. Edited worms are tested for the GFP expression and intensity. Nematodes with reactivated non-coding RNA that is functional down-regulates the GFP gene expression compared to control animals that express dysfunctional non-coding RNA or reactivated non-coding RNA that is processed into non-GFP-specific siRNAs. GFP expression is assessed by microscopy analysis, qRT-PCR, RNA-seq and small RNA-seq.

### Example 6

### Knock-Down of Endogenous Gene Via GEiGS System in C. elegans

Changes in the C. elegans genome are designed, to generate non-coding RNAs to target the endogenous UNC-22 gene. These sequences, together with extended homologous arms in the context of the genomic loci, are generated, and named DONOR. Guide RNAs are introduced in the CRISPR/CAS9 vector system to generate a DNA cleavage in the desired loci. These are co-introduced to the plants with the DONOR vectors via gene bombardment protocol, to introduce desired modifications through Homologous DNA Repair (HDR). C. elegans population is transformed with these two sequences to generate a population of C. elegans that harbors the required change in their genome. Nematodes are analyzed on NGM plates under a dissecting microscopy 24-72 hours post injection. “Twitching” phenotype is recorded as an evidence for knockdown of UNC-22. In addition, these nematodes are collected for analysis of UNC-22 expression levels by qRT-PCR, RNA-seq and small RNA analysis.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

What is claimed is:

1. A method of generating an RNA molecule having a silencing activity in a cell, the method comprising:
(a) identifying nucleic acid sequences encoding RNA molecules exhibiting a predetermined sequence homology range, not including complete identity, with respect to nucleic acid sequences encoding RNA molecules engaged with RNA-induced silencing complex (RISC);
(b) determining transcription of said nucleic acid sequences encoding said RNA molecules so as to select transcribable nucleic acid sequences encoding said RNA molecules exhibiting said predetermined sequence homology range;
(c) determining processability into small RNAs of transcripts of said transcribable nucleic acid sequences encoding said RNA molecules exhibiting said predetermined sequence homology range so as to select transcribable nucleic acid sequences encoding said RNA molecules exhibiting said predetermined sequence homology range, wherein said RNA molecules are aberrantly processed;
(d) modifying a nucleic acid sequence of said transcribable nucleic acid sequences encoding said aberrantly processed RNA molecules exhibiting said predetermined sequence homology range so as to impart processability into small RNAs that are engaged with RISC and are complementary to a first target RNA,
thereby generating the RNA molecule having the silencing activity in the cell.

2. The method of claim 1, wherein said RNA molecules of step (a) encoded by the identified nucleic acid sequences exhibit a predetermined sequence homology range, not including complete identity, with respect to RNA molecules that are engaged with—and/or that are processed into molecules engaged with RISC.

3. The method of claim 1 or 2, wherein imparting processability in step (d) comprises imparting canonical processing relative to an RNA molecule encoded by a nucleic acid sequence of said nucleic acid sequences encoding RNA molecules engaged with RNA-induced silencing complex (RISC).

4. The method of any one of claims 1-3, further comprising determining the genomic location of said nucleic acid sequences encoding said RNA molecules exhibiting said predetermined sequence homology range of step (a).

5. The method of claim 4, wherein said genomic location is in a non-coding gene, optionally within an intron of a non-coding gene.

6. The method of claim 4, wherein said genomic location is in a coding gene, optionally within an exon of coding gene, optionally within an exon encoding an untranslated region (UTR) of a coding gene, or optionally within an intron of a coding gene.

7. The method of any one of claims 1-6, wherein step (b) and/or (c) are affected by alignment of small RNA expression data to a genome of said cell and determining the amount of reads that map to each genomic location.

8. The method of claim 7, wherein said alignment of said small RNAs is alignment to a predetermined location in said genome of said cell with no mismatches.

9. The method of any one of claims 1-8, wherein said modifying said nucleic acid sequence of said transcribable nucleic acid sequences imparts a structure of said aberrantly processed RNA molecules, which results in processing of said RNA molecules into small RNAs that are engaged with RISC.

10. The method of any one of claims 1-9, wherein said modifying said nucleic acid sequence of said transcribable nucleic acid sequences encoding said aberrantly processed RNA molecules exhibiting said predetermined sequence homology range is effected at nucleic acids other than those corresponding to the binding site to said first target RNA.

11. The method of any one of claims 1-10, wherein said processability is effected by cellular nucleases selected from the group consisting of Dicer, Argonaute, tRNA cleavage enzymes, and Piwi-interacting RNA (piRNA) related proteins.

12. The method of any one of claims 1-11, wherein modifying in step (d) comprises introducing into the cell a DNA editing agent which reactivates silencing activity in said aberrantly processed RNA molecule towards said first target RNA, thereby generating an RNA molecule having a silencing activity in the cell.

13. The method of any one of claims 1-12, further comprising modifying the specificity of said RNA molecule having the silencing activity in the cell, wherein said DNA editing agent redirects a silencing specificity of said RNA molecule towards a target RNA of interest, said target RNA of interest being distinct from said first target RNA, thereby modifying said specificity of said RNA molecule having said silencing activity in said cell.

14. The method of any one of claims 1-13, wherein the identified nucleic acid sequences encoding RNA molecules of step (a) are homologous to genes encoding silencing RNA molecules whose silencing activity and/or processing into small silencing RNA is dependent on their secondary structure.

15. The method of claim 14, wherein a silencing RNA molecule whose silencing activity and/or processing into small silencing RNA is dependent on secondary structure is selected from the group consisting of: microRNA (miRNA), short-hairpin RNA (shRNA), small nuclear RNA (snRNA or U-RNA), small nucleolar RNA (snoRNA), Small Cajal body RNA (scaRNA), transfer RNA (tRNA), ribosomal RNA (rRNA), repeat-derived RNA, autonomous and non-autonomous transposable and retro-transposable element-derived RNA, autonomous and non-autonomous transposable and retro-transposable element RNA and long non-coding RNA (lncRNA).

16. A genetically modified cell comprising a genome comprising a polynucleotide sequence encoding an RNA molecule having a nucleic acid sequence alteration which results in processing of said RNA molecules into small RNAs that are engaged with RISC, said processing of said RNA molecules being absent from a wild type cell of the same origin devoid of said nucleic acid sequence alteration.

17. The genetically modified plant of claim 16, wherein processing is canonical processing.

18. The genetically modified cell of claim 16 or 17, wherein said RNA molecule has a silencing activity.

19. The method of any one of claims 1-13, or genetically modified cell of any one of claims 16-18, wherein said RNA molecule is selected from the group consisting of a microRNA (miRNA), a small interfering RNA (siRNA), a short hairpin RNA (shRNA), a Piwi-interacting RNA (piRNA), phased small interfering RNA (phasiRNA), trans-acting siRNA (tasiRNA), a transfer RNA fragment (tRF), a small nuclear RNA (snRNA), transposable and/or retro-transpossable derived RNA, autonomous and non-autonomous transposable and/or retro-transpossable RNA.

20. The method of any one of claims 1-15 or 19, wherein said method further comprises introducing into the cell donor oligonucleotides.

21. The method of any one of claims 12-15, 19 or 20, wherein said DNA editing agent comprises at least one sgRNA.

22. The method of any one of claims 12-15, 19-20 or 21, wherein said DNA editing agent does not comprise an endonuclease.

23. The method of any one of claims 12-15, 19-20 or 21, wherein said DNA editing agent comprises an endonuclease.

24. The method of any one of claims 12-15 or 19-23, wherein said DNA editing agent is of a DNA editing system selected from the group consisting of a meganuclease, a zinc finger nucleases (ZFN), a transcription-activator like effector nuclease (TALEN), CRISPR-endonuclease, dCRISPR-endonuclease, and a homing endonuclease.

25. The method of any one of claims 23 or 24, wherein said endonuclease comprises Cas9.

26. The method of any one of claims 12-15 or 19-25, wherein said DNA editing agent is applied to the cell as DNA, RNA or RNP.

27. The method of any one of claims 13-15 or 19-26, wherein said target RNA of interest is endogenous or exogenous to said cell.

28. The method of any one of claims 13-15 or 19-27, wherein said specificity of said RNA molecule is determined phenotypically by determination of at least one phenotype selected from the group consisting of a cell size, a growth rate/inhibition, a cell shape, a cell membrane integrity, a tumor size, a tumor shape, a pigmentation of an organism, a size of an organism, a crop yield, metabolic profile, a fruit trait, a biotic stress resistance, an abiotic stress resistance, an infection parameter, and an inflammation parameter.

29. The method of any one of claims 13-15 or 19-28, or genetically modified cell of any one of claims 16-18 or 19 wherein said cell is a eukaryotic cell.

30. The method or genetically modified cell of claim 29, wherein said eukaryotic cell is obtained from a eukaryotic organism selected from the group consisting of a plant, a mammal, an invertebrate, an insect, a nematode, a bird, a reptile, a fish, a crustacean, a fungi and an algae.

31. The method or genetically modified cell of claim 29, wherein said eukaryotic cell is a plant cell.

32. The method or genetically modified cell of claim 31, wherein said plant cell is a protoplast.

33. A plant cell generated according to the method of any one of claims 1-15 or 19-32.

34. A plant comprising the plant cell of claim 33.

35. The plant of claim 34, wherein said plant is non-transgenic.

36. A method of producing a plant with reduced expression of a target gene, the method comprising:
(a) breeding the plant of claim 34 or 35; and
(b) selecting for progeny plants that have reduced expression of said target RNA of interest, or progeny that comprise a silencing specificity in said RNA molecule towards said target RNA of interest, and which do not comprise said DNA editing agent,
thereby producing said plant with reduced expression of a target gene.

37. A method of producing a plant comprising an RNA molecule having a silencing activity towards a target RNA of interest, the method comprising:
(a) breeding the plant of claim 34 or 35; and
(b) selecting for progeny plants that comprise said RNA molecule having said silencing activity towards said target RNA of interest, or progeny that comprise a silencing specificity in said RNA molecule towards said target RNA of interest, and which do not comprise said DNA editing agent,
thereby producing the plant comprising the RNA molecule having the silencing activity towards the target RNA of interest.

38. A method producing a plant or plant cell of claim 34 or 35 comprising growing the plant or plant cell under conditions which allow propagation.

39. The method of claim 36 or 37, wherein said breeding comprises crossing or selfing.

40. A seed of the plant of any one of claims 34 or 35, or of the plant produced by any one of claims 36-39.

41. The method or genetically modified cell of claim 29, wherein said eukaryotic cell is a human cell.

42. The method or genetically modified cell of claim 41, wherein said nucleic acid sequences encoding RNA molecules are selected from the group consisting of the nucleic acid sequences as set forth in any of SEQ ID NOs. 352 to 392.

43. The method or genetically modified cell of claim 41 or 42, wherein said eukaryotic cell is a totipotent stem cell.

44. A method of treating a disease in a subject in need thereof, the method comprising generating an RNA molecule having a silencing activity and/or specificity according to the method of any one of claims 1-15, 19-32 or 41-43, wherein said RNA molecule comprises a silencing activity towards a transcript of a gene associated with an onset or progression of the disease, thereby treating the subject.

45. A method of introducing silencing activity to a first RNA molecule in a cell, the method comprising:
(a) selecting a first nucleic acid sequence within said cell, wherein:
i. said first nucleic acid sequence is transcribed into said first RNA molecule within the cell;
ii. the sequence of said first RNA molecule has a partial homology to the sequence of a second RNA molecule, excluding sequence identity; wherein said second RNA molecule is processable to a third RNA molecule having a silencing activity; and wherein said second RNA molecule is encoded by a second nucleic acid sequence in said cell; and
iii. said first RNA molecule is not processable, or is processable differently than the second RNA molecule, such that the first RNA molecule is not processed to an RNA molecule having a silencing activity of the same nature as the third RNA molecule;
(b) modifying the first nucleic acid sequence such that it encodes a modified first RNA molecule, said modified first RNA molecule being processable to a fourth RNA in the same way that said second RNA molecule is processable to the third RNA molecule, such that the fourth RNA molecule has a silencing activity of the same nature as the third RNA molecule,
thereby introducing a silencing activity to the first RNA molecule.

46. The method of claim 45, wherein said second RNA molecule is an RNA molecule which has a secondary structure that enables it to be processed into an RNA having a silencing activity, optionally wherein said silencing activity is mediated through engaging RISC.

47. The method of claim 46, wherein said RNA molecule which has a secondary structure that enables it to be processed into an RNA having a silencing activity is selected from the group consisting of: microRNA (miRNA), short-hairpin RNA (shRNA), small nuclear RNA (snRNA or URNA), small nucleolar RNA (snoRNA), Small Cajal body RNA (scaRNA), transfer RNA (tRNA), ribosomal RNA (rRNA), repeat-derived RNA, autonomous and non-autonomous transposable and retro-transposable element-derived RNA, autonomous and non-autonomous transposable and retro-transposable element RNA and long non-coding RNA (lncRNA).

48. The method of claim 46, wherein said first nucleic acid sequence results in a secondary structure which enables the modified first RNA molecule to be processed into the fourth RNA molecule.

49. The method of claim 48, wherein said modifying the first nucleic acid sequence comprises modifying the sequence such that the modified first RNA molecule has essentially the same secondary structure as that of the second RNA molecule, optionally a secondary structure which is at least 95%, 96%, 97%, 98%, 99%, 99.5%, 99.9% or 100% identical to the secondary structure of the second RNA molecule.

50. The method of claim 45, wherein said first nucleic acid molecule is a gene from H. sapiens, wherein the gene is selected from the group consisting of the genes having the sequences set forth in any of SEQ ID NOs. 352 to 392.
